SEQUENCE LISTING (19 sequences)

SEQ ID NO: 1 - DNA, 30 bp, synthetic construct
  source 1..30: mol_type = other DNA
cggcagtcaagcctcttctcgttcctcatc

SEQ ID NO: 2 - DNA, 30 bp, synthetic construct
  source 1..30: mol_type = other DNA
caracattttgctctcaagctggttcaatc

SEQ ID NO: 3 - DNA, 36 bp, synthetic construct
  source 1..36: mol_type = other DNA
ttaataacgctactaatgttgttattaaagtctgtg

SEQ ID NO: 4 - DNA, 33 bp, synthetic construct
  source 1..33: mol_type = other DNA
taagaaaaggctgagagacatattcaaaagtgc

SEQ ID NO: 5 - DNA, 19 bp, synthetic construct
  source 1..19: mol_type = other DNA
aggcggcagtcaagcctct

SEQ ID NO: 6 - DNA, 23 bp, synthetic construct
  source 1..23: mol_type = other DNA
aagcctcagcagcagatttctta

SEQ ID NO: 7 - DNA, 43 bp, synthetic construct
  source 1..43: mol_type = other DNA
tgccagccattctagcaggagaagtctcatcacgtagtcgcaa

SEQ ID NO: 8 - DNA, 42 bp, synthetic construct
  source 1..42: mol_type = other DNA
ggcggtgatgctgctcttgctttttgttggcctttaccagac

SEQ ID NO: 9 - DNA, 60 bp, synthetic construct
  source 1..60: mol_type = other DNA
tccgcagcttgcaacacgggcggtgatgctgctcttgctttttgttggcctttaccagac

SEQ ID NO: 10 - DNA, 91 bp, synthetic construct
  source 1..91: mol_type = other DNA
ttttttttttccccccccccggcggtgatgctgctcttgctttttgttggcctttaccagacattttgctctcaagctggttcaatctgtc

SEQ ID NO: 11 - DNA, 55 bp, synthetic construct
  source 1..55: mol_type = other DNA
ttttttttttccatctgttccagtcattgcgagcttggcctaatcacggtcatag

SEQ ID NO: 12 - DNA, 30 bp, synthetic construct
  source 1..30: mol_type = other DNA
ctatgaccgtgattaggccaagctcgcaat

SEQ ID NO: 13 - DNA, 54 bp, synthetic construct
  source 1..54: mol_type = other DNA
ttttttttttttgatgttgttgtgcacgacttaaggcgcttgcgccttaagtcg

SEQ ID NO: 14 - DNA, 54 bp, synthetic construct
  source 1..54: mol_type = other DNA
ttttttttttttgatgttgttgcatgcgacttaaggcgcttgcgccttaagtcg

SEQ ID NO: 15 - DNA, 54 bp, synthetic construct
  source 1..54: mol_type = other DNA
ttttttttttttgatgttgttgatgccgacttaaggcgcttgcgccttaagtcg

SEQ ID NO: 16 - DNA, 54 bp, synthetic construct
  source 1..54: mol_type = other DNA
ttttttttttttgatgttgttggcatcgacttaaggcgcttgcgccttaagtcg

SEQ ID NO: 17 - DNA, 55 bp, synthetic construct
  source 1..55: mol_type = other DNA
tttttttttttttttttttttttttctatgaccgtgattaggccaagctcgcaat

SEQ ID NO: 18 - DNA, 55 bp, synthetic construct
  source 1..55: mol_type = other DNA
tttttttttttttttttttttttttattgcgagcttggcctaatcacggtcatag

SEQ ID NO: 19 - DNA, 54 bp, synthetic construct
  source 1..54: mol_type = other DNA
tttttttttttttttttttttttttgcacttaaggcgctttgcgccttaagtgc

(19) United States
(12) Patent Application Publication
(10) Pub. No.: US 2023/0366822 A1
(43) Pub. Date: Nov. 16, 2023

(54) PREPARING A SAMPLE FOR EVANESCENT WAVE IMAGING

(71) Applicant: 454 Corporation, Guilford, CT (US)

(72) Inventors: Jonathan M. Rothberg, Miami Beach, FL (US); Isaac Bean, Colorado Springs, CO (US); William A. Hansen, Clinton, CT (US); Jose Camara, Saratoga, CA (US); Lawrence C. West, Las Vegas, NV (US); Henry Kemble, Paris (FR); Lindsay Schneider, Niantic, CT (US); David Honeybun, Sturminster Newton (GB); Jaime Scott Zahorian, Guilford, CT (US)

(73) Assignee: 454 Corporation, Guilford, CT (US)

(21) Appl. No.: 18/309,769

(22) Filed: Apr. 28, 2023

Related U.S. Application Data
(60) Provisional application No. 63/337,044, filed on Apr. 29, 2022.

(51) Int. Cl. G01N 21/64; G02B 21/00

(52) U.S. Cl. CPC G01N 21/648; G02B 21/0052

(57) ABSTRACT

The disclosure is directed to a method for preparing a sample for evanescent wave imaging, comprising: isothermally amplifying a target nucleic acid present in the sample in a reservoir to produce one or more amplicons, wherein the one or more amplicons are immobilized to a bottom surface of the reservoir; and contacting the one or more amplicons with an aqueous solution comprising sequencing reagents comprising a pool of 3′-unblocked protected nucleotides.

### CROSS-REFERENCE TO RELATED APPLICATIONS

This application claims the benefit under 35 U.S.C. § 119(e) of U.S. Provisional Patent Application No. 63/337,044 filed on Apr. 29, 2022, entitled “METHODS AND DEVICES FOR NUCLEIC ACID SEQUENCING,” the entire contents of which are incorporated by reference herein in their entirety.

### FIELD

The present application is generally directed to methods and devices for nucleic acid sequencing.

### REFERENCE TO AN ELECTRONIC SEQUENCE LISTING

The contents of the electronic sequence listing (F090170000US05-SEQ-RJP.xml; Size: 18,239 bytes; and Date of Creation: Apr. 28, 2023) are herein incorporated by reference in their entirety.

### BACKGROUND

Nucleic acid sequencing technology can be used to detect and characterize pathogens, diagnose diseases, and learn about the genetic background or disease predisposition of a subject. Existing nucleic acid sequencing methods and devices often require bulky specialized equipment, costly reagents, and expert personnel to operate. Accordingly, there is a need for low-cost, easily operated nucleic acid sequencing methods and devices.

### SUMMARY OF THE INVENTION

The disclosure is directed to improved methods and devices for nucleic acid sequencing. The disclosure is directed, in part, to a method of nucleic acid sequencing comprising using evanescent wave imaging to determine the identity of a nucleotide incorporated into an elongating sequencing primer using a substrate polynucleotide as a template. In some embodiments, the incorporated nucleotide is a protected nucleotide that prevents further extension by a polymerase until a condition is met (e.g., the protected nucleotide is exposed to ultraviolet (UV) light), which allows for reversible termination of elongation of the sequencing primer.

Accordingly, in one aspect, the disclosure is directed to a method for preparing a sample for evanescent wave imaging, comprising: isothermally amplifying a target nucleic acid present in the sample in a reservoir to produce one or more amplicons, wherein the one or more amplicons are immobilized to a bottom surface of the reservoir; and contacting the one or more amplicons with an aqueous solution comprising sequencing reagents comprising a pool of 3′-unblocked protected nucleotides.

In some embodiments, the method comprises coupling the reservoir with a component of an evanescent wave imaging apparatus. In some embodiments, coupling positions the reservoir above a detector. In some embodiments, coupling aligns the one or more immobilized amplicons within view of the detector.

In some embodiments, isothermally amplifying a target nucleic acid sequence comprises using RPA, LAMP, RCA, or WildFire to amplify the target nucleic acid sequence.

It should be understood that features described in connection with any embodiment of the present technology may be incorporated or utilized in another embodiment or combination of embodiments of the present technology.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

The patent or application file contains at least one drawing executed in color. Copies of this patent or patent application publication with color drawing(s) will be provided by the Office upon request and payment of the necessary fee.

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way. It should be understood that one or more features shown and/or described for an embodiment of the present disclosure may be used in combination with one or more features shown and/or described for another embodiment of the present disclosure.

FIG. 1A shows a schematic illustration of an exemplary device for nucleic acid sequencing, according to some embodiments.

FIG. 1B depicts selected elements of FIG. 1A to illustrate light propagation within a substrate of the device, according to some embodiments.

FIG. 1C shows a schematic illustration of an exemplary device for nucleic acid sequencing including a surrounding light-blocking structure, according to some embodiments.

FIG. 1D shows a schematic illustration of an exemplary device for nucleic acid sequencing, illustrating how certain components may be removable, according to some embodiments.

FIG. 1E shows a schematic illustration of an exemplary device for nucleic acid sequencing with optional heat sink and optical filter components, according to some embodiments.

FIG. 1F shows a schematic illustration of an exemplary device for nucleic acid sequencing with optional isolation layer and light blocking components, according to some embodiments.

FIG. 1G shows a block diagram of an illustrative system suitable for practicing nucleic acid sequencing techniques, according to some embodiments.

FIG. 2A shows structures of exemplary protected nucleotides comprising a photocleavable terminating moiety comprising a 2-nitrobenzyl group, according to some embodiments.

FIG. 2B shows an exemplary scheme for synthesis of an exemplary protected nucleotide, according to some embodiments.

FIGS. 3A-3B show, according to some embodiments, an exterior view (FIG. 3A) and an interior view (FIG. 3B) of an exemplary nucleic acid sequencing device comprising a reservoir and an evanescent wave imaging apparatus.

FIG. 4A shows an interior view of a portion of an exemplary nucleic acid sequencing device comprising a reservoir and an evanescent wave imaging apparatus comprising a plurality of heat sinks and light sources, an optical imaging system, an internal housing, a processing system, and a fan, according to some embodiments.

FIG. 4B shows an interior view of a portion of an exemplary evanescent wave imaging apparatus comprising a processing system, a fan, and power converters, according to some embodiments.

FIG. 4C shows an interior view of a portion of an exemplary nucleic acid sequencing device comprising a reservoir and an evanescent wave imaging apparatus comprising a plurality of heat sinks and light sources, an optical imaging system, an internal housing, a processing system, and power converters, according to some embodiments.

FIG. 4D shows a top-down view of a portion of an exemplary evanescent wave imaging apparatus comprising reservoir alignment openings, a plurality of heat sinks, a fan, and power converters, according to some embodiments.

FIGS. 5A-5G show, according to some embodiments, individual components of an exemplary evanescent wave imaging apparatus. FIG. 5A shows a top outer housing, FIG. 5B shows a bottom outer housing, FIG. 5C shows an inner housing, FIG. 5D shows an optical imaging system, FIG. 5E shows a heat sink and associated set of light sources, FIG. 5F shows a fan, and FIG. 5G shows a processing system, according to some embodiments.

FIGS. 6A-6D show an exemplary reservoir, according to some embodiments. FIG. 6A shows a first cross-sectional view of the reservoir, FIG. 6B shows a second cross-sectional view of the reservoir, FIG. 6C shows a bottom view of the reservoir, and FIG. 6D shows a bottom side perspective view of the reservoir, according to some embodiments.

FIGS. 7A-7C show an exemplary reservoir, according to some embodiments. FIG. 7A shows a bottom side perspective of the reservoir. FIG. 7B shows a top side perspective of reservoir alignment features of the bottom component opening of the reservoir. FIG. 7C shows a top side perspective of the assembled exemplary reservoir with a cap.

FIG. 8 shows an exemplary workflow for sequencing a target nucleic acid from a sample using methods and devices described herein.

FIG. 9 shows exemplary user interface materials for use in the workflow of FIG. 9.

FIG. 10 shows, according to some embodiments, a schematic illustration of various steps of solid phase RT-RPA amplification.

FIG. 11 shows a schematic illustration of various steps of an initial liquid phase of solid phase LAMP amplification and of a subsequent solid phase of solid phase LAMP amplification, according to some embodiments.

FIG. 12 shows a flow chart for an exemplary method of nucleic acid sequencing, according to some embodiments.

FIG. 13 shows a flow chart for an exemplary method of nucleic acid sequencing, according to some embodiments.

FIG. 14A shows an image of components of an exemplary nucleic acid sequencing device, with select components labeled, as used in the Examples.

FIG. 14B shows, according to some embodiments, a first view of an injection-molded reservoir comprising an injection-molded top component, a thermoplastic elastomer (TPE) overmold, and an injection-molded bottom component.

FIG. 14C shows, according to some embodiments, a second view of the injection-molded reservoir of FIG. 14B.

FIG. 15 shows images captured using evanescent wave imaging of an exemplary substrate and spots on the surface of the substrate.

FIG. 16 shows images captured using evanescent wave imaging showing sequential incorporation of individual protected nucleotides on the surface of an exemplary substrate and photocleavage of the photocleavable moiety of the incorporated nucleotides.

FIG. 17 shows a graph of mean fluorescence intensity over cycle count monitoring incorporation of protected A or G nucleotides into primers on the surface of an exemplary substrate and photocleavage of the photocleavable moiety of the incorporated nucleotides.

FIG. 18 shows a graph of mean fluorescence intensity over cycle count monitoring incorporation of three protected A nucleotides into primers on the surface of an exemplary substrate and photocleavage of the photocleavable moiety of the incorporated nucleotides.

FIG. 19 shows, according to some embodiments, a schematic illustration of various steps of solid phase RCA amplification.

FIG. 20 shows a graph of fluorescence intensity over time monitoring incorporation of a protected nucleotide into primers on the surface of an exemplary substrate and photocleavage of the photocleavable moiety of the incorporated nucleotide.

FIG. 21 shows a graph of fluorescence intensity over time monitoring photocleavage of the photocleavable moiety of the incorporated nucleotide from FIG. 20.

FIG. 22 shows, according to some embodiments, an exemplary chemical structure of MCP4.

FIG. 23 shows, according to some embodiments, an exemplary chemical structure of a bisphosphonate-containing polymer.

FIG. 24 shows, according to some embodiments, an exemplary chemical structure of a bisphosphonate-containing small molecule.

FIG. 25 shows, according to some embodiments, an exemplary chemical structure of a bisphosphonate-containing small molecule.

FIG. 26A shows, according to some embodiments, an image of fluorescently labeled oligonucleotides bound to a sapphire substrate by an alendronate-derived molecule.

FIG. 26B shows, according to some embodiments, a plot of gray value v. distance (pixels) for the fluorescently labeled oligonucleotides of FIG. 26A.

FIG. 26C shows, according to some embodiments, images and plots of the fluorescently labeled oligonucleotides of FIG. 26A after being heated to 75° C. for 5 minutes in 1 mL TE Buffer.

FIG. 26D shows, according to some embodiments, images and plots of the fluorescently labeled oligonucleotides of FIG. 26A after being heated to 75° C. for 5 minutes in 1 mL water.

FIG. 26E shows, according to some embodiments, images and plots of the fluorescently labeled oligonucleotides of FIG. 26A after being heated to 75° C. for 5 minutes in 1 mL 100 mM NaOH.

FIG. 26F shows, according to some embodiments, images and plots of the fluorescently labeled oligonucleotides of FIG. 26A after being heated to 75° C. for 5 minutes in 1 mL 1 M NaOH.

FIG. 26G shows, according to some embodiments, images and plots of the fluorescently labeled oligonucleotides of FIG. 26A after being heated to 75° C. for 5 minutes in 1 mL 1 M HCl.

FIG. 27A shows, according to some embodiments, an exemplary mask comprising 4, 8, 16, and 32 μm diameter openings on a 50 μm pitch.

FIG. 27B shows, according to some embodiments, a zoomed-in view of the upper left corner of the mask of FIG. 27A.

FIG. 27C shows, according to some embodiments, optical profilometer measurements for wells prepared using the mask of FIG. 27A.

FIG. 27D shows, according to some embodiments, optical images of wells prepared using the mask of FIG. 27A.

FIG. 28A shows, according to some embodiments, an exemplary mask comprising a 12×12 array of 50 μm diameter wells on a 200 μm pitch.

FIG. 28B shows, according to some embodiments, optical profilometer measurements for wells prepared using the mask of FIG. 28A.

FIG. 29A shows, according to some embodiments, an exemplary mask comprising 5 μm diameter openings in a hexagonally packed array with 12 μm spacing between any two adjacent openings.

FIG. 29B shows, according to some embodiments, a zoomed-in view of the upper left corner of the mask of FIG. 29A.

FIG. 29C shows, according to some embodiments, optical profilometer measurements for wells prepared using the mask of FIG. 29A.

FIG. 29D shows, according to some embodiments, optical profilometer measurements for wells prepared with sloped sidewalls using the mask of FIG. 29A.

FIG. 30A shows, according to some embodiments, an exemplary workflow for coating a quartz substrate with a layer of MCP4 and conjugating oligonucleotides to the MCP4 layer.

FIG. 30B shows, according to some embodiments, images from five sequencing cycles (e.g., cycles of incorporating a protected nucleotide) performed using substrates prepared according to the workflow of FIG. 30A.

FIG. 30C shows, according to some embodiments, purity histograms from five sequencing cycles (e.g., cycles of incorporating a protected nucleotide) performed using substrates prepared according to the workflow of FIG. 30A.

FIG. 31A shows, according to some embodiments, an exemplary chemical structure of a protected dATP.

FIG. 31B shows, according to some embodiments, an exemplary chemical structure of a protected dGTP.

FIG. 31C shows, according to some embodiments, an exemplary chemical structure of a protected dCTP.

FIG. 31D shows, according to some embodiments, an exemplary chemical structure of a protected dTTP.

FIG. 32 is, according to some embodiments, an exemplary schematic showing conversion of dGTP to HOMedGTP via UV exposure.

FIG. 33 shows, according to some embodiments, exemplary analysis output showing a first peak (left) corresponding to Alexa Fluor 532 labeled primer and a second peak (right) corresponding to HOMedGTP incorporation product.

FIG. 34 shows, according to some embodiments, exemplary HOMedGTP quantities (fmol) for bare sapphire reservoirs, sapphire reservoirs comprising a 2 μm-thick CYTOP® layer, bare quartz reservoirs, and quartz reservoirs comprising a 2 μm-thick CYTOP® layer.

FIG. 35A shows, according to some embodiments, an exemplary nucleotide sequence of the Dark nucleotide In Situ Cleanup System (DISCS) reagent.

FIG. 35B shows, according to some embodiments, an exemplary nucleotide sequence of the DISCS reagent.

FIG. 36 shows, according to some embodiments, exemplary plots demonstrating removal by DISCS of HOMedGTP produced in a bare quartz reservoir by a 3 second UV exposure.

FIG. 37 shows, according to some embodiments, a schematic illustration of an exemplary sequencing cycle.

FIG. 38A shows, according to some embodiments, images from five sequencing cycles.

FIG. 38B shows, according to some embodiments, purity histograms from five sequencing cycles.

### DETAILED DESCRIPTION

The disclosure is directed, in part, to the discovery that an evanescent wave produced via total internal reflection of light within a substrate can be used to determine the identity of a nucleotide (e.g., a protected nucleotide) incorporated into an elongating sequencing primer (e.g., using a substrate polynucleotide immobilized to a substrate as a template). The disclosure is further directed, in part, to the discovery that an evanescent wave produced via total internal reflection can be used to control reversible termination of elongation of a sequencing primer by a polymerase (e.g., to reverse the elongation terminating effects of incorporation of a protected nucleotide into the sequencing primer). The disclosure is directed, in part, to the combination and application of these two discoveries to methods and devices. Methods and devices described herein may enable the use of evanescent wave imaging to rapidly sequence a nucleic acid using affordable quantities of reagents and economical equipment. In some embodiments, methods and devices disclosed herein may require only layperson levels of expertise in molecular biology laboratory techniques or sequencing technology. In some embodiments, methods of sequencing and end-user operation of devices of the disclosure do not require fluid manipulation and/or do not require wash steps, which may be costly, cumbersome, and/or impractical.

### Device Overview

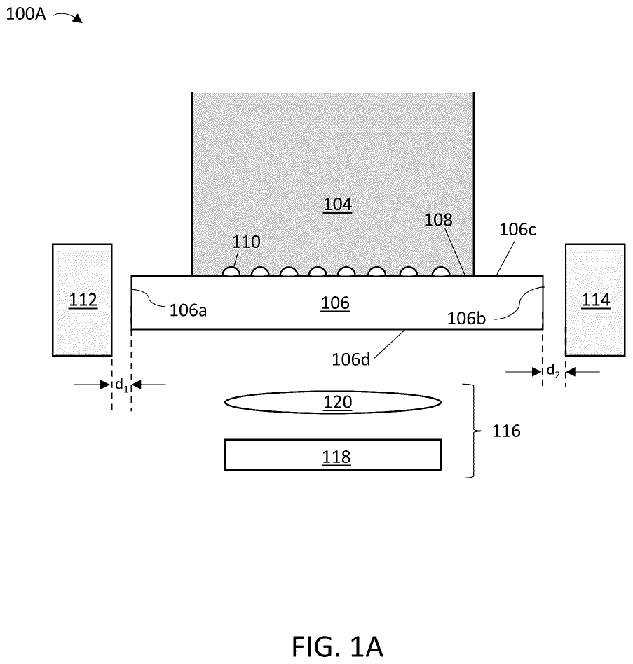

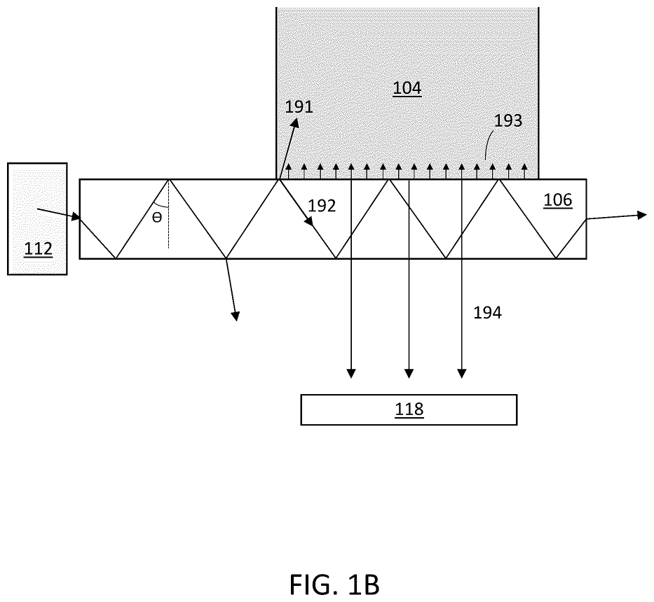

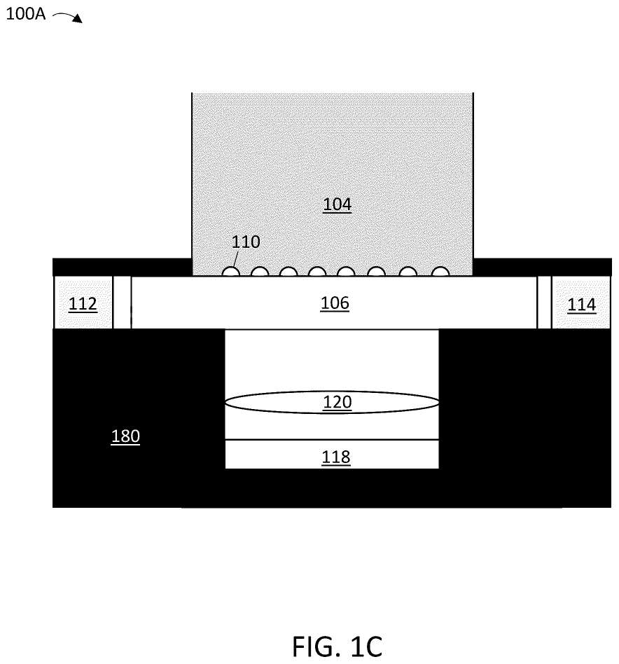

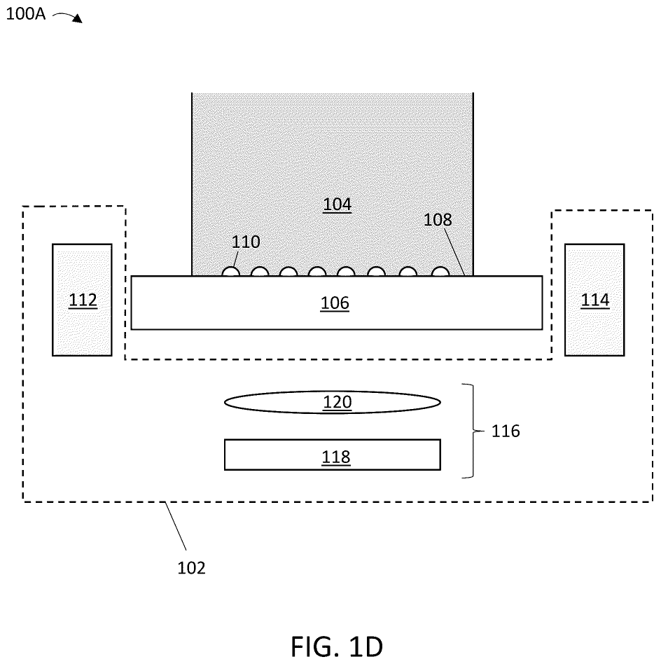

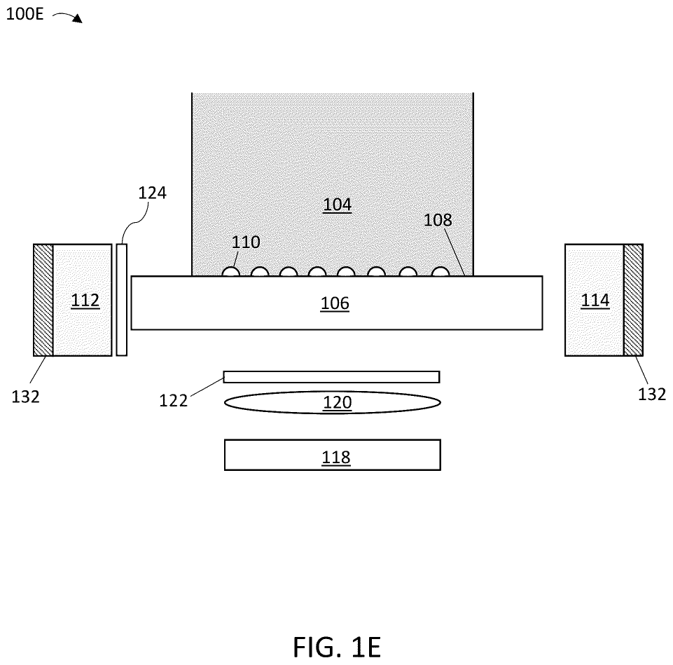

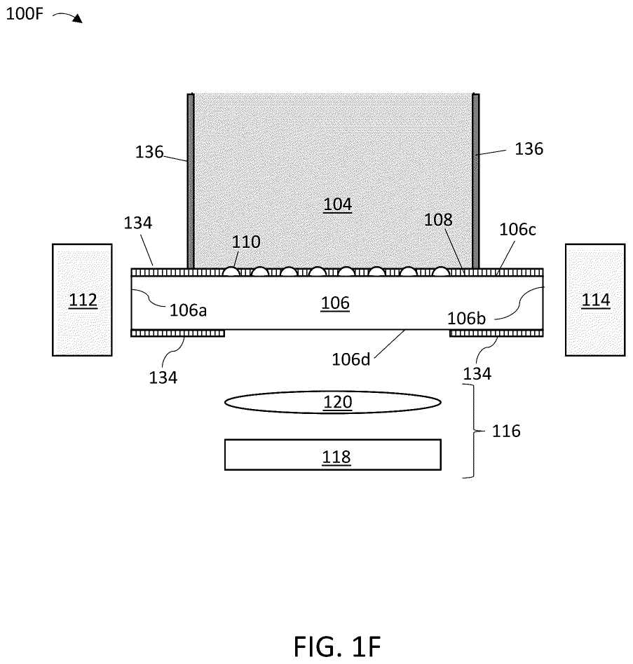

The disclosure is directed, in part, to devices for nucleic acid sequencing. FIGS. 1A-1G show views of exemplary devices, according to various embodiments, and are not intended to be limiting in any respect.

FIG. 1A shows a schematic illustration of an exemplary device 100A for nucleic acid sequencing, according to some embodiments. In the example of FIG. 1A, device 100A comprises reservoir 104, substrate 106, and light sources 112 and 114. One or more substrate polynucleotides 110 are attached to bottom surface 108 of reservoir 104. In operation, substrate 106 may be optically transparent and act as a waveguide for light emitted by the light sources 112 and/or 114. The light entering substrate 106 may be transmitted or reflected when it is incident on a surface of the substrate, depending on the incident angle of the light to the surface. For angles greater than a critical angle to the surface normal, the light may be completely reflected back into the substrate (total internal reflection). When total internal reflection (TIR) occurs, evanescent waves may be produced outside of the substrate, extending a short distance away from the substrate.

As a result of this process, evanescent waves may be produced within the reservoir 104 in a region near to surface 106c of substrate 106. As described further below, the evanescent waves may produce a variety of effects, including acting as an excitation light, causing cleavage of a photocleavable terminating moiety, and/or any other desired effect. Light produced from within reservoir 104 as a result of such effects may travel through substrate 106 and be focused by lens 120 onto image sensor 118. The device 100A may therefore perform analysis of (e.g., identification of) one or more nucleotides within the reservoir by efficiently directing light from light sources 112 and/or 114 into substrate 106, thereby producing evanescent waves within reservoir 104, which cause through one or more physical processes the production of additional light, at least some of which is received by image sensor 118.

To further illustrate the use of substrate 106 as a waveguide, FIG. 1B depicts selected elements of FIG. 1A. In particular, substrate 106 is shown with larger dimensions than depicted in FIG. 1A, and the light source 114, polynucleotides 110, and lens 120 are omitted, for purposes of illustration. In the example of FIG. 1B, light from light source 112 may enter substrate 106 on the left side (i.e., through surface 106a) as shown. When a light ray is incident upon any of the six surfaces of the substrate, the light may be transmitted or reflected at the boundary, depending on the incident angle θ as shown in FIG. 1B, and depending on the relative indexes of refraction of the substrate and the material adjacent to the surface of the substrate. The incident angle is measured relative to the normal to the surface, and light rays with comparatively low incident angles may be transmitted out of the substrate (e.g., producing light ray 191). When light rays have an incident angle above a threshold, and the substrate has a higher refractive index than the material adjacent to the surface of the substrate, the light may be reflected back into the bulk of the substrate (e.g., producing light ray 192). This behavior is a result of Snell's law, wherein an incident angle above a critical angle

$${θ}_{crit} = arc sin ( \frac{{n}_{2}}{{n}_{1}} ) ,$$

with n₁ being the refractive index of substrate 106 and n₂ being the refractive index of the material next to substrate 106, may result in total internal reflection of the light, rather than transmission into another medium.

When total internal reflection occurs, a standing electromagnetic field 193 (which may also be referred to herein as an evanescent wave, or evanescent light) may be produced on the side of the boundary with the lower refractive index. Evanescent waves 193 have the same wavelength as the incident light (λ), and have an intensity

$$I = {I}_{0} {e}^{- \frac{z}{d}} ,$$

where z is the distance from the interface, and d is given by:

$$d = \frac{λ}{4 π {n}_{2}} {( \frac{{sin}^{2} θ}{{sin}^{2} {θ}_{crit}} - 1 )}^{- \frac{1}{2}}$$

This decay generally results in a 1/e distance (a “decay length”) that is a fraction of the wavelength of the incident light.

As one non-limiting example, light within a quartz substrate (n₁=1.55) adjacent to water (n₂=1.33) has a critical angle of around 59°. That is, light within the quartz that is incident upon the quartz-water boundary at an angle below 590 will be transmitted (and refracted) into the water (like ray 191), whereas light incident at an angle at or above 590 will be reflected back into the quartz (like ray 192). Ultraviolet light at λ=365 nm and incident at θ=700 has, for instance, a value of d of about 48 nm, leading to a rapid decrease in the evanescent light's intensity over the first few hundred nanometers in the water.

In some embodiments, the device 100A may be configured such that the evanescent wave extends a distance that is greater than or equal to 10 nm, 20 nm, 50 nm, 100 nm, 150 nm, 200 nm, 250 nm, or 300 nm from the bottom surface of the reservoir (or a top surface of the substrate). In some embodiments, the device 100A may be configured such that the evanescent wave extends a distance that is less than or equal to 300 nm, 250 nm, 200 nm, 150 nm, 100 nm, 50 nm, 20 nm, or 10 nm from the bottom surface of the reservoir (or a top surface of the substrate). Any suitable combinations of the above-referenced ranges are also possible (e.g., a distance greater than or equal to 50 nm and less than or equal to 200 nm). In certain embodiments, the device 100A may be configured such that the evanescent wave extends a distance in a range from 10 nm to 50 nm, 10 nm to 100 nm, 10 nm to 150 nm, 10 nm to 200 nm, 10 nm to 250 nm, 10 nm to 300 nm, 50 nm to 100 nm, 50 nm to 150 nm, 50 nm to 200 nm, 50 nm to 250 nm, 50 nm to 300 nm, 100 nm to 200 nm, 100 nm to 250 nm, 100 nm to 300 nm, or 200 nm to 300 nm.

As described further below, the evanescent waves 193 may interact with one or more elements in reservoir 104, thereby producing light 194, which may be referred to herein as “emission light.” At least some of the emission light 194 may pass through substrate 106 and onto an image sensor 118, which may measure the intensity of emission light incident on one or more pixels of the image sensor.

In some embodiments, emission light resulting from excitation of detectable moieties of protected nucleotides incorporated into sequencing primers annealed to substrate polynucleotides 110 may be transmitted through surfaces 106c and surface 106d to image sensor 118.

In some embodiments, substrate 106 may have a comparatively high refractive index, and the aqueous solution of reservoir 104 may have a comparatively low refractive index. Without wishing to be bound by a particular theory, the effective range of useful intensity of the evanescent wave may extend only a limited distance beyond the interface between the high index material and the lower index material into the lower index material (e.g., a limited distance beyond the bottom surface of the reservoir), with an energy of the evanescent wave decreasing exponentially with distance from the interface z, as noted above. An advantage of using an evanescent wave to, for example, determine the identity of a nucleotide incorporated into a sequencing primer annealed to a substrate polynucleotide and control the reversible termination of elongation of the sequencing primer, is that the limited distance of the evanescent wave can selectively excite a photoactive moiety (e.g., a detectable moiety, a photocleavable terminating moiety) in a small volume immediately adjacent to a bottom surface of the reservoir (e.g., a volume or reaction region containing immobilized substrate polynucleotides, which may be annealed to sequencing primers including recently incorporated nucleotides). As such, the probability of detecting emitted light from detectable moieties that are not incorporated into a sequencing primer annealed to a substrate polynucleotide 110 immobilized to bottom surface 108 may be relatively low. In addition, the cleaving of free moieties in solution within the reservoir may be minimized.

In some embodiments, light source 114 emits photocleavage light such that the photocleavage light enters substrate 106 through second surface 106b of substrate 106. The photocleavage light may have a peak wavelength in the UV range and/or the visible range of the electromagnetic spectrum. In some embodiments, substrate 106 transmits the photocleavage light from second surface 106b of substrate 106 to reservoir 104, where an evanescent wave at the interface of substrate 106 and the aqueous solution of reservoir 104 imparts energy to (e.g., illuminates) a portion of the aqueous solution of reservoir 104 within a limited distance of bottom surface 108 (i.e., within a limited distance of the interface between substrate 106 and the aqueous solution of reservoir 104). In some cases, the evanescent wave cleaves a photocleavable terminating moiety of a protected nucleotide incorporated into a sequencing primer annealed to substrate polynucleotide 110 immobilized to bottom surface 108 such that the photocleavable terminating moiety is released from the protected nucleotide. In some such cases, a polymerase may resume nucleic acid synthesis and may further incorporate one or more nucleotides into the sequencing primer annealed to substrate polynucleotide 110 immobilized on bottom surface 108. Returning to FIG. 1A, the manner in which light from light sources 112 and/or 114 may produce evanescent waves within reservoir 104, which may cause the production and measurement of emission light from reservoir 104, may now be appreciated. Various physical processes may produce emission light as a result of the evanescent waves being produced within the reservoir, and various examples are described below. In addition, illustrative examples of suitable structures for the substrate polynucleotides are described further below.

According to some embodiments, light source 112 and/or light source 114 may each comprise one or more LEDs. In some cases, an LED within the light source may have a flat emission surface, such as a chip on board LED. LEDs may be uncoated and/or arranged on a raw die, and any number of LEDs may be included in either light source. Flat surface LEDs may be beneficial in that they increase the light efficiency of the device 100A by decreasing the amount of light that is emitted from the LED but does not enter the substrate 106. The distance from the LED to the substrate—d₁ or d₂ for light source 112 and 114, respectively—may accordingly be ideally minimized to improve efficiency. In some embodiments, distances d₁ and d₂ may be small though non-zero to provide good efficiency of light input to the substrate while allowing the substrate to be removed from the device, as described further below. In some embodiments, the distances d₁ and d₂ may be zero. For instance, light sources 112 and/or 114 may be in direct physical contact with a surface of substrate 106, or may in some cases be arranged within a recess of substrate 106.

According to some embodiments, each of light sources 112 and 114 may emit light having a peak wavelength in the visible range (e.g., 400 nm to 700 nm), or may emit light having a peak wavelength in the UV range (e.g., 100 nm to 400 nm). In some cases, each of light sources 112 and 114 may emit at least some light in both the visible and UV ranges. For example, a blue GaN LED may emit light with a peak wavelength of around 430 nm, but may emit at least some UV light below 400 nm (as well as some blue light at close to 500 nm). According to some embodiments, light source 112 may emit light primarily in the visible range (e.g., with a peak wavelength between 450 nm and 600 nm), whereas light source 114 may emit light primarily in the UV range (e.g., with a peak wavelength between 300 nm and 400 nm). In some embodiments, each of light sources 112 and 114 may comprise a blue LED, a UV-A LED, a UV-B LED or a UV-C LED.

According to some embodiments, substrate 106 may comprise quartz (e.g., crystalline quartz or fused quartz), optical glass (e.g., crown glass), fused silica, borosilicate glass, sapphire, or combinations thereof. In some embodiments, substrate 106 comprises single crystalline sapphire and has an orientation such that a surface on which substrate polynucleotides are immobilized is an a-plane surface, a c-plane surface, or an r-plane surface. Since sapphire has a relatively higher refractive index than glass (e.g., at least 1.7), this may permit sapphire substrates to achieve total internal reflection of light with a greater variety of aqueous solutions compared to glass substrates. Stated differently, aqueous solutions having a relatively higher refractive index may be used with sapphire substrates rather than glass substrates in order to achieve total internal reflection due to the higher refractive index of sapphire compared with the refractive index of glass.

According to some embodiments, substrate 106 is substantially planar, e.g., a substantially planar disc or a substantially planar rectangular prism (e.g., a slide). In some embodiments, the substrate has a thickness of greater than or equal to 0.1 mm, 0.2 mm, 0.3 mm, 0.4 mm, 0.5 mm, 0.6 mm, 0.7 mm, 0.8 mm, 0.9 mm, 1.0 mm, or 1.5 mm. In some embodiments, the substrate has a thickness of less than or equal to 1.5 mm, 1.0 mm, 0.9 mm, 0.8 mm, 0.7 mm, 0.6 mm, 0.5 mm, 0.4 mm, 0.3 mm, 0.2 mm, or 0.1 mm. Any suitable combinations of the above-referenced ranges are also possible (e.g., a thickness of greater than or equal to 0.4 mm and less than or equal to 0.6 mm).

According to some embodiments, substrate 106 is a planar disc and light sources 112 and 114 are arranged to direct light into curved sides of the disc. For example, the surfaces 106a and 106b may represent sides of the disc, whereas surfaces 106c and 106d may represent top and bottom faces of the disc, respectively. According to some embodiments, substrate 106 is a rectangular prism and the surfaces 106a and 106b may represent two different sides of the prism, with two additional sides of the prism not being shown in the figure. These additional sides may be arranged adjacent to additional light sources, or may in some cases include a reflective coating to redirect light back into the substrate. Surfaces 106c and 106d may represent top and bottom faces of the rectangular prism, respectively.

According to some embodiments, at least a portion (and, in some cases, substantially all) of top surface 106c of substrate 106 forms a part of the bottom surface of reservoir 104 and is in contact with the aqueous solution of reservoir 104. In some embodiments, at least a portion of top surface 106c of substrate 106 is not in contact with the aqueous solution of reservoir 104. In certain embodiments, bottom surface 106d of substrate 106 faces toward an optical imaging system 116 comprising image sensor 118. In certain embodiments, a reaction region (e.g., a region of substrate 106 where substrate polynucleotides 110 are immobilized) may be aligned, or substantially aligned, with a sensor region (e.g., a region comprising pixels) of image sensor 118 of optical imaging system 116. For example, the reaction region may be arranged directly above image sensor 118.

According to some embodiments, at least one of the one or more surfaces of substrate 106 is polished (e.g., to facilitate coupling with a light source). In certain embodiments, a first surface (e.g., 106a) and a second surface (e.g., 106b) are polished. The second surface may be positioned opposite or adjacent to the first surface. In certain embodiments, at least three surfaces of the substrate are polished. In certain embodiments, at least four surfaces of the substrate are polished. In certain embodiments, at least four outer edges, the top surface, and the bottom surface of the substrate are polished.

According to some embodiments, substrate 106 may have a refractive index higher than the refractive index of a liquid held within reservoir 104, such that light within the substrate may undergo total internal reflection when incident on a surface of the substrate adjacent to the liquid (e.g., surface 106c). Similarly, substrate 106 may have a refractive index higher than the refractive index of one or more materials directly contacting the surface of substrate 106 outside of the reservoir. Although no such materials are shown in the example of FIG. 1A, additional embodiments described below include examples of such materials.

According to some embodiments, substrate 106 has a refractive index of greater than or equal to 1.45, 1.46, 1.47, 1.48, 1.49, 1.50, 1.51, 1.52, 1.53, 1.54, 1.55, 1.56, 1.57, 1.58, 1.59, 1.60, 1.61, 1.62, 1.63, 1.64, 1.65, 1.66, 1.67, 1.68, 1.69, 1.70, 1.71, 1.72, 1.73, 1.74, 1.75, 1.76, 1.77, 1.78, 1.80, 1.82, 1.84, 1.85, or 1.86. According to some embodiments, substrate 106 has a refractive index of less than or equal to 1.86, 1.85, 1.84, 1.83, 1.82, 1.81, 1.80, 1.79, 1.78, 1.77, 1.76, 1.75, 1.74, 1.73, 1.72, 1.71, 1.70, 1.69, 1.68, 1.67, 1.66, 1.65, 1.64, 1.63, 1.62, 1.61, 1.60, 1.59, 1.58, 1.57, 1.56, 1.55, 1.54, 1.53, 1.52, 1.51, 1.50, 1.49, 1.48, 1.47, 1.46, or 1.45. Any suitable combinations of the above-referenced ranges are also possible (e.g., a refractive index of greater than or equal to 1.50 and less than or equal to 1.60; or greater than or equal to 1.60 and less than or equal to 1.80).

According to some embodiments, substrate 106 does not substantially absorb light emitted by light sources 112 and/or 114. Additionally, or alternatively, substrate 106 does not substantially absorb emission light (e.g., produced by detectable moieties of protected nucleotides). In some embodiments, “does not substantially absorb” light means that the substrate transmits at least 70%, 75%, 80%, 85%, 90%, 95%, 99%, or 100% of the relevant light (e.g., at a given wavelength or range of wavelengths). In some embodiments, the substrate does not substantially absorb light having a peak wavelength of at least 280 nm, 300 nm, 320 nm, 350 nm, 365 nm, 367 nm, 380 nm, 400 nm, 450 nm, 500 nm, 550 nm, 600 nm, 650 nm, and/or 700 nm. In some embodiments, the substrate does not substantially absorb light having a peak wavelength in a range of 280-300 nm, 280-350 nm, 280-365 nm, 280-400 nm, 280-450 nm, 280-500 nm, 280-550 nm, 280-600 nm, 280-650 nm, 280-700 nm, 300-365 nm, 300-400 nm, 300-450 nm, 300-500 nm, 300-550 nm, 300-600 nm, 300-650 nm, 300-700 nm, 365-400 nm, 365-450 nm, 365-500 nm, 365-550 nm, 365-600 nm, 365-650 nm, 365-700 nm, 400-450 nm, 400-500 nm, 400-550 nm, 400-600 nm, 400-650 nm, 400-700 nm, 450-500 nm, 450-550 nm, 450-600 nm, 450-650 nm, 450-700 nm, 500-550 nm, 500-600 nm, 500-650 nm, 500-700 nm, 550-600 nm, 550-650 nm, 550-700 nm, and/or 600-700 nm.

In some embodiments, at least a portion of top surface 106c of substrate 106 and/or at least a portion of bottom surface 106d of substrate 106 has a relatively low average surface roughness, such as a root-mean-square (RMS) average surface roughness of less than or equal to 1 nm, 0.9 nm, 0.8 nm, 0.7 nm, 0.6 nm, 0.5 nm, 0.4 nm, 0.3 nm, 0.25 nm, 0.2 nm, 0.1 nm, or 0.05 nm. In some embodiments, the RMS average surface roughness of the at least a portion of a top surface 106c of the substrate and/or at least a portion of the bottom surface 106d of the substrate 106 is greater than or equal to 0.05 nm, 0.1 nm, 0.25 nm, 0.5 nm, or 1 nm. Any suitable combinations of the above-referenced ranges are also possible (e.g., an average surface roughness of greater than or equal to 0.05 nm and less than or equal to 0.5 nm). The RMS surface roughness of the top surface of the substrate may, for example, be measured using atomic force microscopy (AFM) or otherwise.

According to some embodiments, reservoir 104 may be formed from a vessel that is attached (e.g., glued) to substrate 106. In some embodiments, the reservoir may instead be formed by attaching walls to substrate 106.

According to some embodiments, device 100A comprises an intermediary substance positioned between substrate 106 and reservoir 104. The intermediary substance may be, or may comprise, a liquid (e.g., an aqueous solution, an organic solution), a glue, and/or a paste. According to some embodiments, the intermediary substance has a refractive index of greater than or equal to 1.40, 1.37, 1.35, 1.33, 1.30, 1.25, 1.20, 1.15, 1.10, 1.05, or 1.00. According to some embodiments, the intermediary substance has a refractive index of less than or equal to 1.00, 1.05, 1.10, 1.15, 1.20, 1.25, 1.30, 1.33, 1.35, 1.37, or 1.40. Any suitable combinations of the above-referenced ranges are also possible (e.g., a refractive index of greater than or equal to 1.10 and less than or equal to 1.30). In some embodiments, an absolute value of a difference between the refractive index of the intermediary substance and the refractive index of a solution contained in reservoir 104 is less than or equal to 0.5, 0.4, 0.3, 0.2, or 0.1. In some embodiments, the intermediary substance is or comprises an isolation layer.

According to some embodiments, the substrate polynucleotides may be directly arranged on substrate 106 or may be arranged over an additional substrate material. For example, a portion of the surface of the substrate can be activated by one or more surface-activating agents (e.g., such as gold). As a further example, a substrate surface can comprise a plurality of layers of materials and/or functional groups, with different portions of the substrate surface comprising different numbers of layers (e.g., a first portion may have two layers (a first and a second layer) where a second (e.g., adjacent) portion may have one layer (e.g., just the first layer). For instance, an etched gold layer may be formed on the substrate 106 in selected locations and the substrate polynucleotides deposited onto the gold.

As shown in FIG. 1A, reservoir 104 comprises a cavity configured to contain a volume of liquid (e.g., an aqueous solution), wherein an interior bottom surface of the cavity is formed from at least a portion of top surface 106c of substrate 106. In some embodiments, reservoir 104 may be formed by arranging walls over the top of the substrate 106.

In some embodiments, the aqueous solution of reservoir 104 comprises a pool of nucleotides. The pool of nucleotides may comprise a plurality of protected nucleotides, where each protected nucleotide comprises a photocleavable terminating moiety and a detectable moiety (e.g., a fluorophore). In some embodiments, the photocleavable terminating moiety of a protected nucleotide is configured to be cleaved upon exposure to photocleavage light having a first wavelength. In some embodiments, the detectable moiety of the protected nucleotide is configured to be excited upon exposure to excitation light having a second wavelength and to subsequently emit emission light having a third wavelength. In some embodiments, the aqueous solution of reservoir 104 comprises one or more polymerases (e.g., one or more DNA polymerases). In some embodiments, the aqueous solution of reservoir 104 further comprises a sample (e.g., a biological sample) comprising a target nucleic acid (e.g., a nucleic acid capable of hybridizing to a substrate polynucleotide 110). In some embodiments, the photocleavage light may be used as an excitation light. In some embodiments, the excitation light may have the same wavelength as the photocleavage light but may have a shorter pulse duration (i.e., pulse width) and/or lower intensity than the photocleavage light. In some such embodiments, a short and/or weak pulse of excitation light may result in sufficient fluorescence for identification of one or more incorporated bases but may not result in cleavage of photocleavable terminating moieties or of a significant number of photocleavable terminating moieties.

In reservoir 104, a target nucleic acid of a sample may hybridize to one or more substrate polynucleotides 110, and a polymerase may elongate one or more substrate polynucleotides 110 by using the target nucleic acid as a template (e.g., to produce a pool of elongated substrate polynucleotide daughter strands, e.g., amplicons).

According to some embodiments, the above-described process of producing evanescent waves in reservoir 104 may be performed (e.g., by operating light sources 112 and/or 114) such that the evanescent waves excite a detectable moiety of a protected nucleotide incorporated into a sequencing primer annealed to substrate polynucleotide 110 immobilized to bottom surface 108 of reservoir 104. The detectable moiety may emit emission light having one or more characteristics (e.g., wavelength, intensity, lifetime decay, pulse width) that may identify a type of the incorporated protected nucleotide. In some embodiments, a device comprising an evanescent wave imaging apparatus is configured such that one or more light sources (e.g., light sources 112 and/or 114) produce, as a result of total internal reflection within a substrate, an evanescent wave that excites a detectable moiety of a protected nucleotide incorporated into a sequencing primer annealed to substrate polynucleotide 110 immobilized to bottom surface 108 of reservoir 104. The one or more light sources may emit excitation light having one or more characteristics (e.g., wavelength, intensity, lifetime decay, pulse width) and that produces an evanescent wave that effectively excites a detectable moiety, causing it to emit emission light having one or more characteristics (e.g., wavelength, intensity, lifetime decay, pulse width) that may be analyzed to identify a type of the incorporated protected nucleotide. In some embodiments, the excitation light that produces an evanescent wave that effectively excites a detectable moiety does not reverse termination of elongation of a sequencing primer or does not substantially reverse termination of elongation of a sequencing primer. In other embodiments, the excitation light that produces an evanescent wave that effectively excites a detectable moiety does reverse termination of elongation of a sequencing primer (e.g., by cleaving a photocleavable terminating moiety of a protected nucleotide incorporated into the sequencing primer). In some such embodiments, one or more characteristics of the excitation light (e.g., intensity or pulse width) are configured to mitigate (e.g., decrease or minimize) reversing termination of elongation of a sequencing primer (e.g., by cleaving a photocleavable terminating moiety of a protected nucleotide incorporated into the sequencing primer).

According to some embodiments, the above-described process of producing evanescent waves in reservoir 104 may be performed (e.g., by operating light sources 112 and/or 114) to reverse termination of elongation of a sequencing primer (e.g., by cleaving a photocleavable terminating moiety of a protected nucleotide incorporated into the sequencing primer). In certain instances, for example, at least one of light sources 112 and 114 are configured to emit light having a peak wavelength in the UV range of the electromagnetic spectrum. In some embodiments, the photocleavable termination moiety attached to a protected nucleotide incorporated in a sequencing primer may cause termination of elongation of the sequencing primer by a polymerase, and subsequent exposure of the sequencing primer (annealed to substrate polynucleotide 110) to an evanescent wave produced by at least one of light sources 112 and 114 may cleave the photocleavable termination moiety and reverse such termination, thus enabling elongation of the sequencing primer to resume. In some embodiments, a device comprising an evanescent wave imaging apparatus is configured such that one or more light sources (e.g., light sources 112 and/or 114) produce, as a result of total internal reflection within a substrate, an evanescent wave that reverses termination of elongation of a sequencing primer (e.g., by cleaving a photocleavable terminating moiety of a protected nucleotide incorporated into the sequencing primer). The one or more light sources may emit excitation light having one or more characteristics (e.g., wavelength, intensity, lifetime decay, pulse width) and that produces an evanescent wave that effectively excites a photocleavable termination moiety attached to a protected nucleotide incorporated in a sequencing primer, causing cleavage of the photocleavable termination moiety and reversing termination, thus enabling elongation of the sequencing primer to resume. In some embodiments, the excitation light that produces an evanescent wave that effectively excites the photocleavable termination moiety is UV light. In some embodiments, the excitation light that produces an evanescent wave that effectively excites the photocleavable termination moiety does not excite a detectable moiety of a protected nucleotide incorporated into a sequencing primer annealed to a substrate polynucleotide or does not substantially excite a detectable moiety of a protected nucleotide incorporated into a sequencing primer annealed to a substrate polynucleotide. In other embodiments, the excitation light that produces an evanescent wave that effectively excites the photocleavable termination moiety does excite a detectable moiety of a protected nucleotide incorporated into a sequencing primer annealed to a substrate polynucleotide.

In the example of FIG. 1A, device 100A comprises an optical imaging system 116A, which comprises image sensor 118 and lens 120. In some embodiments, pixels of image sensor 118 may be arranged directly beneath substrate polynucleotides 110 such that emission light that is directly downward from the substrate polynucleotides in reservoir 104 will be incident on pixels of the image sensor 118. In some embodiments, the area of the active region of image sensor 118 may be larger than the area of the region comprising the substrate polynucleotides 110, in which case lens 120 may be configured to spread the light outwards onto the image sensor. In some embodiments, the area of the active region of image sensor 118 may be smaller than the area of the region comprising the substrate polynucleotides 110, in which case lens 120 may be configured to converge the light inwards onto the image sensor.

In some embodiments, lens 120 is a single lens. In certain embodiments, lens 120 is a compound lens. In certain embodiments, lens 120 is a finite conjugate microscope objective lens. In some such embodiments where lens 120 is a single lens, magnification and focusing may be interdependent (e.g., magnification may be fixed once focusing is achieved).

In some embodiments, lens 120 comprises two or more lenses. In some embodiments, for example, lens 120 comprises an upper lens 120A and a lower lens 120B. In certain embodiments, lens 120A is an infinity-corrected lens (e.g., positioned at its focal length from the substrate, looking down) and lens 120B is an infinity-corrected lens (e.g., positioned at its focal length from the sensor, looking up (infinity side towards lens 120A)). In some such embodiments, each lens may be positioned a precise distance from the sensor or the substrate, and the distance between lens 120A and lens 120B may have little to no impact on focus. In certain cases, this may facilitate manufacturing and/or may allow insertion of filters of varying thicknesses and/or optical lengths between lens 120A and lens 120B without impacting focus. The magnification in some such embodiments may be given by the ratio of focal lengths of lens 120A and lens 120B. In certain embodiments, lens 120A is a microscope objective lens and lens 120B is a tube lens.

According to some embodiments, image sensor 118 may comprise any suitable component or components suitable for detecting light intensity of received emission light, and may comprise any number of pixels that may each detect received light intensity. In some embodiments, the image sensor may produce image sensor data over time indicating, for each pixel of the sensor, an intensity of light received. The image sensor may comprise pixels with different filters or otherwise configured to receive particular frequency bands of light. For instance, the pixels of image sensor 118 may comprise red, green, and blue filters in a Bayer pattern. Non-limiting examples of sensors that may be used as image sensor 118 include a Canon® single-photon avalanche diode (SPAD) sensor and a Sony® IMX447 sensor. The generated image sensor data may be transmitted or otherwise communicated to a suitable computing device, an example of which is described below.

In some embodiments, light source 114 may be omitted and light source 112 may be configured to emit light having a first peak wavelength, and the light having the first peak wavelength may be used to both excite a detectable moiety of a protected nucleotide and reverse termination of elongation of a sequencing primer by a polymerase (e.g., by cleaving a photocleavable terminating moiety of the protected nucleotide). The function of identification versus reverse termination by the same light source can be separable by the intensity or pulse time of the light source. A short or weak pulse may not cause reverse termination, but sufficient fluorescence for identification. In some embodiments, light source 112 may be configured to emit light having a first peak wavelength and light having a second peak wavelength. In some such embodiments, light having the first peak wavelength may be used to excite a detectable moiety of the protected nucleotide and light having the second peak wavelength may be used to reverse termination of elongation of a sequencing primer, annealed to substrate polynucleotide, by a polymerase (e.g., by cleaving a photocleavable terminating moiety of the protected nucleotide).

In some embodiments, device 100A further comprises one or more additional light sources (not shown in FIG. 1A) in addition to light sources 112 and 114. In some embodiments, the device further comprises a third light source. In certain cases, the third light source is arranged proximate to the substrate and configured to direct light into the substrate. In some instances, the third light source is positioned adjacent to a side of substrate 106 not shown in FIG. 1A. In certain cases, the third light source produces light that has a different wavelength spectrum from light produced by light sources 112 and 114. In some embodiments, the device further comprises a fourth light source. In certain cases, the fourth light source is arranged proximate to the substrate and configured to direct light into the substrate. In some instances, the fourth light source is positioned adjacent to a side of substrate 106 not shown in FIG. 1A (and, in some cases, different from the side along which the third light source is positioned). In certain cases, the fourth light source produces light that has a different wavelength spectrum from light produced by light sources 112 and 114 and the third light source. In some embodiments, device 100A comprises a light source configured to emit ultraviolet radiation (e.g., one of light source 112, light source 114, the third light source, and the fourth light source, or a separate fifth light source). The light source configured to emit ultraviolet radiation may be arranged proximate to the substrate and configured to direct light into the substrate.

While in the example of FIG. 1A the depicted elements are shown without surrounding structure, it may be appreciated that in general the device 100A may be implemented within a housing or other structure that contains these elements and blocks light originating outside of the device from entering the device to a significant extent. FIG. 1C depicts an example of such an arrangement, wherein structure 180 is arranged around substrate 106, light sources 112 and 114, lens 120, and image sensor 118, according to some embodiments. Such a surrounding structure may be provided in any of the embodiments described herein, including any of the described embodiments of device 100A in addition to the additional device embodiments 110E and 100F described below.

In some embodiments, certain parts of the device 100A may be configured to be removable from the device. This may be advantageous in that certain elements may be considered consumable and replaceable, whereas other elements may be re-used. FIG. 1D depicts one illustrative example of how a portion of device 100A may be removable. In the example of FIG. 1D, the elements within evanescent wave imaging apparatus 102 may be considered to be an integral part of the device, whereas the reservoir 104 and substrate 106 may be configured to be removably inserted into the apparatus. As such, the same light sources, lens, and image sensor may be re-used with multiple different instances of the reservoir and substrate. The reservoir and substrate may be inserted into the apparatus separately or as a single unit.

In some embodiments, reservoir 104 and/or substrate 106 may comprise one or more features configured to facilitate accurate insertion of reservoir 104 and/or substrate 106 into evanescent wave imaging apparatus 102 (e.g., to facilitate alignment of substrate 106 with light sources 112 and/or 114 of apparatus 102). In some embodiments, apparatus 102 may comprise one or more reservoir alignment features (e.g., guide rails) (not shown in FIG. 1D) configured to guide insertion of reservoir 104 and/or substrate 106 into apparatus 102. A reservoir alignment feature may, in some cases, be a feature (e.g., a guide rail) having a particular shape configured to fit into a corresponding reservoir alignment opening in the evanescent wave imaging apparatus. In some cases, the reservoir comprises one, two, three, four, five, six, or more reservoir alignment features. In operation, reservoir 104 and substrate 106 may be inserted into apparatus 102 such that first surface 106a of substrate 106 is aligned with light source 112 and second surface 106b of substrate 106 is aligned with light source 114.

In some embodiments, for example, the reservoir comprises one or more reservoir alignment features. A reservoir alignment feature may, in some cases, be a feature (e.g., a guide rail) having a particular shape configured to fit into a corresponding reservoir alignment opening in the evanescent wave imaging apparatus.

In some embodiments, reservoir 104 comprises, or is coupled to, one or more magnets. These magnets may, for instance, be attached to some part of the reservoir outside of the interior of the vessel (e.g., attached to an exterior wall or housing). In some cases, the one or more magnets may be arranged proximate to one or more magnets in apparatus 102, wherein the magnets are arranged to attract one another (e.g., opposing poles of permanent magnets may be arranged in the reservoir and in a housing of the apparatus). In some cases, one or more magnets coupled to the reservoir may advantageously secure the reservoir in a configuration in which the substrate is aligned with one or more light sources (e.g., one or more first light sources and/or one or more second light sources) of apparatus 102. Additionally, or alternatively, the one or more magnets coupled to the reservoir 104 may provide a force to draw the reservoir against mechanical alignment features during insertion of the reservoir into the device. In some embodiments, the reservoir comprises one or more gaskets. The gaskets may be formed from any suitable material, such as silicone.

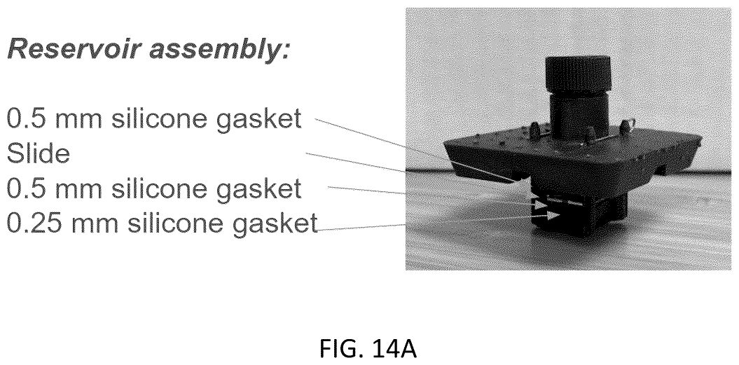

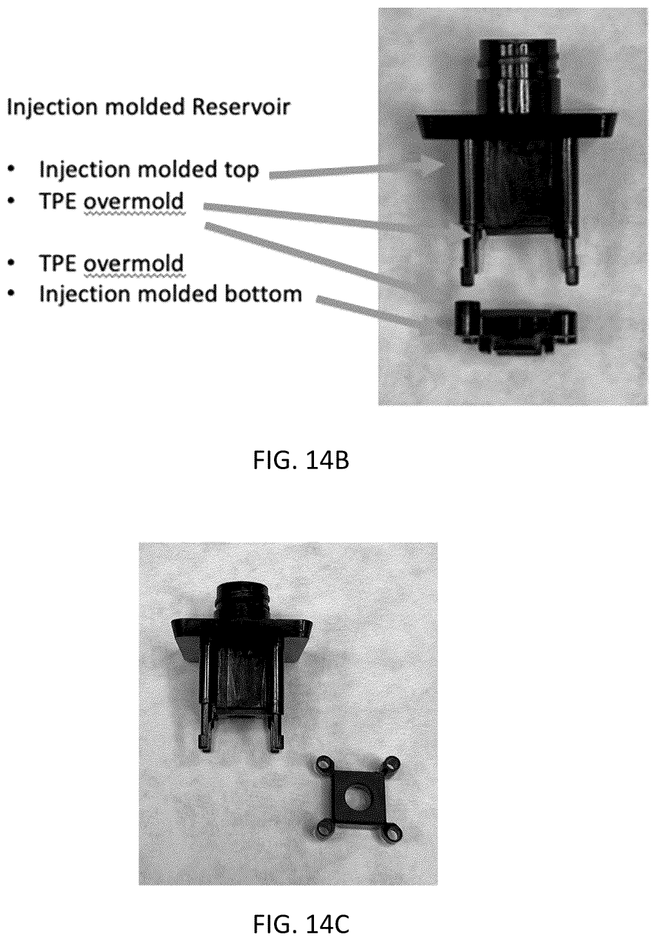

FIGS. 14B-14C show, according to some embodiments, images of exemplary injection-molded reservoirs comprising an injection-molded top component, a thermoplastic elastomer (TPE) overmold, and an injection-molded bottom component. The injection-molded reservoir of FIGS. 14B-14C may be mass fabricated.

In some embodiments, a top surface of the reservoir comprises one or more features (e.g., a handle) to facilitate user handling of the reservoir (e.g., to allow a user to easily insert the reservoir into apparatus 102 and/or remove the reservoir from the apparatus).

In some embodiments, the device 100A may comprise additional elements not shown in FIG. 1A. These elements may include heat sinks, optical filters, an isolation layer, and light blocking layers. These illustrative elements are shown in one of FIGS. 1E and 1F and described below. It may be appreciated that any suitable combination of these elements may be included in a device for nucleic acid sequencing, and these elements are not limited to being implemented in the particular combinations shown.

In the example of FIG. 1E, the device 100E includes optical filters 122 and 124, in addition to heat sinks 132. The other elements of device 100E are the same as shown in FIG. 1A and described above. Illustrative device 100E includes optical filter 122 positioned between substrate 106 and lens 120. Optical filter 122 may be, or may comprise, a longpass filter, a shortpass filter, a bandpass filter, a notch filter, or a combination thereof. For example, optical filter 122 may comprise a longpass filter configured to transmit light having a wavelength above a particular threshold (e.g., 450 nm, 500 nm, etc.). In such an implementation, emission light emitted from a detectable moiety of a protected nucleotide may be transmitted through substrate 106 and may be incident on optical filter 122, which may transmit incident light (e.g., incident emission light) above the particular threshold while blocking incident light below the particular threshold (e.g., incident UV excitation light). In some embodiments, optical filter 122 may exhibit a periodic transmission spectrum that may exhibit comparatively low transmission at one or more wavelengths to be blocked (e.g., usually the excitation wavelength) and comparatively high transmission at one or more wavelengths to be sensed by the image sensor (e.g., the emission wavelength). In some embodiments, optical filter 122 is integrated onto bottom surface 106d of substrate 106 by coating the substrate with one or more thin film optical filters.

Illustrative device 100E includes optical filter 124 positioned between light source 112 and substrate 106. In some embodiments, some (or, in some cases, substantially all) light emitted by light source 112 may pass through one or more optical filters 124 prior to entering substrate 106 through first surface 106a of substrate 106. Optical filter 124 may be, or may comprise, a longpass filter, a shortpass filter, a bandpass filter, a notch filter, or a combination thereof. For example, light source 112 may produce a broad range of wavelengths of light (e.g., UV and/or visible light), and optical filter 124 may block a portion of the broad range of wavelengths from entering first surface 106a of substrate 106. In embodiments comprising light source 114, one or more optical filters 124 may additionally or alternatively be positioned between light source 114 and substrate 106. In embodiments comprising one or more additional light sources, one or more optical filters may additionally or alternatively be positioned between those light sources and substrate 106.

According to some embodiments, optical filters 122 and/or 124 may be an absorptive filter or a dichroic filter. In some embodiments, optical filters 122 and/or 124 may comprise one or more layers of a dielectric material and/or a metal. In some embodiments, optical filters 122 and/or 124 may comprise two or more layers of materials have different refractive indices. In some embodiments, optical filters 122 and/or 124 may comprise a volume of water.

In some embodiments, light source 112 and/or 114 (and/or any additional light source that is present) is operably coupled with one or more excitation light optical filters (e.g., optical filter 124) sufficient to block an undesired subset of the light source's spectrum of light (e.g., a subset sufficient to excite a detectable moiety and/or a photocleavable terminating moiety of a protected nucleotide). As used herein, “operably coupled” describes a relationship between two objects where the two objects are positioned and/or configured to function together. In some embodiments, operably coupled refers to two objects positioned and/or configured to transmit light from one to the other. In some embodiments, operably coupled refers to two objects positioned and/or configured to transfer heat from one to the other. Position may refer to absolute position (e.g., relative to an axis of a device or apparatus) or relative position (e.g., the positions of the operably coupled objects to one another. Configured may refer to any relevant property of either or both objects (e.g., transmission or absorbance spectra, refractive index, heat capacity, size (e.g., length, width, or thickness)).

Illustrative device 100E includes heat sinks 132 in thermal communication with light source 112 and light source 114. The heat sinks 132 may be configured to dissipate heat generated by the respective light source and/or other components of apparatus 102E, to prevent the one or more light sources and/or other components from overheating and/or adversely affecting the contents of reservoir 104 (e.g., substrate nucleotides 110). In some embodiments, heat sinks 132 may be configured to maintain reservoir 104 at a selected temperature to control one or more reactions within reservoir 104. In certain embodiments, for example, heat sinks 132 may be connected to a heating element (e.g., a resistive element) and a temperature sensor (not shown in FIG. 1E). In some cases, a controller in communication with the heating element and the temperature sensor may be configured to maintain temperature at a desired set point.

Turning to FIG. 1F, illustrative device 100F includes isolation layer 134 and light blocking layer 136. The isolation layer is configured to optically isolate the solution in reservoir 104 from evanescent light where it is present. Without this layer, light that is incident upon the upper boundary of the substrate 106 at an incident angle below the critical angle and that is transmitted into the reservoir may adversely affect the chemistry within the reservoir. For instance, such light being transmitted into the reservoir may trigger unwanted luminescence events that may lead to erroneous measurements by image sensor 118 and/or may lead to undesired cleaving of free terminators in the reservoir, which may lower the signal-to-noise ratio of incorporated terminators and over time decrease the availability of them for incorporation. The purpose of the isolation layer 134 is therefore to optically separate such light rays so that they are separated from the evanescent waves. According to some embodiments, the isolation layer 134 may be optically transparent.

According to some embodiments, isolation layer 134 may be arranged over substrate 106 both within reservoir 104 and outside of the reservoir, as shown in FIG. 1F. In some embodiments, isolation layer 134 may cover the entirety of the interior bottom surface of the reservoir 104 except for the substrate polynucleotides 110. Isolation layer 134 may be thick enough to contain the energy of the evanescent waves, but thin enough to be compatible with fabrication processes to form gaps in the isolation layer for the substrate polynucleotides 110. In certain embodiments, isolation layer 134 may not be present on bottom surface of substrate 106. In certain embodiments, isolation layer may not be present outside of reservoir 104.

According to some embodiments, isolation layer 134 may have a thickness of greater than or equal to 100 nm, 250 nm, 500 nm, 750 nm, 1 μm, 2 μm, 3 μm, 4 μm, 5 μm, 10 μm, 50 μm, 100 μm, 500 μm, 1 mm, 1.5 mm, 2 mm, 2.5 mm, 3 mm, 3.5 mm, 4 mm, 4.5 mm, or 5 mm. According to some embodiments, isolation layer 134 may have a thickness of less than or equal to 5 mm, 4.5 mm, 4 mm, 3.5 mm, 3 mm, 2.5 mm, 2 mm, 1.5 mm, 1 mm, 500 μm, 100 μm, 50 μm, 10 μm, 5 μm, 4 μm, 3 μm, 2 μm, 1 μm, 750 nm, 500 nm, 250 nm, or 100 nm. Any suitable combinations of the above-referenced ranges are also possible (e.g., a thickness of greater than or equal to 500 nm and less than or equal to 2 μm).

In some cases, the thickness of the isolation layer 134 may be relatively thick compared to the decay length of the evanescent waves (e.g., the value of d in the equation above). For instance, in some embodiments, the thickness of the isolation layer 134 may be greater than or equal to 2, 3, 4, 5, 6, 7, 8, 9, 10, 11, or 12 times the decay length of the evanescent waves. In some embodiments, the thickness of the isolation layer 134 may be less than or equal to 12, 11, 10, 9, 8, 7, 6, 5, 4, 3, or 2 times the decay length of the evanescent waves. Any suitable combinations of the above-referenced ranges are also possible (e.g., a thickness greater than or equal to 2 times the decay length, and less than or equal to 10 times the decay length).

According to some embodiments, isolation layer 134 may include an exterior coating or other structure configured to absorb light passing through the isolation layer. Such an absorbing coating may be arranged only on the portion of the isolation layer 134 that is exterior to the reservoir. In some embodiments, the device may comprise a gasket and/or O-ring in contact with the isolation layer (and outside of the reservoir) to block light passing through the isolation layer.

According to some embodiments, isolation layer 134 may have a refractive index equal or higher than the refractive index of the fluid in the reservoir. It may be desirable, however, for the refractive index to be close to the refractive index of the fluid in the reservoir, so as to maintain optical homogeneity of the isolation layer 134 across the entire surface of 106c, minimize scattering and so the isolation layer is not visible to the imaging system.

According to some embodiments, isolation layer 134 may have a refractive index of greater than or equal to 1.33, 1.34, 1.35, 1.36, 1.37, 1.38, 1.39, or 1.40. According to some embodiments, isolation layer 134 has a refractive index of less than or equal to 1.40, 1.39, 1.38, 1.37, 1.36, 1.35, 1.34, or 1.33. Any suitable combinations of the above-referenced ranges are also possible (e.g., a refractive index of greater than or equal to 1.33 and less than or equal to 1.36). According to some embodiments, isolation layer 134 may directly contact substrate 106. For instance, the isolation layer may be welded or otherwise directly attached to the substrate. Alternatively, the isolation layer 134 may be attached to the substrate via one or more wetting layers.

Illustrative device 100F includes light blocking layer 136, which may be arranged to block light from light source 112 and/or light source 114 (and, if present, any additional light sources) from entering reservoir 104 except through bottom surface 108. One or more light blocking layers 136 may comprise a light-absorbing structure and/or a light-absorbing coating.

FIG. 1G depicts an illustrative system that comprises any of the nucleic acid sequencing devices described above and shown in any of FIGS. 1A-1F. In the example of FIG. 1G, the device 125 (e.g., device 100A, 100E, 100F) is operably coupled via any suitable wireless and/or wired connection(s) to processing system 126, which comprises one or more processors 128 coupled to one or more memory devices 130. Processing system 126 may be configured to control device 125 by operating one or more light sources (e.g., activating, deactivating, etc.), operating an image sensor to capture image sensor data (e.g., controlling exposure time), and/or operating one or more heating and/or cooling devices. In some embodiments, processing system 126 may be configured to receive said image sensor data from the image sensor. Processing system 126 may thereby analyze image sensor data to identify or otherwise quantify nucleotides within the reservoir of the device. For instance, processing system 126 may analyze image sensor data generated based on received emission light to identify a protected nucleotide (e.g., as one of A, C, G, T, or U) based on one or more characteristics (e.g., wavelength, intensity, lifetime decay, pulse width) of the emission light.

In some embodiments, one or more memory devices 130 comprise at least one non-transitory computer-readable storage medium storing instructions that, when executed by the one or more processors 128, cause the one or more processors to control various aspects of operation of device 125. For example, the instructions may comprise a module for controlling light source 112, a module for controlling light source 114, a module for controlling optical imaging system 116a, etc. In some embodiments, the instructions may comprise a module for controlling operation of each function of device 125. In some embodiments, a main module may control interoperability of some or all of the modules stored in one or more memory devices 130.

In some embodiments, processing system 126 may be configured to store and/or process data received from one or more components of device 125 (e.g., image sensor 118). In some embodiments, one or more computer processors 128 may be configured to receive image data provided by image sensor 118 and to cause the image data to be stored in one or more memory devices 130 and/or to be processed by a detection module stored in one or more memory devices 130. The detection module may, for example, identify a type of a protected nucleotide incorporated in a sequencing primer annealed to substrate polynucleotide 110 based on a characteristic (e.g., wavelength, intensity, lifetime decay, pulse width) of light emitted by a detectable moiety of the protected nucleotide.

In some embodiments, one or more components of processing system 126 are positioned within a housing of device 125. In certain instances, all components of processing system 126 are positioned within a housing of device 125. In some embodiments, one or more components of processing system 126 are positioned outside a housing of device 125. In each case, one or more components of processing system 126 may be connected via wires or wirelessly to one or more other components of device 125 (e.g., image sensor 118). In each case, one or more components of processing system 126 may be connected via wires or wirelessly to an external processing system (e.g., an external laptop or desktop computer). Examples of wireless protocols that may be used for communication of electronic signals include, but are not limited to, Wi-Fi (e.g., any of the IEEE 802.11 family of protocols), Bluetooth®, Zigbee and other IEEE 802.15.4-based protocols, cellular protocols, and the like.

Further examples and descriptions are provided below for various embodiments and features of the various elements described above.

### Reservoir

As described above, a nucleic acid sequencing device may comprise a reservoir. Below are described various features of such a reservoir, including the contents of a reservoir during operation of the device. The below description may be applied to any suitable embodiment described above in relation to FIGS. 1A-1G, including any of the above description relating to reservoir 104 and its features.

### Nucleotides

In some embodiments, the reservoir of a nucleic sequencing device comprises an aqueous solution. In certain cases, the aqueous solution of the reservoir comprises a pool of nucleotides. A nucleotide can include a nucleobase (e.g., adenine, cytosine, guanine, thymine, uracil), a sugar (e.g., ribose, deoxyribose), and one or more phosphate groups (e.g., a triphosphate group). When a nucleotide comprises one or more phosphate groups, a first phosphate group positioned closest to the sugar (e.g., directly bonded to the sugar) may be referred to as an alpha-phosphate group. When a nucleotide comprises two or more phosphate groups, a second phosphate group directly bonded to the first phosphate group may be referred to as a beta-phosphate group. When a nucleotide comprises three or more phosphate groups, a third phosphate group directly bonded to the second phosphate group may be referred to as a gamma-phosphate group.

In some embodiments, the pool of nucleotides comprises one or more types of nucleotides. As used herein, “type” of nucleotide refers to the nucleobases that characterize the nucleotides of DNA and RNA. Types of nucleotides include, but are not limited to, adenine, cytosine, guanine, thymine, and uracil nucleotides. In certain embodiments, the pool of nucleotides comprises adenine, cytosine, guanine, and thymine nucleotides. In certain embodiments, the pool of nucleotides comprises adenine, cytosine, guanine, and uracil nucleotides. In some embodiments, the concentration of a type of nucleotide (and, in some cases, each type of nucleotide) is at least 10 nM, at least 20 nM, at least 50 nM, at least 100 nM, at least 200 nM, at least 500 nM, at least 600 nM, or at least 1000 nM. In some embodiments, the concentration of a type of nucleotide (and, in some cases, each type of nucleotide) is in a range from 10-50 nM, 10-100 nM, 10-200 nM, 10-500 nM, 10-600 nM, 10-1000 nM, 50-100 nM, 50-200 nM, 50-500 nM, 50-600 nM, 50-1000 nM, 100-200 nM, 100-500 nM, 100-600 nM, 100-1000 nM, 200-500 nM, 200-600 nM, 200-1000 nM, 500-1000 nM, or 600-1000 nM. In some embodiments, the total concentration of all types of nucleotides (e.g., all types of protected nucleotides) in the pool is at least 40 nM, at least 80 nM, at least 200 nM, at least 400 nM, at least 800 nM, at least 1000 nM, at least 2000 nM, or at least 4000 nM. In some embodiments, the total concentration of all types of nucleotide in the pool is in a range from 40-80 nM, 40-200 nM, 40-400 nM, 40-800 nM, 40-1000 nM, 40-2000 nM, 40-4000 nM, 80-400 nM, 80-800 nM, 80-1000 nM, 80-2000 nM, 80-4000 nM, 200-400 nM, 200-800 nM, 200-1000 nM, 200-2000 nM, 200-4000 nM, 400-800 nM, 400-1000 nM, 400-2000 nM, 400-4000 nM, 800-2000 nM, 800-4000 nM, 1000-2000 nM, 1000-4000 nM, or 2000-4000 nM.

Protected Nucleotides

In some embodiments, the pool of nucleotides comprises one or more protected nucleotides. In certain cases, one, two, three, four or more types of nucleotides in the pool are protected nucleotides. As used herein, a protected nucleotide refers to a nucleotide that terminates elongation after it is incorporated into an elongating polynucleotide (e.g., a sequencing primer). A protected nucleotide may comprise one or more covalent modifications relative to a reference nucleotide (e.g., a naturally occurring or canonical nucleotide) that prevent a polymerase from incorporating a further nucleotide into the elongating polynucleotide. In some embodiments, the one or more covalent modifications comprise attachment of a protecting moiety to the nucleotide or replacement of one or more atoms of the reference nucleotide (e.g., a naturally occurring or canonical nucleotide) with a protecting moiety. In certain embodiments, a protected nucleotide comprises a protecting moiety at a site that is not replacing an atom of or occluding the 3′—OH group. Such a protected nucleotide is referred to herein as a 3′-unblocked nucleotide. In some instances, for example, the protecting moiety is attached to and/or replaces one or more atoms of an aromatic ring of a nucleobase of a protected nucleotide. Without wishing to be bound by a particular theory, a 3′-unblocked protected nucleotide may terminate elongation by inhibition or disruption of a polymerase by the protecting moiety.

According to some embodiments, a protecting moiety comprises a photocleavable terminating moiety and a detectable moiety. An exemplary structure of a protected nucleotide comprising a protecting moiety is shown in Formula I:

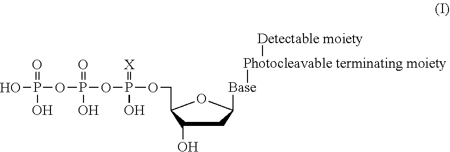

where X represents a heteroatom (e.g., O, S). In certain embodiments, the protecting moiety further comprises a linker between the photocleavable terminating moiety and the detectable moiety.

In some embodiments, the presence of a photocleavable terminating moiety in a protected nucleotide may terminate elongation after incorporation of the protected nucleotide into an elongating polynucleotide (e.g., a sequencing primer) by inhibiting or disrupting a polymerase. For example, and without wishing to be bound by a particular theory, the photocleavable terminating moiety may interfere with the conformation of a polymerase active site. In some embodiments, the presence of a photocleavable terminating moiety in the absence of a detectable moiety may be sufficient to terminate elongation of an elongating polynucleotide. In other embodiments, the presence of the photocleavable terminating moiety and the detectable moiety may be necessary for the protected nucleotide to terminate elongation.

In some embodiments, the termination of elongation by incorporation of a protected nucleotide comprising a photocleavable terminating moiety is reversible. In some embodiments, termination is reversed by removing the photocleavable terminating moiety from the protected nucleotide. In some embodiments, cleaving the photocleavable terminating moiety results in a nucleotide that is identical to a reference nucleotide (e.g., a naturally occurring or canonical nucleotide). In some embodiments, cleaving the photocleavable terminating moiety results in a nucleotide that differs by one or more atoms from a reference nucleotide (e.g., a naturally occurring or canonical nucleotide). In some cases, the resulting nucleotide is competent for extension by a polymerase.

In some embodiments, at least a portion of the photocleavable terminating moiety can be cleaved upon exposure to electromagnetic radiation. In certain instances, the photocleavable terminating moiety is cleaved upon exposure to light having a peak wavelength in the UV range of the electromagnetic spectrum and/or the visible light range of the electromagnetic spectrum. For example, and without wishing to be bound by a particular theory, a photocleavable terminating moiety may absorb one or more wavelengths of electromagnetic radiation (e.g., UV light, visible light) to enter an excited state and undergo photochemistry that results in the cleavage of one or more bonds (e.g., one or more covalent bonds). In some cases, cleavage of the photocleavable terminating moiety releases a detectable moiety from a protected nucleotide.

In some embodiments, the photocleavable terminating moiety has a structure according to Formula II:

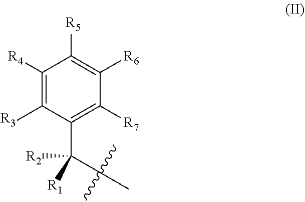

where R₁ and R₂ are each independently selected from the group consisting of H, CF₃, CN, a C₁-C₁₂straight chain or branched alkyl, a C₂-C₁₂ straight chain or branched alkenyl or polyenyl, a C₂-C₁₂ straight chain or branched alkynyl or polyalkynyl, a C₁-C₁₂ ether, and an aromatic group (e.g., a phenyl, a naphthyl, a pyridine), with the proviso that at least one of R₁ and R₂ is CF₃, CN, a C₁-C₁₂ straight chain or branched alkyl, a C₂-C₁₂ straight chain or branched alkenyl or polyenyl, a C₂-C₁₂ straight chain or branched alkynyl or polyalkynyl, a C₁-C₁₂ ether, or an aromatic group (e.g., a phenyl, a naphthyl, a pyridine); and R₃, R₄, R₆, and R₇ are each independently selected from the group consisting of H, OCH₃, NO₂, CN, a halide, a C₁-C₁₂ straight chain or branched alkyl, a C₂-C₁₂ straight chain or branched alkenyl or polyenyl, a C₂-C₁₂ straight chain or branched alkynyl or polyalkynyl, and an aromatic group (e.g., a phenyl (e.g., C₆H₆), a thiophenyl (e.g., S—C₆H₆), a naphthyl, a pyridine); and R₅ comprises a C₁-C₆ alkynyl or polyalkynyl, —C(O)NH—, —C(O)O—, —NH—, —S, S(O)ₙ where n is 0, 1 or 2, —O—, —OP(O)(OH)O—, —OP(O)(O—)O—, arenediyl, heteroarenediyl, an azo, a C₁-C₁₂ straight chain or branched alkyl, and/or a C₂-C₁₂ straight chain or branched alkenyl or polyenyl. In certain embodiments, one of R₁ and R₂ is selected from the group consisting of, but not limited to, methyl, ethyl, propyl, isopropyl, tert-butyl, phenyl, CF₃, CN, C₆H₆, 2-nitrophenyl, and 2,6-dinitrophenyl.

In some embodiments, the photocleavable terminating moiety is, or comprises, a coumarin.

In certain embodiments, the photocleavable terminating moiety is attached (e.g., covalently attached) to a nucleobase of a nucleotide through an ether, ester, carboxylic acid, benzyl amine, benzyl ether, carbamate, carbonate, 2-(o-nitrophenyl)ethyl carbamate, or 2-(o-nitrophenyl)ethyl carbonate linkage. In some embodiments, photo-induced cleavage of the photocleavable terminating moiety of a protected nucleotide cleaves the linkage between the photocleavable terminating moiety and the nucleobase of the nucleotide. In certain embodiments, a product of the photo-induced cleavage step is a hydroxymethyl nucleotide.

In some embodiments, a photocleavable terminating moiety comprises one or more substituent groups that alter a photochemical property of the photocleavable terminating moiety. The photochemical property may be, e.g., the absorption spectra of the photocleavable terminating moiety, e.g., the peak excitation wavelength of the photocleavable terminating moiety. Substituent groups, e.g., electron-donating groups or electron-withdrawing groups, are known in the art, as are methods for modifying a photocleavable terminating moiety with said substituent groups to, e.g., produce a bathochromic or hypsochromic shift, e.g., in the peak excitation wavelength. In some embodiments, one or more of R₁₋₆ in Formula II may comprise a substituent group that alters a photochemical property of the photocleavable terminating moiety.

In some embodiments, the photocleavable terminating moiety is positioned proximal to the nucleotide (e.g., proximal to the nucleobase), such that cleavage of the photocleavable terminating moiety leaves no scar or minimal scar on the nucleotide. A scar, as used in this context, refers to any covalent modification relative to a reference nucleotide (e.g., a naturally occurring or canonical nucleotide) remaining after cleavage of a photocleavable terminating moiety. Without wishing to be bound by a particular theory, scars may inhibit or prevent relief of termination of elongation, e.g., by inhibiting or disrupting polymerase.

In some embodiments, a protecting moiety comprises a detectable moiety. A detectable moiety may comprise any compound or functional group that is attachable to another chemical structure and is readily detectable by a means known to one of skill in the art. In some embodiments, a detectable moiety is fluorescent (i.e., a fluorophore). In some embodiments, a detectable moiety has a color (i.e., a colorimetric moiety).

Non-limiting examples of suitable detectable moieties include: ⅚-Carboxyrhodamine 6G, 5-Carboxyrhodamine 6G, 6-Carboxyrhodamine 6G, 6-TAMRA, Abberior® STAR 440SXP, Abberior® STAR 470SXP, Abberior® STAR 488, Abberior® STAR 512, Abberior® STAR 520SXP, Abberior® STAR 580, Abberior® STAR 600, Abberior® STAR 635, Abberior® STAR 635P, Abberior® STAR RED, Alexa Fluor® 350, Alexa Fluor® 405, Alexa Fluor® 430, Alexa Fluor® 480, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610-X, Alexa Fluor® 633, Alexa Fluor® 647, Alexa Fluor® 660, Alexa Fluor® 680, Alexa Fluor® 700, Alexa Fluor® 750, Alexa Fluor® 790, AMCA, ATTO 390, ATTO 425, ATTO 465, ATTO 488, ATTO 495, ATTO 514, ATTO 520, ATTO 532, ATTO 542, ATTO 550, ATTO 565, ATTO 590, ATTO 610, ATTO 620, ATTO 633, ATTO 647, ATTO 647N, ATTO 655, ATTO 665, ATTO 680, ATTO 700, ATTO 725, ATTO 740, ATTO Oxa12, ATTO Rho101, ATTO Rho11, ATTO Rho12, ATTO Rho13, ATTO Rho14, ATTO Rho3B, ATTO Rho6G, ATTO Thio12, BD Horizon™ V450, BODIPY® 493/501, BODIPY® 530/550, BODIPY® 558/568, BODIPY® 564/570, BODIPY® 576/589, BODIPY® 581/591, BODIPY® 630/650, BODIPY® 650/665, BODIPY® FL, BODIPY® FL-X, BODIPY® R6G, BODIPY® TMR, BODIPY® TR, CAL Fluor® Gold 540, CAL Fluor® Green 510, CAL Fluor® Orange 560, CAL Fluor® Red 590, CAL Fluor® Red 610, CAL Fluor® Red 615, CAL Fluor® Red 635, Cascade® Blue, CF™350, CF™405M, CF™405S, CF™488A, CF™514, CF™532, CF™543, CF™546, CF™555, CF™568, CF™594, CF™620R, CF™633, CF™633-V1, CF™640R, CF™640R-V1, CF™640R-V2, CF™660C, CF™660R, CF™680, CF™680R, CF™680R-V1, CF™750, CF™770, CF™790, Chromeo™ 642, Chromis 425N, Chromis 500N, Chromis 515N, Chromis 530N, Chromis 550A, Chromis 550C, Chromis 550Z, Chromis 560N, Chromis 570N, Chromis 577N, Chromis 600N, Chromis 630N, Chromis 645A, Chromis 645C, Chromis 645Z, Chromis 678A, Chromis 678C, Chromis 678Z, Chromis 770A, Chromis 770C, Chromis 800A, Chromis 800C, Chromis 830A, Chromis 830C, Cy®3, Cy®3.5, Cy®3B, Cy®5, Cy®5.5, Cy®7, DyLight® 350, DyLight® 405, DyLight® 415-Col, DyLight® 425Q, DyLight® 485-LS, DyLight® 488, DyLight® 504Q, DyLight® 510-LS, DyLight® 515-LS, DyLight® 521-LS, DyLight® 530-R2, DyLight® 543Q, DyLight® 550, DyLight® 554-R0, DyLight® 554-R1, DyLight® 590-R2, DyLight® 594, DyLight® 610-B1, DyLight® 615-B2, DyLight® 633, DyLight® 633-B1, DyLight® 633-B2, DyLight® 650, DyLight® 655-B1, DyLight® 655-B2, DyLight® 655-B3, DyLight® 655-B4, DyLight® 662Q, DyLight® 675-B1, DyLight® 675-B2, DyLight® 675-B3, DyLight® 675-B4, DyLight® 679-C5, DyLight® 680, DyLight® 683Q, DyLight® 690-B1, DyLight® 690-B2, DyLight® 696Q, DyLight® 700-B1, DyLight® 700-B1, DyLight® 730-B1, DyLight® 730-B2, DyLight® 730-B3, DyLight® 730-B4, DyLight® 747, DyLight® 747-B1, DyLight® 747-B2, DyLight® 747-B3, DyLight® 747-B4, DyLight® 755, DyLight® 766Q, DyLight® 775-B2, DyLight® 775-B3, DyLight® 775-B4, DyLight® 780-B1, DyLight® 780-B2, DyLight® 780-B3, DyLight® 800, DyLight® 830-B2, Dyomics-350, Dyomics-350XL, Dyomics-360XL, Dyomics-370XL, Dyomics-375XL, Dyomics-380XL, Dyomics-390XL, Dyomics-405, Dyomics-415, Dyomics-430, Dyomics-431, Dyomics-478, Dyomics-480XL, Dyomics-481XL, Dyomics-485XL, Dyomics-490, Dyomics-495, Dyomics-505, Dyomics-510XL, Dyomics-511XL, Dyomics-520XL, Dyomics-521XL, Dyomics-530, Dyomics-547, Dyomics-547P1, Dyomics-548, Dyomics-549, Dyomics-549P1, Dyomics-550, Dyomics-554, Dyomics-555, Dyomics-556, Dyomics-560, Dyomics-590, Dyomics-591, Dyomics-594, Dyomics-601XL, Dyomics-605, Dyomics-610, Dyomics-615, Dyomics-630, Dyomics-631, Dyomics-632, Dyomics-633, Dyomics-634, Dyomics-635, Dyomics-636, Dyomics-647, Dyomics-647P1, Dyomics-648, Dyomics-648P1, Dyomics-649, Dyomics-649P1, Dyomics-650, Dyomics-651, Dyomics-652, Dyomics-654, Dyomics-675, Dyomics-676, Dyomics-677, Dyomics-678, Dyomics-679P1, Dyomics-680, Dyomics-681, Dyomics-682, Dyomics-700, Dyomics-701, Dyomics-703, Dyomics-704, Dyomics-730, Dyomics-731, Dyomics-732, Dyomics-734, Dyomics-749, Dyomics-749P1, Dyomics-750, Dyomics-751, Dyomics-752, Dyomics-754, Dyomics-776, Dyomics-777, Dyomics-778, Dyomics-780, Dyomics-781, Dyomics-782, Dyomics-800, Dyomics-831, eFluor® 450, Eosin, FITC, Fluorescein, HiLyte™ Fluor 405, HiLyte™ Fluor 488, HiLyte™ Fluor 532, HiLyte™ Fluor 555, HiLyte™ Fluor 594, HiLyte™ Fluor 647, HiLyte™ Fluor 680, HiLyte™ Fluor 750, IRDye® 680LT, IRDye® 750, IRDye® 800CW, JOE, LightCycler® 640R, LightCycler® Red 610, LightCycler® Red 640, LightCycler® Red 670, LightCycler® Red 705, Lissamine Rhodamine B, Napthofluorescein, Oregon Green® 488, Oregon Green® 514, Pacific Blue™, Pacific Green™, Pacific Orange™, PET, PF350, PF405, PF415, PF488, PF505, PF532, PF546, PF555P, PF568, PF594, PF610, PF633P, PF647P, Quasar® 570, Quasar® 670, Quasar® 705, Rhodamine 123, Rhodamine 6G, Rhodamine B, Rhodamine Green, Rhodamine Green-X, Rhodamine Red, ROX, Seta™ 375, Seta™ 470, Seta™ 555, Seta™ 632, Seta™ 633, Seta™ 650, Seta™ 660, Seta™ 670, Seta™ 680, Seta™ 700, Seta™ 750, Seta™ 780, Seta™ APC-780, Seta™ PerCP-680, Seta™ R-PE-670, Seta™ 646, SeTau 380, SeTau 425, SeTau 647, SeTau 405, Square 635, Square 650, Square 660, Square 672, Square 680, Sulforhodamine 101, TAMRA, TET, Texas Red®, TMR, TRITC, Yakima Yellow™, Zenon®, Zy3, Zy5, Zy5.5, and Zy7.

In some embodiments, each type of protected nucleotide in the pool of nucleotides comprises a different detectable moiety. As a non-limiting example, adenine nucleotides (e.g., dATPs) may comprise a first detectable moiety, cytosine nucleotides (e.g., dCTPs) may comprise a second detectable moiety, guanine nucleotides (e.g., dGTPs) may comprise a third detectable moiety, and thymine or uracil nucleotides (e.g., dTTPs, dUTPs) may comprise a fourth detectable moiety. In some embodiments, the first, second, third, and/or fourth detectable moieties (e.g., fluorophores) each have distinct excitation and emission spectra, e.g., with distinct excitation and emission peaks. In some embodiments, the first, second, third, and/or fourth detectable moieties absorb UV light. In some embodiments, the first, second, third, and/or fourth detectable moieties absorb visible light. In some embodiments, the first, second, third, and/or fourth detectable moieties emit UV light. In some embodiments, the first, second, third, and/or fourth detectable moieties emit visible light. In some embodiments, the first, second, third, and/or fourth detectable moieties are selected such that light emission of each detectable moiety can be distinguished from the other detectable moieties of other types of nucleotides in the pool of nucleotides, e.g., using a device described herein. In some embodiments, one or more (e.g., all) detectable moieties absorb UV light and emit visible light. Without wishing to be bound by a particular theory, use of UV light to excite a detectable moiety and visible light to detect the detectable moiety, or the use of visible light to excite a detectable moiety and UV light to detect the detectable moiety, may allow a device of the disclosure to more easily distinguish excitation light from emission light, due to their substantially different wavelengths. In some embodiments, the peak excitation wavelength for each detectable moiety is separated from the peak excitation wavelength of each other detectable moiety by at least 5 nm, 10 nm, 15 nm, 20 nm, 25 nm, 30 nm, 35 nm, 40 nm, 45 nm, 50 nm, 55 nm, 60 nm, 65 nm, or 70 nm. In some embodiments, the peak emission wavelength for each detectable moiety is separated from the peak emission of each other detectable moiety by at least 5 nm, 10 nm, 15 nm, 20 nm, 25 nm, 30 nm, 35 nm, 40 nm, 45 nm, 50 nm, 55 nm, 60 nm, 65 nm, or 70 nm. In some embodiments, the peak excitation wavelength for each detectable moiety is separated from the peak excitation wavelength of a photocleavable terminating moiety by at least 5 nm, 10 nm, 15 nm, 20 nm, 25 nm, 30 nm, 35 nm, 40 nm, 45 nm, 50 nm, 55 nm, 60 nm, 65 nm, or 70 nm.

In some embodiments, two types of protected nucleotides in the pool comprise detectable moieties having excitation spectra such that light from a single light source is capable of inducing fluorescence of both detectable moieties. In some embodiments, three types of protected nucleotides in the pool comprise detectable moieties having excitation spectra such that light from a single light source is capable of inducing fluorescence of the three detectable moieties. In some embodiments, four (e.g., all) types of protected nucleotides in the pool comprise detectable moieties having excitation spectra such that light from a single light source is capable of inducing fluorescence of the four (e.g., all) detectable moieties.

In some embodiments, the first, second, third, and/or fourth fluorescent moieties comprise any combination of: Alexa Fluor® 350, Alexa Fluor® 405, ATTO 390, Alexa Fluor® 430, Alexa Fluor® 488, Alexa Fluor® 532, CF®430, CF®594, Alexa Fluor® 555, Alexa Fluor® 568, and ATTO 647N. In some embodiments, the pool of nucleotides comprises a protected adenine nucleotide (e.g., dATP) comprising any detectable moiety described herein. In certain instances, the pool of nucleotides comprises a protected adenine nucleotide (e.g., dATP) comprising a detectable moiety comprising Alexa Fluor® 350, Alexa Fluor® 405, ATTO 390, Alexa Fluor® 430, Alexa Fluor® 488, Alexa Fluor® 532, CF™430, CF™594, Alexa Fluor® 555, Alexa Fluor® 568, and/or ATTO 647N. In some embodiments, the pool of nucleotides comprises a protected guanine nucleotide (e.g., dGTP) comprising any detectable moiety described herein. In certain instances, the pool of nucleotides comprises a protected guanine nucleotide (e.g., dGTP) comprising a detectable moiety comprising Alexa Fluor® 350, Alexa Fluor® 405, ATTO 390, Alexa Fluor® 430, Alexa Fluor® 488, Alexa Fluor® 532, CF™430, CF™594, Alexa Fluor® 555, Alexa Fluor® 568, and/or ATTO 647N. In some embodiments, the pool of nucleotides comprises a protected thymine nucleotide (e.g., dTTP) comprising any detectable moiety described herein. In certain instances, the pool of nucleotides comprises a protected thymine nucleotide (e.g., dTTP) comprising a detectable moiety comprising Alexa Fluor® 350, Alexa Fluor® 405, ATTO 390, Alexa Fluor® 430, Alexa Fluor® 488, Alexa Fluor® 532, CF™430, CF™594, Alexa Fluor® 555, Alexa Fluor® 568, and/or ATTO 647N. In some embodiments, the pool of nucleotides comprises a protected cytosine nucleotide (e.g., dCTP) comprising any detectable moiety described herein. In certain instances, the pool of nucleotides comprises a protected cytosine nucleotide (e.g., dCTP) comprising Alexa Fluor® 350, Alexa Fluor® 405, ATTO 390, Alexa Fluor® 430, Alexa Fluor® 488, Alexa Fluor® 532, CF™430, CF™594, Alexa Fluor® 555, Alexa Fluor® 568, and/or ATTO 647N. In some embodiments, the first, second, third, and fourth fluorescent moieties comprise Alexa Fluor® 488, Alexa Fluor® 532, CF®594, and ATTO 647N, respectively.

In some embodiments, a device or method described herein uses fluorescence resonance energy transfer (FRET) to detect or identify an analyte, e.g., determine the identity of an incorporated nucleotide. FRET refers to a distance-dependent transfer of energy between light-absorbing/emitting molecules. Without wishing to be bound by a particular theory, FRET is characterized by excitation of electrons of a donor molecule by a light source, the transfer of excited state energy from those donor electrons by dipole-dipole interactions to electrons of an acceptor molecule, and emission of a lower energy (e.g., longer wavelength) photon from the acceptor molecule as its electrons relax. FRET partner molecule, as used herein, refers to either a FRET acceptor molecule or a FRET donor molecule. In some embodiments, the FRET partner molecule is part of a label that binds an analyte in the sample. In some embodiments, one or more detectable moieties are selected to be or comprise FRET partner molecule. As described herein, a plurality of detectable moieties (e.g., FRET partner molecules) can be selected to emit wavelengths that are distinguishable and individually detectable, enabling determination of the identities of protected nucleotides comprising said detectable moieties (e.g., FRET partner molecules). In some embodiments, the reservoir comprises a FRET partner molecule. In some embodiments, the FRET partner molecule is attached to a polymerase (e.g., described herein). In some embodiments, the FRET donor molecule is attached to a substrate construct (e.g., to an analyte binding agent, e.g., substrate polynucleotide) or a sequencing primer. In some embodiments, the FRET donor molecule is attached to the substrate (e.g., the top surface of the substrate). In some embodiments, the FRET donor molecule is situated to be within 200 A, within 175 A, within 150 A, within 125 A, within 100 A, within 90 A, within 80 A, within 70 A, within 60 A, within 50 A, within 40 A, within 30 A, within 25 A, within 20 A, within 15 A, within 10 A, or within 5 A of an incorporated protected nucleotide (e.g., comprising a detectable moiety comprising a FRET acceptor molecule).

In an exemplary embodiment utilizing FRET, a polymerase comprises a FRET donor molecule and absorbs light produced by one or more light sources. One or more light sources may be operated by an evanescent wave imaging apparatus so that light from the one or more light sources is absorbed by a FRET donor molecule. As a result of this absorption, emission light may be produced and analyzed to determine the identity of an incorporated molecule. In such exemplary embodiments, the pool of protected nucleotides comprises a nucleotide comprising a FRET partner molecule (e.g., a FRET acceptor molecule). Light from the one or more light sources is transmitted by total internal reflection through the substrate and into the reservoir via the evanescent wave to be absorbed by the FRET donor molecule, which transfers energy via FRET to the incorporated protected nucleotide comprising the FRET acceptor molecule, and the emission of the FRET acceptor molecule can be detected using a detector (e.g., as described herein). Without wishing to be bound by a particular theory, use of FRET may make emission from a detectable moiety easier to detect by, e.g., amplifying the signal and/or shifting the emission from a detectable moiety away from the light of the one or more light sources.

In some embodiments, a protecting moiety comprises a linker connecting a photocleavable terminating moiety and a detectable moiety. In certain embodiments, the linker is a bifunctional linker comprising a first end configured to attach (e.g., covalently attach) to the photocleavable terminating moiety and a second end configured to attach (e.g., covalently attach) to the detectable moiety. In some embodiments, the linker comprises one or more of the following groups: —C(O)NH—, —C(O)O—, —NH—, —S—, —S(O)ₙ where n is 0, 1 or 2, —O—, —OP(O)(OH)O—, —OP(O)(O—)O—, alkanediyl, alkenediyl, alkynediyl, arenediyl, heteroarenediyl, azo, or combinations thereof. In some instances, the linker comprises one or more pendant side chains and/or pendant functional groups. Non-limiting examples of suitable pendant moieties include solubilizing groups, such as —SO₃H and —SO₃.

In some embodiments, the protected nucleotide comprises one or more additional modifications relative to a reference nucleotide. In some embodiments, the one or more additional modifications comprise substituting an oxygen atom of at least one phosphate group of a protected nucleotide with a sulfur atom. In certain instances, an oxygen atom of an alpha-phosphate group of a protected nucleotide is substituted with a sulfur atom. Without wishing to be bound by a particular theory, a nucleotide with said substitution (also referred to as an alpha-thio nucleotide) may exhibit reduced chew-back from residual exonuclease activity in a polymerase.

In some embodiments, the one or more additional modifications comprise addition of one or more biological or chemical moieties. Examples of suitable moieties for modifying nucleotides include, but are not limited to, fluorophores, radioisotopes, chromophores, purification tags (e.g., polyHis, FLAG, biotin, etc.), barcoding molecules, haptens (e.g., FITC, digoxigenin (DIG), fluorescein, bovine serum albumin (BSA), dinitrophenyl, oxazole, pyrazole, thiazole, nitroaryl, benzofuran, triperpene, urea, thiourea, rotenoid, coumarin, etc.), extension blocking groups, and combinations thereof.

In some embodiments, an evanescent wave imaging apparatus, reservoir, and/or photocleavable terminating moiety are configured such that the photocleavable terminating moiety has a molar extinction coefficient of at least 500 cm⁻¹M⁻¹, 750 cm⁻¹M⁻¹, 1000 cm⁻¹M⁻¹, 1500 cm⁻¹M⁻¹, 2000 cm⁻¹M⁻¹, 2500 cm⁻¹M⁻¹, 3000 cm⁻¹M⁻¹, 3500 cm⁻¹M⁻¹, 4000 cm⁻¹M⁻¹, 4500 cm⁻¹M⁻¹, 5000 cm⁻¹M⁻¹, 5500 cm⁻¹M⁻¹, 6000 cm⁻¹M⁻¹, 6500 cm⁻¹M⁻¹, 7000 cm⁻¹M⁻¹, 7500 cm⁻¹M⁻¹, 8000 cm⁻¹M⁻¹, 8500 cm⁻¹M⁻¹, 9000 cm⁻¹M⁻¹, 9500 cm⁻¹M⁻¹, or 10,000 cm⁻¹M⁻¹. In some embodiments, the photocleavable terminating moiety has a molar extinction coefficient in a range from 500-1000 cm⁻¹M⁻¹, 500-2000 cm⁻¹M⁻¹, 500-5000 cm⁻¹M⁻¹, 500-10,000 cm⁻¹M⁻¹, 1000-5000 cm⁻¹M⁻¹, 1000-10,000 cm⁻¹M⁻¹, or 5000-10,000 cm⁻¹M⁻¹. For example, the photocleavable terminating moiety and one or more light sources of the evanescent wave imaging apparatus may be selected such that the photocleavable terminating moiety exhibits desirable absorption properties.

In some embodiments, the evanescent wave imaging apparatus, reservoir, and/or photocleavable terminating moiety are configured to photochemically cleave the photocleavable terminating moiety at a quantum yield of at least 0.1, 0.11, 0.12, 0.13, 0.14, 0.15, 0.16, 0.17, 0.18, 0.19, 0.2, 0.25, 0.3, 0.35, 0.4, 0.45, 0.5, 0.55, 0.6, 0.65, or 0.7 photocleavable terminating moieties cleaved per photon absorbed. In some embodiments, the quantum yield is in a range from 0.1-0.2, 0.1-0.3, 0.1-0.4, 0.1-0.5, 0.1-0.6, 0.1-0.7, 0.2-0.3, 0.2-0.4, 0.2-0.5, 0.2-0.6, 0.2-0.7, 0.3-0.5, 0.3-0.6, 0.3-0.7, 0.4-0.6, 0.4-0.7, 0.5-0.7, or 0.6-0.7 photocleavable terminating moieties cleaved per photon absorbed. For example, the photocleavable terminating moiety and one or more light sources may be selected such that the photocleavable terminating moiety exhibits desirable photochemical reaction parameters.

In some embodiments, a protected nucleotide has a structure according to Formula III:

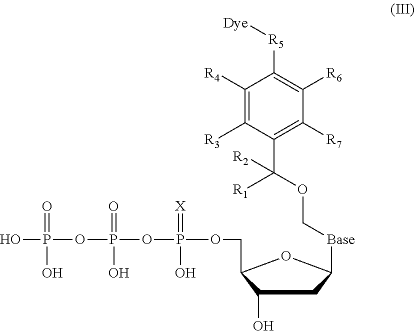

wherein Base is a nucleobase (e.g., adenine, cytosine, guanine, thymine, uracil); X is a heteroatom (e.g., sulfur, oxygen); R₁ and R₂ are each independently selected from the group consisting of H, CF₃, CN, a C₁-C₁₂ straight chain or branched alkyl, a C₂-C₁₂ straight chain or branched alkenyl or polyenyl, a C₂-C₁₂ straight chain or branched alkynyl or polyalkynyl, a C₁-C₁₂ ether, and an aromatic group (e.g., a phenyl, a naphthyl, a pyridine), with the proviso that at least one of R₁ and R₂ is CF₃, CN, a C₁-C₁₂ straight chain or branched alkyl, a C₂-C₁₂ straight chain or branched alkenyl or polyenyl, a C₂-C₁₂ straight chain or branched alkynyl or polyalkynyl, a C₁-C₁₂ ether, or an aromatic group (e.g., a phenyl, a naphthyl, a pyridine); R₃, R₄, R₆, and R₇ are each independently selected from the group consisting of H, OCH₃, NO₂, CN, a halide, a C₁-C₁₂ straight chain or branched alkyl, a C₂-C₁₂ straight chain or branched alkenyl or polyenyl, a C₂-C₁₂ straight chain or branched alkynyl or polyalkynyl, and an aromatic group (e.g., a phenyl (e.g., C₆H₆), a thiophenyl (e.g., S—C₆H₆), a naphthyl, a pyridine); and R₅ comprises a C₁-C₆ alkynyl or polyalkynyl, —C(O)NH—, —C(O)O—, —NH—, —S—, —S(O)ₙ where n is 0, 1 or 2, —O—, —OP(O)(OH)O—, —OP(O)(O—)O—, arenediyl, heteroarenediyl, an azo, a C₁-C₁₂ straight chain or branched alkyl, and/or a C₂-C₁₂ straight chain or branched alkenyl or polyenyl. In certain embodiments, R₁ is CF₃, CN, C₆H₆, or tert-butyl. In certain embodiments, R₃ is NO₂. In certain embodiments, R₅ comprises a C₂-C₁₂ alkyne, an amide, and/or an amine. In certain embodiments, R₆ is OMe or S—C₆H₆.

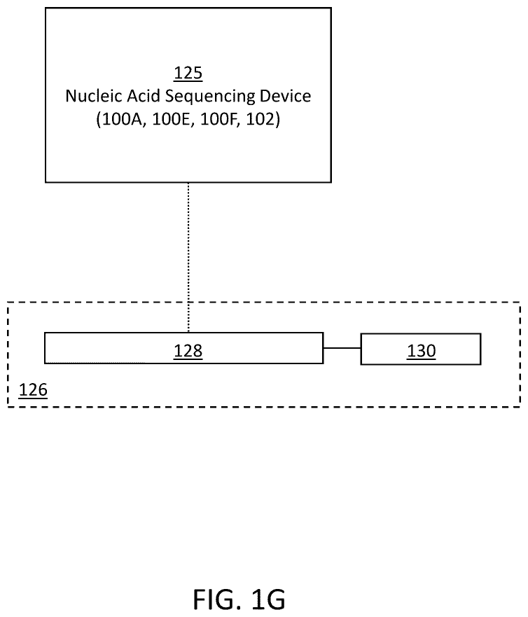

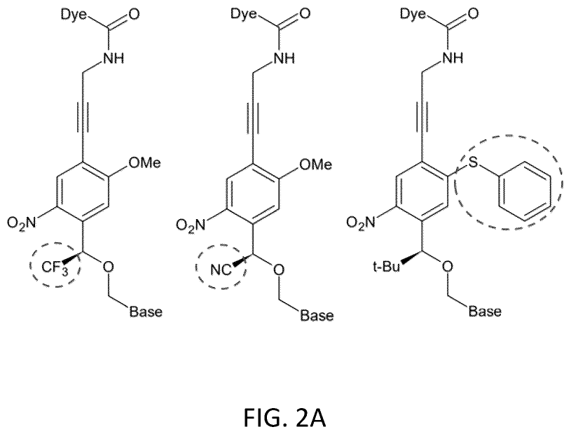

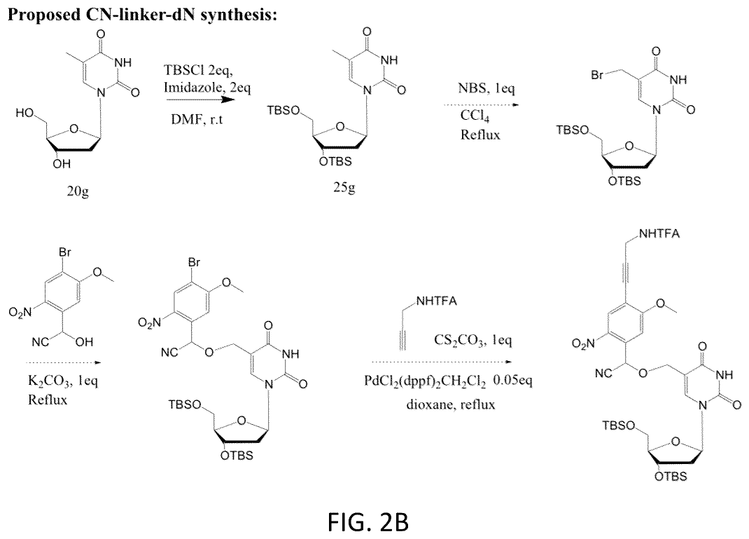

In certain embodiments, R₁ is CF₃, R₂ is H, R₃ is NO₂, R₄ is H, R₅ is C₂CH₂NH(O)C R₆ is OMe, and R₇ is H. In certain embodiments, R₁ is CN, R₂ is H, R₃ is NO₂, R₄ is H, R₅ is C₂CH₂NH(O)C—, R₆ is OMe, and R₇ is H. In certain embodiments, R₁ is tert-butyl, R₂ is H, R₃ is NO₂, R₄ is H, R₅ is —C₂CH₂NH(O)C—, R₆ is S—C₆H₆, and R₇ is H. Three non-limiting, exemplary structures according to Formula III are shown in FIG. 2A. FIG. 2B shows an exemplary scheme of a synthesis of an exemplary protected nucleotide comprising a photocleavable terminating moiety of Formula II, where R₁ is CN and R₆ is OMe.

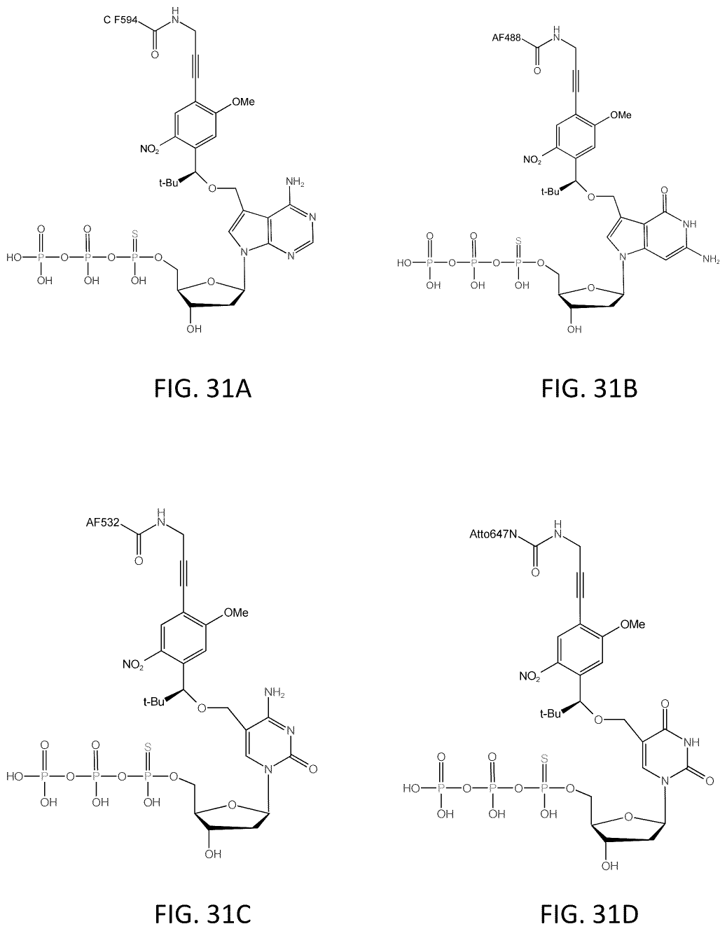

In some embodiments, a protected nucleotide is a protected dATP having the chemical structure shown in FIG. 31A. In some embodiments, a protected nucleotide is a protected dGTP having the chemical structure shown in FIG. 31B. In some embodiments, a protected nucleotide is a protected dCTP having the chemical structure shown in FIG. 31C. In some embodiments, a protected nucleotide is a protected dTTP having the chemical structure shown in FIG. 31D. In certain embodiments, a pool of nucleotides comprises protected dATPs having the chemical structure shown in FIG. 31A, protected dGTPs having the chemical structure shown in FIG. 31B, protected dCTPs having the chemical structure shown in FIG. 31C, and/or protected dTTPs having the chemical structure shown in FIG. 31D. Although each chemical structure shown in FIGS. 31A-31D is associated with a particular fluorescent moiety (e.g., CF®594 for the protected dATP, Alexa Fluor® 488 for the protected dGTP, Alexa Fluor® 532 for the protected dCTP, and Atto647N for the protected dTTP), any suitable fluorescent moiety (including any disclosed herein) may be associated with any protected nucleotide described herein.

Other suitable protected nucleotides are disclosed in U.S. Pat. No. 7,897,737, issued Mar. 1, 2011, entitled “3′-OH Unblocked, Nucleotides and Nucleosides, Base Modified with Photocleavable, Terminating Groups and Methods for Their Use in DNA Sequencing”; U.S. Pat. No. 7,965,352, issued Jun. 21, 2011, and entitled “3′-OH Unblocked, Nucleotides and Nucleosides, Base Modified with Photocleavable, Terminating Groups and Methods for Their Use in DNA Sequencing”; U.S. Pat. No. 8,361,727, issued Jan. 29, 2013, and entitled “3′-OH Unblocked, Nucleotides and Nucleosides, Base Modified with Photocleavable, Terminating Groups and Methods for Their Use in DNA Sequencing”; U.S. Pat. No. 8,969,535, issued Mar. 3, 2015, and entitled “Photocleavable Labeled Nucleotides and Nucleosides and Methods for Their Use in DNA Sequencing”; U.S. Pat. No. 7,893,227, issued Feb. 22, 2011, and entitled “3′-OH Unblocked Nucleotides and Nucleosides Base Modified with Non-Cleavable, Terminating Groups and Methods for Their Use in DNA Sequencing”; U.S. Pat. No. 8,198,029, issued Jun. 12, 2012, and entitled “3′-OH Unblocked Nucleotides and Nucleosides Base Modified with Non-Cleavable, Terminating Groups and Methods for Their Use in DNA Sequencing”; U.S. Pat. No. 8,148,503, issued Apr. 3, 2012, and entitled “Nucleotides and Nucleosides and Methods for Their Use in DNA Sequencing”; U.S. Pat. No. 8,497,360, issued Jul. 30, 2013, and entitled “Nucleotides and Nucleosides and Methods for Their Use in DNA Sequencing”; U.S. Pat. No. 8,877,905, issued Nov. 4, 2014, and entitled “Nucleotides and Nucleosides and Methods for Their Use in DNA Sequencing”; U.S. Pat. No. 9,200,319, issued Dec. 1, 2015, and entitled “Nucleotides and Nucleosides and Methods for Their Use in DNA Sequencing”; U.S. Pat. No. 8,889,860, issued Nov. 18, 2014, and entitled “3′-OH Unblocked, Fast Photocleavable Terminating Nucleotides and Methods for Nucleic Acid Sequencing”; U.S. Pat. No. 9,399,798, issued Jul. 26, 2016, and entitled “3′-OH Unblocked, Fast Photocleavable Terminating Nucleotides and Methods for Nucleic Acid Sequencing”; U.S. Pat. No. 9,689,035, issued Jun. 27, 2017, and entitled “3′-OH Unblocked, Fast Photocleavable Terminating Nucleotides and Methods for Nucleic Acid Sequencing”; U.S. Pat. No. 10,041,115, issued Aug. 7, 2018, and entitled “3′-OH Unblocked, Fast Photocleavable Terminating Nucleotides and Methods for Nucleic Acid Sequencing”; and U.S. Pat. No. 11,001,886, issued May 11, 2021, and entitled “5-Methoxy, 3′-OH Unblocked, Fast Photocleavable Terminating Nucleotides and Methods for Nucleic Acid Sequencing,” all of which are herein incorporated by reference in their entireties.

### Solution Phase Polynucleotides

In some embodiments, the reservoir comprises an aqueous solution. In certain cases, the aqueous solution of the reservoir comprises one or more solution phase polynucleotides. “Solution phase polynucleotide” refers to an oligomer, probe, or plurality of nucleobase residues that is present in the aqueous solution and not immobilized to a surface of the substrate, wherein the solution phase polynucleotide has binding specificity to a target nucleic acid sequence and/or daughter strand. Solution phase polynucleotides may include one or more primers that facilitate, e.g., amplification and/or sequencing of a target nucleic acid (e.g., RPA primers, LAMP primers, RCA primers (e.g., padlock probes), a WildFire forward or reverse primer, or sequencing primers). In some embodiments, a solution phase polynucleotide comprises a primer capable of annealing to a daughter strand, e.g., proximal to the 3′ end of a daughter strand. For example, a target nucleic acid may anneal to a substrate polynucleotide, which is elongated by a polymerase to produce a daughter strand (e.g., an amplicon), which can then bind to a solution phase polynucleotide (e.g., at the 3′ end of the daughter strand) which is elongated to produce a further daughter strand. In some embodiments, evanescent wave imaging is used to sequence a daughter strand as it is synthesized (as described herein), e.g., using a substrate polynucleotide as template.

As described elsewhere herein, in some embodiments, a method comprises nucleic acid amplification, e.g., recombinase polymerase amplification (RPA), loop-mediated amplification (LAMP), rolling circle amplification (RCA), or WildFire. In some embodiments, a device described herein configured to employ an amplification method comprises one or more solution phase polynucleotides compatible with a selected amplification methodology.

In some embodiments, a method described herein may comprise or a device described herein may be configured to employ LAMP amplification. In some such embodiments, a spot may comprise a pool of substrate constructs comprising substrate polynucleotides corresponding to one type of LAMP primer (e.g., FIP or BIP). In other such embodiments, a spot may comprise a pool of substrate constructs comprising substrate polynucleotides comprising a nucleic acid sequence complementary to a daughter strand produced by LAMP amplification but not a LAMP primer. In some embodiments, the aqueous solution comprises solution phase polynucleotides comprising LAMP primers not including those immobilized as part of a substrate construct. In some embodiments, the aqueous solution comprises solution phase polynucleotides comprising all LAMP primers for a given LAMP amplification (including solution phase versions of the one or more LAMP primers immobilized in substrate constructs). In some embodiments, a spot may comprise a pool of substrate constructs comprising substrate polynucleotides corresponding to two types of LAMP primer (e.g., FIP and BIP).

In some embodiments, a method described herein may comprise or a device described herein may be configured to employ RPA amplification. In some such embodiments, a spot may comprise a pool of substrate constructs comprising substrate polynucleotides corresponding to a forward or reverse primer with complementarity to a target nucleic acid. In some embodiments, the aqueous solution comprises solution phase polynucleotides comprising RPA primers not including those immobilized as part of a substrate construct. In some embodiments, the aqueous solution comprises solution phase polynucleotides comprising all RPA primers for a given RPA amplification (including solution phase versions of the one or more RPA primers immobilized in substrate constructs). In some embodiments, a spot may comprise a pool of substrate constructs comprising substrate polynucleotides corresponding to two types of RPA primer (e.g., forward and reverse RPA primers).

### Polymerases

In some embodiments, the reservoir of a nucleic sequencing device comprises an aqueous solution. In certain cases, the aqueous solution of the reservoir comprises a polymerase. In some embodiments, the polymerase is a DNA polymerase. Exemplary polymerases for use in the methods and devices of the disclosure include, but are not limited to, a Therminator™ polymerase, a Bacillus stearothermophilus (Bst) polymerase, a Bacillus Smithii (Bsm) polymerase, a Geobacillus sp. M (GspM) polymerase, a Thermodesulfatator indicus (Tin) polymerase, a Staphylococcus aureus (Sau) DNA polymerase, and a Taq DNA polymerase. In each case, the DNA polymerase may be a wild type polymerase or a mutant polymerase.

In some embodiments, the polymerase is capable of incorporating a modified nucleotide (e.g., a protected nucleotide) into an elongating polynucleotide (e.g., a sequencing primer annealed to a substrate polynucleotide). Some polymerases may exhibit an incorporation bias for incorporating naturally occurring or canonical nucleotides into an elongating polynucleotide relative to modified nucleotides when both modified and unmodified nucleotides are present in a solution. Without wishing to be bound by a particular theory, protecting moieties may inhibit or disrupt the structure (e.g., the active site geometry) of the polymerase, resulting in an incorporation bias against incorporation of the protected nucleotide. Incorporation bias can result in inefficient or low activity of a polymerase when extending using a protected nucleotide, which can have deleterious effects on the overall sequencing process. As used herein, incorporation bias refers to a ratio of the IC50 value (i.e., the nucleotide concentration at which the number of moles of primer equals that of the incorporated product) for the protected nucleotide to the IC50 value for the reference nucleotide (i.e., the naturally occurring or canonical nucleotide).

In some embodiments, a polymerase for use in the devices and methods of the disclosure does not exhibit incorporation bias against a modified nucleotide (e.g., a protected nucleotide described herein). In some embodiments, a polymerase for use in the devices and methods of the disclosure exhibits an incorporation bias of less than 30, 28, 26, 24, 22, 20, 18, 16, 14, 12, 10, 9, 8, 7, 6, 5, 4, 3, 2, 1.5, 1.2, or 1.1 against a modified nucleotide (e.g., a protected nucleotide described herein). In some embodiments, a polymerase is selected such that it does not exhibit sufficient incorporation bias to interfere with nucleic acid sequencing.

In some embodiments, a first polymerase is present during a first phase of a method described herein (e.g., during nucleic acid amplification) and a second polymerase is present during a second phase (e.g., during sequencing using evanescent wave imaging), wherein the first and second polymerases are different polymerases. In some embodiments, the polymerase present during a nucleic acid amplification is different from the polymerase present during sequencing using evanescent wave imaging. In some embodiments, the polymerase present during sequencing does not exhibit incorporation bias against a modified nucleotide or an incorporation bias less than a threshold value described herein, and the polymerase present during nucleic acid amplification may exhibit incorporation bias against a modified nucleotide.

In some embodiments, the pool of nucleotides has a relatively low percentage of unprotected nucleotides. Without wishing to be bound by a particular theory, one way to mitigate or eliminate the effects of a polymerase's incorporation bias for incorporating naturally occurring or canonical nucleotides into an elongating polynucleotide relative to modified nucleotides when both modified and unmodified nucleotides are present in a solution comprises decreasing the level of or eliminating unmodified nucleotides. In some embodiments, the pool of nucleotides comprises less than 5%, 4%, 3%, 2%, 1%, 0.5%, or 0.1% unprotected nucleotides (e.g., relative to the total molar concentration of nucleotides in the aqueous solution, e.g., the pool of nucleotides).

In some embodiments, a first pool of nucleotides is present during a first phase of a method described herein (e.g., during nucleic acid amplification) and a second pool of nucleotides is present during a second phase (e.g., during sequencing using evanescent wave imaging), wherein the first and second pools of nucleotides are different. In some embodiments, the pool of nucleotides present during nucleic acid amplification is different from the pool of nucleotides present during sequencing using evanescent wave imaging. In some embodiments, the pool of nucleotides present during nucleic acid amplification comprises greater than 5% (e.g., comprises at least 50%, 75%, or 100%) unmodified nucleotides. In some embodiments, the pool of nucleotides present during sequencing comprises less than 5%, 4%, 3%, 2%, 1%, 0.5%, or 0.1% unprotected nucleotides.

In some embodiments, a polymerase (e.g., a polymerase present during a first phase and/or a second phase) lacks 5′ to 3′ exonuclease activity. Some wild type polymerases possess 5′ to 3′ exonuclease activity to, for example, enable digestion of RNA primers remaining on single-stranded DNA as a polymerase elongates a replicating strand. Some methods of nucleic acid amplification (e.g., LAMP, WildFire, Rolling Circle Amplification) comprise strand displacement, however, and 5′ to 3′ exonuclease activity may interfere with such methods (e.g., by digesting elongated daughter strands).

In some embodiments, a polymerase (e.g., a polymerase present during a first phase and/or a second phase) has 3′ to 5′ exonuclease activity (also referred to as proofreading activity). In some embodiments, the polymerase lacks 3′ to 5′ exonuclease activity. Without wishing to be bound by a particular theory, 3′ to 5′ exonuclease activity in naturally occurring polymerases may remove erroneously incorporated nucleotides. Due to structural differences between protected nucleotides and naturally occurring or canonical nucleotides, protected nucleotides may be removed by 3′ to 5′ exonuclease activity at a higher rate than unprotected nucleotides. In some embodiments, the polymerase lacks 3′ to 5′ exonuclease activity capable of removing the protected nucleotide incorporated into the sequencing primer. In some embodiments, a protected nucleotide is removed at a rate at least 2, 5, 10, 20, 50, 100, 200, 500, or 1000-fold less than the rate at which a similar unprotected nucleotide is removed by a polymerase's 3′ to 5′ exonuclease activity.

In some embodiments, one or more biochemical parameters of a polymerase are configured such that the polymerase extends a sequencing primer annealed to a substrate polynucleotide by one nucleotide at least 75%, 80%, 85%, 90%, 95%, 96%, 97%, 98%, 99%, or 100% of the time termination of elongation is reversed. In some embodiments, the polymerase has a relatively high rate constant of incorporation (kₚₒₗ) for a given nucleotide (e.g., a protected nucleotide). In some embodiments, the polymerase has relatively high catalytic efficiency for a given nucleotide (e.g., a protected nucleotide). As used herein, catalytic efficiency is expressed as a ratio of the rate constant of incorporation of a nucleotide (kₚₒₗ) to the nucleotide ground state binding equilibrium constant (k_{D}).

In certain cases, the aqueous solution of the reservoir comprises a reverse transcriptase. Exemplary reverse transcriptases for use in the methods and devices of the disclosure include, but are not limited to, HIV-1 reverse transcriptase, Moloney murine leukemia virus (M-MLV) reverse transcriptase, and avian myeloblastosis virus (AMV) reverse transcriptase. In each case, the reverse transcriptase may be a wild type or mutant reverse transcriptase. In some embodiments, the aqueous solution of the reservoir comprises a polymerase described herein and a reverse transcriptase, e.g., in method or device employing a reverse transcription LAMP or reverse transcription RPA methodology.

### Biological Samples

In some embodiments, the reservoir of a nucleic sequencing device comprises an aqueous solution. In some cases, the aqueous solution of the reservoir comprises a biological sample. The disclosure is directed, in part, to detecting and/or sequencing a target nucleic acid that may be present in a biological sample. In some embodiments, a biological sample is obtained from a subject (e.g., a human subject, an animal subject). Exemplary biological samples include bodily fluids (e.g. mucus, saliva, blood, serum, plasma, amniotic fluid, sputum, urine, cerebrospinal fluid, lymph, tear fluid, feces, or gastric fluid), cell scrapings (e.g., a scraping from the mouth or interior cheek), exhaled breath particles, tissue extracts, culture media (e.g., a liquid in which a cell, such as a pathogen cell, has been grown), environmental samples, agricultural products or other foodstuffs, and their extracts. In some embodiments, a biological sample comprises blood, saliva, mucous, urine, feces, cerebrospinal fluid (CSF), and/or tissue. In some embodiments, the biological sample comprises a nasal secretion. In certain instances, for example, the sample is an anterior nares specimen. In some embodiments, the sample comprises an oral secretion (e.g., saliva).

The biological sample, in some embodiments, is collected from a subject who is suspected of having a disease the nucleic sequencing device is configured to detect, such as a coronavirus (e.g., COVID-19) and/or an influenza virus (e.g., influenza type A or influenza type B). A subject may be any mammal, for example a human, non-human primate (e.g., monkey, chimpanzee, ape, etc.), dog, cat, pig, horse, hamster, guinea pig, rat, mouse, etc. In some embodiments, a subject is a human.

### Additional Agents

In some embodiments, the aqueous solution comprises one or more additional agents that facilitate sequencing using evanescent wave imaging. The one or more additional agents may include, one or more reactive oxygen scavengers.

In some embodiments, the aqueous solution comprises one or more reactive oxygen scavengers. Without wishing to be bound by a particular theory, photochemical excited states generated by excitation of detectable moieties or photocleavable termination moieties described herein can, in the presence of molecular oxygen, lead to the generation of reactive oxygen species (ROSs), which can damage reagents used in the methods and devices described herein or components of an apparatus or device described herein (e.g., the substrate or a molecule immobilized thereto). For example, ROSs may deleteriously affect (e.g., inactivate) polymerase, substrate polynucleotides, or sequencing primers for use in the methods and devices of the disclosure. A number of reactive oxygen scavenger compounds are known to those of skill in the art, and include compounds that specifically react with particular ROSs as well as more generally and various reducing agents. These include, but are not limited to: dithiothreitol (DTT), azide (e.g., sodium azide), pyruvate (e.g., sodium pyruvate), mannitol, carboxy-PTIO (e.g., as available from Sigma-Aldrich), Trolox (e.g., as available from Hoffman-LaRoche), α-tocopherol, Ebselen, uric acid, Tiron, DMSO, dimethylthiourea (DMTU), MgSO₄, ascorbic acid, N-acetyl cysteine (NAC), one or more reactive oxygen scavenging enzymes (e.g., catalase, glucose oxidase, protocatechuate dioxygenase, or pyranose oxidase), and manganese (III)-tetrakis(4-benzoic acid)porphyrin (MnTBAP).

### Substrate

As described above, a nucleic acid sequencing device may comprise a substrate that may act as a waveguide during operation of the device. Below are described various features of such a substrate, including spots that may be deposited on the surface of a substrate. The below description may be applied to any suitable embodiment described above in relation to FIGS. 1A-1G, including any of the above description relating to substrate 106 and its features.

In some embodiments, an evanescent wave imaging apparatus is operably coupled to and/or comprises a substrate. The substrate may be capable of transmitting one or more wavelengths of light emitted by one or more light sources of the evanescent wave imaging apparatus. In some cases, the substrate transmits light from the one or more light sources by total internal reflection, and an evanescent wave emanates a limited distance from a top surface of the substrate. In certain embodiments, at least a portion of the substrate is part of the reservoir. In some instances, at least a portion of a top surface of the substrate forms at least part of a bottom surface of the reservoir and is in contact with an aqueous solution contained in the reservoir.

As described above, the substrate may have a plurality of surfaces. In some embodiments, the substrate has a top surface, a bottom surface, and one or more outer edges separating the top and bottom surfaces (e.g., 106a, 106b). For example, a substrate that is a planar rectangular prism may have a top surface, a bottom surface, and four outer edges. As a further example, a substrate that is a planar disc may have a top surface, a bottom surface, and a single curved outer edge. For a planar disc substrate having a single curved outer edge that may form a circular perimeter of the substrate, the phrase “plurality of outer edges” refers to a segment of the curved outer edge of the circular perimeter (e.g., at least 25%, 30%, 35%, 40%, 45%, 50%, 55%, 60%, 65%, 70%, or 75% of the circumference). A substrate that is planar comprises a top surface and a bottom surface that each have an area that exceeds an area of each of the one or more outer edges (and, in some cases, exceed the total area of the one or more outer edges).

In some embodiments, at least a portion (and, in some cases, substantially all) of the top surface of the substrate forms a part of the bottom surface of the reservoir and is in contact with the aqueous solution. In some embodiments, at least a portion of the top surface of the substrate is not in contact with the aqueous solution of the reservoir. In some cases, the bottom surface of the substrate (e.g., fourth surface 106d of substrate 106) faces away from the reservoir. In certain embodiments, the bottom surface of the substrate faces toward an optical imaging system comprising an image sensor. In certain embodiments, a reaction region (e.g., a region of a substrate where substrate polynucleotides are immobilized) may be substantially aligned with a sensor region (e.g., a region comprising pixels) of the image sensor of the optical imaging system. In certain embodiments, at least a portion of a reaction region may not be aligned with a sensor region of the image sensor of the optical imaging system.

### Spots

According to some embodiments, at least a portion of a top surface of the substrate (e.g., the portion forming at least a part of the bottom surface of the reservoir) comprises a plurality of spots. As used herein, the term “spot” refers to a discrete component that provides a physical and/or functional site. For example, in some embodiments, a spot comprises one or more substrate constructs (e.g., comprising a substrate polynucleotide) immobilized at a site. In some embodiments, a spot comprises a spacer (e.g., an inert spacer) configured to separate a first substrate construct from a second substrate construct.

In some embodiments, a spot on a surface (e.g., a top surface) of a substrate has a diameter (i.e., largest dimension) of about 500 μm or less, about 200 μm or less, about 100 μm or less, about 80 μm or less, about 50 μm or less, about 20 μm or less, about 10 μm or less, about 5 m or less, about 3 μm or less, about 2 μm or less, or about 1 μm or less. In certain embodiments, a spot on a surface (e.g., a top surface) of a substrate has a diameter (i.e., largest dimension) in a range from 1-5 μm, 1-10 μm, 1-20 μm, 1-50 μm, 1-80 μm, 1-100 μm, 1-200 μm, 1-500 μm, 10-20 μm, 10-50 μm, 10-80 μm, 10-100 μm, 10-200 μm, 10-500 μm, 20-50 μm, 20-80 μm, 20-100 μm, 20-200 μm, 20-500 μm, 50-100 μm, 50-200 μm, 50-500 μm, 100-200 μm, 100-500 μm, or 200-500 μm.

In some embodiments, a spot on a surface (e.g., a top surface) of a substrate has an area of at least about 1 μm², about 1.5 μm², about 2 μm², about 2.5 μm², about 3 μm², about 3.5 μm², about 4 μm², about 4.5 μm², about 5 μm², about 6 μm², about 7 μm², about 8 μm², about 9 μm², about 10 μm², about 20 μm², about 50 μm², about 100 μm², about 250 μm², about 500 μm², about 1000 μm², about 2000 μm², about 5000 μm², or about 7500 μm², or a range defined by any of the two preceding values. Alternatively or additionally, in some embodiments, a spot on a surface of a substrate may have an area of no more than about 3 μm², about 3.5 μm², about 4 μm², about 4.5 μm², about 5 μm², about 6 μm², about 7 μm², about 8 μm², about 9 μm², about 10 μm², about 20 μm², about 50 μm², about 100 μm², about 250 μm², about 500 μm², or about 1000 μm². In some embodiments, each spot on a surface of a substrate has an area of at least or no more than any two preceding values, or a range defined by two preceding values.

In some embodiments, a plurality of spots are arranged in a pattern on a surface (e.g., a top surface) of the substrate. In some embodiments, the pattern is a micro-scale or nano-scale pattern, and the spots of the pattern are separated by micrometer-scale or nanometer-scale distances or structures having micrometer-scale or nanometer-scale dimensions. In some embodiments, the pattern comprises a plurality of spots that form an array. In some embodiments, an array comprises spots separated by a distance of about 1000 μm or less, 800 μm or less, 500 μm or less, 250 μm or less, 100 μm or less, 50 μm or less, 36 μm or less, 20 μm or less, 10 μm or less, 5 μm or less, 3 μm or less, 2 μm or less, or 1 μm or less. In some embodiments, an array comprises spots separated by a distance in a range from 1-5 μm, 1-10 μm, 1-20 μm, 1-50 μm, 1-100 μm, 1-250 μm, 1-500 μm, 1-1000 μm, 5-10 μm, 5-20 μm, 5-50 μm, 5- 100 μm, 5-250 μm, 5-500 μm, 5-1000 μm, 10-20 μm, 10-50 μm, 10-100 μm, 10-250 μm, 10-500 m, 10-1000 μm, 20-50 μm, 20-100 μm, 20-250 μm, 20-500 μm, 20-1000 μm, 50-100 μm, 50-250 μm, 50-500 μm, 50-1000 μm, 100-250 μm, 100-500 μm, 100-1000 μm, or 500-1000 μm. In some embodiments, the distance between a first spot and a second spot refers to the distance between the center of the first spot and the center of the second spot.

In some embodiments, an array of spots (e.g., the distance separating the spots of an array from one another) is configured according to the nucleic acid amplification methods and sequencing methods to be used. For example, the substrate polynucleotides and/or the read lengths used in amplicon sequencing methods may be longer than the substrate polynucleotides and/or read lengths used in shotgun sequencing methods, and the spots of an array may be spaced to ensure amplification or sequencing in a first spot does not interfere with amplification or sequencing in a second spot based in part on the aforementioned lengths. In some embodiments, the distance separating the spots of an array from one another is at least 100 μm, at least 120 μm, at least 140 μm, at least 160 μm, at least 180 μm, at least 200 μm, at least 220 μm, at least 240 μm, at least 260 μm, at least 280 μm, or at least 300 μm (e.g., 100-300 μm, 100-250 μm, 100-200 μm, 100-150 μm, 150-300 μm, 150-250 μm, 150-200 μm, 200-300 μm, 200-250 μm, or 250-300 μm) in a device of the disclosure (e.g., a device configured for amplicon sequencing methods). In some embodiments, the distance separating the spots of an array from one another is at least 3 μm, at least 3.5 μm, at least 4 μm, at least 4.5 μm, at least 5 μm, at least 5.5 μm, at least 6 μm, at least 6.5 μm, at least 7 μm, at least 7.5 μm, at least 8 μm, at least 8.5 μm, or at least 9 μm (e.g., 3-9 μm, 3-8 μm, 3-7 μm, 3-6 μm, 3-5 μm, 3-4 μm, 4-9 μm, 4-8 μm, 4-7 μm, 4-6 μm, 4-5 μm, 5-9 μm, 5-8 μm, 5-7 μm, 5-6 μm, 6-9 μm, 6-8 μm, 6-7 μm, 7-9 μm, 7-8 μm, or 8-9 μm) in a device of the disclosure (e.g., a device configured for shotgun sequencing methods).

In some embodiments, a substrate comprises a surface (e.g., a top surface) comprising at least about 9, about 10, about 100, about 144, about 900, about 1×10³, about 1×10⁴, about 1×10⁵, about 1×10⁶, about 1×10⁷, about 1×10⁸, about 1×10⁹ or more spots. In some embodiments, the substrate comprises a surface (e.g., a top surface) comprising about 10 to 100, 10 to 1×10³, 10 to 1×10⁴, 10 to 1×10⁵, 10 to 1×10⁶, 10 to 1×10⁷, 10 to 1×10⁸, 10 to 1×10⁹, 100 to 1×10³, 100 to 1×10⁴, 100 to 1×10⁵, 100 to 1×10⁶, 100 to 1×10⁷, 100 to 1×10⁸, 100 to 1×10⁹, 1×10³ to 1×10⁴, 1×10³ to 1×10⁵, 1×10³ to 1×10⁶, 1×10³ to 1×10⁷, 1×10³ to 1×10⁸, 1×10³ to 1×10⁹, 1×10⁴ to 1×10⁵, 1×10⁴ to 1×10⁶, 1×10⁴ to 1×10⁷, 1×10⁴ to 1×10⁸, 1×10⁴ to 1×10⁹, 1×10⁵ to 1×10⁶, 1×10⁵ to 1×10⁷, 1×10⁵ to 1×10⁸, 1×10⁵ to 1×10⁹, 1×10⁶ to 1×10⁷, 1×10⁶ to 1×10⁸, 1×10⁶ to 1×10⁹, 1×10⁷ to 1×10⁸, 1×10⁷ to 1×10⁹, or 1×10⁸ to 1×10⁹ spots.

In some embodiments, a surface of the substrate comprises a plurality of regions. Each region may comprise one or more portions. For example, the top surface of the substrate may comprise a reservoir region in contact with the aqueous solution of the reservoir and one or more peripheral regions that are not in contact with the aqueous solution of the reservoir. In some embodiments, the reservoir region comprises a reaction region comprising a plurality of spots (e.g., the reaction region may be patterned). In some embodiments, the reservoir region comprises an inactive region that does not comprise spots (e.g., the inactive region may not be patterned). In some embodiments, the inactive region is an interstitial space of the reaction region. In some embodiments, a region of the surface of the substrate (e.g., the reservoir region) comprises two or more portions, wherein each portion is patterned and comprises a plurality of spots. In some embodiments, a first portion of the reservoir region comprises a first layer and a second layer, and a second portion of the reservoir comprises a first layer and does not comprise a second layer. Without wishing to be bound by a particular theory, patterns of spots can be produced on the surface of a substrate by selectively applying the first layer and/or second layer; such selective application can provide reactive sites or functionalized surface sites for the attachment of, e.g., substrate constructs, in desired locations on the surface. In some embodiments, additional layers (e.g., a third layer, a fourth layer, and so forth) are applied to a portion comprising the first layer and the second layer, and each additional layer may provide further pluralities of functional groups (e.g., different from the first and/or second pluralities of functional groups).

In some embodiments, the surface of at least a portion of a first region of the substrate is hydrophilic. For example, at least a portion of a first region (e.g., a first layer) may comprise a plurality of hydrophilic functional groups. In some embodiments, the surface of at least a portion of a first region of the substrate is hydrophobic. The surface of at least a portion of the second region may be hydrophilic or hydrophobic. In some embodiments, a first and a second region, or portions of either thereof, have opposite characteristics with regard to hydrophobicity and hydrophilicity; for example, a first portion of a region may be hydrophilic and a second portion of the region that is an interstitial space of the first portion may be hydrophobic.

In some embodiments, at least a portion of a surface (e.g., a top surface, a reaction region) of the substrate is treated with one or more surface-activating agents.

In certain embodiments, at least a portion of the surface (e.g., a top surface, a reaction region) is coated with a molecule (e.g., a polymer, a small molecule) comprising one or more surface-active moieties and one or more bioconjugation moieties. Surface-active moieties generally refer to moieties configured to bind to or otherwise interact with a surface or coating. Non-limiting examples of suitable surface-active moieties include silane moieties, phosphonate moieties, and bisphosphonate moieties. In some instances, for example, a silane moiety may bind to a surface or coating comprising a silicon oxide (e.g., quartz). In some instances, a phosphonate or bisphosphonate moiety may bind to a surface or coating comprising a zirconium oxide, a titanium oxide, a tantalum oxide, and/or an aluminum oxide. Bioconjugation moieties generally refer to moieties configured to bind to or otherwise interact with one or more biomolecules (e.g., amine-modified oligonucleotides, azide-modified oligonucleotides). Non-limiting examples of suitable bioconjugation moieties include azide moieties, N-hydroxysuccinimide (NHS) moieties, amine moieties, alkyne moieties, and strained alkyne moieties (e.g., dibenzocyclooctyne (DBCO) moieties). In some instances, for example, a bioconjugation moiety may bind to an amine, azide, alkyne, or strained alkyne (e.g., DBCO) moiety of a modified oligonucleotide. In certain embodiments, a bioconjugation moiety comprises a click chemistry functional group configured to bind an oligonucleotide comprising a corresponding click chemistry functional group.

In certain embodiments, the material of the substrate is silica-based (e.g., quartz). In some embodiments, the surface (e.g., the top surface) of a substrate comprising a silica-based material is chemically modified (e.g., by attachment of one or more functional groups), also referred to herein as surface activation. In some embodiments, surface activation comprises application of one or more layers comprising one or more surface-activating agents to a region or portion of a region of a surface of the substrate. In some embodiments, the surface-activating agent comprises an organosilane compound. In some embodiments, the surface-activating agent comprises (3-aminopropyl)-triethoxysilane (APTES), (3-aminopropyl)-trimethoxysilane (APTMS), (3-aminopropyl)-diethoxy-methylsilane (APDEMS), (3-aminopropyl)-dimethyl-ethoxysilane (APDMES), 7-methacryloxypropyltrimethoxysilane (also known as “Bind Silane” or “Crosslink Silane”), monoethoxydimethylsilylbutanal, 3-mercaptopropyl-trimethoxysilane, and/or 3-glycidyloxypropyl trimethoxysilane. In some embodiments, the surface-activating agent comprises a chlorosilane compound (e.g., a mono-, di-, or tri-chlorosilane compound).

In an exemplary preparation of an exemplary surface of a substrate (e.g., a silica-based substrate), the exemplary surface may be treated (e.g., by exposing the substrate to a plasma cycle under vacuum and an O₂ stream) to form a plurality of hydroxyl groups on the surface. In some embodiments, the hydroxylated surface may be silanized by an aminosilane (e.g., APTES) to form a plurality of amine functional groups on the surface. The amine functional groups may undergo further treatment (e.g., through reaction with one or more N-hydroxysuccinimide (NHS)-containing compounds). In certain non-limiting embodiments, for example, the amine functional groups may be reacted with compounds comprising an NHS ester and one or more click chemistry functional groups to introduce a plurality of click chemistry functional groups (e.g., dibenzocyclooctyne (DBCO), trans-cyclooctene (TCO)) at the silanized sites. In some embodiments, the click chemistry functional groups may be conjugated to corresponding click chemistry functional groups on the substrate polynucleotides (e.g., azide-modified substrate polynucleotides). In certain embodiments, sulfo-NHS-acetate may be used to block free amines on the surface (e.g., to reduce or prevent dNTP binding to the surface), which may advantageously reduce background noise.

In some embodiments, a substrate (e.g., a silicon oxide-based substrate) comprises one or more layers comprising a silane-containing molecule (e.g., a polymer, a small molecule) on at least a portion of a surface of the substrate. In some embodiments, one or more layers comprising a silane-containing molecule (e.g., a polymer, a small molecule) are deposited on at least a portion of a surface of a substrate. The silane-containing molecule may comprise one or more silane moieties and one or more bioconjugation moieties. In certain embodiments, the silane-containing molecule is a polymer comprising one or more silane moieties and one or more bioconjugation moieties (e.g., azide moieties, amine moieties, NHS moieties, DBCO moieties). In some cases, the polymer may comprise an acrylate and/or methacrylate polymer. In some instances, the silane-containing polymer is a copolymer of N,N-dimethylacrylamide (DMA), acryloyloxysuccinimide (NAS), and 3-(trimethoxysilyl)propyl methacrylate (MAPS). Non-limiting examples of suitable silane-containing polymers include MCP4 (Lucidant Polymers) and MCP2 (Lucidant Polymers).

Some aspects are directed to a method of preparing a substrate (e.g., a silicon oxide-based substrate) for sequencing with one or more layers comprising a silane-containing molecule. In some embodiments, the method comprises cleaning at least a portion of a surface of the substrate (e.g., to obtain a relatively clean, defect-free surface). In certain embodiments, cleaning at least the portion of the substrate comprises mechanically polishing at least the portion of the substrate. In certain embodiments, cleaning at least the portion of the substrate comprises sonicating at least the portion of the substrate in one or more solvents (e.g., isopropanol and/or acetone). In certain embodiments, cleaning at least the portion of the substrate comprises washing at least the portion of the substrate with water (e.g., ultrapure, deionized water).

In some embodiments, the method comprises activating at least the portion of the substrate. In certain embodiments, activating the portion of the substrate comprises exposing the portion of the substrate to plasma (e.g., O₂ plasma).

In some embodiments, the method comprises depositing one or more layers of a silane-containing molecule (e.g., a polymer, a small molecule) on the activated portion of the substrate. The silane-containing molecule may be deposited according to any deposition method. Non-limiting examples of suitable deposition methods include spin coating, sputtering, electron beam deposition, thermal evaporation, chemical vapor deposition, atomic layer deposition, and pulsed laser deposition. In certain instances, the method comprises spin coating.

In some embodiments, the method comprises spotting an oligonucleotide-containing solution on the one or more layers of the silane-containing molecule. In certain embodiments, the spots may be arranged in an array or other regular arrangement. In certain embodiments, the spots may be irregularly arranged. In some cases, the spotting may be performed at relatively low humidity. In some cases, the relative humidity during spotting may be about 50% or less, 45% or less, 40% or less, 35% or less, or 30% or less. In certain cases, the relative humidity during spotting may be in a range from 30% to 35%, 30% to 40%, 30% to 45%, 30% to 50%, 35% to 45%, 35% to 50%, or 40% to 50%.

In some embodiments, the method comprises reacting a surface of the one or more layers of the silane-containing molecule with one or more passivating agents. The one or more passivating agents may react with one or more moieties of the silane-containing molecule, which may advantageously reduce background noise. In certain embodiments, the one or more passivating agents comprise ethanolamine. In certain embodiments, the one or more passivating agents comprise an mPEG-amine. In certain embodiments, the one or more passivating agents comprise a betaine comprising a primary amine for NHS coupling (e.g., a sulfonate and/or carboxylate-based betaine).

In some embodiments, a substrate (e.g., a silicon oxide-based substrate, an aluminum oxide-based substrate) comprises one or more first layers comprising a zirconium oxide, titanium oxide, tantalum oxide, and/or aluminum oxide on at least a portion of a surface of the substrate. In certain embodiments, one or more first layers comprising a zirconium oxide, titanium oxide, tantalum oxide, and/or aluminum oxide are deposited on at least a portion of a surface of a substrate. In some cases, the one or more first layers are in direct physical contact with the substrate. In some cases, one or more intervening layers are positioned between the substrate and the one or more first layers.

In some embodiments, the substrate further comprises one or more second layers comprising a polymer and/or a small molecule configured to react with the one or more first layers on at least a portion of the one or more first layers. In some embodiments, one or more second layers comprising a polymer and/or a small molecule configured to react with the one or more first layers may be deposited on at least a portion of the one or more first layers. The polymer and/or small molecule may comprise one or more surface-active moieties (e.g., phosphonate, bisphosphonate, and/or silane moieties) and one or more bioconjugation moieties (e.g., azide, amine, NHS, and/or DBCO moieties).

Some aspects may be directed to method of preparing a substrate (e.g., a silicon oxide-based substrate, an aluminum oxide-based substrate) for sequencing with one or more first layers comprising a zirconium oxide, titanium oxide, tantalum oxide, and/or aluminum oxide. In some embodiments, the method comprises cleaning at least a portion of a surface of the substrate (e.g., to obtain a relatively clean, defect-free surface). In certain embodiments, cleaning at least the portion of the substrate comprises mechanically polishing at least the portion of the substrate. In certain embodiments, cleaning at least the portion of the substrate comprises sonicating at least the portion of the substrate in one or more solvents (e.g., isopropanol and/or acetone). In certain embodiments, cleaning at least the portion of the substrate comprises washing at least the portion of the substrate with water (e.g., ultrapure, deionized water).

In some embodiments, the method comprises activating the portion of the substrate. In certain embodiments, activating the portion of the substrate comprises exposing the portion of the substrate to plasma (e.g., O₂ plasma). In certain embodiments, activating the portion of the substrate comprises exposing the portion of the substrate to an oxidizing acid (e.g., nitric acid).

In some embodiments, the method comprises depositing one or more first layers comprising a zirconium oxide, titanium oxide, tantalum oxide, and/or aluminum oxide on at least a portion of a surface of the substrate. The substrate may comprise a silicon oxide (e.g., quartz) material or an aluminum oxide (e.g., sapphire) material. In certain embodiments, the zirconium oxide is a zirconium alkoxide. A non-limiting example of a suitable zirconium alkoxide is zirconium (IV) propoxide. In some embodiments, the one or more first layers are in direct physical contact with the substrate. In some embodiments, one or more intervening layers are positioned between the substrate and the one or more first layers.

In some embodiments, the method comprises depositing one or more second layers comprising a polymer and/or a small molecule configured to react with the one or more first layers on at least a portion of the one or more first layers. The polymer and/or small molecule may be deposited according to any deposition method. Non-limiting examples of suitable deposition methods include spin coating, sputtering, electron beam deposition, thermal evaporation, chemical vapor deposition, atomic layer deposition, and pulsed laser deposition. In certain instances, the method comprises spin coating. In some embodiments the polymer and/or small molecule comprise one or more surface-active moieties (e.g., phosphonate, bisphosphonate, and/or silane moieties) and one or more bioconjugation moieties (e.g., azide, amine, NHS, and/or DBCO moieties). In certain embodiments, for example, the polymer comprises an inert backbone, one or more side chains comprising one or more surface-active moieties (e.g., phosphonate moieties, bisphosphonate, and/or silane moieties), and one or more side chains comprising one or more bioconjugation moieties (e.g., azide, amine, NHS, and/or DBCO moieties). The backbone may be any suitable inert backbone. Examples of inert backbones include, but are not limited to, hydroxyethyl acrylamide and dimethylacrylamide. A non-limiting example of a suitable polymer is a polymer having the chemical structure shown in FIG. 23. In some cases, the molecular weight of the polymer may be adjusted to improve manufacturability and/or synthesis results. As one example, bisphosphonate content of the polymer may be increased to achieve a thicker, gel-like coating. As another example, azide content of the polymer may be increased to increase oligonucleotide surface density. In certain embodiments, the small molecule comprises one or more surface-active moieties (e.g., phosphonate, bisphosphonate, and/or silane moieties) separated from one or more bioconjugation moieties (e.g., azide, amine, NHS, and/or DBCO moieties) by one or more spacer atoms (e.g., carbon, carbon and oxygen such as polyethylene glycol). In certain cases, the small molecule may be derived from alendronate.

In some embodiments, the method comprises spotting an oligonucleotide-containing solution on the one or more second layers of the polymer and/or small molecule. In certain embodiments, the spots may be arranged in an array or other regular arrangement. In certain embodiments, the spots may be irregularly arranged. In some cases, the spotting may be performed at relatively low humidity. In some cases, the relative humidity during spotting may be about 50% or less, 45% or less, 40% or less, 35% or less, or 30% or less. In certain cases, the relative humidity during spotting may be in a range from 30% to 35%, 30% to 40%, 30% to 45%, 30% to 50%, 35% to 45%, 35% to 50%, or 40% to 50%.

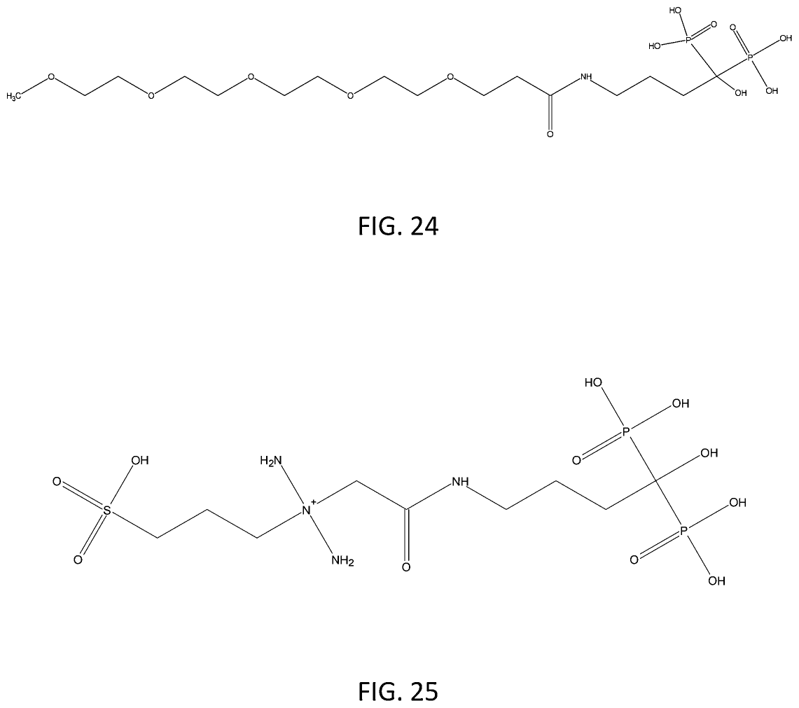

In some cases, the method comprises reacting a surface of the one or more second layers comprising the polymer and/or small molecule with one or more passivating agents. Non-limiting examples of passivating agents include a compound having a chemical structure as shown in FIG. 24 and a compound having a chemical structure as shown in FIG. 25. The compound having the chemical structure as shown in FIG. 24 is derived from alendronate and a short methoxy-polyethyleneglycol carboxylic acid. The compound having the chemical structure shown in FIG. 25 is derived from alendronate and comprises a zwitterionic moiety. In some embodiments, the one or more passivating agents comprise one or more carbohydrate molecules comprising one or more phosphonate and/or bisphosphonate moieties. A non-limiting example of a carbohydrate-based passivating agent is chitosan decorated with alendronate.

In some embodiments, the surface-activating agent comprises atomic gold. In certain embodiments, one or more layers comprising atomic gold may be deposited on at least a portion of a substrate. In certain cases, the one or more layers comprising atomic gold may facilitate thiol conjugation.

In some embodiments, a surface of a substrate is prepared using spin coating. In some embodiments, a surface of the substrate is prepared using a stamping methodology. In some embodiments, a surface of the substrate is prepared using a transfer methodology.

### Spot Contents

In some embodiments, the top surface of the substrate comprises a plurality of spots. In some embodiments, each spot of the plurality comprises a pool of substrate constructs. In some embodiments, each spot comprises a single pool of substrate constructs. In some embodiments, the substrate constructs of a pool are immobilized to the top surface of the substrate in a spot. The disclosure is directed, in part, to devices comprising a substrate, wherein one or more substrate constructs are immobilized to a surface of the substrate (e.g., the top surface, e.g., the reservoir region, e.g., one or more spots).

In some embodiments, a substrate construct comprises a tether and an analyte binding agent. As used herein, a “tether” refers to any agent capable of immobilizing an analyte binding agent (e.g., a substrate polynucleotide) to a surface (e.g., a top surface of the substrate). Analyte binding agents may be any structure capable of binding to an analyte in a sample (e.g., a biological sample from a subject) to facilitate detection and/or identification of the analyte. In some embodiments, an analyte binding agent is an antigen binding domain, e.g., an antibody or a antigen binding portion thereof (e.g., an scFv). In some embodiments, an analyte binding agent is a nucleic acid; nucleic acid analyte binding agents are also referred to herein as substrate polynucleotides. In some embodiments, an analyte binding agent binds to an analyte that is present in a sample from a subject (e.g., a protein or nucleic acid associated with a pathogenic infection in the subject). In some embodiments, an analyte binding agent binds to a label added to the sample, e.g., the analyte binding agent may be a secondary antibody that binds to a label that is a primary antibody with specificity to an analyte present in a biological sample. In some embodiments, an analyte binding agent binds to a tagged or complementary sequence of a target nucleic acid analyte, e.g., the analyte binding agent may be a substrate polynucleotide complementary to a tag sequence or a daughter strand produced by amplification of a target nucleic acid.

In some embodiments, a substrate construct comprises a tether and a substrate polynucleotide. “Substrate polynucleotide” refers to an oligomer, probe, or plurality of nucleobase residues that has binding specificity to a target nucleic acid sequence and/or daughter strand and that is part of a substrate construct. Substrate polynucleotides are generally combined with tethers to form substrate constructs. In some embodiments, a substrate polynucleotide comprises the sequence of a target nucleic acid or a sequence complementary to a target nucleic acid or a portion of either thereof, e.g., after being elongated. “Substrate constructs” are reagents for detection of analytes. In some embodiments, detection and/or identification of an analyte comprises sequencing a target nucleic acid, and a substrate construct is a reagent for template-directed synthesis of daughter strands. “Daughter strand” refers to the product of template-directed elongation of a substrate polynucleotide or of a solution phase polynucleotide by a polymerase; for example, a substrate polynucleotide extended by a polymerase is both a daughter strand and a substrate polynucleotide. An “amplicon” refers to a daughter strand produced in the context of a nucleic acid amplification method. In some embodiments, substrate polynucleotides or substrate constructs are provided in the form of libraries. In some embodiments, substrate constructs contain: a substrate polynucleotide capable of complementary binding to a target nucleic acid, and either a tether or tether attachment sites to which a tether may be bonded.

### Exemplary Substrate Constructs are Provided Herein

In some embodiments, a spot comprises a pool of substrate constructs (prior to amplifying or sequencing a target nucleic acid) comprising a plurality of substrate polynucleotides having the same nucleic acid sequence. For example, each substrate polynucleotide of a pool may comprise a primer for use in an amplification method described herein, e.g., a forward primer or reverse primer for RPA, or a LAMP primer (e.g., FIP or BIP).

In some embodiments, a spot comprises a pool of substrate constructs comprising a plurality of substrate polynucleotides each comprising a different nucleic acid sequence. For example, in some embodiments, a spot comprises a pool of substrate constructs containing two different substrate polynucleotides. In some embodiments, the two different substrate polynucleotides comprise primers capable of binding to different portions of the same target nucleic acid, e.g., a forward and reverse primer. In some embodiments, the pool of substrate constructs (prior to amplifying or sequencing a target nucleic acid) comprises two groups of substrate polynucleotides, wherein each group has a different nucleic acid sequence than the other. For example, a first group of substrate polynucleotides may comprise a first primer for use in an amplification method described herein, e.g., a forward primer or reverse primer for RPA or a LAMP primer (e.g., FIP or BIP), and a second group of substrate polynucleotides may comprise a second primer for use in an amplification method described herein different than the first (e.g., the reverse primer if the first primer was a forward primer, or vice versa), e.g., a forward primer or reverse primer for RPA or a LAMP primer (e.g., FIP or BIP).

In some embodiments, the nucleic acid sequence of the substrate polynucleotides of a pool of substrate constructs in a first spot is different from the nucleic acid sequence of the substrate polynucleotides of a pool of substrate constructs in a second spot. In some embodiments, the nucleic acid sequence of the substrate polynucleotides of a pool of substrate constructs in a spot is different from the nucleic acid sequence of the substrate polynucleotides of the pools of substrate constructs in each other spot, e.g., in a given portion of the region or in the entire reservoir region.

In some embodiments, the reservoir region comprises a plurality of spots, wherein each spot contains a pool of substrate constructs containing substrate polynucleotides having the same nucleic acid sequence. In some embodiments, in the remaining spots of the reservoir region (besides the aforementioned plurality) the nucleic acid sequence of the substrate polynucleotides of a pool of substrate constructs in a spot is different from the nucleic acid sequence of the substrate polynucleotides of the pools of substrate constructs in each other spot. For example, in a method or device configured for sequencing using an amplicon methodology, the reservoir region may comprise one or more (e.g., 2-4) spots that function as controls, with each spot containing substrate polynucleotides having the same nucleic acid sequence, and the remaining spots containing substrate polynucleotides having distinct nucleic acid sequences.

In some embodiments, the reservoir region comprises a plurality of portions, and each portion comprises a set of spots wherein each spot of a set comprises a pool of substrate constructs comprising substrate polynucleotides having different nucleic acid sequences than those of the pools of substrate constructs in each other spot of the set. In some embodiments, there is nucleic acid sequence overlap between spots in different sets, e.g., a spot in a first set comprises substrate polynucleotides having the same nucleic acid sequence as a spot in a second set. In some such embodiments, two or more portions of the plurality (e.g., each portion) comprise spots having pools of substrate constructs comprising substrate polynucleotides having the same nucleic acid sequence. For example, each portion may comprise a spot having a pool of substrate constructs with substrate polynucleotides having the same nucleic acid sequence (e.g., a control spot in each set).

In a further example, in some embodiments the nucleic acid sequence of the substrate polynucleotides of a pool of substrate constructs in each spot is different from the nucleic acid sequence of the substrate polynucleotides of a pool of substrate constructs in each other spot in the reservoir region. For example, an exemplary top surface of a substrate may comprise 10-50 spots, wherein each spot comprises a pool of substrate constructs comprising substrate polynucleotides, wherein the nucleic acid sequence of the substrate polynucleotides in a spot is different from the nucleic acid sequence of the substrate polynucleotides in each other spot.

In some embodiments, a method described herein employs or a device described herein is configured for an amplicon methodology. An amplicon methodology refers to sequencing where the nucleic acid sequences of the substrate polynucleotides of the substrate constructs are selected to detect and/or determine the sequence of one or more target nucleic acids or daughter strands having one or more specific nucleic acid sequences. In some such embodiments, each spot comprises a pool of substrate constructs comprising substrate polynucleotides capable of binding to a single nucleic acid sequence of a target nucleic acid and/or daughter strand. In such a fashion, each spot may be configured for detection and/or determination of the sequence of a target nucleic acid or daughter strand having a single nucleic acid sequence. In some embodiments, a reservoir region comprises multiple spots configured for detection and/or determination of the sequence of a given nucleic acid sequence. In other embodiments, a reservoir region comprises a single spot configured for detection and/or determination of the sequence of a given nucleic acid sequence (e.g., a single spot for each nucleic acid sequence to be detected or sequenced).

In some embodiments, a plurality of spots (e.g., in a portion of a region or the reservoir region) comprise pools of substrate constructs comprising substrate polynucleotides comprising identical nucleic acid sequences. Such duplication may provide redundancy, where the sequencing information provided by one spot is verifiable or statistically validated by the sequencing information provided by each other spot of the plurality, and may provide control information, e.g., for the validity of data obtained from a portion of the reservoir region or the reservoir region. In some embodiments, spots having substrate polynucleotides with identical nucleic acid sequences are useful for methods employing shotgun sequencing methodology.

In some embodiments, a method described herein employs or a device described herein is configured for a shotgun methodology. A shotgun methodology refers to sequencing where a plurality of nucleic acid sequences that are not known prior to sequencing or not pre-selected are detected and/or sequenced. In some embodiments, a shotgun methodology comprises providing a target nucleic acid comprising one or more tag nucleic acids. In some such embodiments, the reservoir region comprises a plurality of spots, each comprising a pool of substrate constructs, wherein a plurality of spots (e.g., all spots) comprise substrate polynucleotides comprising identical nucleic acid sequences. For example, in some embodiments employing a shotgun methodology, each spot comprises substrate polynucleotides having identical nucleic acid sequences complementary to a tag sequence (e.g., a paired end tag) ligated to target nucleic acid sequences, thereby allowing any sequence comprising the tag to hybridize to a substrate polynucleotide. In some such embodiments, the concentration of target nucleic acid in the reservoir is configured to ensure one or fewer target nucleic acids contact a given spot on the top surface of the substrate.

A tether may be coupled to the substrate based on covalent or non-covalent interactions between the tether and the substrate. In some embodiments, non-covalent interactions may be selected from, but are not limited to, hydrogen bonds, hydrophobic interactions, electrostatic/ionic interactions, and van der Waals interactions. Methods for immobilizing a tether to a substrate include, but are not limited to a) chemisorption (e.g. thiol-gold); b) physical absorption (e.g., on nitrocellulose, amine, PAAH, poly(1-lysine, or diazonium ion surface); c) covalent immobilization (e.g., amines, amino, or hydrazide-modified DNA oligo nucleotides on carboxyl (with carbodiimide), aldehyde, isothiocyanate, or epoxide modified surfaces, hydrazide disulphide coupling, thiols-maleimide, thiol-mercaptosilane, or thiols-acrylamide); or d) affinity-binding (e.g. avidin or streptavidin to biotin interaction). In some embodiments, a tether comprises a polymer. In some embodiments, a tether has a generally linear dimension. In some embodiments, a tether comprises ends capable of concatenating with other tethers. Polymers suitable as tethers include, but are not limited to: polyethylene glycols, polyglycols, polypyridines, polyisocyanides, polyisocyanates, poly(triarylmethyl) methacrylates, polyaldehydes, polypyrrolinones, polyureas, polyglycol phosphodiesters, polyacrylates, polymethacrylates, polyacrylamides, polyvinyl esters, polystyrenes, polyamides, polyurethanes, polycarbonates, polybutyrates, polybutadienes, polybutyrolactones, polypyrrolidinones, polyvinylphosphonates, polyacetamides, polysaccharides, polyhyaluranates, polyamides, polyimides, polyesters, polyethylenes, polypropylenes, polystyrenes, polycarbonates, polyterephthalates, polysilanes, polyurethanes, polyethers, polyamino acids, polyglycines, polyprolines, N-substituted polylysine, polypeptides, side-chain N-substituted peptides, poly-N-substituted glycine, peptoids, side-chain carboxyl-substituted peptides, homopeptides, oligonucleotides, ribonucleic acid oligonucleotides, deoxynucleic acid oligonucleotides, oligonucleotides modified to prevent Watson-Crick base pairing, oligonucleotide analogs, polycytidylic acid, polyadenylic acid, polyuridylic acid, polythymidine, polyphosphate, polynucleotides, polyribonucleotides, polyethylene glycol-phosphodiesters, peptide polynucleotide analogues, threosyl-polynucleotide analogues, glycol-polynucleotide analogues, morpholino-polynucleotide analogues, locked nucleotide oligomer analogues, polypeptide analogues, branched polymers, comb polymers, star polymers, dendritic polymers, random, gradient and block copolymers, anionic polymers, cationic polymers, polymers forming stem-loops, rigid segments and flexible segments.

### Substrate Polynucleotides

The methods and devices described herein are, in some embodiments, intended to sequence one or more target nucleic acid sequences in human or animal subjects (e.g., subjects having or suspected of having a pathogenic infection). In certain embodiments, a test sample is obtained from a subject who has been infected by, or is suspected of having been infected by, one or more pathogens.

As used herein, the terms “subject” and “patient” are used interchangeably to refer to the human or animal subject from whom a sample was obtained. Where the subject self-collects the sample and directly practices the methods, the subject may in some cases also be referred to as a “user.” A “pathogen” is any organism capable of causing disease and may include viruses, bacterium, protozoans, prions, viroids, parasite, and/or fungi.

In some embodiments, the methods and devices described herein are configured to sequence one or more target nucleic acid sequences from a human or animal subject. In some embodiments, such a configuration comprises providing substrate polynucleotides having a nucleic acid sequence complementary to the one or more target nucleic acid sequences or to a daughter strand produced, e.g., by amplification of the one or more target nucleic acid sequences (e.g., a substrate polynucleotide having a nucleic acid sequence complementary to a daughter strand and identical to a target nucleic acid sequence or a portion thereof). In some embodiments, a target nucleic acid sequence comprises a nucleic acid sequence associated with one or more pathogens (e.g., genomic nucleic acid of a pathogen or mRNA encoding pathogen expression products), with a cancer, or with human or animal genomic sequence associated with a genetic disease or predisposition for a disease.

In some embodiments, substrate polynucleotides are configured to facilitate amplification of the one or more target nucleic acids. For example, a spot may comprise a pool of substrate constructs comprising substrate polynucleotides having the nucleic acid sequence of one of the primers used in an amplification method. In some embodiments, substrate polynucleotides are configured to hybridize to one or more daughter strands produced during the amplification of the one or more target nucleic acids. For example, a spot may comprise a pool of substrate constructs comprising substrate polynucleotides having a nucleic acid sequence complementary to a daughter strand produced by an amplification method, and optionally the substrate polynucleotide (or a daughter strand produced by elongation of the substrate polynucleotide) does not participate in the amplification method.

In some embodiments, a substrate polynucleotide comprises a sequence complementary to a target nucleic acid or daughter strand. In some embodiments, the substrate polynucleotide comprises the nucleic acid sequence of one of the primers used in an amplification method or a portion of the nucleic acid sequence of one of the primers.

In some embodiments, a substrate polynucleotide comprises one or more spacer sequences that are not complementary to a target nucleic acid or daughter strand. In some embodiments, a spacer sequence is used to adjust the length of a substrate polynucleotide, e.g., to ensure the substrate polynucleotide is accessible to a reagent of the aqueous solution (e.g., a polymerase, recombinase, or reverse transcriptase) or sufficiently within the range of the evanescent wave. In some embodiments, a substrate polynucleotide does not comprise a spacer sequence so that the substrate polynucleotide remains sufficiently within the range of the evanescent wave.

In some embodiments, a substrate polynucleotide is at least 10, 15, 20, 25, 30, 35, 40, 45, 50, 55, 60, 65, 70, 75, 80, 85, 90, 95, or 100 bases long. In some embodiments, a substrate polynucleotide is no more than 10, 15, 20, 25, 30, 35, 40, 45, 50, 55, 60, 65, 70, 75, 80, 85, 90, 95, 100, 110, 120, 130, 140, 150, or 200 bases long. In some embodiments, a substrate polynucleotide is 10-200, 10-150, 10-100, 10-90, 10-80, 10-70, 10-60, 10-50, 10-40, 10-30, 10-20, 20-200, 20-150, 20-100, 20-90, 20-80, 20-70, 20-60, 20-50, 20-40, 20-30, 30-200, 30-150, 30-100, 30-90, 30-80, 30-70, 30-60, 30-50, 30-40, 40-200, 40-150, 40-100, 40-90, 40-80, 40-70, 40-60, 40-50, 50-200, 50-150, 50-100, 50-90, 50-80, 50-70, 50-60, 60-200, 60-150, 60-100, 60-90, 60-80, 60-70, 70-200, 70-150, 70-100, 70-90, 70-80, 80-200, 80-150, 80-100, 80-90, 90-200, 90-150, 90-100, 100-200, 100-150, or 150-200 bases long. In some embodiments, a substrate polynucleotide comprises a nucleic acid sequence complementary to a target nucleic acid or daughter strand, and the nucleic acid sequence is at least 10, 15, 20, 25, 30, 35, 40, 45, 50, 55, 60, 65, 70, 75, 80, 85, 90, 95, or 100 bases long. In some embodiments, a substrate polynucleotide comprises a nucleic acid sequence complementary to a target nucleic acid or daughter strand, and the nucleic acid sequence is no more than 10, 15, 20, 25, 30, 35, 40, 45, 50, 55, 60, 65, 70, 75, 80, 85, 90, 95, or 100 bases long. In some embodiments, a substrate polynucleotide comprises a nucleic acid sequence complementary to a target nucleic acid or daughter strand, and the nucleic acid sequence is 10-100, 10-90, 10-80, 10-70, 10-60, 10-50, 10-40, 10-30, 10-20, 20-100, 20-90, 20-80, 20-70, 20-60, 20-50, 20-40, 20-30, 30-100, 30-90, 30-80, 30-70, 30-60, 30-50, 30-40, 40-100, 40-90, 40-80, 40-70, 40-60, 40-50, 50-100, 50-90, 50-80, 50-70, 50-60, 60-100, 60-90, 60-80, 60-70, 70-100, 70-90, 70-80, 80-100, 80-90, 90-200, 90-150, 90-100, 100-200, 100-150, or 150-200 bases long.

In some embodiments, the one or more pathogens comprise a viral pathogen. Non-limiting examples of viral pathogens include coronaviruses, influenza viruses, rhinoviruses, parainfluenza viruses (e.g., parainfluenza 1-4), enteroviruses, adenoviruses, respiratory syncytial viruses, and metapneumoviruses. In certain embodiments, the viral pathogen is SARS-CoV-2. In some embodiments, the viral pathogen is a variant of SARS-CoV-2. In certain instances, the variant of SARS-CoV-2 is SARS-CoV-2 D614G, a SARS-CoV-2 variant of B.1.1.7 lineage (e.g., 20B/501Y.V1 Variant of Concern (VOC) 202012/01), a SARS-CoV-2 variant of B.1.351 lineage (e.g., 20C/501Y.V2), a SARS-CoV-2 variant of P.1 lineage, a SARS-CoV-2 variant of B1.1.617.2 lineage, a SARS-CoV-2 variant of B.1.427 lineage, a SARS-CoV-2 variant of B1.1.429 lineage, a SARS-CoV-2 variant of B.1.525 lineage, a SARS-CoV-2 variant of B.1.526 lineage, a SARS-CoV-2 variant of B.1.617.1 lineage, a SARS-CoV-2 variant of B.1.16.3 lineage, a SARS-CoV-2 variant of P.2 lineage, a SARS-CoV-2 variant of B.1.1.529 lineage, a SARS CoV-2 variant of C.37 lineage, a SARS-CoV-2 variant of B.1.621 lineage, or a SARS-CoV-2 variant of B.1.621.1 lineage.

In certain embodiments, templates for amplification of a SARS-CoV-2 nucleic acid sequence are selected from regions of the virus's nucleocapsid (N) gene, envelope (E) gene, membrane (M) gene, and/or spike (S) gene. In some instances, templates were selected from regions of the SARS-CoV-2 nucleocapsid (N) gene to maximize inclusivity across known SARS-CoV-2 strains and minimize cross-reactivity with related viruses and genomes that may be presence in the sample. In some embodiments, templates were selected from one or more regions of the SARS-CoV-2 spike (S) gene and from one or more regions of the SARS-COV-2 nucleocapsid gene. In certain embodiments, templates were selected from one or more regions of the SARS-CoV-2 spike (S) gene that do not include G142, V143, Y144, Y145, E156, F157, and/or R158. In certain embodiments, templates were selected from one or more regions of the SARS-CoV-2 nucleocapsid (N) gene that do not include R203 and/or G204. In certain embodiments, the viral pathogen is an influenza virus. The influenza virus may be an influenza A virus (e.g., H1N1, H3N2) or an influenza B virus.

Other viral pathogens include, but are not limited to, adenovirus; Herpes simplex, type 1; Herpes simplex, type 2; encephalitis virus; papillomavirus (e.g., human papillomavirus); Varicella zoster virus; Epstein-Barr virus; human cytomegalovirus; human herpesvirus, type 8; BK virus; JC virus; smallpox; polio virus; hepatitis A virus; hepatitis B virus; hepatitis C virus; hepatitis D virus; hepatitis E virus; human immunodeficiency virus (HIV); human bocavirus; parvovirus B19; human astrovirus; Norwalk virus; coxsackievirus; rhinovirus; Severe acute respiratory syndrome (SARS) virus; yellow fever virus; dengue virus; West Nile virus; Guanarito virus; Junin virus; Lassa virus; Machupo virus; Sabia virus; Crimean-Congo hemorrhagic fever virus; Ebola virus; Marburg virus; measles virus; mumps virus; rubella virus; Hendra virus; Nipah virus; Rabies virus; rotavirus; orbivirus; Coltivirus; Hantavirus; Middle East Respiratory Coronavirus; Zika virus; norovirus; Chikungunya virus; and Banna virus.

In some embodiments, a viral pathogen comprises a Coronavirinae pathogen. In some embodiments, the Coronavirinae pathogen comprises an Alphacoronavirus, Betacoronavirus, Gammacoronavirus, Deltacoronavirus, Human coronavirus 229E, Human coronavirus NL63, Human coronavirus OC43, Human coronavirus HKU1, Middle East Respiratory Syndrome coronavirus (e.g., MERS-CoV), Severe acute respiratory coronavirus (e.g., SARS-CoV), or Severe acute respiratory syndrome coronavirus 2 (e.g., SARS-CoV-2) pathogen. In some embodiments, the Coronavirinae pathogen causes a pathogenic infection (e.g., a viral disease) in a subject. In some embodiments, the pathogenic infection is a coronavirus disease. In some embodiments, the coronavirus disease is Coronavirus disease 2019 (e.g., COVID-19). In some embodiments, the coronavirus disease is a variant of COVID-19. In some embodiments, the coronavirus disease is Middle East Respiratory Syndrome (MERS). In some embodiments, the coronavirus disease is Severe acute respiratory syndrome (SARS). In some embodiments, the coronavirus disease is Human coronavirus OC43 (HCoV-OC43). In some embodiments, the coronavirus disease is Human coronavirus HKU1 (HCoV-HKU1). In some embodiments, the coronavirus disease is Human coronavirus 229E (HCoV-229E). In some embodiments, the coronavirus disease is Human coronavirus NL63 (HCoV-NL63).

In some embodiments, a viral pathogen comprises an Orthomyxoviridae pathogen. In some embodiments, the Orthomyxoviridae pathogen comprises an Alphainfluenzavirus, Betainfluenzavirus, Deltainfluenzavirus, or Gammainfluenzavirus pathogen. In some embodiments, the Alphainfluenzavirus pathogen comprises an Influenza virus A pathogen. In some embodiments, the Betainfluenzavirus pathogen comprises an Influenza virus B pathogen.

In some embodiments, the Gammainfluenzavirus pathogen comprises an Influenza virus C pathogen. In some embodiments, the Orthomyxoviridae pathogen causes a pathogenic infection (e.g., a viral disease) in a subject. In some embodiments, the pathogenic infection is an influenza virus disease. In some embodiments, the influenza virus disease is Influenza A. In some embodiments, the Influenza A virus is of the subtype H1N1, H2N2, H3N2, H5N1, H7N7, H1N2, H9N2, H7N2, H7N3, or H10N7. In some embodiments, the influenza virus disease is Influenza B. In some embodiments, the Influenza B virus is of the lineage Victoria or Yamagata. In some embodiments, the influenza virus disease is Influenza C.

In some embodiments, the one or more pathogens comprise a bacterial pathogen. Non-limiting examples of bacterial pathogens include Gram-positive bacteria and Gram-negative bacteria. Bacterial pathogens include, but are not limited to, Acinetobacter baumannii, Bacillus anthracis, Bacillus subtilis, Bordetella pertussis, Borrelia burgdorferi, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, coagulase Negative Staphylococcus, Corynebacterium diphtheria, Enterococcus faecalis, Enterococcus faecium, Escherichia coli, enterotoxigenic Escherichia coli (ETEC), enteropathogenic E. coli, E. coli O157:H7, Enterobacter sp., Francisella tularensis, Haemophilus influenzae, Helicobacter pylori, Klebsiella pneumoniae, Legionella pneumophila, Leptospira interrogans, Listeria monocytogenes, Moraxella catarralis, Mycobacterium leprae, Mycobacterium tuberculosis, Mycoplasma pneumoniae, Neisseria gonorrhoeae, Neisseria meningitides, Preteus mirabilis, Proteus sps., Pseudomonas aeruginosa, Rickettsia rickettsii, Salmonella typhi, Salmonella typhimurium, Serratia marcesens, Shigella flexneri, Shigella sonnei, Staphylococcus aureus, Staphylococcus epidermidis, Staphylococcus saprophyticus, Streptococcus agalactiae, Streptococcus mutans, Streptococcus pneumoniae, Streptococcus pyogenes, Treponema pallidum, Vibrio cholerae, and Yersinia pestis.

In some embodiments, the one or more pathogens comprise a fungal pathogen. Non-limiting examples of fungal pathogens include, but are not limited to, Ascomycota (e.g., Fusarium oxysporum, Pneumocystis jirovecii, Aspergillus spp., Coccidioides immitis/posadasii, Candida albicans), Basidiomycota (e.g., Filobasidiella neoformans, Trichosporon), Microsporidia (e.g., Encephalitozoon cuniculi, Enterocytozoon bieneusi), and Mucoromycotina (e.g., Mucor circinelloides, Rhizopus oryzae, Lichtheimia corymbifera).

In some embodiments, the one or more pathogens comprise a protozoan pathogen. Non-limiting examples of protozoan pathogens include, but are not limited to, Entamoeba histolytica, Giardia lambila, Trichomonas vaginalis, Trypanosoma brucei, T. cruzi, Leishmania donovani, Balantidium coli, Toxoplasma gondii, Plasmodium spp., and Babesia microti.

In some embodiments, the one or more pathogens comprise a parasitic pathogen. Non-limiting examples of parasitic pathogens include, but are not limited to, Acanthamoeba, Anisakis, Ascaris lumbricoides, botfly, Balantidium coli, bedbug, Cestoda, chiggers, Cochliomyia hominivorax, Entamoeba histolytica, Fasciola hepatica, Giardia lamblia, hookworm, Leishmania, Linguatula serrata, liver fluke, Loa loa, Paragonimus, pinworm, Plasmodium falciparum, Schistosoma, Strongyloides stercoralis, mite, tapeworm, Toxoplasma gondii, Trypanosoma, whipworm, and Wuchereria bancrofti.

In some embodiments, the methods and devices of the present disclosure are configured to sequence a target nucleic acid sequence of an animal pathogen. As will be understood, an animal pathogen may be considered a human pathogen in certain instances, for example in cases where a pathogen originating in a non-human animal infects a human. Examples of animal pathogens include, but are not limited to, bovine rhinotracheitis virus, bovine herpesvirus, distemper, parainfluenza, canine adenovirus, rhinotracheitis virus, calicivirus, canine parvovirus, Borrelia burgdorferi (Lyme disease), Bordetella bronchiseptica (kennel cough), canine parainfluenza, leptospirosis, feline immunodeficiency virus, feline leukemia virus, Dirofilaria immitis (heartworm), feline herpesvirus, Chlamydia infections, Bordetella infections, equine influenza, rhinopneumonitis (equine herpesevirus), equine encephalomyelitis, West Nile virus (equine), Streptococcus equi, tetanus (Clostridium tetani), equine protozoal myeloencephalitis, bovine respiratory disease complex, clostridial disease, bovine respiratory syncytial virus, bovine viral diarrhea, Haemophilus somnus, Pasteurella haemolytica, and Pastuerella multocida.

In some cases, a subject may be infected with a single type of pathogen or with multiple types of pathogens simultaneously. A “pathogenic infection” may encompass any of a viral infection, a bacterial infection, protozoan infection, prion disease, viroid infection, parasitic infection, or fungal infection. Any pathogenic infection may be detected using the rapid diagnostic tests, systems, and methods of the present disclosure.

In some embodiments, a pathogenic infection comprises any one of: African sleeping sickness, Amebiasis, Ascariasis, Bronchitis, Candidiasis, Chickenpox, Cholera, Coronavirus, Human coronavirus OC43 (HCoV-OC43), Human coronavirus HKU1 (HCoV-HKU1), Human coronavirus 229E (HCoV-229E), Human coronavirus NL63 (HCoV-NL63), Middle East Respiratory Syndrome (MERS), Severe acute respiratory syndrome (SARS), Coronavirus disease 2019 (COVID-19), Cryptosporidiosis, Dengue fever, Diphtheria, Elephantiasis, Gastric ulcers, Giardiasis, Gonorrhea, Hepatitis A, Hepatitis B, Hepatitis C, Herpes simplex 1, Herpes simplex 2, Hookworm, Influenza, Influenza A, Influenza A(H1N1), Influenza A(H2N2), Influenza A(H3N2), Influenza A(H5N1), Influenza A(H7N7), Influenza A(H1N2), Influenza A(H9N2), Influenza A(H7N2), Influenza A(H7N3), Influenza A(H10N7), Influenza B, Influenza B(Victoria), Influenza B(Yamagata), Influenza C, Leprosy, Malaria, Measles, Meningitis, Mononucleosis, Mumps, Pertussis, Pneumonia, Poliomyelitis, Ringworm, Riverblindness, Rubella, Schistosomiasis, Smallpox, Strep throat, Trachoma, Trichuriasis, Tuberculosis, and Typhoid fever.

In some embodiments, the methods and devices of the present disclosure are applied to a subject who is suspected of having a pathogenic infection or disease, but who has not yet been diagnosed as having such an infection or disease. A subject may be “suspected of having” a pathogenic infection or disease when the subject exhibits one or more signs or symptoms of such an infection or disease. Such signs or symptoms are well known in the art and may vary, depending on the nature of the pathogen and the subject. Signs and symptoms of disease may generally include any one or more of the following: fever, chills, cough (e.g., dry cough), generalized fatigue, sore throat, runny nose, nasal congestion, muscle aches, difficulty breathing (shortness of breath), congestion, runny nose, headaches, nausea, vomiting, diarrhea, loss of smell and/or taste, skin lesions (e.g., pox), or loss of appetite. Other signs or symptoms of disease are specifically contemplated herein. As a non-limiting example, symptoms of coronaviruses (e.g., COVID-19) may include, but are not limited to, fever, cough (e.g., dry cough), generalized fatigue, sore throat, runny nose, nasal congestion, muscle aches, loss of smell and/or taste, and difficulty breathing (shortness of breath). As a non-limiting example, symptoms of influenza may include, but are not limited to, fever, chills, muscle aches, cough, sore throat, runny nose, nasal congestion, and generalized fatigue.

A subject may also be “suspected of having” a pathogenic infection or disease despite exhibiting no signs or symptoms of such an infection or disease (e.g., the subject is asymptomatic). Pathogenic infections can be highly transmissible. In some embodiments, an asymptomatic subject is suspected of having a pathogenic infection or disease due to known contact with an individual having or suspected of having a pathogenic infection or disease (e.g., an individual who tested positive as having a pathogenic infection or disease). In some embodiments, an asymptomatic subject is suspected of having a pathogenic infection or disease due to known contact with an individual having or suspected of having a pathogenic infection or disease within the preceding two-week (e.g., 14 day) time period. In some embodiments, an asymptomatic subject is suspected of having a pathogenic infection or disease due to known contact with an individual who tested positive as having a pathogenic infection or disease within the preceding two-week (e.g., 14 day) time period.

In some embodiments, the devices and methods of the present disclosure are configured to sequence a target nucleic acid sequence of a cancer cell. Cancer cells have unique mutations found in tumor cells and absent in normal cells. For example, the devices and methods of the present disclosure may be configured to sequence a target nucleic acid sequence encoding a cancer neoantigen, a tumor-associated antigen (TAA), and/or a tumor-specific antigen (TSA). Examples of TAAs include, but are not limited to, MelanA (MART-I), gplOO (Pmel 17), tyrosinase, TRP-I, TRP-2, MAGE-I, MAGE-3, BAGE, GAGE-I, GAGE-2, p15(58), CEA, RAGE, NY-ESO (LAGE), SCP-I, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, alpha-fetoprotein, β-HCG, BCA225, BTAA, CA 125, CA 15-3 (CA 27.29BCAA), CA 195, CA 242, CA-50, CAM43, CD68VKP1, CO-029, FGF-5, G250, Ga733 (EpCAM), HTgp-175, M344, MA-50, MG7-Ag, MOV18, NB/70K, NY-CO-I, RCAS1, SDCCAG16, TA-90 (Mac-2 binding protein/cyclophilin C-associated protein), TAAL6, TAG72, TLP, and TPS5. Neoantigens, in some embodiments, arise from tumor proteins (e.g., tumor-associated antigens and/or tumor-specific antigens). In some embodiments, the neoantigen comprises a polypeptide comprising an amino acid sequence that is identical to a sequence of amino acids within a tumor antigen or oncoprotein (e.g., Her2, E7, tyrosinase-related protein 2 (Trp2), Myc, Ras, or vascular endothelial growth factor (VEGF)). In some embodiments, the amino acid sequence comprises at least 10, at least 15, at least 20, at least 25, at least 30, at least 35, at is least 40, at least 45, at least 50, at least 75, at least 100, at least 150, at least 200, or at least 250 amino acids. In some embodiments, the amino acid sequence comprises 10-250, 50-250, 100-250, or 50-150 amino acids.

In some cases, a target nucleic acid is associated with one or more polymorphisms (e.g., single-nucleotide polymorphisms (SNPs)). In certain embodiments, the devices and methods of the present disclosure may be used to detect the presence or absence of a polymorphism of a target nucleic acid, which may affect medical treatment. In some instances, the devices and methods of the present disclosure may be used to profile the polymorphisms of certain genes (e.g., certain drug metabolism genes) to improve patient care.

In some embodiments, the devices and methods of the present disclosure are configured to examine a subject's predisposition to certain types of cancer based on specific genetic mutations. As an example, mutations in BRCA1 and/or BRCA2 may indicate that a subject is at an increased risk of breast cancer, as compared to a subject who does not have mutations in the BRCA1 and/or BRCA2 genes. In some instances, the devices and methods of the present disclosure are configured to detect a target nucleic acid sequence comprising a mutation in BRCA1 and/or BRCA2. Other genetic mutations that may be screened according to the diagnostic devices, systems, and methods provided herein include, but are not limited to, BARD1, BRIP1, TP53, PTEN, MSH2, MLH1, MSH6, NF1, PMS1, PMS2, EPCAM, APC, RB1, MEN1, MEN2, and VHL. Further, determining a subject's genetic profile may help guide treatment decisions, as certain cancer drugs are indicated for subjects having specific genetic variants of particular cancers. For example, azathioprine, 6-mercaptopurine, and thioguanine all have dosing guidelines based on a subject's thiopurine methyltransferase (TPMT) genotype (see, e.g., The Pharmacogeneomics Knowledgebase, pharmgkb.org).

In some embodiments, the methods and devices of the present disclosure are configured to detect a target nucleic acid sequence associated with a genetic disorder. Non-limiting examples of genetic disorders include hemophilia, sickle cell anemia, α-thalassemia, β-thalassemia, Duchene muscular dystrophy (DMD), Huntington's disease, severe combined immunodeficiency, Marfan syndrome, hemochromatosis, and cystic fibrosis. In some embodiments, the target nucleic acid sequence is a portion of nucleic acid from a genomic locus of at least one of the following genes: CFTR, FMR1, SMN1, ABCB 11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASP A, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CUT A, CLN3, CLN5, CLN6, CLN8, CLRN1, CNGB3, COL27A1, COL4A3, COL4A4, COL4A5, COL7A1, CPS1, CPT1A, CPT2, CRB 1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESC02, ETFA, ETFDH, ETHE1, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBAI, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAM A3, LAMB3, LAMC2, LCA5, LDLR, LDLRAP1, LHX3, LIFR, LIP A, LOXHD1, LPL, LRPPRC, MAN2B1, MCOLN1, MED 17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OP A3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMHD1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The methods and devices described herein may also be used to test water or food for contaminants (e.g., for the presence of one or more bacterial toxins). Bacterial contamination of food and water can result in foodborne diseases, which contribute to approximately 128,000 hospitalizations and 3000 deaths annually in the United States (CDC, 2016). In some cases, the diagnostic tests, systems, and methods described herein may be used to detect one or more toxins (e.g., bacterial toxins). In particular, bacterial toxins produced by Staphylococcus spp., Bacillus spp., and Clostridium spp. account for the majority of foodborne illnesses. Non-limiting examples of bacterial toxins include toxins produced by Clostridium botulinum, C. perfringens, Staphylococcus aureus, Bacillus cereus, Shiga-toxin-producing Escherichia coli (STEC), and Vibrio parahemolyticus. Exemplary toxins include, but are not limited to, aflatoxin, cholera toxin, diphtheria toxin, Salmonella toxin, Shiga toxin, Clostridium botulinum toxin, endotoxin, and mycotoxin. By testing a potentially contaminated food or water sample using the devices and methods described herein, one can determine whether the sample contains the one or more bacterial toxins. In some embodiments, the diagnostic tests, systems, or methods may be operated or conducted during a food production process to ensure food safety prior to consumption.

### Evanescent Wave Imaging

As used herein, evanescent wave imaging refers to a collection of techniques for manipulating one or more photo-active moieties (e.g., a detectable moiety, a photocleavable terminating moiety) using an evanescent wave produced by total internal reflection and producing one or more images using the emission of the one or more photo-active moieties. For instance, any of the illustrative devices of FIGS. 1A-1F may be operated to perform evanescent wave imaging. In some embodiments, a suitable device may be operated to perform evanescent wave imaging and thereby detect the fluorescence of a detectable moiety. A suitable device (e.g., one or more processors) may determine the identity of the detectable moiety (e.g., a protected nucleotide comprising the detectable moiety) based on measurements of the fluorescence. In some embodiments, a suitable device is operated to perform evanescent wave imaging in order to cleave a photocleavable linker, e.g., thereby relieving termination of elongation of a sequencing primer annealed to a substrate polynucleotide as described herein. An evanescent wave imaging apparatus refers to an apparatus capable of performing evanescent wave imaging, examples of which are described above.

### Substrate Illumination

As described above, a nucleic acid sequencing device (e.g., a device comprising an evanescent wave imaging apparatus) may comprise one or more light sources. Below are described various features of such light sources. The below description may be applied to any suitable embodiment described above in relation to FIGS. 1A-1G, including any of the above description relating to light source 112, light source 114 and their described features.

In some embodiments, an evanescent wave imaging apparatus comprising one or more first light sources may be configured to: (i) determine the identity of a nucleotide (e.g., a protected nucleotide) incorporated into a sequencing primer annealed to a substrate polynucleotide by exciting a detectable moiety of the nucleotide (e.g., protected nucleotide); (ii) reverse termination of elongation of a sequencing primer annealed to a substrate polynucleotide; and/or (iii) reverse termination of elongation of a sequencing primer. In some embodiments, a first group of one or more light sources may be operated by an evanescent wave imaging apparatus to produce emission light that may be analyzed to determine the identity of a nucleotide incorporated into a sequencing primer annealed to a substrate polynucleotide, and a second group of one or more light sources may be operated by the evanescent wave imaging apparatus to produce emission light that may be analyzed to reverse termination of elongation of a sequencing primer.

In some embodiments, a first group of one or more first light sources may be operated by an evanescent wave imaging apparatus to produce emission light that may be analyzed to determine the identity of a nucleotide (e.g., a protected nucleotide) incorporated into a sequencing primer, whereas a second group of one or more second light sources may be operated by the evanescent wave imaging apparatus to produce emission light that may be analyzed to reverse termination of elongation of a sequencing primer, and at least one light source is a member of both the first group and the second group.

Each of the above techniques may comprise emitting light from one or more light sources having a desired wavelength (or wavelength band), power density, and/or pulse duration suitable to have a desired effect within the reservoir, such as exciting a detectable moiety of a protected nucleotide (e.g., to induce fluorescence of a fluorophore), inducing cleavage of a photocleavable terminating moiety (e.g., of a protected nucleotide).

In some embodiments, a light source may comprise a light-emitting diode (LED), a laser diode, an incandescent bulb, and/or a fluorescent lamp. Non-limiting examples of suitable LEDs include LEDs having the emission properties described herein (e.g., LA UY20WP1, LA UY42WP1, LA SB20WP6, LA TB37WP6, LA CB43FP6, LA SG20WP6, LA YL20WP5, LA UR20WP5) and commercially available LEDs from Osram (Munich, Germany), Luxeon (Lethbridge, Alberta, Canada), and Lumileds (Amsterdam, Netherlands). Any light source meeting the criteria taught by the present disclosure may be suitable for use in the devices and methods of the present disclosure.

In some embodiments, a light source emits light having a peak wavelength in the visible range of the electromagnetic spectrum. Light having a peak wavelength in the visible range of the electromagnetic spectrum generally refers to light having a wavelength in a range from 400 to 700 nm. In certain embodiments, one or more light sources emit light having a peak wavelength in a range of 400-450 nm, 400-500 nm, 400-550 nm, 400-600 nm, 400-650 nm, 400-700 nm, 450-500 nm, 450-550 nm, 450-600 nm, 450-650 nm, 450-700 nm, 460-490 nm, 500-550 nm, 500-600 nm, 500-650 nm, 500-700 nm, 550-600 nm, 550-650 nm, 550-700 nm, 570-590 nm, 600-650 nm, or 600-700 nm. In some instances, one or more light sources emit light having a peak wavelength of about 400 nm, 445 nm, 450 nm, 460 nm, 470 nm, 480 nm, 490 nm, 495 nm, 496 nm, 500 nm, 512 nm, 520 nm, 525 nm, 527 nm, 550 nm, 570 nm, 585 nm, 590 nm, 600 nm, 605 nm, 610 nm, 625 nm, 645 nm, 650 nm, and/or 700 nm.

In some embodiments, a light source emits a spectrum of light comprising a plurality of wavelengths (including, for example, wavelengths in the UV and/or visible ranges).

In some embodiments, an evanescent wave imaging apparatus may be configured to operate one or more light sources to emit light continuously in response to input provided by a user to the apparatus (e.g., via toggling of an on/off button). In some embodiments, an evanescent wave imaging apparatus may be configured to operate one or more light sources to emit light for a predetermined period of time (e.g., 1 millisecond, 1 second, 10 seconds, 30 seconds, 1 minute, etc.) in response to one or more input signals (e.g., from a controller). In some embodiments, an evanescent wave imaging apparatus may be configured to operate one or more light sources to emit pulses of light at a predetermined rate for a predetermined total period of time and/or with a predetermined number of pulses in response to one or more input signals (e.g., from a controller). As an illustrative example, the light source may be controlled to emit 20 pulses of light at a rate of one pulse every millisecond in response to one or more input signals. In some embodiments, an intensity of light emitted by a light source may be controlled by a controller according to a computer program executed by a processor of the controller. Additional details of light source operation are provided below.

Light Source Optical Filters

As described above, in some embodiments, one or more optical filters (e.g., optical filter 124) are positioned between a light source (e.g., 112A) and a substrate (e.g., 106). In certain cases, at least a portion (and, in some cases, substantially all) of light emitted by the light source passes through the one or more excitation-light optical filters prior to entering the substrate. As an illustrative, non-limiting example, an exemplary light source may produce a broad range of wavelengths of light (e.g., both UV and visible light). As part of configuring said exemplary light source to determine the identity of a nucleotide incorporated into a sequencing primer, an excitation-light optical filter may be operably coupled to the exemplary light source (e.g., to block UV light and transmit visible light).

As described herein, in some embodiments, the reservoir (e.g., 104) may comprise an aqueous solution comprising a pool of nucleotides comprising one or more detectable moieties (e.g. fluorescent moieties) and one or more photocleavable terminating moieties. In some embodiments, the first, second, third, and/or fourth fluorescent moieties fluoresce upon absorption of a wavelength in a range of longer wavelengths (e.g., visible light), and at least one photocleavable terminating moiety cleaves upon absorption of a wavelength in a range of shorter wavelengths (e.g., UV light). In some embodiments, the evanescent wave imaging apparatus may be configured to prevent or mitigate (e.g., decrease or minimize) transmission of light into the substrate that, as a result of total internal reflection within a substrate, produces an evanescent wave that excites both a detectable moiety and a photocleavable terminating moiety, e.g., by comprising one or more light sources that emit light only of longer wavelengths or only of shorter wavelengths or by comprising one or more excitation-light optical filters (e.g., a longpass, shortpass, or bandpass filter). In some embodiments, an evanescent wave imaging apparatus may be configured such that one or more light sources emit only longer wavelengths, e.g., only visible light, and may not be operably coupled to an excitation-light optical filter (e.g., to block light that might reverse termination of elongation of a sequencing primer). In other embodiments, an evanescent wave imaging apparatus may be configured such that one or more light sources emit a range of wavelengths encompassing both the longer and shorter wavelength ranges (e.g., UV and visible light) and may be operably coupled to an excitation-light optical filter (e.g., to block light that might reverse termination of elongation of a sequencing primer).

In some embodiments, the first, second, third, and/or fourth fluorescent moieties absorb a range of wavelengths that substantially overlaps with a range of wavelengths absorbed by a photocleavable terminating moiety (e.g., at least one fluorescent moiety and the photocleavable terminating moiety both absorb UV light). In some such embodiments, the substantial overlap of wavelength ranges results in some portion of an excitation spectrum for the relevant fluorescent moiety(ies) that does not significantly excite the photocleavable terminating moiety. In some such embodiments, the evanescent wave imaging apparatus may be configured to prevent or mitigate (e.g., decrease or minimize) transmission of light into the substrate that, as a result of total internal reflection within a substrate, produces an evanescent wave that excites both a detectable moiety and a photocleavable terminating moiety, e.g., by comprising one or more light sources that emit light only of wavelengths of the portion of an excitation spectrum for the relevant fluorescent moiety(ies) that does not significantly excite the photocleavable terminating moiety or by comprising one and/or more excitation-light optical filters. In some such embodiments, an evanescent wave imaging apparatus may comprise one or more light sources operably coupled to an excitation-light optical filter, e.g., to block light that might, as a result of total internal reflection within a substrate, produces an evanescent wave that excites both a detectable moiety and a photocleavable terminating moiety and reverse termination of elongation of a sequencing primer. In some embodiments, the excitation-light optical filter blocks the wavelengths of the excitation spectrum of the photocleavable terminating moiety and transmits wavelengths of the excitation spectrum of one or more detectable moieties. In other embodiments, the substantial overlap of wavelength ranges results in essentially no portion of the excitation spectrum for the relevant fluorescent moiety(ies) that does not significantly excite the photocleavable terminating moiety. In some such embodiments, the evanescent wave imaging apparatus comprises one or more light sources operably coupled to an excitation-light optical filter, e.g., to block light that might, as a result of total internal reflection within a substrate, produces an evanescent wave that excites both a detectable moiety and a photocleavable terminating moiety and reverse termination of elongation of a sequencing primer. In some such embodiments, the power density and duration of one or more light pulses may be selected to mitigate (e.g., decrease or minimize) relieving of reversible termination of elongation of a sequencing primer while providing light sufficient to, as a result of total internal reflection within a substrate, produce an evanescent wave that excites a detectable moiety. Optical filters may also or alternately be operably coupled to one or more light sources to decrease, minimize, or prevent light from the one or more light sources from reaching a detector (e.g., ensuring that light reaching the detector is fluorescence emission).

In some embodiments, a light source is operably coupled to a longpass optical filter. For example, a light source may be operably coupled to a longpass optical filter that blocks light having a wavelength below about 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm (and optionally transmits light above said wavelength). In some embodiments, a light source is operably coupled to a shortpass optical filter. For example, a light source may be operably coupled to a shortpass optical filter that blocks light having a wavelength above about 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm (and optionally transmits light below said wavelength).

In some embodiments, a light source is operably coupled to a bandpass optical filter. For example, a light source may be operably coupled to a bandpass optical filter that blocks light having a wavelength above about 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm and light having a wavelength below about 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm (and optionally transmits light between said wavelengths).

In some embodiments, a light source is operably coupled to a longpass optical filter that blocks UV light, e.g., light below a wavelength of about 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm. In some embodiments, a light source configured to reverse termination of elongation of a sequencing primer is operably coupled to a shortpass optical filter that blocks light above a wavelength of about 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, or 600 nm.

Power and Pulse Duration

In some embodiments, a light source produces light of sufficient power density to produce, as a result of total internal reflection within a substrate, an evanescent wave that induces a reaction that produces fluorescence of a detectable moiety. In some embodiments, the light source produces light at a power density of at least 0.5 W/cm², 1 W/cm², 2 W/cm², 3 W/cm², 4 W/cm², 5 W/cm², 6 W/cm², 7 W/cm², 8 W/cm², 9 W/cm², 10 W/cm², 20 W/cm², 30 W/cm², 40 W/cm², 50 W/cm², 60 W/cm², 70 W/cm², 80 W/cm², 90 W/cm², or 100 W/cm². In some embodiments, the light source produces light at a power density of no more than 0.5 W/cm², 1 W/cm², 2 W/cm², 3 W/cm², 4 W/cm², 5 W/cm², 6 W/cm², 7 W/cm², 8 W/cm², 9 W/cm², 10 W/cm², 20 W/cm², 30 W/cm², 40 W/cm², 50 W/cm², 60 W/cm², 70 W/cm², 80 W/cm², 90 W/cm², or 100 W/cm². In some embodiments, the light source produces light at a power density in a range from 0.5 W/cm² to 1 W/cm², 0.5 W/cm² to 3 W/cm², 0.5 W/cm² to 5 W/cm², 0.5 W/cm² to 10 W/cm², 0.5 W/cm² to 20 W/cm², 0.5 W/cm² to 50 W/cm², 0.5 W/cm² to 100 W/cm², 1 W/cm² to 3 W/cm², 1 W/cm² to 5 W/cm², 1 W/cm² to 10 W/cm², 1 W/cm² to 20 W/cm², 1 W/cm² to 50 W/cm², 1 W/cm² to 100 W/cm², 5 W/cm² to 10 W/cm², 5 W/cm² to 20 W/cm², 5 W/cm² to 50 W/cm², 5 W/cm² to 100 W/cm², 10 W/cm² to 20 W/cm², 10 W/cm² to 50 W/cm², 10 W/cm² to 100 W/cm², 20 W/cm² to 50 W/cm², 20 W/cm² to 100 W/cm², or 50 W/cm² to 100 W/cm².

In some embodiments, a light source produces a pulse of light having a duration sufficient to produce, as a result of total internal reflection within a substrate, an evanescent wave that induces detectable fluorescence of a detectable moiety. In some embodiments, the light source produces a pulse of light having a duration of no more than about 5000 milliseconds (ms), 4000 ms, 3000 ms, 2000 ms, 1000 ms, 500 ms, 200 ms, 100 ms, 50 ms, 20 ms, 19 ms, 18 ms, 17 ms, 16 ms, 15 ms, 14 ms, 13 ms, 12 ms, 11 ms, 10 ms, 9 ms, 8 ms, 7 ms, 6 ms, 5 ms, 4 ms, 3 ms, 2 ms, 1 ms, 0.9 ms, 0.8 ms, 0.7 ms, 0.6 ms, 0.5 ms, 0.4 ms, 0.3 ms, 0.2 ms, 0.1 ms, 0.09 ms, 0.08 ms, 0.07 ms, 0.06 ms, or 0.05 ms.

In some embodiments, a light source produces a pulse of light of sufficient power density and duration to produce, as a result of total internal reflection within a substrate, an evanescent wave that induces detectable fluorescence of a detectable moiety. In some embodiments, a light source produces a pulse of light of sufficient power density and duration to produce, as a result of total internal reflection within a substrate, an evanescent wave that induces cleavage of a photocleavable terminating moiety (e.g., of a protected nucleotide incorporated into a sequencing primer). Without wishing to be bound by a particular theory, an evanescent wave imaging apparatus may be configured such that a shorter pulse duration and/or lower power density is used to produce, as a result of total internal reflection within a substrate, an evanescent wave that induces detectable fluorescence of a detectable moiety and a longer pulse duration and/or higher power density is used to produce, as a result of total internal reflection within a substrate, an evanescent wave that induces cleavage of a photocleavable terminating moiety. In general, reversing termination of elongation while (e.g., in the process of) determining the identity of an incorporated nucleotide should be avoided as this can decrease the synchronization of extension across the sequencing primers annealed to the pool of substrate polynucleotides. Configuring an evanescent wave imaging apparatus to utilize different power and duration of pulse to produce an evanescent wave is one way, amongst several described herein, to decrease the likelihood of reversing termination while inducing detectable fluorescence of a detectable moiety.

In some embodiments, a light source produces light of a sufficient power density to produce, as a result of total internal reflection within a substrate, an evanescent wave that induces cleavage of a photocleavable terminating moiety (e.g., of a protected nucleotide incorporated into a sequencing primer). In some embodiments, the light source produces light at a power density of at least 0.5 W/cm², 0.75 W/cm², 1 W/cm², 1.25 W/cm², 1.5 W/cm², 1.75 W/cm², 2 W/cm², 2.25 W/cm², 2.5 W/cm², 2.75 W/cm², 3 W/cm², 3.25 W/cm², 3.5 W/cm², 3.75 W/cm², 4 W/cm², 4.5 W/cm², 5 W/cm², 5.5 W/cm², 6 W/cm², 6.5 W/cm², 7 W/cm², 7.5 W/cm², 8 W/cm², 8.5 W/cm², 9 W/cm², 9.5 W/cm², 10 W/cm², 11 W/cm², 12 W/cm², 13 W/cm², 14 W/cm², 15 W/cm², 16 W/cm², 17 W/cm², 18 W/cm², 19 W/cm², 20 W/cm², 30 W/cm², 40 W/cm², 50 W/cm², 60 W/cm², 70 W/cm², 80 W/cm², 90 W/cm², or 100 W/cm².

In some embodiments, the light source may be configured to produce one or more pulses of light each having a duration of at least about 1, 2, 3, 4, 5, 6, 7, 8, 9, 10, 20, 50, 100, 150, 200, 250, 300, 350, 400, 450, 500, 600, 700, 800, 900, 1000, 2000, 3000, 4000, or 5000 milliseconds (ms). In some embodiments, the light source produces a pulse of light having a duration of no more than 5, 10, 20, 50, 100, 150, 200, 250, 300, 350, 400, 450, 500, 600, 700, 800, 900, 1000, 2000, 3000, 4000, or 5000 milliseconds (ms).

In some embodiments, a light source may be configured to produce one or more pulses of light of sufficient power density and having sufficient duration to produce, as a result of total internal reflection within a substrate, an evanescent wave that induces cleavage of a photocleavable terminating moiety (e.g., of a protected nucleotide incorporated into a sequencing primer). Without wishing to be bound by a particular theory, there may be a direct correlation between the power density of light and the rate at which termination of elongation of a sequencing primer is reversed. Additionally, the longer the duration of the pulse of light the greater the likelihood of reversing termination. However, a longer pulse duration also provides increased opportunity for asynchronous extensions of sequencing primers. In some embodiments, an evanescent wave imaging apparatus is configured to operate a light source to produces a pulse of light having a duration long enough to produce, as a result of total internal reflection within a substrate, an evanescent wave that sufficiently reverses termination but short enough to avoid unnecessary asynchronous extensions. In general, decreasing the duration of the pulse of light used to produce, as a result of total internal reflection within a substrate, an evanescent wave that reverses termination (e.g., by rapidly inducing cleavage of a photocleavable linker with a high power density and short duration pulse of light) is desirable to maintain synchronization of extension across a pool of sequencing primers.

Coupling for TIR

The one or more light sources may be coupled to the substrate in a manner sufficient to produce total internal reflection of the light emitted by the one or more light sources. In some embodiments, total internal reflection of light from the one or more light sources in the substrate results in an evanescent wave whose energy is present in a portion of the thin layer at an inner surface of the reservoir. In some embodiments, light from the one or more light sources only enters or only appreciably enters the reservoir as an evanescent wave. Restricting exposure of the reservoir to a light source can be accomplished by many means, e.g., by positioning an opaque blocking element (e.g., a rubber gasket) at the border of an outer edge of the substrate to obstruct entry of light from the light source to the reservoir.

Without wishing to be bound by a particular theory, the distance (e.g., di, d₂) between a light source and a substrate illuminated by the light source influences the power density of the light that enters the substrate and also influences the power density of the evanescent wave produced at an interface of the substrate. In general, the closer the light source is to the substrate, the higher the power density of the light entering the substrate and the higher the power density of the evanescent wave produced. In some embodiments, the light source is no more than 1 mm, 0.9 mm, 0.8 mm, 0.7 mm, 0.6 mm, 0.5 mm, 0.4 mm, 0.3 mm, 0.2 mm, 0.1 mm, or 0.05 mm from the substrate. In some embodiments, the light source is at least 0.01 mm, 0.02 mm, 0.03 mm, 0.04 mm, 0.05 mm, 0.1 mm, 0.2 mm, 0.3 mm, 0.4 mm, 0.5 mm, 0.6 mm, 0.7 mm or 0.8 mm from the substrate. In some embodiments, the light source is not in contact with the substrate. In certain embodiments, a gap between the light source and the substrate is filled with a fluid (e.g., air, water, adhesive, oil). In some embodiments, the light source is in contact (e.g., direct physical contact) with the substrate.

In some embodiments, the one or more light sources inject light that internally are at a variety of angles of incidence relative to the normal to the surfaces of an incident surface (e.g., 106c, 106d) of the substrate. As discussed above, a substrate's capacity for total internal reflection depends upon the refractive index of the substrate and the refractive index or indices of the material surrounding the substrate. Without wishing to be bound by a particular theory, light incident to a substrate face at an angle below the value of the critical angle may escape the substrate (i.e., may not be internally reflected). Light leakage from the substrate can interfere with evanescent wave imaging, e.g., by one or a combination of: interacting with the detector, inducing fluorescence of detectable moieties on non-incorporated nucleotides in the reservoir, or reversing termination of elongation of a sequencing primer in an undesired manner (e.g., during determination of the identity of an incorporated nucleotide). In some embodiments, a nucleic acid sequencing device (e.g., a device comprising an evanescent wave imaging apparatus) comprises one or more light leakage mitigation mechanisms. Light leakage mitigation mechanisms include, but are not limited to: configuring the distance (e.g., d₁ and/or d₂) between the one or more light sources and the substrate to be high enough to prevent a majority or all of light having angles that would cause leakage from adjacent faces from entering the substrate; selecting a sufficiently high refractive index material for the substrate to decrease or prevent leakage of light having angles below the value of the critical angle (by decreasing the critical angle); and/or configuring the evanescent wave imaging apparatus to comprise a light sink (e.g., as described herein).

In general, increasing the distance between the light source and the substrate may increase the amount of light incident on the surface of the substrate that has an incident angle above the critical angle, which may reduce the light that would escape from adjacent surfaces by refraction. However, as described above, increasing the distance between the light source and the substrate may also decrease the power density of the light that enters the substrate and consequently may reduce the power density of the evanescent wave produced. As discussed herein, the refractive index of the material of the substrate may influence the degree to which light having angles below the value of the critical angle can leak from the substrate and which angles of light may do so. Without wishing to be bound by a particular theory, a sufficiently high refractive index substrate material can allow coupling of a light source to the substrate at a closer distance to the substrate, which may enable more power density to be delivered to the substrate and thus may result in a higher power-density evanescent wave to be produced, which may enable the use of a lower-power light source. Without wishing to be bound by a particular theory, a sufficiently high power density light source can be coupled at a farther distance to a substrate to compensate for a substrate having a lower refractive index, e.g., to compensate for the lower refractive index material of the substrate admitting more light having angles that would escape from the adjacent surfaces. However, using a greater distance sufficient to prevent the injected light from having rays steeper than the critical angle will greatly decrease coupling efficiency. A better approach may be to use a smaller gap between a Lambertian light source such as an LED and edge of the solid 106, then optically isolate (e.g., divert, extract, and/or absorb) the small percentage of steep rays with an isolation layer 134 as described above.

For example, a nucleic acid sequencing device may comprise one or more light sources coupled to a substrate such that the one or more light sources may be at least about 0.6 mm from the substrate. In some embodiments, coupling the light source to the substrate above a threshold distance apart, e.g., above about 0.6 mm, effectively mitigates light leakage of light having angles that would escape from the adjacent surfaces.

As a further example, a nucleic acid sequencing device may comprise one or more light sources coupled to a substrate such that the one or more light sources are less than about 0.6 mm from the substrate and the material of the substrate has a sufficiently high refractive index to decrease or prevent leakage of light having angles below the critical angle (e.g., a refractive index of about 1.6, 1.63, 1.66, 1.70, 1.78, or higher). In some embodiments, coupling the light source to the substrate wherein the substrate material has a refractive index above a threshold value, e.g., above about 1.6, 1.63, 1.66, 1.70, or 1.78 effectively mitigates leakage of light having angles below the critical angle.

As a further example, a nucleic acid sequencing device may comprise one or more light sources coupled to a substrate such that the one or more light sources are less than about 0.6 mm from the substrate. In this example, the device may be configured to comprise a light sink (e.g., as described herein). In some embodiments, configuring the device to comprise a light sink effectively mitigates light leakage of light having angles that would escape from the adjacent surfaces.

The examples of light leakage mitigation mechanisms in combination with light source/substrate couplings provided herein are not exhaustive and all combinations are contemplated by the present disclosure. For example, in some embodiments a nucleic acid sequencing device comprises a light sink and one or more light sources coupled to a substrate such that the one or more light sources are at least about 0.6 mm from the substrate. As a further example, in some embodiments a nucleic acid sequencing device comprises a light sink and a substrate comprising a material having a refractive index above a threshold value, e.g., above about 1.6, 1.63, 1.66, 1.70, or 1.78.

Light Sources: Numbers and Arrangement

In some embodiments, an evanescent wave imaging apparatus comprises a plurality of light sources. In some embodiments, the plurality of light sources comprises a first set of at least one light sources comprising at least one light source that emits excitation light having one or more characteristics (e.g., wavelength, intensity, lifetime decay, pulse width) and that produces an evanescent wave that effectively excites a detectable moiety, and a second set of at least one light sources comprising at least one light source that emits excitation light having one or more characteristics (e.g., wavelength, intensity, lifetime decay, pulse width) and that produces an evanescent wave that effectively cleaves a photocleavable terminating moiety (e.g., of a protected nucleotide incorporated into the sequencing primer). In some embodiments, the first set of at least one light sources is the same as the second set of at least one light sources. For example, an evanescent wave imaging apparatus may comprise a single light source that emits excitation light having one or more characteristics (e.g., wavelength, intensity, lifetime decay, pulse width) and that produces an evanescent wave that effectively excites a detectable moiety and emits excitation light having one or more characteristics (e.g., wavelength, intensity, lifetime decay, pulse width) and that produces an evanescent wave that effectively cleaves a photocleavable terminating moiety (e.g., of a protected nucleotide incorporated into the sequencing primer). As a further example, an evanescent wave imaging apparatus may comprise a plurality of light sources that emit excitation light having one or more characteristics (e.g., wavelength, intensity, lifetime decay, pulse width) and that produces an evanescent wave that effectively excites one or more detectable moieties and a single light source that emits excitation light having one or more characteristics (e.g., wavelength, intensity, lifetime decay, pulse width) and that produces an evanescent wave that effectively cleaves a photocleavable terminating moiety (e.g., of a protected nucleotide incorporated into the sequencing primer).

In some embodiments, an evanescent wave imaging apparatus comprises 1, 2, 3, 4, 5, 6, 7, 8, 9, 10, 11, 12, 13, 14, 15, 16, 18, 20, 24, 28, 32, 36, 40, 44, 48, 50, or 60 light sources. In some embodiments, an evanescent wave imaging apparatus comprises at least 1, 2, 3, 4, 5, 6, 7, 8,9, 10, 11, 12, 13, 14, 15, 16, 18, 20, 24, 28, 32, 36, 40, 44, 48, 50, or 60 light sources and no more than 60, 50, 48, 44, 40, 36, 32, 28, 26, 24, 20, 19, 18, 17, or 16 light sources. In some embodiments, an evanescent wave imaging apparatus comprises a plurality of light sources that emit excitation light that produces an evanescent wave that effectively excites one or more detectable moieties. In some embodiments, an evanescent wave imaging apparatus comprises 1, 2, 3,4, 5, 6,7, 8, 9, 10, 11, 12, 13, 14, 15, 16, 18, 20, 24, 28, 32, 36, 40, 44, 48, 50, or 60 light sources that emit excitation light that produces an evanescent wave that effectively excites one or more detectable moieties. In some embodiments, an evanescent wave imaging apparatus comprises at least 1, 2, 3, 4, 5, 6, 7, 8, 9, 10, 11, 12, 13, 14, 15, 16, 18, 20, 24, 28, 32, 36, 40, 44, 48, 50, or 60 light sources and no more than 60, 50, 48, 44, 40, 36, 32, 28, 26, 24, 20, 19, 18, 17, or 16 light sources that emit excitation light that produces an evanescent wave that effectively excites one or more detectable moieties. In some embodiments, an evanescent wave imaging apparatus comprises a plurality of light sources that emits excitation light that produces an evanescent wave that effectively cleaves a photocleavable terminating moiety (e.g., of a protected nucleotide incorporated into the sequencing primer). In some embodiments, an evanescent wave imaging apparatus comprises 1, 2, 3, 4, 5, 6, 7, 8, 9, 10, 11, 12, 13, 14, 15, 16, 18, 20, 24, 28, 32, 36, 40, 44, 48, 50, or 60 light sources that emits excitation light that produces an evanescent wave that effectively cleaves a photocleavable terminating moiety (e.g., of a protected nucleotide incorporated into the sequencing primer). In some embodiments, an evanescent wave imaging apparatus comprises at least 1, 2, 3, 4, 5, 6, 7, 8, 9, 10, 11, 12, 13, 14, 15, 16, 18, 20, 24, 28, 32, 36, 40, 44, 48, 50, or 60 light sources and no more than 60, 50, 48, 44, 40, 36, 32, 28, 26, 24, 20, 19, 18, 17, or 16 light sources that emits excitation light that produces an evanescent wave that effectively cleaves a photocleavable terminating moiety (e.g., of a protected nucleotide incorporated into the sequencing primer).

In some embodiments, the one or more light sources (e.g., that emit excitation light that produces an evanescent wave that effectively excites one or more detectable moieties) emit visible light. In some embodiments, the one or more light sources emit light having a wavelength (e.g., a peak wavelength) of about 400 nm, 445 nm, 450 nm, 460 nm, 470 nm, 480 nm, 490 nm, 495 nm, 496 nm, 500 nm, 512 nm, 520 nm, 525 nm, 527 nm, 550 nm, 570 nm, 585 nm, 590 nm, 600 nm, 605 nm, 610 nm, 625 nm, 645 nm, 650 nm, and/or 700 nm. In some embodiments, the one or more light sources emit light having a wavelength in a range from 450-490 nm, 440-600 nm, 400-650 nm. In some embodiments, the one or more light sources emit light at a power density of at least 0.1 W/cm², at least 0.5 W/cm², at least 1 W/cm², at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm².

In some embodiments, the one or more light sources (e.g., that emit excitation light that produces an evanescent wave that effectively cleaves a photocleavable terminating moiety (e.g., of a protected nucleotide incorporated into the sequencing primer)) emit UV light. In some embodiments, the one or more light sources emit light having a wavelength (e.g., a peak wavelength) of about 365 nm. In some embodiments, the one or more light sources emit light at a power density of at least 20 W/cm².

In some embodiments, an evanescent wave imaging apparatus comprises a first light source that emits excitation light that produces an evanescent wave that effectively excites a first detectable moiety (e.g., comprised in a nucleotide (e.g., a first type of nucleotide) incorporated into a sequencing primer). In some embodiments, the first light source also emits excitation light that produces an evanescent wave that effectively excites a second detectable moiety (e.g., comprised in a nucleotide (e.g., a second type of nucleotide) incorporated into a sequencing primer), wherein the second detectable moiety is different from the first detectable moiety. In some embodiments, the first light source also emits excitation light that produces an evanescent wave that effectively excites a third detectable moiety (e.g., comprised in a nucleotide (e.g., a third type of nucleotide) incorporated into a sequencing primer) wherein the third detectable moiety is different from the first and second detectable moieties. In some embodiments, the first light source also emits excitation light that produces an evanescent wave that effectively excites a fourth detectable moiety (e.g., comprised in a nucleotide (e.g., a fourth type of nucleotide) incorporated into a sequencing primer) wherein the fourth detectable moiety is different from the first, second, and third detectable moieties.

In some embodiments, an evanescent wave imaging apparatus comprises a first light source that emits excitation light that produces an evanescent wave that effectively excites a first detectable moiety (e.g., comprised in a nucleotide (e.g., a first type of nucleotide)) and a second detectable moiety (e.g., comprised in a nucleotide (e.g., a second type of nucleotide)), wherein the first and second detectable moieties are different from one another. In some embodiments, an evanescent wave imaging apparatus comprises a second light source that emits excitation light that produces an evanescent wave that effectively excites a third detectable moiety (e.g., comprised in a nucleotide (e.g., a third type of nucleotide) and a fourth detectable moiety (e.g., comprised in a nucleotide (e.g., a fourth type of nucleotide) wherein the third and fourth detectable moieties are different from one another and from the first and second detectable moieties.

In some embodiments, an evanescent wave imaging apparatus comprises a first light source that emits excitation light that produces an evanescent wave that effectively excites a first detectable moiety (e.g., comprised in a nucleotide (e.g., a first type of nucleotide)), a second detectable moiety (e.g., comprised in a nucleotide (e.g., a second type of nucleotide)), and a third detectable moiety (e.g., comprised in a nucleotide (e.g., a third type of nucleotide)), wherein the first, second, and third detectable moieties are different from one another. In some embodiments, an evanescent wave imaging apparatus comprises a second light source that emits excitation light that produces an evanescent wave that effectively excites a fourth detectable moiety (e.g., comprised in a nucleotide (e.g., a fourth type of nucleotide)) wherein the fourth detectable moiety is different from the first, second, and third detectable moieties.

In some embodiments, an evanescent wave imaging apparatus comprises a first light source that emits excitation light that produces an evanescent wave that effectively excites a first detectable moiety (e.g., comprised in a nucleotide (e.g., a first type of nucleotide)) and a second detectable moiety (e.g., comprised in a nucleotide (e.g., a second type of nucleotide)), wherein the first and second detectable moieties are different from one another. In some embodiments, an evanescent wave imaging apparatus comprises a second light source that emits excitation light that produces an evanescent wave that effectively excites a third detectable moiety (e.g., comprised in a nucleotide (e.g., a third type of nucleotide)), wherein the third detectable moiety is different from the first and second detectable moieties. In some embodiments, an evanescent wave imaging apparatus comprises a third light source that emits excitation light that produces an evanescent wave that effectively excites a fourth detectable moiety (e.g., comprised in a nucleotide (e.g., a fourth type of nucleotide)) wherein the fourth detectable moiety is different from the first, second, and third detectable moieties.

In some embodiments, an evanescent wave imaging apparatus comprises a first light source that emits excitation light that produces an evanescent wave that effectively excites a first detectable moiety (e.g., comprised in a nucleotide (e.g., a first type of nucleotide)). In some embodiments, the evanescent wave imaging apparatus comprises a second light source that emits excitation light that produces an evanescent wave that effectively excites a second detectable moiety (e.g., comprised in a nucleotide (e.g., a second type of nucleotide)), wherein the first and second detectable moieties are different from one another. In some embodiments, the evanescent wave imaging apparatus comprises a third light source that emits excitation light that produces an evanescent wave that effectively excites a third detectable moiety (e.g., comprised in a nucleotide (e.g., a third type of nucleotide)), wherein the third detectable moiety is different from the first and second detectable moieties. In some embodiments, the evanescent wave imaging apparatus comprises a fourth light source that emits excitation light that produces an evanescent wave that effectively excites a fourth detectable moiety (e.g., comprised in a nucleotide (e.g., a fourth type of nucleotide)) wherein the fourth detectable moiety is different from the first, second, and third detectable moieties.

In some embodiments, an evanescent wave imaging apparatus comprises one or more duplicate light sources, i.e., a similarly configured light source in addition to an explicitly recited light source. For example, an evanescent wave imaging apparatus may comprise a first light source that emits excitation light that produces an evanescent wave that effectively excites a first detectable moiety (e.g., comprised in a nucleotide (e.g., a first type of nucleotide)), and one or more duplicate light sources that similarly emit excitation light that produces an evanescent wave that effectively excites the first detectable moiety. In some embodiments, duplicate light sources provide additional power (e.g., a stronger evanescent wave) and improvements (e.g., improved excitation of the detectable moiety, e.g., that improve operation of the apparatus to determine nucleotide identity (e.g., a better signal/noise ratio or improved temperature management). The disclosure contemplates any and all combinations of duplicate light sources and explicitly recited light sources; in any of the aforementioned or below embodiments, duplicate light sources may be included in the evanescent wave imaging apparatus in addition to a first, second, third, or fourth light sources.

In some embodiments, one or more light sources is coupled with the substrate along an outer edge of the substrate. In some embodiments, one or more light sources are coupled to an outer edge of the substrate such that light from the one or more light sources enters the substrate by entering through that outer edge. In some embodiments, the one or more light sources may be coupled to an outer edge of the substrate such that light from the one or more light sources may substantially (e.g., only) enter the substrate by entering through that outer edge.

In some embodiments, the one or more light sources that emit excitation light that produces an evanescent wave that effectively excites one or more detectable moieties are coupled to an outer edge of the substrate. In some embodiments, the one or more light sources that emit excitation light that produces an evanescent wave that effectively excites one or more detectable moieties are coupled to the same outer edge of the substrate. In some embodiments, the one or more light sources that emit excitation light that produces an evanescent wave that effectively cleaves a photocleavable terminating moiety (e.g., of a protected nucleotide incorporated into the sequencing primer) are coupled to an outer edge of the substrate. In some embodiments, the one or more light sources that emit excitation light that produces an evanescent wave that effectively cleaves a photocleavable terminating moiety may be coupled to the same outer edge of the substrate. In some embodiments, the one or more light sources that emit excitation light that produces an evanescent wave that effectively excites one or more detectable moieties are coupled to an outer edge of the substrate, and the one or more light sources that emit excitation light that produces an evanescent wave that effectively cleaves a photocleavable terminating moiety are coupled to a different outer edge of the substrate. In some embodiments, the one or more light sources that emit excitation light that produces an evanescent wave that effectively cleaves a photocleavable terminating moiety are coupled to a plurality of outer edges of the substrate. In some embodiments, the one or more light sources that emit excitation light that produces an evanescent wave that effectively cleaves a photocleavable terminating moiety may be coupled to a plurality of outer edges of the substrate and the one or more light sources that emit excitation light that produces an evanescent wave that effectively excites one or more detectable moieties are coupled to a different outer edge of the substrate. In some embodiments, the one or more light sources that emit excitation light that produces an evanescent wave that effectively cleaves a photocleavable terminating moiety and the one or more light sources that emit excitation light that produces an evanescent wave that effectively excites one or more detectable moieties are coupled to the same outer edge(s) of the substrate.

In some embodiments, one or more light sources that emit excitation light that produces an evanescent wave that effectively cleaves a photocleavable terminating moiety are coupled to a first outer edge of a substrate and one or more light sources that emit excitation light that produces an evanescent wave that effectively cleaves a photocleavable terminating moiety are coupled to a second, opposing outer edge of the substrate. In some embodiments, one or more light sources that emit excitation light that produces an evanescent wave that effectively excites one or more detectable moieties are coupled to a first outer edge and a second, opposing outer edge of the substrate, and one or more light sources that emit excitation light that produces an evanescent wave that effectively cleaves a photocleavable terminating moiety are coupled to a third outer edge and a fourth, opposing outer edge of the substrate. In some embodiments, the first and second outer edges of the substrate are orthogonal to the third and fourth outer edges, respectively.

In another embodiment, the one or more light sources that emit excitation light that produces an evanescent wave that effectively excites one or more detectable moieties are coupled to an outer edge of the substrate and at least one (e.g., all) of the one or more light sources that emit excitation light that produces an evanescent wave that effectively cleaves a photocleavable terminating moiety are coupled to the same outer edge of the substrate. In an exemplary embodiment, the one or more light sources that emit excitation light that produces an evanescent wave that effectively excites one or more detectable moieties are coupled to an outer edge of the substrate and the one or more light sources that emit excitation light that produces an evanescent wave that effectively cleaves a photocleavable terminating moiety are coupled to the same outer edge of the substrate. In a further exemplary embodiment, the one or more light sources that emit excitation light that produces an evanescent wave that effectively excites one or more detectable moieties are coupled to two or more (e.g., two, three, or four) outer edges of the substrate and the one or more light sources that emit excitation light that produces an evanescent wave that effectively cleaves a photocleavable terminating moiety are coupled to the same two or more outer edges of the substrate.

Temperature Regulation

In some embodiments, an evanescent wave imaging apparatus comprises one or more heat sinks (e.g., 132). A heat sink may be a component capable of absorbing heat from another component of an apparatus or device. In some embodiments, a heat sink is capable of dissipating absorbed heat, e.g., in a manner that directs the heat away from at least one other component of the apparatus or device. Numerous examples of heat sinks are known to those of skill in the art and may be used in the apparatuses and devices of the disclosure. In some embodiments, a heat sink may be configured to maintain one or more components of the apparatus or device (e.g., a reservoir) at a selected temperature to control one or more reactions within the reservoir. In certain embodiments, for example, one or more heat sinks may be connected to a heating element (e.g., a resistive element) and a temperature sensor. In some cases, a controller in communication with the heating element and the temperature sensor may be configured to maintain temperature at a desired set point.

In some embodiments, a heat sink comprises a high-thermal-conductivity material. Non-limiting examples of suitable high-thermal-conductivity materials include aluminum, aluminum alloys, copper, and copper alloys. In some embodiments, a heat sink comprises one or more features configured to increase the surface area of the heat sink (e.g., to increase the area of the heat sink exposed to a cooling fluid, e.g., air). In certain cases, for example, a heat sink comprises one or more fins. In certain instances, a heat sink comprises a plurality of fins. In some embodiments, a heat sink comprises one or more fluid channels configured to enable a cooling fluid (e.g., air) to flow therein. In some instances, the cooling fluid may carry heat away from an apparatus component (e.g., a light source) and/or may cool the apparatus component via conduction. In some embodiments, a cooling fluid (e.g., air) may be pumped through the one or more fluid channels.

In some embodiments, a heat sink is operably coupled (e.g., in thermal communication) with one or more components of an evanescent wave imaging apparatus. In certain embodiments, a heat sink is operably coupled (e.g., in thermal communication) with one or more light sources (e.g., one or more first light sources, one or more second light sources) of the evanescent wave imaging apparatus. The one or more light sources may, in some instances, comprise one or more LEDs. In some embodiments, a heat sink is operably coupled (e.g., in thermal communication) with a plurality (and, in some cases, all) of the light sources of the evanescent wave imaging apparatus. In certain embodiments, a plurality of heat sinks is operably coupled (e.g., in thermal communication) with a plurality (and, in some cases, all) of the light sources of the evanescent wave imaging apparatus (e.g., such that each light source is operably coupled with its own heat sink). In some instances, a heat sink that is operably coupled with one or more components of an evanescent wave imaging apparatus is in direct physical contact with the one or more components. In some embodiments, an evanescent wave imaging apparatus comprises one or more fans.

Without wishing to be bound by a particular theory, one or more reagents contained in the reservoir and immobilized on the surface of the substrate may be sensitive to changes in temperature, and one or more components (e.g., one or more light sources) of the evanescent wave imaging apparatus may produce heat. By incorporating one or more heat sinks, devices and methods of the present disclosure may decrease disruption of nucleic acid amplification and/or sequencing due to changes in temperature caused by the accumulation and/or leakage of heat from the one or more components. The one or more components that generate heat may include one or more light sources, heaters (e.g., Peltier element) and/or other electronic components. In some embodiments, an evanescent wave imaging apparatus comprises one or more heaters, e.g., configured to regulate the temperature of the reservoir.

Light Modifiers

In some embodiments, a nucleic acid sequencing device comprises one or more isolation layers and/or light blocking layers (e.g., 134, 136). As discussed above, an isolation layer may have an advantage of optically isolating the solution in a reservoir from evanescent light where it is present; whereas a light blocking layer may have an advantage of inhibiting light from entering and/or exit at least a portion of a substrate, a reservoir, and/or an optical imaging system.

In some embodiments, an isolation layer is configured to divert, extract, and/or absorb light that is incident upon the upper boundary of the substrate at an incident angle below the critical angle and that could otherwise be transmitted into the reservoir (e.g., that could adversely affect the chemistry within the reservoir). In some embodiments, an isolation layer is configured to optically isolate the solution in the reservoir from an evanescent wave emanating from the substrate (e.g., except at spots, e.g., positioned in wells or voids in the isolation layer). According to some embodiments, the isolation layer may be optically transparent. In some embodiments, the isolation layer has a transmission rate for visible light (e.g., light having a wavelength in a range from 400 to 700 nm) of at least 85%, 90%, 95%, 98%, or 99%. In certain embodiments, the isolation layer has a transmission rate for visible light in a range from 85% to 90%, 85% to 95%, 85% to 98%, 85% to 99%, 90% to 95%, 90% to 98%, 90% to 99%, 95% to 98%, or 95% to 99%. In some embodiments, the isolation layer has a transmission rate for ultraviolet (UV) light of at least 80%, 85%, 90%, 95%, 98%, or 99%. In certain embodiments, the isolation layer has a transmission rate for UV light in a range from 80% to 85%, 80% to 90%, 80% to 95%, 80% to 98%, 80% to 99%, 85% to 90%, 85% to 95%, 85% to 98%, 85% to 99%, 90% to 95%, 90% to 98%, 90% to 99%, 95% to 98%, or 95% to 99%.

In some embodiments, an isolation layer comprises a polymer. In certain embodiments, the polymer comprises CYTOP®, BIO-133 (see, e.g., Han et al. Lab Chip. 2021 Apr. 20; 21(8):1549-1562, and also as available from My Polymers, Ness Ziona, Israel), NOR-133 (Norland Products, Jamesburg, NJ; https://www.norlandproducts.com/adhesives/NOA133.html), and/or AF1601 Amorphous Fluoropolymer Solution.

In some embodiments, light passing through an isolation layer does not enter the reservoir. In some embodiments, light passing through an isolation layer does not contact an image sensor.

According to some embodiments, isolation layer 134 may include an absorbing structure, such as an exterior coating or other structure configured to absorb light passing through the isolation layer. Such a structure or coating may comprise one or more gaskets, O-rings, or the like, which may for instance comprise silicone or polyoxymethylene (Delrin). This absorbing structure may be placed on the outer perimeter of solid surfaces 106c and 106d and/or in the surface region between the light source and the reservoir contact, and may be of sufficient surface area to greatly absorb all undesired rays in the distance of the filter. If the lower side 106d of the solid 106 is not in contact with a higher index material, then the lower side 106d may not need an isolation layer 134 or absorbing structure. In this case, an upper absorbing structure on side 106c may be more efficient to assure sufficient attenuation.

In certain embodiments, a portion of the reaction region of the substrate comprises an isolation layer. In an exemplary configuration, the isolation layer covers the reaction region except for a plurality (e.g., an array) of wells or voids where the isolation layer is absent (e.g., has been removed, e.g., drilled or bored through). In certain embodiments, portions of the isolation layer may have been removed by etching (e.g., reactive ion etching (RIE)). In some embodiments, a spot is situated in a well or void on the surface of the substrate. In an exemplary configuration, the wells containing spots are the only portion of the reaction region not coated by an isolation layer. A well or void may have any cross-sectional shape (e.g., circular, elliptical, hexagonal, star-shaped, or square, rectangular, or other quadrilateral). A well or void may have straight or sloped sidewalls. Without wishing to be bound by a particular theory, the disclosure provides devices and methods that utilize the evanescent wave produced by total internal reflection to selectively manipulate one or more molecules in proximity to the surface of the substrate. An isolation layer configured to cover the reaction region of the substrate surface can optically isolate the evanescent wave from locations not designated, e.g., for spots, e.g., for sequencing of target nucleic acids. Decreasing exposure of the aqueous solution to light may decrease damage to reagents (e.g., sequencing reagents, such as protected nucleotides) and/or may decrease background or noise detected by the detector. In some embodiments, an isolation layer used in the reaction region decreases binding of aqueous solution components (e.g., nucleotides, polymerase, or solution phase polynucleotides) to the surface of the substrate.

In some embodiments, the isolation layer (e.g., wells and voids in the isolation layer) is configured according to the nucleic acid amplification methods and sequencing methods to be used. For example, the substrate polynucleotides and/or the read lengths used in amplicon sequencing methods may be longer than the substrate polynucleotides and/or read lengths used in shotgun sequencing methods, and the diameter and/or spacing of the wells or voids in the isolation layer may contribute to ensuring separation of the contents of one spot from another. In some embodiments, the wells or voids in the isolation layer are at least 1 μm, at least 2 μm, at least 3 μm, at least 4 μm, at least 5 μm, at least 8 μm, at least 10 μm, at least 12 μm, at least 14 μm, at least 16 μm, at least 18 μm, at least 20 μm, at least 22 μm, at least 24 μm, at least 26 μm, at least 28 μm, at least 30 μm, at least 32 μm, or at least 50 μm in diameter (e.g., 1-50 μm, 1-30 μm, 1-25 μm, 1-20 μm, 1-15 μm, 1-10 μm, 4-50 μm, 4-30 μm, 4-25 μm, 4-20 μm, 4-15 μm, 4-10 μm, 10-50 μm, 10-30 μm, 10-25 μm, 10-20 μm, 10-15 μm, 15-50 μm, 15-30 μm, 15-25 μm, 15-20 μm, 20-50 μm, 20-30 μm, 20-25 μm, 25-50 μm, 25-30 μm, or 30-50 μm in diameter) in a device of the disclosure (e.g., a device configured for amplicon sequencing methods). In some embodiments, the wells or voids in the isolation layer are at least 0.1 μm, at least 0.25 μm, at least 0.5 μm, at least 0.75 μm, at least 1 μm, at least 1.25 μm, at least 1.5 μm, at least 1.75 μm, at least 2 μm, at least 2.5 μm, or at least 3 μm in diameter (e.g., 0.1-3 μm, 0.1-2 μm, 0.1-1.5 μm, 0.1-1 μm, 0.1-0.5 μm, 0.5-3 μm, 0.5-2 μm, 0.5-1.5 μm, 0.5-1 μm, 1-3 μm, 1-2 μm, 1-1.5 μm, 1.5-3 μm, 1.5-2 μm, or 2-3 μm in diameter) in a device of the disclosure (e.g., a device configured for shotgun sequencing methods).

In some embodiments, an array of wells or voids (e.g., the distance separating the wells of an array from one another) is configured according to the nucleic acid amplification methods and sequencing methods to be used. For example, the substrate polynucleotides and/or the read lengths used in amplicon sequencing methods may be longer than the substrate polynucleotides and/or read lengths used in shotgun sequencing methods, and the wells of an array may be spaced to ensure amplification or sequencing in a first spot does not interfere with amplification or sequencing in a second spot based in part on the aforementioned lengths. In some embodiments, the distance separating the wells of an array from one another is at least 1 μm, 2 μm, 3 μm, 4 μm, 5 μm, 6 μm, 7 μm, 8 μm, 9 μm, 10 μm, 12 μm, 15 μm, 20 μm, 25 μm, 30 μm, 40 μm, 50 μm or 100 μm in a device of the disclosure. In some embodiments, the distance separating the wells of an array from one another is in a range from 1-5 μm, 1-10 μm, 1-20 μm, 1-50 μm, 1-100 μm, 5-10 μm, 5-20 μm, 5-50 μm, 5-100 μm, 10-20 μm, 10-50 μm, 10-100 μm, 20-50 μm, 20-100 μm, or 50-100 μm. In some embodiments, the distance separating the wells of an array from one another is at least 100 μm, at least 120 μm, at least 140 μm, at least 160 μm, at least 180 μm, at least 200 μm, at least 220 μm, at least 240 μm, at least 260 μm, at least 280 μm, or at least 300 μm (e.g., 100-300 μm, 100-250 μm, 100-200 μm, 100-150 μm, 150-300 μm, 150-250 μm, 150-200 μm, 200-300 μm, 200-250 μm, or 250-300 μm) in a device of the disclosure. In some embodiments, the distance separating the wells of an array from one another refers to the center-to-center distance between adjacent wells.

In some embodiments, the distance separating the wells of an array from one another is at least 1.5, 2, 3, 4, 5, 6, 7, 8, 9, or 10 times the average diameter (e.g., largest cross-section dimension) of the wells. In some embodiments, the distance separating the wells of an array from one another is 1.5 to 3, 1.5 to 5, 1.5 to 8, 1.5 to 10, 3 to 5, 3 to 8, 3 to 10, 5 to 8, or 5 to 10 times the average diameter (e.g., largest cross-sectional dimension) of the wells.

In some embodiments, the isolation layer may have a refractive index of greater than or equal to 1.33, 1.34, 1.35, 1.36, 1.37, 1.38, 1.39, or 1.40. According to some embodiments, isolation layer 134 has a refractive index of less than or equal to 1.40, 1.39, 1.38, 1.37, 1.36, 1.35, 1.34, or 1.33. Any suitable combinations of the above-referenced ranges are also possible. In certain embodiments, the isolation layer has a refractive index of about 1.33, 1.34, 1.35, 1.36, 1.37, 1.38, 1.39, or 1.40.

In some embodiments, the isolation layer used in the reaction region comprises a light-absorbing element that can be fashioned into features on the surface with microscale dimensions (e.g., structures separating spots where the structures are 1-5, 1-4, 1-3, or 1-2 μm in width). In some embodiments, the isolation layer used in the reaction region comprises a light-absorbing element that does not chemically interact (e.g., that is inert relative to) an adjacent device component, e.g., the light-absorbing element coupled to a peripheral region. In some embodiments, the isolation layer used in the reaction region does not fluoresce, e.g., when exposed to light from a light source described herein, e.g., when exposed to ultraviolet or visible light (e.g., light from 365-700 nm). In some embodiments, the isolation layer used in the reaction region comprises a light-absorbing element that is not denatured or destroyed by another substrate or reservoir region preparation step, e.g., is not denatured or destroyed by an O₂ plasma preparation or temperatures of 100-200° C.

In some embodiments, an isolation layer may be formed on at least a portion of the reaction region of the substrate prior to immobilization of substrate polynucleotides to the reaction region. In an illustrative embodiment, CYTOP® may be deposited on a substrate and etched to form wells, as described herein. In some cases, the CYTOP®-coated substrate may then be exposed to plasma (e.g., O₂ plasma). In some cases, at least a portion of the substrate (e.g., wells of the reaction region) may then be subjected to a surface treatment (e.g., coated with one or more layers of a silane-containing polymer or small molecule). In some cases, oligonucleotides may then be conjugated to the silane-containing polymer or small molecule.

Some embodiments are directed to a method of preparing a plurality of wells in an isolation layer. In some embodiments, the method comprises masking at least a portion of a reaction region of a substrate with a layer of a removable material (e.g., a photoresist or other soluble material). In some embodiments, the method comprises depositing a layer of a coating material on unmasked portions of the reaction region. The coating material may be deposited on the unmasked portions according to any deposition method. Non-limiting examples of suitable deposition methods include spin coating, sputtering, electron beam deposition, thermal evaporation, chemical vapor deposition, atomic layer deposition, and pulsed laser deposition. In some instances, spin-coating technologies may be used to coat unmasked portions of the substrate with a layer of the coating material having a thickness that may be controlled based on spin time, spin velocity, and/or viscosity of the coating material. Suitable spin-coating technologies may include those commonly used in semiconductor manufacturing. In some embodiments, the coating material comprises a polymer (e.g., CYTOP®, BIO-133, NOR-133, AF 1601 Amorphous Fluoropolymer Solution). In certain embodiments, the polymer is treated with additives to block transmission of light of a range corresponding to one or more first light sources and/or one or more second light sources.

In some embodiments, the method comprises removing the removable material from the reaction region (e.g., via use of solvents) after the layer of coating material has been spun onto the unmasked portions of the reaction regions. In some cases, the remaining coating material may serve as one or more isolation layers.

In some embodiments, the layer of coating material is spun in a manner to cover a reaction region of the substrate, forming an isolation layer across the reaction region of the substrate except in a plurality of patterned holes, which may allow penetration of the evanescent wave into the reservoir. In some embodiments, substrate polynucleotides are positioned in the plurality of patterned holes. In some embodiments, the remaining coating material may undergo further processing to, for example, harden and/or densify the coating material. In some embodiments, the coating material may comprise a polymer (e.g., a polymer treated with additives to block transmission of light of a range corresponding to one or more first light sources and/or one or more second light sources).

Some embodiments are directed to a method of preparing a plurality of wells in an isolation layer. In certain embodiments, the method comprises depositing a layer of a coating material on at least a portion of the substrate (e.g., a reaction region of the substrate). Non-limiting examples of suitable deposition methods include spin coating, sputtering, electron beam deposition, thermal evaporation, chemical vapor deposition, atomic layer deposition, and pulsed laser deposition. In certain instances, the deposition method comprises spin coating, and the thickness of the layer of coating material may be controlled based on spin time, spin velocity, and/or viscosity of the coating material. In some embodiments, the coating material comprises a polymer (e.g., CYTOP®, BIO-133, NOR-133, AF 1601 Amorphous Fluoropolymer Solution). In certain embodiments, polymer is treated with additives to block transmission of light of a range corresponding to one or more first light sources and/or one or more second light sources.

In some embodiments, a layer of a removable material (e.g., a photoresist or other soluble material) is deposited on at least a portion of the layer of coating material. Non-limiting examples of suitable deposition methods include spin coating, spray coating, roller coating, and transfer coating. In certain instances, the deposition method comprises spin-coating, and the thickness of the layer of the removable material may be controlled based on spin time, spin velocity, and/or viscosity of the removable material. In certain embodiments, the removable material comprises a photoresist. The photoresist may, in some cases, be a positive photoresist.

### Examples of Suitable Photoresists Include, but are not Limited to, AZ1505 and AZ9260

In some embodiments, the method comprises applying a patterned mask to the layer of the removable material. In some cases, the patterned mask comprises a plurality of patterned holes through which the removable material may be exposed to illumination. In some cases, the locations of the patterned holes may correspond to the desired locations of wells.

In some embodiments, the method comprises exposing the substrate to light (e.g., such that the unmasked portions of the removable material are exposed to the light). In some embodiments, exposure to light may alter the chemical structure of the removable material in unmasked regions such that it becomes more soluble in a photoresist developer. In some embodiments, the method comprises removing the unmasked portions of the removable material (e.g., using a photoresist developer to dissolve the unmasked portions). In some embodiments, the method comprises etching the layer of coating material beneath the unmasked portions of removable material to form wells. In some cases, etching may comprise reactive ion etching (RIE, e.g., using O₂ plasma).

In some cases, the layer of coating material covers a reaction region of the substrate, forming an isolation layer except in a plurality of patterned holes, which may allow penetration of the evanescent wave into the reservoir. In some embodiments, substrate polynucleotides are positioned in the plurality of patterned holes. In some embodiments, the remaining coating material may undergo further processing to, for example, harden and/or densify the coating material.

In some embodiments, the method comprises removing the removable material (e.g., photoresist). In certain cases, the removable material may be removed via use of solvents. In an illustrative, non-limiting example, one or more steps of removing the removable material from a substrate comprise spraying the substrate with acetone, rinsing with deionized water, and drying under nitrogen.

In some embodiments, the isolation layer may be a multilayer coating comprising a light-transmissible layer and an isolation layer. The light-transmissible layer may be a permissive layer and may have a desired refractive index configured to divert incident light rays in a direction away from the reservoir and/or in a direction toward the isolation layer. The isolation layer may prevent reflection of incident light.

### Optical Imaging System

As described above, an evanescent wave imaging apparatus may comprise an optical imaging system positioned below a reservoir and a substrate. Below are described various features of such an optical imaging system. The below description may be applied to any suitable embodiment described above in relation to FIGS. 1A-1G, including any of the above description relating to image sensor 118, lens 120, and/or optical filters 122 and 124, and their described features.

Image Sensor In some embodiments, the optical imaging system comprises an image sensor (e.g., 118) configured to detect light (e.g., emission light emitted by at least one detectable moiety of a protected nucleotide that has been incorporated into a sequencing primer). The image sensor may be any image sensor known in the art. In some cases, for example, the image sensor may be a complementary metal oxide semiconductor (CMOS) image sensor or a charge coupled device (CCD) image sensor. Non-limiting examples of a suitable image sensor include a Sony® IMX447 sensor (approximately 12 megapixels in an area of approximately 6.3 mm×4.7 mm), a Canon® single-photon avalanche diode (SPAD) image sensor (approximately 3 megapixels in an area of approximately 13.2 mm×9.9 mm), and the like.

In certain embodiments, the image sensor is operably coupled to, or comprises, a color filter, which may transmit only light within a particular wavelength band to a given region of the sensor (e.g., to a pixel). A non-limiting example of a suitable color filter is a Bayer color filter. In some embodiments, the color filter may be arranged over pixels of the image sensor such that each pixel may receive primarily red light (“red pixel”), primarily green light (“green pixel”), or primarily blue light (“blue pixel”). Regions of the color filter configured to transmit light of a particular color or wavelength band may be arranged in a regular pattern or array relative to other regions (e.g., as in the Bayer pattern).

As will be appreciated, data processing of large amounts of data (e.g., 12-megapixel images) may be computationally intensive and may limit how quickly a protected nucleotide may be identified. In some embodiments, a processing system (e.g., 126) operably coupled to an image sensor (e.g., 118) may be configured to perform a binning operation to lower a number of computations performed for a captured image. For example, for a color image that captures a full field of view of a reaction region of a substrate (e.g., an area in which all the spots on the substrate are located), the processing system may be programmed to perform binning of the pixels into groups of n pixels of the same color (e.g., n adjacent red pixels, n adjacent blue pixels, and n adjacent green pixels), and each group may be read out as a single output pixel having a value that is an average of the n pixels of the group. In some embodiments, two-by-two (“2×2”) binning may be used for a color image of a relatively low-density array of spots having a relatively larger spot size (e.g., spots having a center-to-center spacing of at least 45 μm and a spot diameter of at least 15 μm) because there is little loss of information by such binning. Other binning techniques known in the art may be used. On the other hand, binning may result in some loss of information for a color image of a relatively denser array of spots having a relatively smaller spot size (e.g., spots having a center-to-center spacing of about 30 μm or less and a spot diameter in a range of about 10 μm to 15 μm); in such cases the processing system may be programmed such that no binning occurs.

In some embodiments, after incorporation of a protected nucleotide in a sequencing primer annealed to a substrate polynucleotide, a detectable moiety of the protected nucleotide may be exposed to excitation light (e.g., by operating an evanescent wave imaging apparatus as described above) and may subsequently emit one or more photons. In some embodiments, each type of protected nucleotide may comprise a detectable moiety configured to emit a particular wavelength of light. For example, a protected nucleotide of a first type (e.g., guanine or G) may comprise a first type of detectable moiety that emits light at a first wavelength upon excitation, whereas a protected nucleotide of a second type (e.g., cytosine or C) may comprise a second type of detectable moiety that emits light at a second wavelength upon excitation, and so on for protected nucleotides of a third type, etc. The image sensor may capture a color image during an incorporation event (e.g., an event associated with production of an evanescent wave or field) and may provide image data of the color image to a processing system (e.g., 126), which may process the image data to associate a wavelength with each pixel or each of multiple groups of pixels of the color image. The processing system may output a pixel-by-pixel (or pixel group-by-pixel group) identification mapping for the incorporation event, which associates each pixel (or each pixel group) with a type of protected nucleotide based on the wavelength of light captured for the pixel (or pixel group). In some embodiments, each pixel (or each pixel group) may be associated with a spot or well where one or more substrate polynucleotides are immobilized on the substrate. In some embodiments, each pixel (or pixel group) of the image sensor may be configured to count a number of photons incident on the pixel (or pixel group) for the captured image, and may correlate the number of counted photons with the number of protected nucleotides incorporated at the spot corresponding to the pixel (or pixel group). As will be appreciated, when the image data indicates that little or no light (e.g., light having an intensity below a predetermined threshold) was captured at a pixel (or pixel group), the processing system may indicate an incorporation error for that pixel (or pixel group).

In some embodiments, the image sensor may capture a sequence of color images corresponding to a sequence of incorporation events that take place on the substrate. Each incorporation event may be followed by a cleaving or termination reversal event, which may enable a next protected nucleotide to be incorporated. The sequence of color images may be processed by the processing system to provide, for each pixel (or each pixel group), a sequence of nucleotide identifications that took place. Each pixel (or pixel group) may be associated with a spot or well where one or more substrate polynucleotides are immobilized on the substrate, and therefore the processing system may identify a sequence of nucleotides incorporated at each spot of the substrate. In some embodiments, the sequence of color images may correspond to video of the incorporation events on the substrate. The term “image data” as used herein may therefore refer at least to data of a single still image, data of a series of still images, or video data.

In some embodiments, the image sensor is a monochrome image sensor and does not operate with a color filter. Such a monochrome image sensor may not have the resolution limitation noted above and therefore may enable, in some embodiments, spot sizes to be on the order of pixel size. In some embodiments, the image sensor may have sufficiently high resolution such that each spot at which at least one substrate polynucleotide is immobilized may be imaged or sensed by fewer than three pixels of the image sensor. In some embodiments, emission light from each spot may be imaged by a single pixel. As will be appreciated, as spot size decreases, fewer photons may be generated at each spot, and therefore a high-sensitivity image sensor may be needed in order to resolve weak signals. A non-limiting example of a suitable high-sensitivity image sensor that may be used is a Canon® SPAD sensor, which may be able to capture 1-megapixel (or greater) images and which may be configured to amplify a single photon at each pixel.

In some embodiments, a monochrome image sensor may be used to confirm that each spot of a plurality of spots on a substrate has undergone an incorporation event successfully. For example, the substrate may have immobilized thereon a plurality of the same substrate polynucleotides all having the same structure. Therefore, for each incorporation event, the same type of protected nucleotide may be incorporated at each spot, and there may be less of need to identify which type of protected nucleotide was incorporated and more of a need to confirm that a successful incorporation occurred at each spot on the substrate. In some embodiments, the image sensor may provide monochrome image data to the processing system, which may process the monochrome image data to determine whether any incorporation errors occurred (e.g., by determining whether a light intensity captured for each spot is above a predetermined threshold). In some embodiments, the processing system may output an error map indicating where and/or which spot(s) on the substrate the nucleotide incorporation was not successful. In some embodiments, for video or for a sequence of monochrome images captured for a sequence of incorporation events, the processing system may process the monochrome image data to determine which spot(s) may have a sequencing error.

According to some embodiments, the distance between a bottom surface (e.g., 106d) of the substrate and the lens (e.g., 120) may be adjusted to produce a variety of desired optical effects. For example, the position of the lens toward the substrate may be adjusted to increase the amount of light captured by the lens, while decreasing the depth of focus. Moving the lens closer to the substrate effectively produces a smaller focal ratio, here being the ratio of the focal length of the lens to the diameter of the lens. According to some embodiments, the focal ratio may be greater than or equal to 2, 3, 4, 5, 6, 7, 8, 9, 10, 11, 12, 13, 14 or 15. According to some embodiments, the focal ratio may be less than or equal to 15,14, 13, 12, 11, 10, 9, 8, 7, 6, 5, 4, 3 or 2. Any suitable combinations of the above-referenced ranges are also possible.

Lenses

In some embodiments, the optical imaging system comprises one or more lenses (e.g., 120) positioned between a substrate and an image sensor (e.g., between fourth surface 106d of substrate 106 and image sensor 118). The one or more lenses may be configured to direct incident emission photons (e.g., photons emitted by a detectable moiety of a protected nucleotide) towards the image sensor or another element of the optical imaging system. In some cases, inclusion of one or more lenses in the optical imaging system may advantageously focus emission photons produced over one area onto an image sensor having a different surface area. For example, a reaction region in which emission photons may be emitted by detectable moieties of protected nucleotides may be larger than a sensor region of the image sensor; the one or more lenses may focus the emission photons from the relatively larger reaction region onto pixels of the comparatively smaller sensor region of the image sensor. Alternatively, the reaction region in which emission photons may be emitted by detectable moieties of protected nucleotides may be smaller than a sensor region of the image sensor. In this case, the one or more lenses may spread the emission photons from the relatively larger reaction region onto pixels of the comparatively larger sensor region of the image sensor.

In certain embodiments, the one or more lenses comprise a compound lens, a relay lens, a plano-convex lens, a microlens array, a concave lens, a focusing lens, and/or a parabolic reflector element. In certain instances, for example, the one or more lenses comprise a relay lens (e.g., a relay lens with a 1:1 magnification). A non-limiting example of a suitable relay lens is an Arducam \sqrt{1/2.5}″ M12 Mount 16 mm Focal Length Camera Lens M2016ZHO1. In some embodiments, for a large reaction region of spots in which substrate polynucleotides are immobilized, which may be of a different size than a size of the sensor region of the image sensor, a focusing lens (or a magnification lens) may be used to focus (or magnify) the reaction region onto the sensor region of the image sensor. For example, a 2:1 focusing lens may focus a comparatively wider field of the reaction region onto the sensor region of the image sensor; or a 1:2 diverging lens may spread a comparatively narrower field of the reaction region onto the sensor region of the image sensor. In certain embodiments, the one or more lenses comprise an infinity-corrected lens. In certain embodiments, the one or more lenses comprise a finite conjugate objective lens.

In some embodiments, the optical imaging system comprises a single lens. In certain embodiments, the lens is a compound lens. In certain embodiments, the lens is a finite conjugate microscope objective lens. In some such embodiments, magnification and focusing may be interdependent (e.g., magnification may be fixed once focusing is achieved).

In some embodiments, the optical imaging system comprises two or more lenses. In certain embodiments, for example, the optical imaging system comprises an upper lens and a lower lens. In certain embodiments, the upper lens is an infinity-corrected lens (e.g., positioned at its focal length from the substrate, looking down) and the lower lens is an infinity-corrected lens (e.g., positioned at its focal length from the sensor, looking up (infinity side towards the upper lens)). In some such embodiments, each lens may be positioned a precise distance from the sensor or the substrate, and the distance between the upper lens and the lower lens may have little to no impact on focus. In certain cases, this may facilitate manufacturing and/or may allow insertion of filters of varying thicknesses and/or optical lengths between the upper lens and the lower lens without impacting focus. The magnification in some such embodiments may be given by the ratio of focal lengths of the upper lens and the lower lens. In certain embodiments, the upper lens is a microscope objective lens and the lower lens is a tube lens.

In some embodiments, the focal length of the lens is greater than or equal to 5 mm, 10 mm, 15 mm, 20 mm, 25 mm, 30 mm, 35 mm, 40 mm, 45 mm, 50 mm, or 55 mm. In some embodiments, the focal length of the lens is less than or equal to 60 mm, 55 mm, 50 mm, 45 mm, 40 mm, 35 mm, 30 mm, 25 mm, 20 mm, 15 mm, or 10 mm. Any suitable combinations of the above-referenced ranges are also possible (e.g., a focal length of greater than or equal to 10 mm and less than or equal to 20 mm; or a focal length of greater than or equal to 20 mm and less than or equal to 30 mm).

Optical Filters for Filtering Emission Light

In some embodiments, the optical imaging system comprises one or more optical filters (e.g., 122) positioned between a substrate (i.e., a bottom surface of the substrate) and a lens (e.g., 120) and/or positioned between a lens and an image sensor. In certain embodiments, the one or more optical filters are operably coupled to the lens. An optical filter refers to a material that selectively transmits a first range of wavelengths and blocks (i.e., is partly or completely opaque to) a second range of wavelengths. Each of the one or more optical filters of the evanescent wave imaging apparatus may independently be an absorptive filter or a dichroic filter. In some embodiments, an optical filter comprises one or more layers of a dielectric material and/or a metal. In certain embodiments, an optical filter comprises two or more layers of materials having different refractive indices. In some embodiments, the optical filter comprises a volume of water.

In some embodiments, one or more optical filters of the optical imaging system are removable. In certain embodiments, the optical imaging system comprises a filter wheel. In some such embodiments, the filter wheel may allow different optical filters to be selected for different light sources. As an illustrative, non-limiting example, a 500 nm longpass filter may be used for 365 nm and 445 nm excitation, and a 570 nm longpass filter may be used for 520 nm excitation. In some embodiments, one or more optical filters of the optical imaging system may be permanently fixed in a particular position within an apparatus (e.g., between a substrate and a lens, between a lens and an image sensor).

In some embodiments, the one or more optical filters operably coupled to the lens comprise a longpass optical filter. For example, at least one of the one or more optical filters may block light having a wavelength of about 700 nm or less, about 650 nm or less, about 600 nm or less, about 590 nm or less, about 550 nm or less, about 500 nm or less, about 450 nm or less, about 400 nm or less, about 365 nm or less, or about 350 nm or less (and optionally transmits light above said wavelength).

In some embodiments, the one or more optical filters operably coupled to the lens comprise a shortpass optical filter. For example, at least one of the one or more optical filters may block light having a wavelength of at least 350 nm, at least 365 nm, at least 400 nm, at least 450 nm, at least 500 nm, at least 550 nm, at least 600 nm, at least 650 nm, or at least 700 nm (and optionally transmits light below said wavelength).

In some embodiments, the one or more optical filters operably coupled to the lens comprise a notch or bandcut optical filter configured to block light having one or more wavelengths within a range of wavelengths and transmit light having one or more wavelengths outside of that range. For example, at least one of the one or more optical filters may transmit light having a wavelength of about 700 nm or less, about 650 nm or less, about 600 nm or less, about 590 nm or less, about 550 nm or less, about 500 nm or less, about 450 nm or less, about 400 nm or less, about 365 nm or less, or about 350 nm or less and also may transmit light having a wavelength of at least 350 nm, at least 365 nm, at least 400 nm, at least 450 nm, at least 500 nm, at least 550 nm, at least 600 nm, at least 650 nm, or at least 700 nm (and blocks light between said wavelengths).

### Processing System

In some embodiments, a nucleic acid sequencing device comprising an evanescent wave imaging apparatus includes or otherwise operates in conjunction with a processing system (e.g., 126) configured to analyze data received from one or more image sensors. In some embodiments, the device is operably coupled, wirelessly and/or by one or more wires, to the processing system. Examples of wireless protocols that may be used for communication of electronic signals include, but are not limited to, Wi-Fi (e.g., any of the IEEE 802.11 family of protocols), Bluetooth®, Zigbee and other IEEE 802.15.4-based protocols, cellular protocols, and the like. In some instances, one or more components of the processing system are housed within a housing of the apparatus.

The processing system may comprise one or more processors (e.g., 128), which may include one or more general purpose processors and/or one or more a specially-adapted processors. For instance, the processing system may comprise a microprocessor (or microcontroller core), a field-programmable gate array (FPGA), an application-specific integrated circuit (ASIC), a custom integrated circuit, a digital signal processor (DSP), or combinations thereof.

In some embodiments, the processing system comprises a memory device (e.g., 130). The memory device may comprise any volatile and/or non-volatile memory, including but not limited to a hard-drive memory (e.g., solid-state-memory drive, magnetic memory, optical-disk drive, etc.), a removable storage medium (e.g., flash/USB memory, optical disk, floppy disk, magnetic memory, etc.), or combinations thereof. In some embodiments, the processing system may comprise at least one communication interface configured to allow the processing system to connect to one or more remote devices (e.g., a smartphone, a tablet, a host computer) in addition to components of the evanescent wave imaging apparatus (e.g., light source(s), image sensor, etc.). A non-limiting example of a suitable processing system is a Raspberry Pi 4B device comprising a processor, memory (RAM), a USB-C power supply, and onboard wireless networking and Bluetooth.

In some embodiments, the processing of data from one or more image sensors may be performed by both a processing system of the nucleic acid sequencing device and a remote computing device connected to the nucleic acid sequencing device through a suitable computer interface. Any suitable computer interface and remote computing device may be used. For example, the computer interface may be a USB interface or a FireWire interface. The remote computing device may be any general purpose computer, such as a laptop or desktop computer. The computer interface may facilitate communication of information between the device and the remote computing device. In other embodiments, the remote computing device may be omitted, and processing of data from one or more image sensors may be performed solely by the processing system of the nucleic acid sequencing device.

In some embodiments, the processing system includes a user interface for controlling operation of the nucleic acid sequencing device. The user interface may be configured to allow a user to input information, such as commands and/or settings used to control the functioning of the nucleic acid sequencing device. In some embodiments, the user interface includes any one or any combination of: buttons, switches, dials, keyboard(s), touchscreen(s), and microphone(s). In some embodiments, the user interface may allow a user to receive feedback on the performance of the device (e.g., based on information obtained from one or more sensors of the device).

In some embodiments, the user interface provides feedback using a speaker to provide audible feedback and/or indicator lights and/or a display screen to provide visual feedback. In some embodiments, the user interface provides output indicating whether an analyte (e.g., a target nucleic acid) was detected in sample. In certain embodiments, for example, one or more processors of the processing system are configured to receive image data provided by an image sensor (e.g., 118) and to cause the image data to be stored in one or more memory devices (e.g., 130) and/or to be processed by a detection module stored in the one or more memory devices (e.g., 130). In some embodiments, the detection module may identify a type of a protected nucleotide incorporated in a sequencing primer annealed to a substrate polynucleotide based on a color of light fluoresced by a detectable moiety of the protected nucleotide.

In some embodiments a module (e.g., a detection module) stored in one or more memory devices (e.g., 130) and/or an external computing device in electronic communication with the processing system (e.g., 126) stores each type of protected nucleotide identified for each sequencing primer and thereby stores a nucleic acid sequence for each sequencing primer. In some embodiments, the sequence data may be exported to an industry-standard format and best fit to multiple collections for phase correction. In some cases, during or after completion of one or more sequencing cycles, each stored nucleic acid sequence of the sequencing primers is compared to a database comprising one or more nucleic acid sequences of one or more target nucleic acids (e.g., nucleic acids of a pathogen). In some embodiments, if one or more nucleic acid sequences of the sequencing primers are at least 90%, 95%, 99%, or 100% identical to one or more nucleic acid sequences of one or more target nucleic acids, the user interface may provide audible or visual feedback indicating that the target nucleic acid is present in a sample. In some such embodiments, the user interface may further provide the name of the organism (e.g., pathogen, e.g., SARS-CoV-2) associated with the target nucleic acid. In some embodiments, the database may comprise nucleic acid sequences of two or more target nucleic acids (e.g., from two or more organisms, e.g., pathogens), and the user interface may provide feedback indicating whether any of the target nucleic acids are present in the sample.

In some embodiments, the nucleic acid sequencing device may be controlled by a companion app (e.g., a smartphone or other portable electronic device application) that controls the device over Bluetooth BLE. The app may allow a user to set parameters, choose a protocol, get notified when a protocol is complete, and/or display a report of protocol results.

### Exemplary Nucleic Acid Sequencing Devices

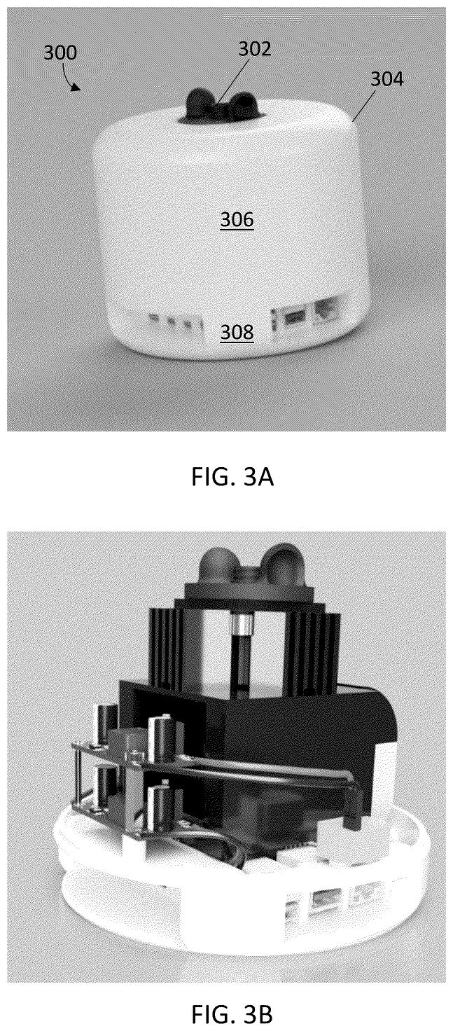

FIGS. 3A-3B show the exterior (FIG. 3A) and interior (FIG. 3B) of exemplary nucleic acid sequencing device 300 comprising reservoir 302 and evanescent wave imaging apparatus 304. As shown in FIG. 3A, evanescent wave imaging apparatus 300 comprises top outer housing 306 and bottom outer housing 308.

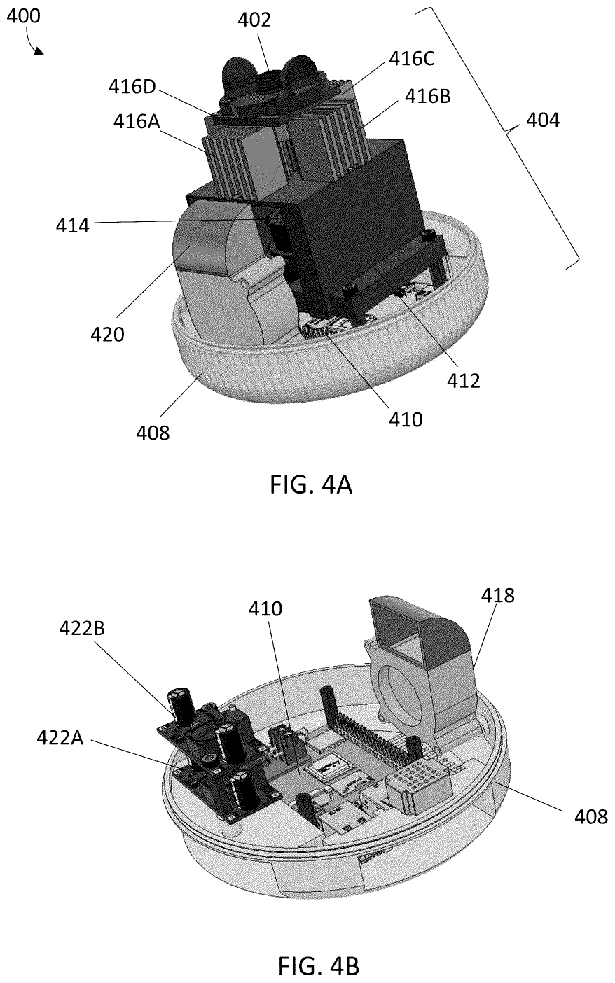

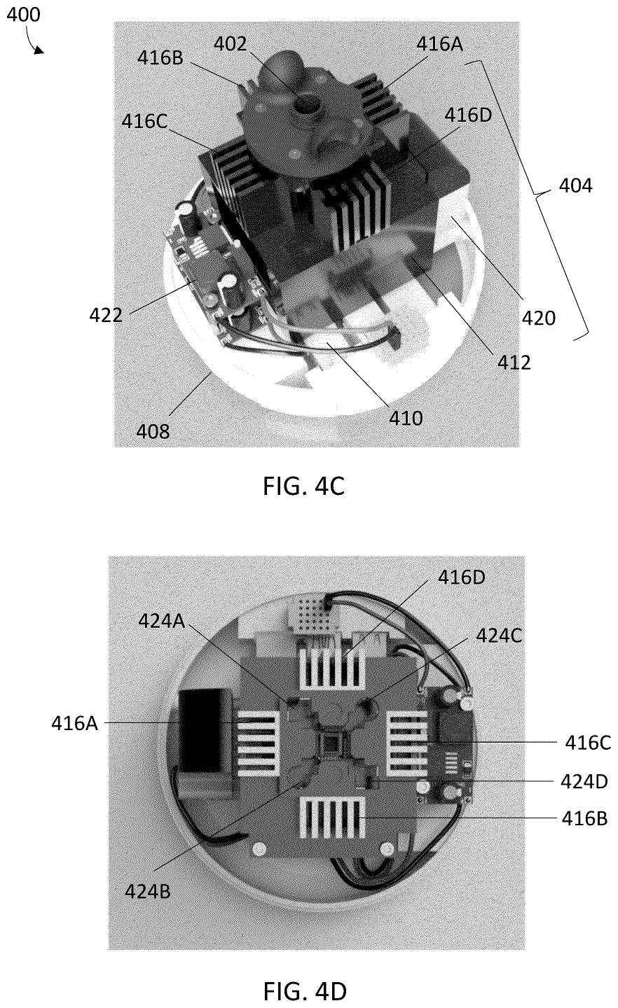

FIGS. 4A-4D show interior views of exemplary nucleic acid sequencing device 400 comprising reservoir 402 and evanescent wave imaging apparatus 404, and components thereof. FIG. 4A shows device 400 without top outer housing 406. In FIG. 4A, evanescent wave imaging apparatus 404 comprises bottom outer housing 408 and processing system 410 positioned on top of bottom outer housing 408. Inner housing 412, which houses optical imaging system 414, is positioned on top of processing system 410. Four heat sinks 416A-D are positioned on top of inner housing 412. Each of four heat sinks 416A-D is in thermal communication with at least one of four sets of light sources 418A-D (not shown in FIG. 4A). Reservoir 402, including a substrate (not shown in FIG. 4A), is positioned on top of heat sinks 416A-D and their associated sets of light sources 418A-D such that the substrate is appropriately aligned with sets of light sources 418A-D. Fan 420 is positioned on top of bottom outer housing 408 and to one side of inner housing 412.

FIG. 4B shows device 400 without reservoir 402, top outer housing 406, inner housing 412, optical imaging system 414, heat sinks 416A-D, or sets of light sources 418A-D. As shown in FIG. 4B, processing system 410 and fan 420 are positioned on bottom outer housing 408 of apparatus 404. FIG. 4B also shows first power converter 422A and second power converter 422B. At least one of first power converter 422A and second power converter 422B may be in electrical communication with light sources 418A-D, and the other of first power converter 422A and second power converter 422B may be in electrical communication with processing system 410 and/or fan 420.

FIG. 4C shows another view of device 400 without top outer housing 406. FIG. 4C shows reservoir 402, bottom housing 408, processing system 410, inner housing 412, heat sinks 416A-D, fan 420, and power converter 422.

FIG. 4D shows a top view of apparatus 404 without outer housing 406. FIG. 4D shows heat sinks 416A-D and reservoir alignment openings 424A-D, which are configured to facilitate alignment of reservoir 402 (e.g., a substrate of reservoir 402) with sets of light sources 418A-D of apparatus 404. Top views of fan 420 and power converter 422 are also visible in FIG. 4D.

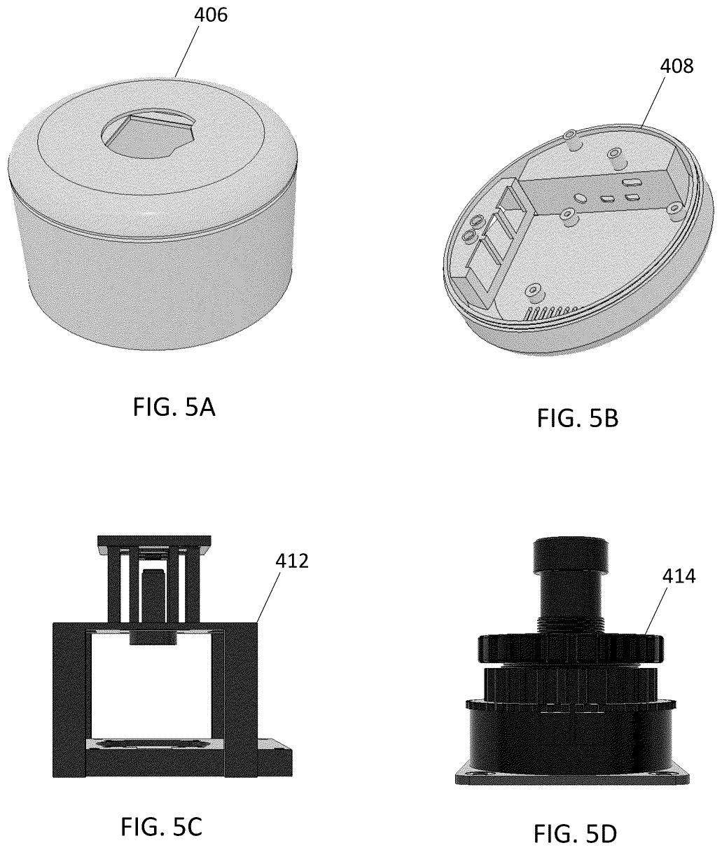

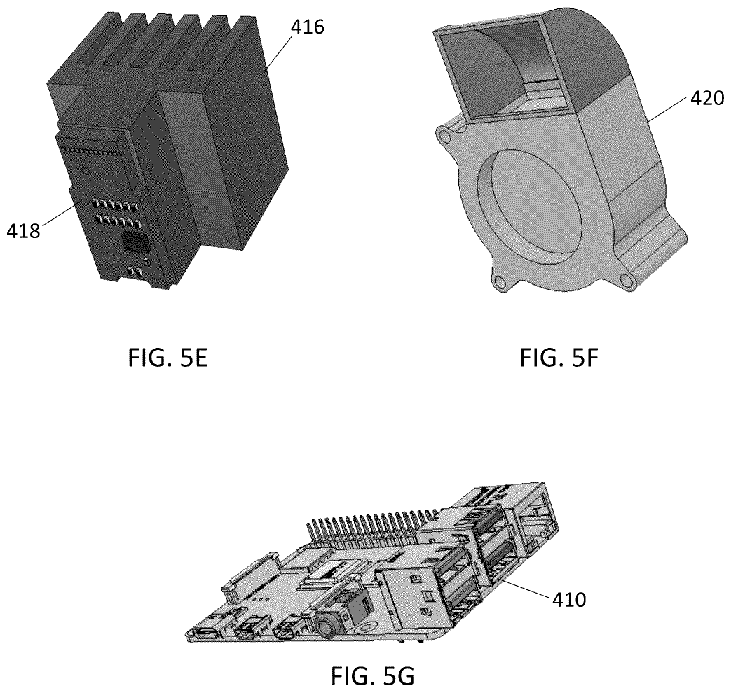

FIGS. 5A-5G show individual components of exemplary apparatus 404 of device 400. FIG. 5A shows top outer housing 406, FIG. 5B shows bottom outer housing 408, FIG. 5C shows inner housing 412, FIG. 5D shows optical imaging system 414, FIG. 5E shows a heat sink 416 and an associated set of light sources 418, FIG. 5F shows fan 420, and FIG. 5G shows processing system 410.

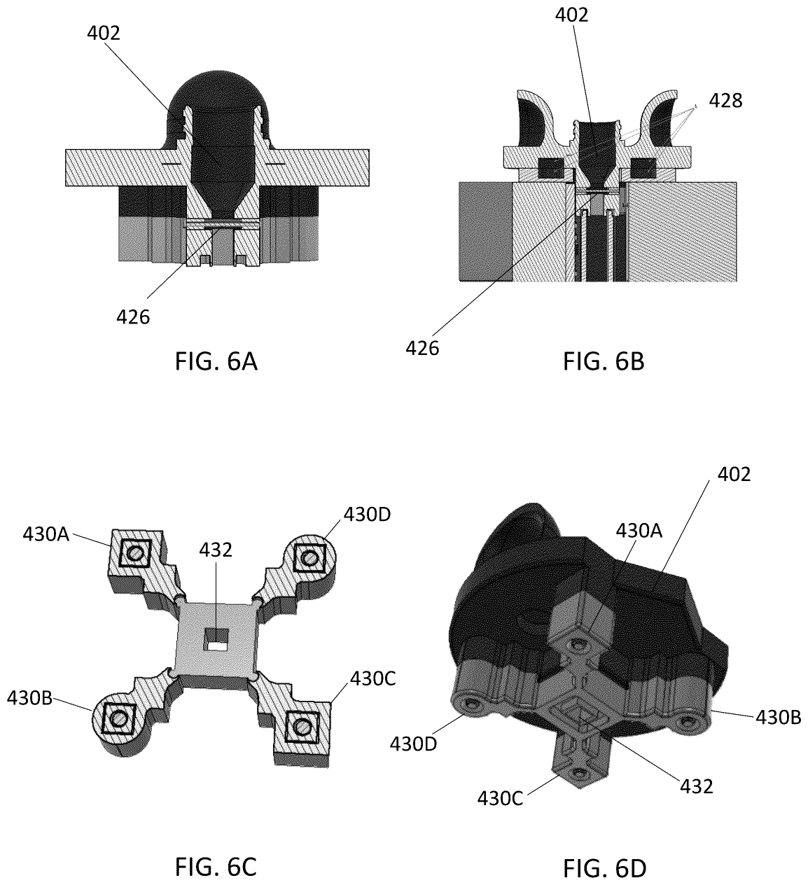

FIGS. 6A-6D show components of reservoir 402 of device 400. FIG. 6A shows a first cross-sectional view of reservoir 402. In FIG. 6A, substrate 426 is shown as forming a bottom surface of reservoir 402. FIG. 6B shows a second cross-sectional view of reservoir 402. In FIG. 6B, it can be seen that reservoir 402 can be connected to apparatus 404 by magnets 428. FIG. 6C shows a bottom view of reservoir 402. FIG. 6C shows reservoir alignment features 430A-D, which are configured to be inserted into reservoir alignment openings 424A-D in apparatus 404. An opening 432 for substrate 426 is also shown in FIG. 6C. FIG. 6D shows a bottom side perspective of reservoir 402, reservoir alignment features 430A-D, and substrate opening 432.

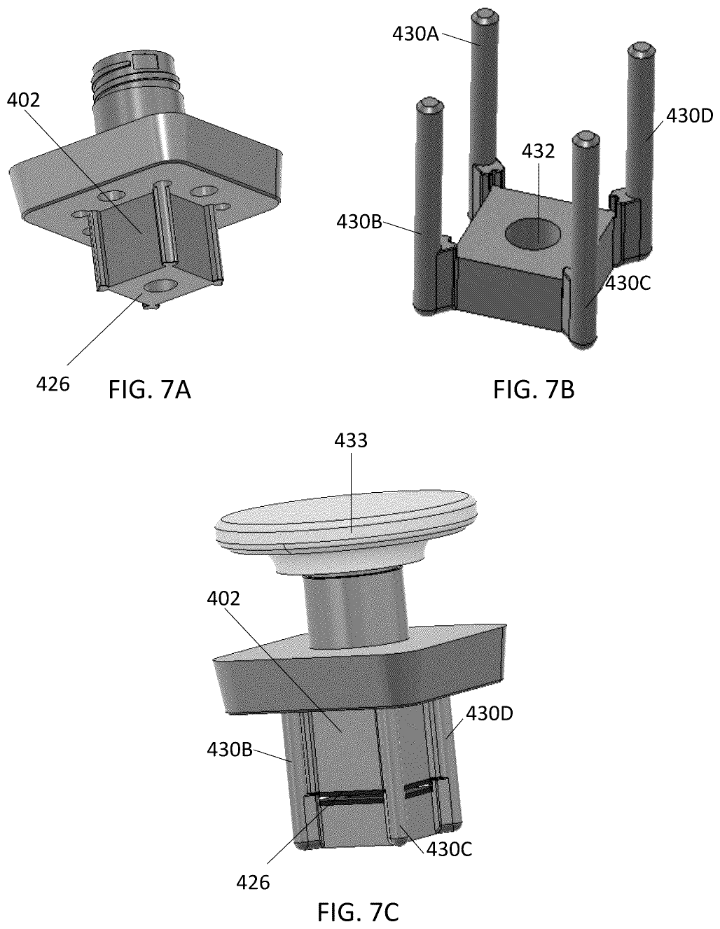

FIGS. 7A-7C show another embodiment of components of reservoir 402 of device 400, substrate 426, opening 432, and reservoir alignment features 430A-D. FIG. 7A show reservoir 402 and substrate 426 shown as a forming the bottom surface of reservoir 402. FIG. 7B shows opening 432 and reservoir alignment features 430A-D. FIG. 7C shows the components of FIGS. 7A and 7B assembled together, with reservoir 402 coupled with opening 432 via reservoir alignment features 430A-D, and substrate 426 situated below the bottom surface of reservoir 402 and above opening 432. FIG. 7C also depicts cap 433 which covers the top of reservoir 402.

In operation, reservoir 402 is inserted into evanescent wave imaging apparatus 404. Reservoir alignment features 430A-D of reservoir 402 are inserted into reservoir alignment openings 424A-D of apparatus 404 to facilitate appropriate alignment of reservoir 402 and apparatus 404 (e.g., appropriate alignment of substrate 426 with sets of light sources 418A-D). Magnets 428 further facilitate appropriate alignment of reservoir 402 and apparatus 404 and provide a connection between reservoir 402 and apparatus 404.

Reservoir 402 may contain an aqueous solution comprising a pool of protected nucleotides and polymerase, and substrate 426 may comprise a pool of substrate polynucleotides immobilized to a top surface of substrate 426. In some embodiments, a substrate polynucleotide immobilized to a top surface of substrate 426 may be contacted by a sequencing primer and a protected nucleotide in reservoir 402 comprising a detectable moiety and a photocleavable terminating moiety. In some embodiments, the substrate polynucleotide is also contacted by a polymerase such that the polymerase incorporates a protected nucleotide into the sequencing primer using the substrate polynucleotide as template. Due to the presence of the photocleavable terminating moiety and/or the detectable moiety, further elongation of the sequencing primer (i.e., further incorporation of one or more protected nucleotides) may be terminated. In some cases, at least one set of light sources 418A-D emits one or more pulses of light having an appropriate peak wavelength and power density to excite the detectable moiety of the incorporated protected nucleotide. The detectable moiety (e.g., a fluorophore) may emit one or more photons, which may be transmitted through substrate 426 to optical imaging system 414. As a result of detecting the emitted light, optical imaging system 414 may send one or more electrical signals to processing system 410. Processing system 410, or a device in wired or wireless communication with processing system 410, may analyze the one or more electrical signals and identify the protected nucleotide. In some embodiments, at least one of light sources 418A-D subsequently emits one or more pulses of light having an appropriate peak wavelength and power density to cleave the photocleavable terminating moiety of the protected nucleotide. Cleavage of the photocleavable terminating moiety may reverse termination of sequencing primer elongation, and a polymerase may further incorporate another protected nucleotide into the sequencing primer.

During operation, air may flow from one or more vents in bottom outer housing 408 through fan 420 and heat sinks 416A-D. In some embodiments, each set of light sources 418A-D is in thermal communication with at least one heat sink of heat sinks 416A-D, and heat may be transferred from light sources 418A-D to heat sinks 416A-D. In some embodiments, heat may exit apparatus 400 through a ventilation loop on the top of apparatus 400 (not shown in FIGS. 4-6).

### Sample Processing

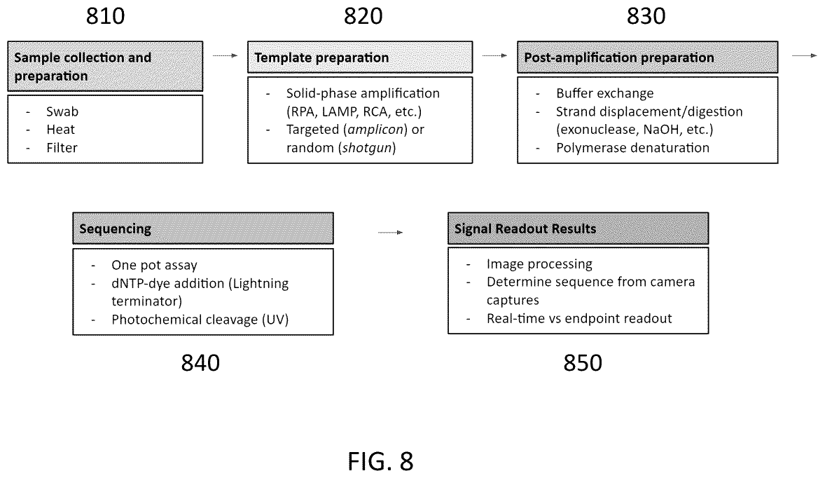

In some embodiments, a sample (e.g., a biological sample) undergoes one or more steps to prepare the sample for evanescent wave imaging. FIG. 8 provides an exemplary workflow for a method of nucleic acid sequencing, according to some embodiments. In the method of the workflow shown in FIG. 8, one or more steps may be omitted, two or more steps may be performed concurrently, and/or one or more additional steps not explicitly shown may be performed before, during, or after one or more steps shown.

As shown in FIG. 8, first step 810 comprises sample collection and preparation. In certain embodiments, first step 810 comprises collecting a sample from a subject (e.g., using a sample-collecting component, such as a nasal swab). In certain embodiments, the collected sample may be processed in one or more heating and/or filtering steps. In some embodiments, second step 820 comprises template preparation (e.g., nucleic acid amplification). In certain embodiments, for example, one or more nucleic acid sequences of a target nucleic acid that may be present in the collected sample may be amplified using an isothermal amplification method (e.g., RPA, LAMP, RCA, etc.). In some embodiments, third step 830 comprises post-amplification preparation. In some cases, for example, third step 830 may prepare a post-amplification reservoir for nucleic acid sequencing (e.g., by inactivating or eliminating one or more reagents from step 820 such that they do not interfere with a subsequent nucleic acid sequencing step). In certain embodiments, third step 830 comprises one or more buffer exchange, strand displacement, digestion (e.g., by an exonuclease, NaOH, etc.), and/or polymerase denaturation steps. In some embodiments, fourth step 840 comprises nucleic acid sequencing. Fourth step 940 may be performed as a one-pot assay, using an evanescent wave imaging apparatus to control incorporation of protected nucleotides into sequencing primers and cleavage of photocleavable terminating moieties of the protected nucleotides. In some embodiments, fifth step 850 comprises delivering signal readout results. In some embodiments, fifth step 850 comprises processing images captured by an image sensor, determining a nucleic acid sequence of a sequencing primer from a sequence of images, and outputting information (e.g., the sequence, the identity of a nucleic acid matching the sequence) to a user. In some cases, such information may be output in real time as sequencing step 840 is being performed. In some cases, such information may be output at an endpoint after completion of sequencing step 840.

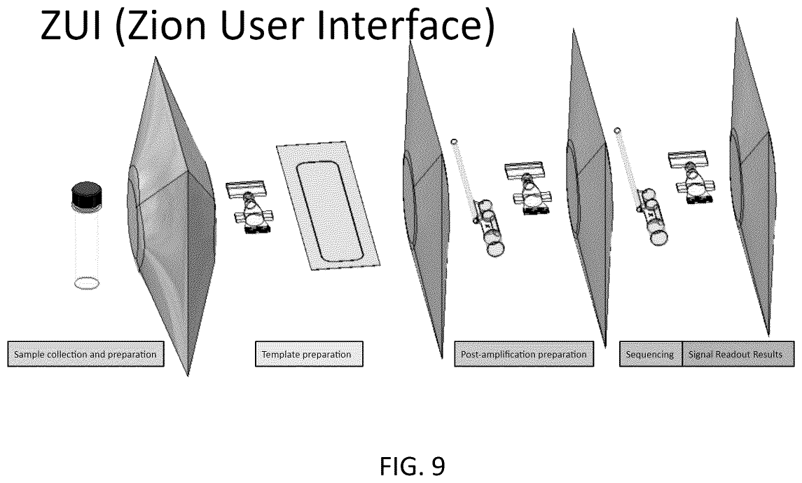

FIG. 9 provides exemplary user interface materials, e.g., for a kit comprising a device described herein, enabling a user to proceed through the exemplary workflow of FIG. 8. In some embodiments, the kit may have a relatively small number of components. In certain embodiments, the kit may consist of one or more reaction tubes, droppers, and/or pipettes.

In some embodiments of the present disclosure, one or more additional agents are added to the sample material to facilitate detection or identification of an analyte. In some embodiments, a label is added to the sample material. In some embodiments, the label comprises an antibody or antigen binding fragment thereof that binds the analyte. In some embodiments, the label comprises a detectable moiety. In some embodiments, the label comprises a FRET acceptor particle. In some embodiments, a plurality of labels are added to the sample, e.g., a label for each analyte to be detected or identified. In some embodiments, the one or more additional agents (e.g., comprising one or more labels) are added to the lysed sample after cell lysis (e.g., as described herein).

### Cell Lysis

In some embodiments, a method for preparing a sample for evanescent wave imaging comprises lysing cells in a sample (e.g., a biological sample collected from a subject). The step of cell lysis may be performed to access the intracellular contents (e.g., nucleic acid molecules) of cells within a sample. Cell lysis generally refers to a method in which the outer boundary or cell membrane of a cell is broken down or destroyed to release intracellular materials (e.g., DNA, RNA, proteins, organelles).

In some instances, the method of preparing a sample for evanescent wave imaging comprises performing a chemical lysis step (e.g., exposing the sample to one or more lysis reagents). In some embodiments, exposing the sample to one or more lysis reagents comprises adding one or more lysis reagents to the sample. The step of adding one or more lysis reagents to the sample may occur in a reservoir described herein or may occur in a separate vessel (e.g., a test tube).

In certain embodiments, the one or more lysis reagents comprise one or more detergents. Without wishing to be bound by a particular theory, a detergent may solubilize membrane proteins and rupture the cell membrane by disrupting interactions between lipids and/or proteins. Non-limiting examples of suitable detergents include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In certain embodiments, the one or more lysis reagents comprise one or more enzymes. Non-limiting examples of suitable enzymes include lysozyme, lysostaphin, zymolase, cellulase, protease, and glycanase. In some embodiments, the one or more lysis reagents comprise a pH-changing reagent (e.g., an acid or base).

In some embodiments, the one or more lysis reagents are active at approximately room temperature (e.g., 20° C.-25° C.). In some embodiments, the one or more lysis reagents are active at elevated temperatures (e.g., at least 37° C., at least 40° C., at least 50° C., at least 60° C., at least 65° C., at least 70° C., at least 80° C., at least 90° C.).

In some embodiments, one or more (and, in some cases, all) of the lysis reagents are in solid form (e.g., lyophilized, dried, crystallized, air jetted). In certain cases, the one or more lysis reagents in solid form are in the form of one or more beads and/or tablets. In some embodiments, the one or more beads and/or tablets are stable at room temperature for a relatively long period of time. In certain embodiments, the one or more beads and/or tablets are stable at room temperature for at least 1 month, at least 3 months, at least 6 months, at least 9 months, at least 1 year, at least 2 years, at least 3 years, at least 4 years, at least 5 years, at least 6 years, at least 7 years, at least 8 years, at least 9 years, or at least 10 years. In some embodiments, the one or more beads and/or tablets are stable at room temperature for 1-3 months, 1-6 months, 1-9 months, 1 month to 1 year, 1 month to 2 years, 1 month to 5 years, 1 month to 10 years, 3-6 months, 3-9 months, 3 months to 1 year, 3 months to 2 years, 3 months to 5 years, 3 months to 10 years, 6-9 months, 6 months to 1 year, 6 months to 2 years, 6 months to 5 years, 6 months to 10 years, 9 months to 1 year, 9 months to 2 years, 9 months to 5 years, 9 months to 10 years, 1-2 years, 1-3 years, 1-4 years, 1-5 years, 1-6 years, 1-7 years, 1-8 years, 1-9 years, 1-10 years, 2-5 years, 2-10 years, 3-5 years, 3-10 years, 4-10 years, 5-10 years, 6-10 years, 7-10 years, 8-10 years, or 9-10 years.

In some instances, the method of preparing a sample for evanescent wave imaging comprises performing a thermal lysis step (e.g., heating the sample). In some cases, exposure of cells to high temperatures can damage the cellular membrane by denaturing membrane proteins, resulting in cell lysis and the release of intracellular material.

In certain embodiments, thermal lysis is performed by applying a lysis heating protocol comprising heating a sample at one or more temperatures for one or more time periods using any heater known in the art. In some embodiments, a lysis heating protocol comprises heating the sample at a first temperature for a first time period. In certain instances, the first temperature is at least 37° C., at least 40° C., at least 50° C., at least 60° C., at least 63.5° C., at least 65° C., at least 70° C., at least 80° C., or at least 90° C. In certain instances, the first temperature is in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the first time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the first time period is in a range from 1 to 3 minutes, 1 to 5 minutes, 1 to 10 minutes, 1 to 15 minutes, 1 to 20 minutes, 1 to 30 minutes, 1 to 30 minutes, 1 to 40 minutes, 1 to 50 minutes, 1 to 55 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 55 minutes, 3 to 60 minutes, 5 to 10 minutes, 5 to 15 minutes, 5 to 20 minutes, 5 to 30 minutes, 5 to 40 minutes, 5 to 50 minutes, 5 to 55 minutes, 5 to 60 minutes, 10 to 20 minutes, 10 to 30 minutes, 10 to 40 minutes, 10 to 50 minutes, 10 to 55 minutes, 10 to 60 minutes, 20 to 30 minutes, 20 to 40 minutes, 20 to 50 minutes, 20 to 55 minutes, 20 to 60 minutes, 30 to 40 minutes, 30 to 50 minutes, 30 to 55 minutes, 30 to 60 minutes, 40 to 50 minutes, 40 to 55 minutes, 40 to 60 minutes, or 50 to 60 minutes.

In some embodiments, a lysis heating protocol comprises heating the sample at a second temperature for a second time period. In certain instances, the second temperature is at least 37° C., at least 40° C., at least 50° C., at least 60° C., at least 63.5° C., at least 65° C., at least 70° C., at least 80° C., or at least 90° C. In certain instances, the second temperature is in a range from 37° C. to 50° C., 37° C. to 60° C., 37° C. to 63.5° C., 37° C. to 65° C., 37° C. to 70° C., 37° C. to 80° C., 37° C. to 90° C., 50° C. to 60° C., 50° C. to 63.5° C., 50° C. to 65° C., 50° C. to 70° C., 50° C. to 80° C., 50° C. to 90° C., 60° C. to 65° C., 60° C. to 70° C., 60° C. to 80° C., 60° C. to 90° C., 65° C. to 80° C., 65° C. to 90° C., 70° C. to 80° C., or 70° C. to 90° C. In certain instances, the second time period is at least 1 minute, at least 2 minutes, at least 3 minutes, at least 4 minutes, at least 5 minutes, at least 10 minutes, at least 15 minutes, at least 20 minutes, at least 30 minutes, at least 40 minutes, at least 50 minutes, at least 55 minutes, or at least 60 minutes. In certain instances, the second time period is in a range from 1 to 3 minutes, 1 to 5 minutes, 1 to 10 minutes, 1 to 15 minutes, 1 to 20 minutes, 1 to 30 minutes, 1 to 30 minutes, 1 to 40 minutes, 1 to 50 minutes, 1 to 55 minutes, 1 to 60 minutes, 3 to 5 minutes, 3 to 10 minutes, 3 to 15 minutes, 3 to 20 minutes, 3 to 30 minutes, 3 to 40 minutes, 3 to 50 minutes, 3 to 55 minutes, 3 to 60 minutes, 5 to 10 minutes, 5 to 15 minutes, 5 to 20 minutes, 5 to 30 minutes, 5 to 40 minutes, 5 to 50 minutes, 5 to 55 minutes, 5 to 60 minutes, 10 to 20 minutes, 10 to 30 minutes, 10 to 40 minutes, 10 to 50 minutes, 10 to 55 minutes, 10 to 60 minutes, 20 to 30 minutes, 20 to 40 minutes, 20 to 50 minutes, 20 to 55 minutes, 20 to 60 minutes, 30 to 40 minutes, 30 to 50 minutes, 30 to 55 minutes, 30 to 60 minutes, 40 to 50 minutes, 40 to 55 minutes, 40 to 60 minutes, or 50 to 60 minutes.

In some embodiments, a lysis heating protocol may comprise heating a sample at one or more additional temperatures for one or more additional time periods.

In one non-limiting embodiment, the first temperature is in a range from 37° C. to 50° C. (e.g., about 37° C.) and the first time period is in a range from 1 minute to 5 minutes (e.g., about 3 minutes), and the second temperature is in a range from 60° C. to 70° C. (e.g., about 65° C.) and the second time period is in a range from 5 minutes to 15 minutes (e.g., about 10 minutes).

Although chemical lysis and thermal lysis are described herein, any suitable method of cell lysis may be used.

### Nucleic Acid Extraction and Purification

Cell lysis generally results in the release of all intracellular materials, including both nucleic acids and other material (e.g., proteins, lipids and other contaminants), from a cell. Following cell lysis, in some embodiments a step of nucleic acid extraction and/or purification is performed to separate the nucleic acid molecules from other cellular material. Methods of nucleic acid extraction and purification include solution-based methods and solid-phase methods.

In some embodiments, a method of nucleic acid extraction and/or purification is a solution-based method. Such methods may comprise mixing lysed sample material with solutions of reagents for purifying RNA and/or DNA. Solution-based methods of nucleic acid extraction and/or purification include, but are not limited to, guanidinium thiocyanate-phenol-chloroform extraction, cetyltrimethylammonium bromide extraction, Chelex® extraction, alkaline extraction, and cesium chloride gradient centrifugation (with ethidium bromide).

In some embodiments, a method of nucleic acid extraction and/or purification is a solid-phase method. Such methods may extract nucleic acid molecules from other cellular material by causing nucleic acids to selectively bind to solid supports, such as beads (e.g., magnetic beads coated with silica), ion-exchange resins, or other materials. In certain embodiments, a chaotropic agent (e.g., molecules that disrupt hydrogen bonding in aqueous solution) is added to the lysed sample material (e.g., to render nucleic acids less soluble and more likely to bind to solid supports). In some embodiments, the lysed sample material (with or without a chaotropic agent) is brought into contact with a solid support. In some cases, the solid support is washed with an alcohol to remove undesired cellular material and other contaminants from the solid support. In some cases, bound nucleic acid molecules are subsequently eluted from the solid support. Elution may, in some embodiments, be accomplished by washing the solid supports with a liquid that re-solubilizes the nucleic acids, thereby freeing the DNA from the support. Solid-phase extraction methods may utilize or comprise spin columns, beads (e.g., magnetic beads), automated nucleic acid extraction systems, liquid handling robots, lab-on-a-chip cartridges, and/or microfluidics.

Other embodiments of the present disclosure do not require a step of nucleic acid extraction and/or purification to separate the nucleic acid molecules from other cellular material. In such embodiments, described elsewhere herein, the nucleic acid molecules of the sample are reverse-transcribed to cDNA and subsequently amplified directly from the specimen in the buffer (e.g., without the need for a separate nucleic acid extraction and purification step).

### Nucleic Acid Amplification

In some embodiments, a method comprises performing a nucleic acid amplification reaction configured to amplify one or more target nucleic acid sequences (e.g., one or more nucleic acid sequences of a target pathogen). In some embodiments, one or more target nucleic acid sequences are amplified prior to contacting a substrate polynucleotide with a sequencing primer and a protected nucleotide. In certain cases, one or more amplification steps occur in a reservoir (e.g., in the aqueous solution of the reservoir). For example, some methods of the disclosure comprise performing nucleic acid amplification in the aqueous solution of the reservoir; such amplification may be followed by production of a pool of daughter strand amplicons immobilized to the surface of the substrate. In certain cases, one or more amplification steps occur outside the reservoir. For example, a nucleic acid amplification of target nucleic acids may occur outside the reservoir, and daughter strand amplicons may be added to the reservoir where a subsequent step produces a pool of daughter strand amplicons immobilized to the surface of the substrate. In certain cases, one or more amplification steps occur on a surface, e.g., the top surface of the substrate.

In some embodiments, amplifying a target nucleic acid sequence comprises performing an isothermal nucleic acid amplification reaction. Non-limiting examples of suitable isothermal amplification methods include recombinase polymerase amplification (RPA), loop-mediated amplification (LAMP), rolling circle amplification (RCA), and WildFire amplification. In certain embodiments, performing the isothermal nucleic acid amplification reaction comprises contacting a sample with one or more nucleic acid amplification reagents (e.g., RPA reagents, LAMP reagents, RCA reagents, or WildFire reagents). In some embodiments, amplification comprises a reverse transcription step (e.g., to reverse transcribe an RNA target nucleic acid) and may be referred to with the prefix RT (e.g., RT-RPA, RT-LAMP). In some embodiments, amplification reagents comprise a reverse transcriptase and/or an RNase (e.g., RNase H).

In some embodiments, performing the isothermal nucleic acid amplification reaction does not comprise heating the sample. In some embodiments, devices of the disclosure do not comprise a heating component. In some embodiments, devices of the disclosure do not comprise a means to cycle a sample or solution (e.g., the aqueous solution of the reservoir) from temperature to temperature. Without wishing to be bound by a particular theory, heating and/or temperature cycling may increase the cost and complexity of performing such methods or producing and using said devices. In some embodiments, performing the isothermal nucleic acid amplification reaction comprises heating the sample for one or more periods of time (e.g., to a single target temperature or single range of target temperatures above the ambient temperature) but not cycling between 3 or more target temperatures (i.e., not thermocycling in the manner of traditional PCR). For example, an isothermal nucleic acid amplification reaction may be performed by establishing and/or maintaining a temperature (e.g., about 30° C., 35° C., 40° C., 45° C., 50° C., 55° C., 60° C., 65° C., or 70° C.) above ambient for the duration of amplification and optionally the duration of sequencing. The disclosure is directed, in part to the discovery that isothermal methods of sequencing (e.g., including isothermal methods of nucleic acid amplification) can be accomplished using evanescent wave imaging, adding to the advantages of the methods and devices of the disclosure.

Methods of amplifying ribonucleic acids (RNA) and deoxyribonucleic acids (DNA) are specifically contemplated herein. In certain instances, for example, a target pathogen is an RNA virus (e.g., a coronavirus, an influenza virus), and therefore has RNA as its genetic material. In some such cases, the target pathogen's RNA may need to be reverse transcribed to DNA prior to amplification.

In some embodiments, performing a nucleic acid amplification reaction configured to amplify one or more target nucleic acid sequences comprises detecting and/or quantifying the nucleic acid amplification reaction. In some embodiments, detecting and/or quantifying the nucleic acid amplification reaction comprises adding a nucleic acid binding dye (e.g., an intercalating and/or fluorescent dye) to the aqueous solution of the reservoir (e.g., as an amplification reagent). In some embodiments, detecting and/or quantifying the nucleic acid amplification reaction comprises monitoring the nucleic acid amplification reaction. In some embodiments, a method described herein comprises proceeding with sequencing a target nucleic acid responsive to the success of the nucleic acid amplification reaction and/or the presence of amplicons comprising the target nucleic acid. For example, monitoring the fluorescence of a nucleic acid binding fluorescent dye during a nucleic acid amplification, e.g., in each spot on the surface of a substrate, can yield information regarding the amplification status and presence of each target nucleic acid to be sequenced across the plurality of spots.

RPA

In some embodiments, a method of sequencing of the disclosure comprises recombinase polymerase amplification (RPA) of one or more nucleic acid sequences. In some embodiments, a device of the disclosure is capable of sequencing a daughter strand (also referred to as an amplicon) comprising a sequence identical to or complementary to a target nucleic acid sequence that was amplified using RPA (e.g., either in the reservoir or prior to being added to the reservoir). In some embodiments, RPA is combined with a reverse transcription reaction and referred to as RT-RPA. Accordingly, in some embodiments, the reservoir (e.g., the aqueous solution) comprises one or more RPA reagents.

In some embodiments, RPA reagents comprise a forward primer, a reverse primer, a recombinase, a single-stranded binding protein, a strand-displacing polymerase, and nucleotides (dNTPs). In some embodiments, RPA reagents comprise a reverse transcriptase (e.g., in addition to a strand-displacing polymerase), e.g., that has strand-displacing activity. In some cases, the forward and reverse primers each form complexes with one or more recombinases, referred to herein as nucleoprotein primers. A non-limiting example of a suitable recombinase is T4 UvsX. The forward and reverse nucleoprotein primers may be capable of binding to complementary target nucleic acids, with the recombinase facilitating strand invasion of double-stranded target nucleic acids. The single-stranded binding protein may bind to a single-stranded target nucleic acid and prevent reannealing or strand migration.

In some embodiments, one or more (and, in some cases, all) RPA agents are present in the reservoir (e.g., the aqueous solution). In certain embodiments, for example, the forward primer is a solution-phase polynucleotide. In certain embodiments, the reverse primer is a solution-phase polynucleotide. In some embodiments, one or more of a recombinase, a single-stranded binding protein, a strand-displacing polymerase, and nucleotides (dNTPs) are also present in solution in the reservoir.

In some embodiments, one or more RPA reagents are immobilized to a top surface of a substrate (i.e., a bottom surface of the reservoir). In some embodiments, one or more RPA primers is present as a substrate polynucleotide in a substrate construct, immobilized to the reaction region of the surface of the substrate, e.g., in a spot. In certain embodiments, for example, one or more reverse RPA primers are immobilized to a surface (e.g., a top surface) of the substrate. In some instances, one or more reverse RPA primers are immobilized to a surface (e.g., a top surface) of the substrate as part of a substrate construct and one or more forward RPA primers are present in the aqueous solution. In some instances, one or more forward RPA primers are immobilized to a surface (e.g., a top surface) of the substrate as part of a substrate construct and one or more reverse RPA primers are present in the aqueous solution.

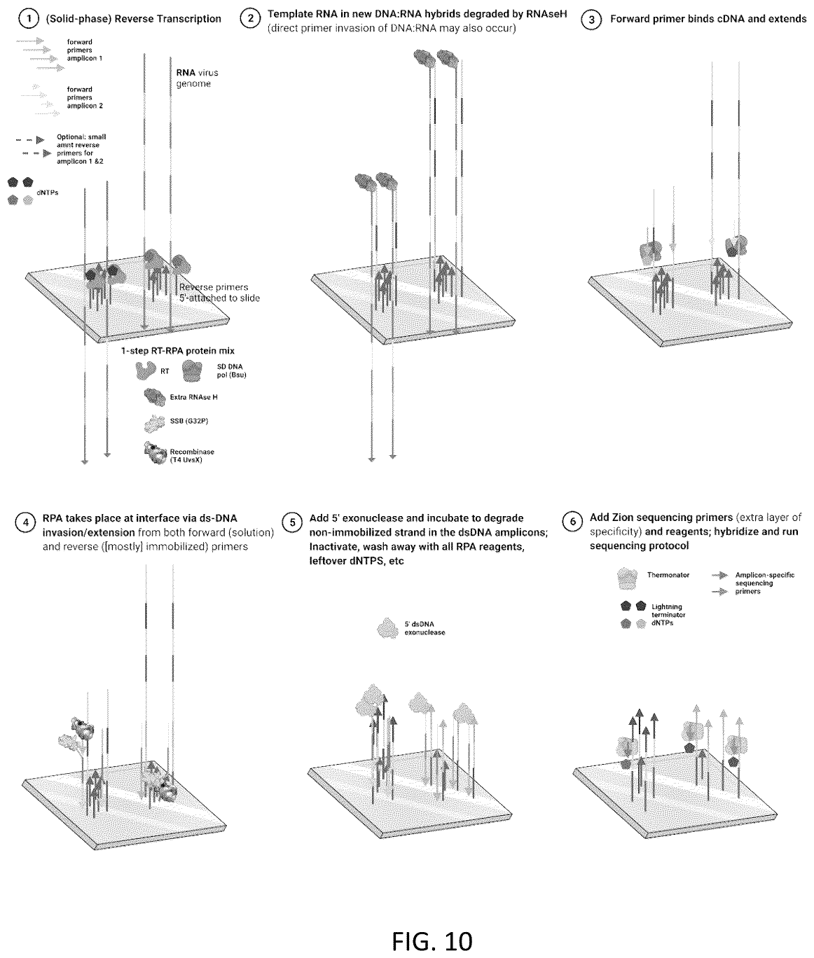

FIG. 10 shows a schematic illustration of an exemplary solid phase reverse transcriptase RPA (RT-RPA) workflow. As shown in FIG. 10, one or more reverse RPA primers may be immobilized to a top surface of the substrate (e.g., a bottom surface of the reservoir) by their 5′ termini. In some instances, one or more inert lateral spacers may be immobilized between two or more immobilized reverse RPA primers. Other RT-RPA reagents, including a reverse transcriptase, a DNA polymerase (e.g., Bsu DNA polymerase), an RNAse H, a single-stranded binding protein (e.g., gene 32 protein (G32P)), a recombinase (e.g., T4 UvsX), and forward RPA primers may be added to the aqueous solution of the reservoir. In certain embodiments, a small amount of reverse RPA primers may also be added to the aqueous solution as solution phase polynucleotides. Target nucleic acid sequences (e.g., single-stranded RNA sequences) complementary to the immobilized reverse RPA primers may hybridize to the immobilized reverse RPA primers. A reverse transcriptase may then elongate the immobilized reverse RPA primer into a daughter strand of complementary DNA (cDNA) using the single-stranded RNA of the target nucleic acid as a template. The single-stranded RNA in the newly generated DNA:RNA hybrids may be degraded by RNAseH, leaving the immobilized cDNA daughter strands. In certain cases, e.g., where RNAse is not included in the RPA reagents, direct primer invasion of DNA:RNA may also occur. Forward RPA primer present in the aqueous solution as a solution phase polynucleotide may bind to the immobilized cDNA daughter strand, and a DNA polymerase (e.g., Bsu DNA polymerase) may extend the forward RPA primers, using the cDNA as a template. RPA can occur, with the recombinase aiding double-stranded DNA invasion by immobilized reverse RPA primers (and optionally the small number of solution phase polynucleotide reverse RPA primers) and solution phase polynucleotide forward RPA primers, and extension by the polymerase.

In certain embodiments, a 5′-exonuclease may be added to degrade the non-immobilized DNA strand in the resulting double-stranded DNA amplicons. In some embodiments, the non-immobilized strand may be separated from the immobilized strand and/or degraded by applying heat and/or an alkaline treatment. In some cases, the degraded DNA strand and the leftover nucleotides and other RPA reagents may be washed away. In some embodiments, completion of amplification results in a plurality of immobilized daughter strands (amplicons) on the surface of the substrate, e.g., ready for a nucleic acid sequencing method described herein.

In some embodiments, reagents for nucleic acid sequencing methods described herein (also referred to as sequencing reagents), including DNA polymerase (e.g., Therminator DNA polymerase), a pool of protected nucleotides (e.g., 3′-unblocked protected nucleotides), and one or more solution phase polynucleotides (e.g., amplicon-specific sequencing primers), may be added to the reservoir, and the immobilized amplicons may be sequenced according to methods described herein. In certain embodiments, one or more reagents for nucleic acid sequencing methods may be added to a reservoir following completion of RPA amplification. In certain embodiments, one or more reagents for nucleic acid sequencing methods may be added to the reservoir prior to initiation of RPA amplification and/or during RPA amplification.

In some cases, RPA primers (e.g., forward RPA primers, reverse RPA primers) may be designed for a shotgun methodology. In some such cases, a method of RPA amplification comprises attaching one or more tag sequences to a target nucleic acid (e.g., attaching a first tag sequence to a first end of a target nucleic acid and a second tag sequence to a second end of a target nucleic acid). In some embodiments, a method of RPA amplification comprises providing one or more target nucleic acids having tag sequences at their 5′ and 3′ ends. In some embodiments, the forward RPA primer is complementary to and capable of binding to a first tag sequence and the reverse RPA primer is complementary to and capable of binding to a second tag sequence. In such cases, an immobilized RPA primer may bind to a target nucleic acid's tag sequence, followed by RPA amplification as described herein, allowing the amplification of any target nucleic acid sequence. In some embodiments, one or more forward RPA primers and one or more reverse RPA primers are immobilized to a surface (e.g., a top surface) of the substrate as part of a substrate construct. In some embodiments, one or both of the forward RPA primer and the reverse RPA primer are also present in solution. In some embodiments, each RPA primer comprises at least 15 bases, at least 20 bases, at least 25 bases, at least 30 bases, at least 35 bases, at least 40 bases, at least 45 bases, at least 50 bases, at least 55 bases, at least 60 bases, at least 65 bases, at least 70 bases, at least 75 bases, at least 80 bases, at least 85 bases, at least 90 bases, at least 95 bases, or at least 100 bases. In certain embodiments, each RPA primer comprises 30-120 bases, 30-100 bases, 30-90 bases, 30-80 bases, 30-70 bases, 40-120 bases, 40-100 bases, 40-90 bases, 40-80 bases, 50-120 bases, 50-100 bases, 50-90 bases, 50-80 bases, 60-120 bases, 60-100 bases, 60-90 bases, or 60-80 bases.

In some cases, RPA primers (e.g., forward RPA primers, reverse RPA primers) may be designed for each target nucleic acid sequence a device or method is configured to detect. In some embodiments, each RPA primer comprises at least 15 bases, at least 20 bases, at least 25 bases, at least 30 bases, at least 35 bases, at least 40 bases, at least 45 bases, or at least 50 bases. In certain embodiments, each RPA primer comprises 15-20 bases, 15-30 bases, 15-40 bases, 15-50 bases, 20-30 bases, 20-40 bases, 20-50 bases, 30-40 bases, 30-50 bases, or 40-50 bases. In some embodiments, each RPA primer does not have any mismatches within 3 bases of its 3′ terminus. In some embodiments, each RPA primer comprises 10 or fewer, 9 or fewer, 8 or fewer, 7 or fewer, 6 or fewer, 5 or fewer, 4 or fewer, 3 or fewer, 2 or fewer, 1 or fewer, or no mismatches. In some embodiments, each mismatch is at least 3 bases, at least 4 bases, at least 5 bases, at least 6 bases, at least 7 bases, at least 8 bases, at least 9 bases, or at least 10 bases from the 3′ terminus. While mismatches more than 3 bases away from the 3′ terminus of the RPA primer have been found to be well tolerated in RPA, multiple mismatches within 3 bases of the 3′ terminus have been found to inhibit the reaction. In some embodiments, one or more RPA primers (e.g., a reverse RPA primer) comprises a vertical spacer (e.g., a poly-T spacer).

In certain embodiments, a target pathogen is SARS-CoV-2, and a target nucleic acid sequence is a nucleic acid sequence of SARS-CoV-2. In some embodiments, forward and reverse RPA primers may be selected from regions of the SARS-CoV-2 nucleocapsid (N) gene and/or its spike (S) gene to maximize inclusivity across known SARS-CoV-2 strains and minimize cross-reactivity with related viruses and genomes likely to be present in the sample.

Exemplary forward and reverse RPA primers covering regions of the SARS-CoV-2 N gene are shown in Table 1:

TABLE 1
Exemplary SARS-COV-2 N Gene RPA Primers

| RPA Primer | Sequence | SEQ ID NO: |
|---|---|---|
| Forward | CGGCAGTCAAGCCTC | 1 |
| Primer 1 | TTCTCGTTCCTCATC | |
| Reverse | CARACATTTTGCTCT | 2 |
| Primer 1 | CAAGCTGGTTCAATC | |

In the primer sequences, “R” represents a purine nucleotide (e.g., A, G).

Exemplary forward and reverse primers covering regions of the SARS-CoV-2 S gene are shown in Table 2:

TABLE 2
Exemplary SARS-COV-2 S Gene RPA Primers

| RPA Primer | Sequence | SEQ ID NO: |
|---|---|---|
| Forward | TTAATAACGCTACTAATG | 3 |
| Primer | TTGTTATTAAAGTCTGTG | |
| Reverse | TAAGAAAAGGCTGAGAGA | 4 |
| Primer | CATATTCAAAAGTGC | |

In some embodiments, the RPA reagents comprise one or more forward primers. In certain embodiments, at least one forward primer is at least 70%, at least 75%, at least 80%, at least 85%, at least 90%, at least 95%, at least 99%, or 100% identical to SEQ ID NOs: 1 or 3. In some embodiments, the one or more forward primers comprise an antigenic tag. In certain embodiments, the concentration of the one or more forward primers is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the one or more forward primers is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents comprise one or more reverse primers. In certain embodiments, at least one reverse primer is at least 70%, at least 75%, at least 80%, at least 85%, at least 90%, at least 95%, at least 99%, or 100% identical to SEQ ID NOs: 2 or 4. In some embodiments, the one or more reverse primers comprise an antigenic tag. In certain embodiments, the concentration of the one or more reverse primers is at least 100 nM, at least 200 nM, at least 300 nM, at least 400 nM, at least 500 nM, at least 600 nM, at least 700 nM, at least 800 nM, at least 900 nM, or at least 1000 nM. In certain embodiments, the concentration of the one or more reverse primers is in a range from 100 nM to 200 nM, 100 nM to 500 nM, 100 nM to 800 nM, 100 nM to 1000 nM, 200 nM to 500 nM, 200 nM to 800 nM, 200 nM to 1000 nM, 500 nM to 800 nM, 500 nM to 1000 nM, or 800 nM to 1000 nM.

In some embodiments, the RPA reagents comprises a forward primer and reverse primer selected from any of SEQ ID NOs: 1-2 or SEQ ID NOs: 3-4.

In some embodiments, the RPA reagents comprise a recombinase enzyme. Non-limiting examples of suitable recombinase enzymes include T4 UvsX protein and T4 UvsY protein. In some embodiments, the concentration (e.g., in the aqueous solution) of the recombinase enzyme is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, at least 0.10 mg/mL, at least 0.11 mg/mL, at least 0.12 mg/mL, at least 0.13 mg/mL, at least 0.14 mg/mL, or at least 0.15 mg/mL. In some embodiments, the concentration of the recombinase enzyme is in a range from 0.01 mg/mL to 0.05 mg/mL, 0.01 mg/mL to 0.1 mg/mL, 0.01 mg/mL to 0.15 mg/mL, 0.05 mg/mL to 0.1 mg/mL, 0.05 mg/mL to 0.15 mg/mL, or 0.10 mg/mL to 0.15 mg/mL.

In some embodiments, the RPA reagents comprise a single-stranded DNA binding protein. A non-limiting example of a suitable single-stranded DNA binding protein is T4 g32P protein. In certain embodiments, the concentration (e.g., in the aqueous solution) of the single-stranded DNA binding protein is at least 0.1 mg/mL, at least 0.2 mg/mL, at least 0.3 mg/mL, at least 0.4 mg/mL, at least 0.5 mg/mL, at least 0.6 mg/mL, at least 0.7 mg/mL, at least 0.8 mg/mL, at least 0.9 mg/mL, or at least 1.0 mg/mL. In certain embodiments, the concentration of the single-stranded DNA binding protein is in a range from 0.1 mg/mL to 0.2 mg/mL, 0.1 mg/mL to 0.5 mg/mL, 0.1 mg/mL to 0.8 mg/mL, 0.1 mg/mL to 1.0 mg/mL, 0.2 mg/mL to 0.5 mg/mL, 0.2 mg/mL to 0.8 mg/mL, 0.2 mg/mL to 1.0 mg/mL, 0.5 mg/mL to 0.8 mg/mL, 0.5 mg/mL to 1.0 mg/mL, or 0.8 mg/mL to 1.0 mg/mL.

In some embodiments, the RPA agents comprise a DNA polymerase (e.g., a Bsu polymerase). In some embodiments, the DNA polymerase is a Bacillus subtilis DNA Polymerase (e.g., Bsu DNA Polymerase Large Fragment), a Staphylococcus aureus DNA Polymerase (e.g., Sau DNA Polymerase I Large Fragment), a Bacillus subtilis phage polymerase (e.g., Phi29), a Pyrococcusfuriosus DNA polymerase (e.g., PFU), or a Thermococcus DNA Polymerase (e.g., Therminator™). In some embodiments, the concentration of the DNA polymerase is at least 0.01 mg/mL, at least 0.02 mg/mL, at least 0.03 mg/mL, at least 0.04 mg/mL, at least 0.05 mg/mL, at least 0.06 mg/mL, at least 0.07 mg/mL, at least 0.08 mg/mL, at least 0.09 mg/mL, or at least 0.1 mg/mL. In certain embodiments, the concentration of the DNA polymerase is in a range from 0.01 mg/mL to 0.02 mg/mL, 0.01 mg/mL to 0.05 mg/mL, 0.01 mg/mL to 0.08 mg/mL, 0.01 mg/mL to 0.1 mg/mL, 0.02 mg/mL to 0.05 mg/mL, 0.02 mg/mL to 0.08 mg/mL, 0.02 mg/mL to 0.1 mg/mL, 0.05 mg/mL to 0.08 mg/mL, 0.05 mg/mL to 0.1 mg/mL, or 0.08 mg/mL to 0.1 mg/mL.

In some embodiments, the RPA reagents comprise deoxyribonucleotide triphosphates (“dNTPs”). In certain embodiments, the RPA reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the RPA agents further comprise an endonuclease and/or a crowding agent. A non-limiting example of a suitable crowding agent is polyethylene glycol (PEG). In some embodiments, the RPA agents do not have an 3′-exonuclease activity (i.e., the enzymes cannot remove the 3′ end of the primer).

Several alternative amplification methods exist that are similar in principle to RPA amplification. These include Recombinase-aided Amplification (RAA) (see, e.g., Qin, Z. et al. BMC Infectious Diseases volume 21, Article number: 248 (2021)) and Helicase-dependent Amplification (HDA) (see, e.g., Cao, Y. et al. Curr Protoc Mol Biol. 2013 Oct. 11; 104:15.11.1-15.11.12). RAA and HDA operate by a similar mechanism to RPA, utilizing a nucleic acid binding protein to facilitate strand invasion of a complementary primer nucleic acid, which in turn allows isothermal amplification of a nucleic acid, but differ in the nucleic acid binding protein employed (i.e., a helicase instead of a recombinase) or in the source of the recombinase. Accordingly, a person of skill in the art will be able to employ the devices and methods described herein with RAA and HDA using the teachings of the application and the state of the art without undue experimentation.

LAMP

In some embodiments, a method of sequencing of the disclosure comprises Loop-Mediated Isothermal Amplification (LAMP) of one or more target nucleic acid sequences. In some embodiments, a device of the disclosure is capable of sequencing a daughter strand (also referred to as an amplicon) comprising a sequence identical to or complementary to a target nucleic acid sequence that was amplified using LAMP (e.g., either in the reservoir or prior to being added to the reservoir). LAMP generally refers to a method of amplifying a target nucleic acid sequence using four or more primers through the creation of a series of stem-loop structures. Due to its use of multiple primers, LAMP may be highly specific for a target nucleic acid sequence. Accordingly, some methods and devices employing LAMP methodology are directed to using amplicon-specific substrate polynucleotides to detect or sequence one or more target nucleic acid sequences. In some embodiments, LAMP is combined with a reverse transcription reaction, and referred to as RT-LAMP. Accordingly, in some embodiments, the reservoir (e.g., the aqueous solution) comprises LAMP reagents. In some embodiments, LAMP reagents comprise LAMP primers, a polymerase, and nucleotides (dNTPs). In some embodiments, LAMP reagents comprise a reverse transcriptase (e.g., in addition to a polymerase).

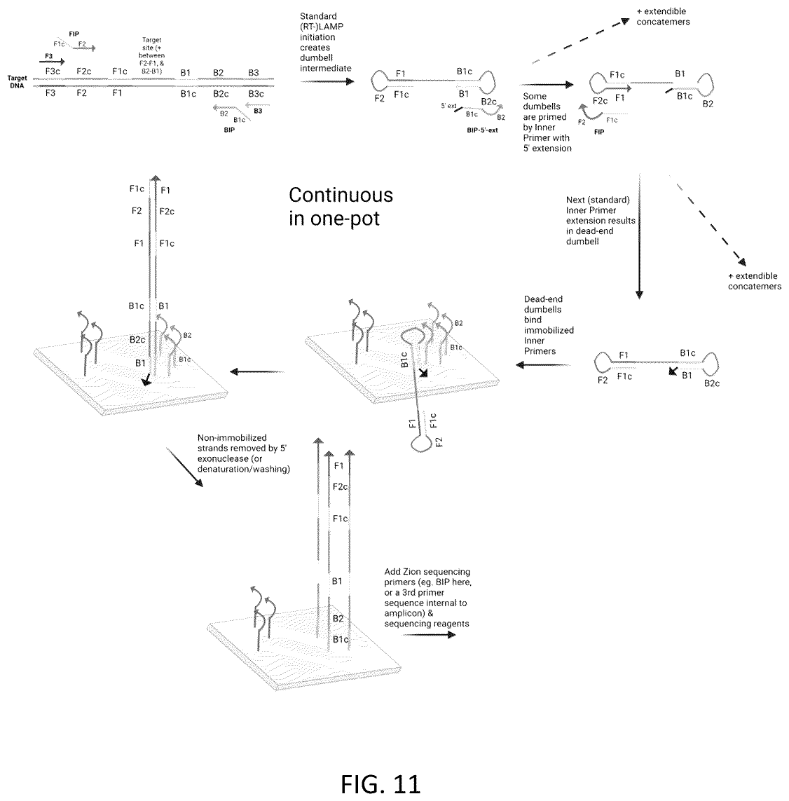

In some embodiments, LAMP reagents comprise four or more primers. In certain embodiments, the four or more primers comprise a forward inner primer (FIP), a backward inner primer (BIP), a forward outer primer (F3), and a backward outer primer (B3). In some cases, the four or more primers target at least six specific regions of a target nucleic acid sequence. In some embodiments, the six regions are represented as (from 5′ to 3′) F3, F2, F1, B1c, B2c, and B3c on a first strand, and B3, B2, B1, F1c, F2c, and F3c on a second strand, wherein F3 is complementary to F3c, F2 is complementary to F2c, and so forth (see, e.g., FIG. 11). In some embodiments, the regions the four or more primers target are present in a target nucleic acid sequence (e.g., a sequence from a biological sample, e.g., a pathogen- or cancer-associated sequence). In some such embodiments, a method of sequencing comprising LAMP amplification sequences a central target sequence (e.g., between F1/F1c and B1c/B1). In some embodiments, a method of sequencing comprising LAMP amplification sequences the sequences between the six regions (e.g., between F3/F3c and F2/F2c, F2/F2c and F1c/F1, B1/B1c and B2/B2c, and B2/B2c and B3/B3c) as well as the central target sequence. In some embodiments, the regions the four or more primers target are present on one or more tag sequences (e.g., added to one or both ends of a target nucleic acid sequence), e.g., in a shotgun methodology described herein. In some embodiments, a spacer sequence is positioned between F1c and B1 and F1 and B1c. In some embodiments, FIP comprises (from 5′ to 3′) the sequences of F1c and F2. See, e.g., FIG. 11. In some embodiments, BIP comprises (from 5′ to 3′) the sequences of B1c and B2. In some embodiments, the F3 primer comprises the sequence of F3. In some embodiments, the B3 primer comprises the sequence of B3. In certain embodiments, the LAMP reagents further comprise a forward loop primer (Loop F or LF) and a backward loop primer (Loop B or LB). In certain cases, the loop primers target cyclic structures formed during amplification and can accelerate amplification.

In some embodiments, one or more LAMP reagents are present in the aqueous solution of the reservoir. In certain embodiments, one or more (and, in some cases, all) LAMP primers are present in the aqueous solution of the reservoir as solution phase polynucleotides.

In some embodiments, one or more LAMP reagents are immobilized to a top surface of a substrate (e.g., a bottom surface of the reservoir). See, e.g., FIG. 11. In some embodiments, one or more LAMP primers is present as a substrate polynucleotide in a substrate construct immobilized to the reaction region of the surface of the substrate (e.g., in a spot). In some embodiments, all of the LAMP primers are present as substrate polynucleotides in substrate constructs immobilized to the reaction region of the surface of the substrate (e.g., in a spot). In certain embodiments, for example, FIP and/or BIP primers are immobilized to a surface of the substrate. In some instances, one or more forward loop primers and/or backward loop primers are immobilized to the surface of the substrate. In some embodiments, the one or more LAMP primers immobilized to the surface of the substrate are also present in the aqueous solution as one or more solution phase polynucleotides. In certain embodiments, a substrate polynucleotide in a substrate construct does not comprise a LAMP primer, but comprises a sequence complementary to an amplicon produced by LAMP amplification.

In some embodiments, a method comprising using LAMP to amplify one or more target nucleic acid sequences comprises an initial liquid phase and a subsequent solid phase. As an illustrative example, FIG. 11 shows a schematic illustration of an exemplary workflow for a LAMP-based amplification method comprising an initial liquid phase and a subsequent solid phase. As shown in the exemplary workflow of FIG. 11, in the initial liquid phase amplification step, a LAMP dumbbell structure may be generated by elongation of two outer primers (F3, B3) and two inner primers (FIP, BIP) by a polymerase after binding to four of the at least six specific regions in the target nucleic acid. In some embodiments, the target nucleic acid is RNA, and a reverse transcriptase is used. At least some of the dumbbell intermediates may serve as a template for a modified inner primer (e.g., FIP or BIP), which may eventually produce a ‘dead-end’ dumbbell incapable of being elongated. A LAMP dumbbell structure (e.g., the ‘dead-end’ LAMP structure) created in solution may then hybridize to FIP or BIP primers immobilized to a surface of the substrate as part of substrate constructs. In the exemplary workflow shown in FIG. 11, the B1C region of an immobilized BIP primer may anneal to a B1 region on the LAMP dumbbell structure and results in synthesis of a complementary strand. As a result, in this exemplary workflow, a DNA amplicon is synthesized and immobilized on the substrate. In some cases such as this example, a 5′ exonuclease is used to digest the non-immobilized strand and leaving a single-stranded daughter strand amplicon on the surface of the substrate. In some embodiments, completion of amplification results in a plurality of immobilized daughter strands (amplicons) on the surface of the substrate, e.g., ready for a nucleic acid sequencing method described herein.

In some embodiments, an inner primer (e.g., FIP or BIP) is present in two different solution phase polynucleotide forms:one with a 5′ extension region that is not complementary to the target nucleic acid or LAMP hybridization regions (e.g., mod-FIP or mod-BIP), and one without the extension (FIP or BIP). Without wishing to be bound by a particular theory, the elongation of mod-FIP or mod-BIP to produce a mod-FIP-amplicon or mod-BIP-amplicon followed by BIP/FIP elongation from the mod-amplicon template produces a LAMP dumbbell structure lacking an extendable 3′ end; such a ‘dead-end’ LAMP structure may more favorably be bound by an immobilized substrate polynucleotide, e.g., because the ‘dead-end’ LAMP structure is less likely to be part of a LAMP concatemer.

In some embodiments, reagents for nucleic acid sequencing methods described herein, including DNA polymerase (e.g., Therminator DNA polymerase), a pool of protected nucleotides (e.g., 3′-unblocked protected nucleotides), and one or more solution phase polynucleotides (e.g., amplicon-specific sequencing primers), may be added to the reservoir, and the immobilized amplicons may be sequenced according to methods described herein. In certain embodiments, one or more reagents for nucleic acid sequencing methods may be added to a reservoir following completion of LAMP amplification. In certain embodiments, one or more reagents for nucleic acid sequencing methods may be added to the reservoir prior to initiation of LAMP amplification and/or during LAMP amplification. In some embodiments, an amplicon-specific sequencing primer comprises some or all of a LAMP primer, e.g., BIP or FIP, as appropriate for the immobilized substrate polynucleotide. In some embodiments, an amplicon-specific sequencing primer does not comprise a LAMP primer and is complementary to a region of the amplicon that does not hybridize to a LAMP primer.

In some embodiments, a nickase recognition site may be inserted in one or more LAMP primers to facilitate initiation of sequencing. In some cases, the nickase recognition site is inserted in the loop region of one or more LAMP primers. In certain embodiments, a nickase recognition site is inserted in a FIP primer between F1C and F2 and/or in a BIP primer between B1C and B2. In some embodiments, a nickase recognition site is positioned proximal to the 3′ end of a LAMP primer, e.g., to initiate sequencing proximal to the target nucleic acid sequence. In some embodiments, a nickase recognition site is positioned 3′ of the F2 region in a FIP primer (e.g., comprising, from 5′ to 3′, F1C and F2) or 3′ of the B2 region in a FIP primer (e.g., comprising, from 5′ to 3′, B1C and B2). In some instances, one primer of a set of LAMP primers (e.g., four or more LAMP primers) comprises a nickase recognition site. In some instances, two primers of a set of LAMP primers comprise a nickase recognition site. In some instances, three or more primers of a set of LAMP primers comprise a nickase recognition site. In certain cases, a nickase recognition site comprises a dUTP. In some embodiments utilizing a dUTP nickase recognition site, only one LAMP primer comprises a dUTP nickase recognition site; a second or further nickase recognition site in a second or further LAMP primer comprises a different nickase recognition site. In some embodiments, a nickase recognition site comprises a 5′-CCTCAGC-3′ sequence, e.g., recognized by a BbvCI nicking endonuclease enzyme available from New England BioLabs.

In some cases, LAMP primers may be designed for each target nucleic acid sequence a device or method is configured to detect. Methods of designing LAMP primers are known in the art. In some embodiments, the target nucleic acid is associated with a pathogen or a cancer. In certain embodiments, the pathogen is SARS-CoV-2, and a target nucleic acid sequence is a nucleic acid sequence of SARS-CoV-2. In some embodiments, LAMP primers may be selected to hybridize with regions of the SARS-CoV-2 nucleocapsid (N) gene and/or its spike (S) gene to maximize inclusivity across known SARS-CoV-2 strains and minimize cross-reactivity with related viruses and genomes likely to be present in the sample. In certain embodiments, four or more (and, in some cases, six) SARS-CoV-2 LAMP primers target the Open Reading Frame 1ab (or f1ab) region of the SARS-CoV-2 genome.

Exemplary LAMP primers covering regions of the SARS-CoV-2 N gene are shown in Table 3:

TABLE 3
Exemplary SARS-COV-2 N Gene LAMP Primers

| | | SEQ |
|---|---|---|
| LAMP | | ID |
| primer | Sequence | NO: |
| F3 | AGGCGGCAGTCAAGCCTCT | 5 |
| B3 | AAGCCTCAGCAGCAGATTTCTTA | 6 |
| FIP | TGCCAGCCATTCTAGCAGGAGAA | 7 |
| | GTCTCATCACGTAGTCGCAA | |
| BIP | GGCGGTGATGCTGCTCTTGCTTT | 8 |
| | TTGTTGGCCTTTACCAGAC | |
| BIP with | tccgcagcttgcaacacgGGCGG | 9 |
| 5′ ext | TGATGCTGCTCTTGCTTTTTGTT | |
| | GGCCTTTACCAGAC | |
| BIP with | /5AmMC6/ttttttttttccccc | 10 |
| T10C10 | cccccGGCGGTGATGCTGCTCTT | |
| spacer + | GCTTTTTGTTGGCCTTTACCAGA | |
| 3′ ext | Cattttgctctcaagctggttca | |
| | atctgtc | |

In some embodiments, the LAMP reagents comprise a FIP and a BIP for one or more target nucleic acid sequences. In some embodiments, the FIP and BIP each have a sequence that is at least 90%, at least 95%, at least 96%, at least 97%, at least 98%, at least 99%, at least 99.5%, or 100% identical to a primer sequence provided in Table 3. In some embodiments, the concentrations of FIP and BIP are each at least 0.5 μM, at least 0.6 μM, at least 0.7 μM, at least 0.8 μM, at least 0.9 μM, at least 1.0 μM, at least 1.1 μM, at least 1.2 μM, at least 1.3 μM, at least 1.4 μM, at least 1.5 μM, at least 1.6 μM, at least 1.7 μM, at least 1.8 μM, at least 1.9 μM, or at least 2.0 μM. In some embodiments, the concentrations of FIP and BIP are each in a range from 0.5 μM to 1 μM, 0.5 μM to 1.5 μM, 0.5 μM to 2.0 μM, 1 μM to 1.5 μM, 1 μM to 2 μM, or 1.5 μM to 2 μM.

In some embodiments, the LAMP reagents comprise an F3 primer and a B3 primer for one or more target nucleic acid sequences. In some embodiments, the F3 primer and the B3 primer each have a sequence that is at least 90%, at least 95%, at least 96%, at least 97%, at least 98%, at least 99%, at least 99.5%, or 100% identical to a primer sequence provided in Table 3. In some embodiments, the concentrations of the F3 primer and the B3 primer are each at least 0.05 μM, at least 0.1 μM, at least 0.15 μM, at least 0.2 μM, at least 0.25 μM, at least 0.3 μM, at least 0.35 μM, at least 0.4 μM, at least 0.45 μM, or at least 0.5 μM. In some embodiments, the concentrations of the F3 primer and the B3 primer are each in a range from 0.05 μM to 0.1 μM, 0.05 μM to 0.2 μM, 0.05 μM to 0.3 μM, 0.05 μM to 0.4 μM, 0.05 μM to 0.5 μM, 0.1 μM to 0.2 μM, 0.1 μM to 0.3 μM, 0.1 μM to 0.4 μM, 0.1 μM to 0.5 μM, 0.2 μM to 0.3 μM, 0.2 μM to 0.4 μM, 0.2 μM to 0.5 μM, 0.3 μM to 0.4 μM, 0.3 μM to 0.5 μM, or 0.4 μM to 0.5 μM.

In some embodiments, the LAMP reagents comprise a forward loop primer and a backward loop primer for one or more target nucleic acid sequences. In some embodiments, the forward loop primer and the backward loop primer each have a sequence that is at least 90%, at least 95%, at least 96%, at least 97%, at least 98%, at least 99%, at least 99.5%, or 100% identical to a primer sequence provided in Table 3. In some embodiments, the concentrations of the forward loop primer and the backward loop primer are each at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, or at least 1.0 μM. In some embodiments, the concentrations of the forward loop primer and the backward loop primer are each in a range from 0.1 μM to 0.2 μM, 0.1 μM to 0.5 μM, 0.1 μM to 0.8 μM, 0.1 μM to 1.0 μM, 0.2 μM to 0.5 μM, 0.2 μM to 0.8 μM, 0.2 μM to 1.0 μM, 0.3 μM to 0.5 μM, 0.3 μM to 0.8 μM, 0.3 μM to 1.0 μM, 0.4 μM to 0.8 μM, 0.4 μM to 1.0 μM, 0.5 μM to 0.8 μM, 0.5 μM to 1.0 μM, or 0.8 μM to 1.0 μM.

In certain embodiments, SARS-CoV-2 LAMP primers comprise SEQ ID NOs. 5-8.

In some embodiments, the LAMP reagents comprise a DNA polymerase with high strand displacement activity. Non-limiting examples of suitable strand-displacing DNA polymerases suitable for use in LAMP or other amplification methods described herein include a DNA polymerase long fragment (LF) of a thermophilic bacterium, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, the concentration of the DNA polymerase is at least 0.1 U/μL, at least 0.2 U/μL, at least 0.3 U/μL, at least 0.4 U/μL, at least 0.5 U/μL, at least 0.6 U/μL, at least 0.7 U/μL, at least 0.8 U/μL, at least 0.9 U/μL, or at least 1.0 U/μL. In some embodiments, the concentration of the DNA polymerase is in a range from 0.1 U/μL to 0.5 U/μL, 0.1 U/μL to 1.0 U/μL, 0.2 U/μL to 0.5 U/μL, 0.2 U/μL to 1.0 U/μL, or 0.5 U/μL to 1.0 U/μL.

In some embodiments, the LAMP reagents comprise deoxyribonucleotide triphosphates (“dNTPs”). In certain embodiments, the LAMP reagents comprise deoxyadenosine triphosphate (“dATP”), deoxyguanosine triphosphate (“dGTP”), deoxycytidine triphosphate (“dCTP”), and deoxythymidine triphosphate (“dTTP”). In certain embodiments, the concentration of each dNTP (i.e., dATP, dGTP, dCTP, dTTP) is at least 0.5 mM, at least 0.6 mM, at least 0.7 mM, at least 0.8 mM, at least 0.9 mM, at least 1.0 mM, at least 1.1 mM, at least 1.2 mM, at least 1.3 mM, at least 1.4 mM, at least 1.5 mM, at least 1.6 mM, at least 1.7 mM, at least 1.8 mM, at least 1.9 mM, or at least 2.0 mM. In some embodiments, the concentration of each dNTP is in a range from 0.5 mM to 1.0 mM, 0.5 mM to 1.5 mM, 0.5 mM to 2.0 mM, 1.0 mM to 1.5 mM, 1.0 mM to 2.0 mM, or 1.5 mM to 2.0 mM.

In some embodiments, the LAMP agents further comprise magnesium sulfate (MgSO₄) and/or betaine.

RCA

In some embodiments, a method of sequencing of the disclosure comprises Rolling Circle Amplification (RCA) of one or more nucleic acid sequences. In some embodiments, a device of the disclosure is capable of sequencing a daughter strand (also referred to as an amplicon) comprising a sequence identical to or complementary to a target nucleic acid sequence that was amplified using RCA (e.g., on the surface of the substrate). RCA refers to a method of amplifying a target nucleic acid using, e.g., a padlock probe primer capable of hybridizing to a target nucleic acid and the creation of a single-stranded circular template. In some embodiments, RCA is combined with a reverse transcriptase reaction and referred to as RT-RCA. Accordingly, in some embodiments, the reservoir (e.g., the aqueous solution) and/or surface of the substrate comprise RCA reagents. In some embodiments, the RCA reagents comprise a polymerase (e.g., a DNA polymerase or RNA polymerase, e.g., a reverse transcriptase), a pool of nucleotides (e.g., dNTPs), and/or a single-strand nucleic acid ligase. In some embodiments, the RCA reagents comprise a DNA polymerase and a reverse transcriptase.

In some embodiments, the RCA reagents comprise one or more padlock probe primers (also referred to herein as padlock probes). In some embodiments, a padlock probe comprises a first sequence complementary to a target nucleic acid and a second sequence complementary to a target nucleic acid sequence. In some embodiments, the first sequence and second sequences are toward the 5′ and 3′ ends of the padlock probe, respectively (e.g., at the 5′ and 3′ ends). In some embodiments, the first sequence is complementary to a 3′ region of the target nucleic acid and the second sequence is complementary to a 5′ region of the target nucleic acid. In some embodiments, the padlock probe comprises the entire target nucleic acid sequence (e.g., non-contiguously), and in other embodiments the padlock probe comprises a portion of the target nucleic acid sequence. In some embodiments, a padlock probe comprises an RCA primer complementarity region comprising a sequence complementary to an RCA primer (e.g., provided as all or a portion of a substrate polynucleotide), e.g., an immobilized RCA primer. In some embodiments, the RCA reagents comprise a forward RCA primer (e.g., a substrate polynucleotide) immobilized to the reservoir region of the surface of the substrate as part of a substrate construct. In some embodiments, the forward RCA primer is complementary to a portion of a padlock probe (the RCA primer complementarity region). In some embodiments, the RCA reagents comprise a reverse RCA primer (e.g., a substrate polynucleotide) immobilized to the reservoir region of the surface of the substrate as part of a substrate construct. In some embodiments, the reverse RCA primer has the same sequence as a portion of a padlock probe (e.g., the RCA primer complementarity region).

The methods described herein may employ and the devices described herein may be configured for RCA amplification configured for shotgun sequencing. In some shotgun embodiments, the RCA reagents comprise a plurality of padlock probes (e.g., a library of padlock probes). In some embodiments, the plurality of padlock probes each comprise first and second sequences that are random sequences. In some embodiments, a plurality of padlock probes collectively may bind to hundreds, thousands, or millions of target nucleic acids. In some such embodiments, each RCA primer complementarity region of the plurality of padlock probe has the same nucleic acid sequence. In some such embodiments, each RCA primer immobilized to the surface of the substrate is capable of binding to each padlock probe of the plurality. In some embodiments, a method or device employing or configured for RCA amplification is configured for single-molecule seeding, e.g., where one or fewer single-stranded circular templates is contacted with a spot.

The methods described herein may employ and the devices described herein may be configured for RCA amplification configured for amplicon-specific sequencing. In some amplicon-specific embodiments, the RCA reagents comprise a plurality of padlock probes, wherein each padlock probe comprises an RCA primer complementarity region that is different from each other padlock probe's RCA primer complementarity region. In some such embodiments, each padlock probe is capable of binding to a different immobilized RCA primer. In some embodiments, a spot contains a pool of substrate polynucleotides comprising one nucleic acid sequence of RCA primer, such that the spot is specific for a single padlock probe.

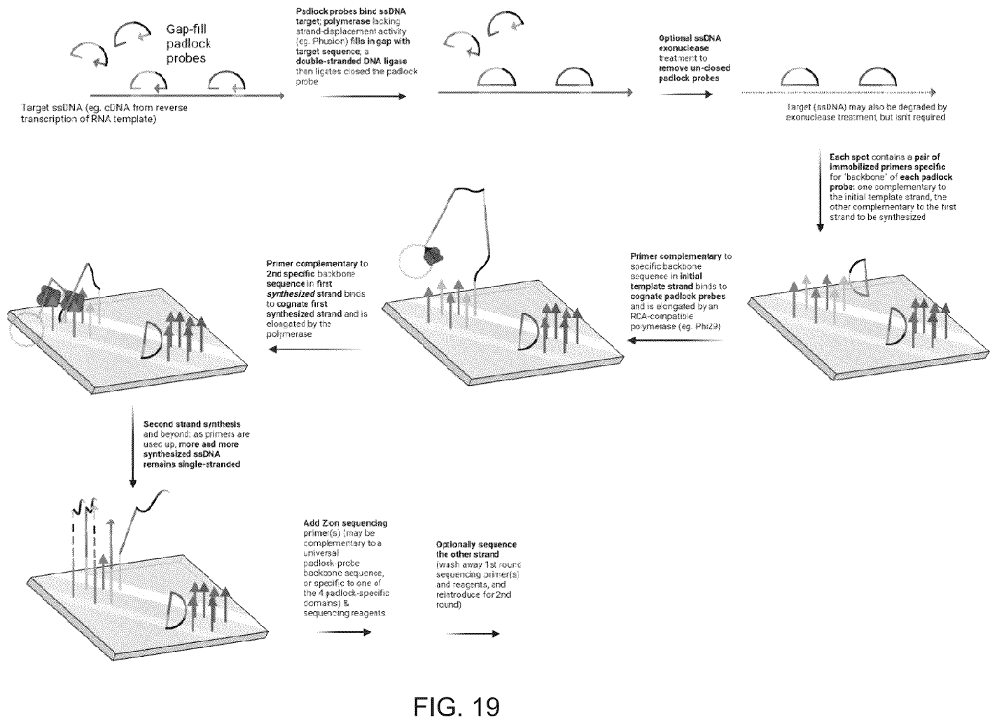

FIG. 19 shows a schematic illustration of an exemplary solid phase reverse transcriptase RCA (RT-RCA) workflow. As shown in FIG. 19, one or more padlock probes may bind to a target nucleic acid (e.g., an RNA target). The padlock probe may comprise a first sequence complementary to a 5′ region of the target nucleic acid and a second sequence complementary to a 3′ region of the target nucleic acid. A polymerase (e.g., reverse transcriptase), e.g., lacking exonuclease activity, may extend the 3′ end of the padlock probe using the target nucleic acid as template, and a ligase may repair the gap between the extended 3′ end and the 5′ end of the padlock probe, forming a single-stranded circular template. The single-stranded circular template may anneal to a forward RCA primer immobilized to the reservoir region of the surface of the substrate. A polymerase (e.g., DNA polymerase) may extend the RCA primer using the single-stranded circular template as a primer, resulting in rolling circle amplification. The elongated linear daughter strand produced from extension of the forward RCA primer comprises multiple copies of the complement sequence to the single-stranded circular template and may anneal to one or more reverse RCA primers immobilized to the surface of the substrate. A polymerase may extend the reverse RCA primers using the elongated linear daughter strand of the forward RCA primer as a template, and the process may repeat, producing a spot comprising a plurality of immobilized daughter strands (amplicons) on the surface of the substrate. In some embodiments, RCA amplification, e.g., after production of a plurality of immobilized daughter strands (amplicons) on the surface of the substrate, comprises a wash step. In some embodiments, the wash step washes away primers (e.g., padlock probes), nucleotides (e.g., naturally occurring nucleotides), or a polymerase (e.g., reverse transcriptase and/or DNA polymerase). In some embodiments, the wash step cleaves or linearizes single-stranded circular templates (e.g., shortening immobilized amplicons).

In some embodiments, reagents for nucleic acid sequencing methods described herein, including DNA polymerase (e.g., Therminator DNA polymerase), a pool of protected nucleotides (e.g., 3′-unblocked protected nucleotides), and one or more solution phase polynucleotides (e.g., amplicon-specific sequencing primers), may be added to the reservoir, and the immobilized amplicons may be sequenced according to methods described herein. In certain embodiments, one or more reagents for nucleic acid sequencing methods may be added to a reservoir following completion of RCA amplification. In certain embodiments, one or more reagents for nucleic acid sequencing methods may be added to the reservoir prior to initiation of RCA amplification and/or during RCA amplification.

In some embodiments, a RCA primer comprises at least 15 bases, at least 20 bases, at least 25 bases, at least 30 bases, at least 35 bases, at least 40 bases, at least 45 bases, at least 50 bases, at least 55 bases, at least 60 bases, at least 65 bases, at least 70 bases, at least 75 bases, at least 80 bases, at least 85 bases, at least 90 bases, at least 95 bases, or at least 100 bases. In certain embodiments, a RCA primer comprises 30-120 bases, 30-100 bases, 30-90 bases, 30-80 bases, 30-70 bases s, 40-120 bases, 40-100 bases, 40-90 bases, 40-80 bases, 50-120 bases, 50-100 bases, 50-90 bases, 50-80 bases, 60-120 bases, 60-100 bases, 60-90 bases, or 60-80 bases.

In some embodiments, a padlock probe comprises a first sequence and/or second sequence that is at least 15 bases, at least 20 bases, at least 25 bases, at least 30 bases, at least 35 bases, at least 40 bases, at least 45 bases, at least 50 bases, at least 55 bases, at least 60 bases, at least 65 bases, at least 70 bases, at least 75 bases, at least 80 bases, at least 85 bases, at least 90 bases, at least 95 bases, or at least 100 bases in length. In certain embodiments, a padlock probe comprises a first sequence and/or second sequence that is 30-120 bases, 30-100 bases, 30-90 bases, 30-80 bases, 30-70 bases, 40-120 bases, 40-100 bases, 40-90 bases, 40-80 bases, 50-120 bases, 50-100 bases, 50-90 bases, 50-80 bases, 60-120 bases, 60-100 bases, 60-90 bases, or 60-80 bases in length. In some embodiments, a padlock probe comprises an RCA primer complementarity region that is least 15 bases, at least 20 bases, at least 25 bases, at least 30 bas bases, at least 35 bases, at least 40 bases, at least 45 bases, at least 50 bases, at least 55 b bases, at least 60 bases, at least 65 bases, at least 70 bases, at least 75 bases, at least 80 bases, at least 85 bases, at least 90 bases, at least 95 bases, or at least 100 bases in length. In certain embodiments, a padlock probe comprises an RCA primer complementarity region that is 30-120 bases, 30-100 bases, 30-90 bases, 30-80 bases, 30-70 bases, 40-120 bases, 40-100 bases, 40-90 bases, 40-80 bases, 50-120 bases, 50-100 bases, 50-90 bases, 50-80 bases, 60-120 bases, 60-100 bases, 60-90 bases, or 60-80 bases in length.

In some cases, RCA amplification may be used in a shotgun methodology. In some such cases, a method of RCA amplification comprises attaching hairpin tag or adaptor sequences to a target nucleic acid (e.g., attaching a first hairpin tag sequence to a first end of a target nucleic acid and a second hairpin tag sequence to a second end of a target nucleic acid). In some embodiments, RNA is reverse transcribed into DNA to produce a suitable target nucleic acid for attachment of hairpin tag or adaptor sequences. In some embodiments, a shotgun method of RCA comprises ligating the open ends of the hairpin adaptors to form a closed nucleic acid structure. In some embodiments, a shotgun method of RCA comprises removing non-closed nucleic acid (e.g., using an exonuclease). In some embodiments, the resultant circular single-stranded DNA is applied to RCA primers as described herein.

WildFire

In some embodiments, a method of sequencing of the disclosure comprises an isothermal monoclonal colony amplification method, also referred to herein as WildFire amplification. In general, WildFire amplification takes advantage of DNA breathing and low melting temperature primers, as well as suitable target nucleic acid concentrations, to produce a spot comprising a clonal expansion of a single target nucleic acid. See, e.g., Ma et al., Proc Natl Acad Sci USA. 2013 Aug. 27; 110(35): 14320-14323, which is hereby incorporated by reference in its entirety. In some embodiments, a device of the disclosure is capable of sequencing a daughter strand (also referred to as an amplicon) comprising a sequence identical to or complementary to a target nucleic acid sequence that was amplified using WildFire amplification (e.g., on the surface of the substrate). In some embodiments, WildFire amplification is combined with a reverse transcription reaction and referred to as RT-WildFire amplification. Accordingly, in some embodiments, the reservoir (e.g., the aqueous solution) comprises WildFire reagents.

In some embodiments, WildFire reagents comprise one or more polymerases. In some embodiments, the one or more polymerase includes a reverse transcriptase. In some embodiments, the one or more polymerase includes at least one DNA polymerase (e.g., a strand-displacing polymerase). Non-limiting examples of suitable strand-displacing DNA polymerases suitable for use in WildFire or other amplification methods described herein include a DNA polymerase long fragment (LF) of a thermophilic bacterium, such as Bacillus stearothermophilus (Bst), Bacillus Smithii (Bsm), Geobacillus sp. M (GspM), or Thermodesulfatator indicus (Tin), or a Taq DNA polymerase. In certain embodiments, the DNA polymerase is Bst LF DNA polymerase, GspM LF DNA polymerase, GspSSD LF DNA polymerase, Tin exo-LF DNA polymerase, or SD DNA polymerase. In each case, the DNA polymerase may be a wild type or mutant polymerase.

In some embodiments, WildFire reagents comprise a forward primer and a reverse primer. In some embodiments, the forward primer and reverse primers are designed for a shotgun methodology. In some such cases, a method of WildFire amplification comprises attaching one or more tag sequences to a target nucleic acid (e.g., attaching a first tag sequence to a first end of a target nucleic acid and a second tag sequence to a second end of a target nucleic acid). In some embodiments, a method of WildFire amplification comprises providing one or more target nucleic acids having tag sequences at their 5′ and 3′ ends. In some embodiments, the forward primer is complementary to and capable of binding to a first tag sequence and the reverse primer is complementary to and capable of binding to a second tag sequence. In some embodiments, either of the forward or reverse primers is immobilized to the surface of the substrate, e.g., as a substrate polynucleotide of a substrate construct, and the other primer is present in the aqueous solution as a solution phase polynucleotide. In such cases, an immobilized primer may bind to a target nucleic acid's tag sequence, followed by WildFire amplification as described herein, allowing the amplification of any target nucleic acid sequence.

In some embodiments, a method or device employing or configured for WildFire amplification is configured to seed discernible colonies on the surface (e.g., the reservoir region) of the substrate. In some embodiments, a substrate configured for WildFire amplification comprises a reservoir region comprising a single large spot, e.g., that encompasses some, most, or all of the reservoir region. In some embodiments, the single large spot comprises a pool of substrate constructs comprising substrate polynucleotides having the same nucleic acid sequence, e.g., that of a forward or reverse primer complementary to a tag sequence. In some embodiments, the single large spot comprises said pool of substrate constructs in a lawn coating the surface of the substrate that the single large spot encompasses. Without wishing to be bound by a particular theory, in some embodiments, WildFire amplification comprises contacting a plurality of target nucleic acids with the pool of substrate constructs of the reservoir region. A given target nucleic acid may be amplified on the surface of the substrate in an expanding ‘colony’ of daughter strand amplicons centered on the location where the target nucleic acid annealed to a substrate polynucleotide. Said colony may expand until reaching the edge of another colony (produced by contact of another target nucleic acid with the pool of substrate constructs), at which point amplification would halt. In some embodiments, the concentration of target nucleic acid and/or the density of the pool of substrate constructs is selected or adjusted to ensure colonies are of a size that can be observed by detection methods and detection components described herein. For example, the concentration of target nucleic acid and/or the density of the pool of substrate constructs may be selected or adjusted to ensure colonies are of a size suitable for evanescent wave imaging. In some embodiments, a discernible colony has a diameter (i.e., largest dimension) of at least 1 μm, 1.5 μm, 2 μm, 2.5 μm, 3 μm, 4 μm, 5 μm, 6 μm, 7 μm, 8 μm, 9 μm, 10 μm, 15 μm, or 20 μm.

In some embodiments, reagents for nucleic acid sequencing methods described herein, including DNA polymerase (e.g., Therminator DNA polymerase), a pool of protected nucleotides (e.g., 3′-unblocked protected nucleotides), and one or more solution phase polynucleotides (e.g., amplicon-specific sequencing primers), may be added to the reservoir, and the immobilized amplicons may be sequenced according to methods described herein. In certain embodiments, one or more reagents for nucleic acid sequencing methods may be added to a reservoir following completion of WildFire amplification. In certain embodiments, one or more reagents for nucleic acid sequencing methods may be added to the reservoir prior to initiation of WildFire amplification and/or during WildFire amplification.

In an exemplary WildFire amplification workflow, a nicked double stranded target nucleic acid (comprising a single-stranded portion) anneals to a forward primer immobilized to the surface of a substrate. A strand-displacing polymerase extends the forward primer using the non-nicked target nucleic acid strand as template, producing a daughter strand amplicon. A second immobilized forward primer (of an adjacent substrate construct) invades the non-nicked target nucleic acid strand:daughter strand duplex, e.g., via DNA breathing, which can also be elongated by a polymerase. Each extended substrate polynucleotide also can be a template for extension from a solution-phase reverse primer, with daughter strands able to exchange to adjacent substrate constructs by strand invasion (e.g., via DNA breathing) of additional immobilized forward primers in adjacent substrate constructs. In some embodiments, the method results in one or more colonies in a spot on the surface of a substrate, wherein each colony comprises immobilized daughter strands that are copies of a single target nucleic acid. In some embodiments, WildFire amplification is followed by a wash step, e.g., that washes away one or more (e.g., all) WildFire reagents. In some embodiments, reagents for nucleic acid sequencing methods described herein are added after the completion of WildFire amplification, and the daughter strands of the colonies are sequenced.

### Sequencing Methods

The disclosure is directed, in part, to methods of sequencing a nucleic acid using evanescent wave imaging (e.g., using a device comprising an evanescent wave imaging apparatus), also referred to herein as sequencing using evanescent wave imaging. Generally, sequencing using evanescent wave imaging comprises using an evanescent wave to selectively manipulate an annealed sequencing primer, an incorporated nucleotide (e.g., a protected nucleotide), a substrate polynucleotide (e.g., functioning as a template for the sequencing primer), and/or a polymerase, thereby enabling control of incorporation of nucleotides into a sequencing primer as well as determination of the identity of the incorporated nucleotides. In some embodiments, sequencing using evanescent wave imaging comprises incorporating a single nucleotide (e.g., a protected nucleotide) into an annealed sequencing primer and reversibly terminating elongation of the annealed sequencing primer. The identity of the incorporated nucleotide may be determined and the reversible termination may be relieved using evanescent wave imaging. In some embodiments, methods of sequencing comprise repeating these steps (e.g., incorporating a single nucleotide, reversibly terminating elongation, determining the identity of the nucleotide, and relieving reversible termination) for a number of cycles (e.g., a preselected number of cycles). In some embodiments, the annealed sequencing primer is elongated using a substrate polynucleotide (e.g., a daughter strand produced by elongation of a substrate polynucleotide) as a template. In some embodiments, a pool of immobilized daughter strands (e.g., present as part of a pool of substrate constructs on the surface of the substrate) is provided using a nucleic acid amplification method described herein.

As used herein, reversible termination of elongation refers to halting extension of a polynucleotide by a polymerase in a manner which prevents further incorporation of nucleotides, where such halting may be relieved (i.e., reversed) by the occurrence of a later event. In some embodiments, reversible termination of elongation is accomplished by the use of a protected nucleotide (e.g., a protected nucleotide described herein).

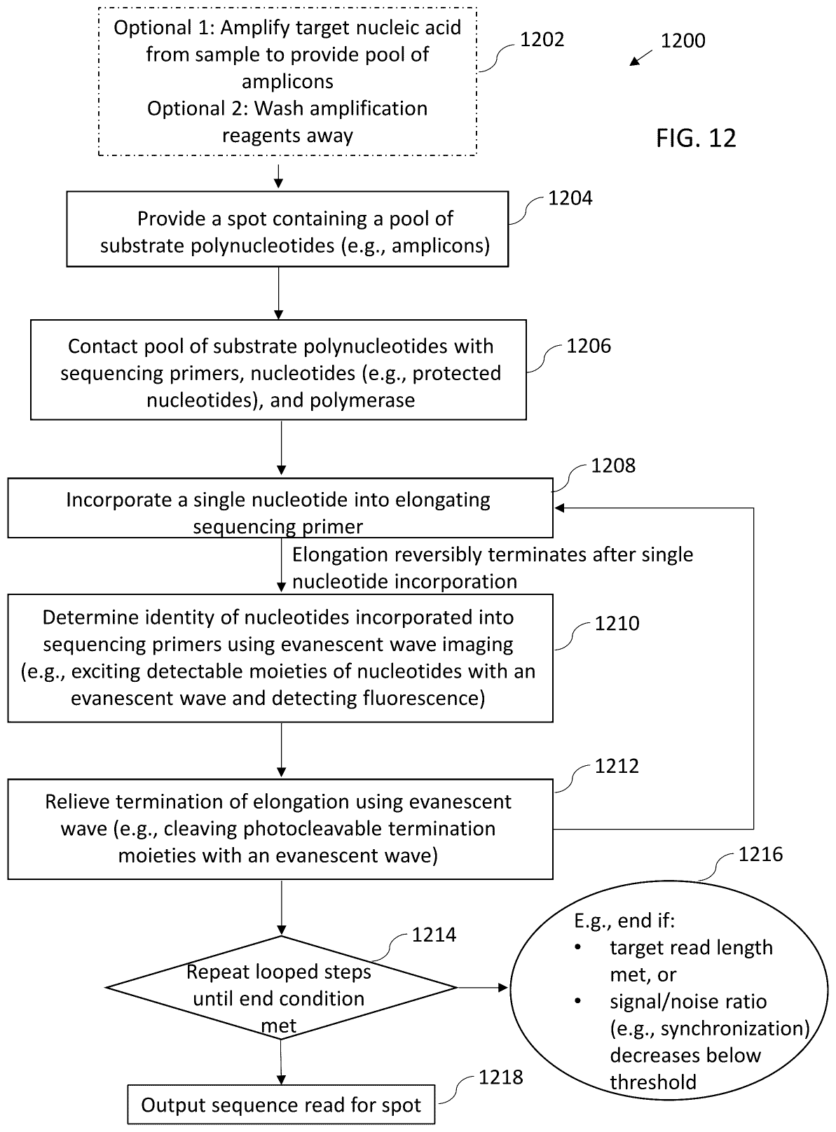

FIG. 12 shows a flow chart 1200 illustrating an exemplary method of nucleic acid sequencing using evanescent wave imaging. In the method of flow chart 1200, one or more acts need not be performed in the order shown; one or more acts may be omitted; two or more acts may be performed concurrently; and/or one or more additional acts not explicitly shown may be performed before, during, or after one or more acts shown.

At act 1202, a target nucleic acid from a sample may optionally be amplified to provide a pool of amplicons (e.g., via RPA, LAMP, RCA, WildFire, or another nucleic acid amplification method described herein). In some cases, amplification reagents may optionally be washed away following completion of amplification.

At act 1204, a method or device described herein may provide a spot comprising a pool of substrate constructs comprising substrate polynucleotides. In some embodiments, the substrate polynucleotides of the spot comprise an amplicon (e.g., provided by a nucleic acid amplification method described herein) with the same or complementary sequence to a target nucleic acid sequence. At act 1206, the pool of substrate constructs comprising substrate polynucleotides may be contacted with sequencing primers, nucleotides (e.g., protected nucleotides), and polymerases such that a plurality of sequencing primers anneal to a plurality of substrate constructs comprising substrate polynucleotides. At act 1208, a polymerase may incorporate a single nucleotide (e.g., a protected nucleotide comprising a photocleavable terminating moiety) into an elongating sequencing primer. In some embodiments, elongation of the sequencing primer terminates after incorporation of the single nucleotide (e.g., the protected nucleotide).

At act 1210, evanescent wave imaging is used to determine the identity of nucleotides incorporated into sequencing primers (e.g., by selectively exciting detectable moieties of protected nucleotides incorporated into sequencing primers and detecting light emitted by the detectable moieties). At act 1212, evanescent wave imaging is used to relieve the reversible termination of elongation of sequencing primers (e.g., by cleaving using the evanescent wave to induce cleavage of photocleavable terminating moieties of protected nucleotides, thereby allowing a polymerase to further incorporate nucleotides, e.g., at the 3′-OH position). Following act 1212, the method may return to act 1208. As shown at act 1214, the looped steps (e.g., incorporation 1208, termination, determination 1210, and relieving termination 1212) may repeat for a number of cycles (e.g., a number of cycles sufficient to sequence the amplicon) until an end condition is met. As shown at act 1216, an end condition may include a target read length being met and/or a signal-to-noise ratio decreasing below a particular threshold. After an end condition is met, at act 1218, an output sequence may be read for a spot.

In some embodiments, sequencing using evanescent wave imaging elongates a plurality of annealed sequencing primers (e.g., annealed to the pool of substrate polynucleotides of a spot) synchronously, wherein each cycle incorporates a nucleotide and determines its identity for each primer of the plurality. In some embodiments, the substrate polynucleotides of a spot are amplicons comprising identical nucleic acid sequences and the elongation of an annealed sequencing primer is synchronous and aligned with the elongation of other annealed sequencing primers of the plurality, such that in a given cycle the same nucleotide is incorporated into each of the plurality of annealed sequencing primers of a given spot. In some embodiments, the emitted light (e.g., fluorescence) detected from a given spot in a given cycle results in the determination of the identity of the nucleotide incorporated. As described elsewhere herein, other mechanisms of using evanescent wave imaging to control reversible termination of elongation are compatible with the methods and devices of the disclosure.

In some embodiments, sequencing using evanescent wave imaging comprises incorporation of a protected nucleotide into an annealed sequencing primer. In some embodiments, a protected nucleotide comprises a detectable moiety and a photocleavable terminating moiety, and elongation of the annealed sequencing primer is terminated due to the presence of the photocleavable terminating moiety. In some such embodiments, an evanescent wave is used to determine the identity of the protected nucleotide incorporated into the annealed sequencing primer while elongation is terminated. In some instances, for example, an evanescent wave is used to expose the annealed sequencing primer and incorporated protected nucleotide to excitation light (e.g., visible light, UV light) such that the detectable moiety of the protected moiety is excited. In certain embodiments, the detectable moiety is a fluorophore, and exposure to the excitation light is sufficient for the detectable moiety to produce a fluorescent emission. In some embodiments, an evanescent wave is subsequently used to cleave the photocleavable terminating moiety of the incorporated protected nucleotide. In some instances, for example, an evanescent wave is used to expose the annealed sequencing primer and incorporated protected nucleotide to photocleavage light (e.g., UV light, visible light) such that the photocleavable terminating moiety is cleaved. In some cases, cleavage of the photocleavable terminating moiety from the protected nucleotide reverses termination of elongation and allows elongation of the annealed sequencing primer to resume.

### Sequencing Reagents and Conditions

In some embodiments, a method of sequencing using evanescent wave imaging comprises using one or more sequencing reagents. Sequencing reagents may include, but are not limited to, a DNA polymerase (e.g., Therminator DNA polymerase), a pool of nucleotides (e.g., protected nucleotides, e.g., 3′-unblocked protected nucleotides), and one or more solution phase polynucleotides (e.g., sequencing primers). Other sequencing reagents may include one or more buffering agents and/or reactive oxygen scavenging agents.

In some embodiments, a method of sequencing using evanescent wave imaging uses one or more sequencing primers. Generally, a sequencing primer for use in a method or device described herein comprises a nucleic acid sequence complementary to a daughter strand, e.g., an amplicon. In some embodiments, a sequencing primer is complementary to a portion of the daughter strand specific to a target nucleic acid. For example, a sequencing primer may be complementary to a nucleic acid sequence associated with a particular pathogen or pathogen variant. In some embodiments, a sequencing primer is complementary to a portion of the daughter strand that is not specific to a single target nucleic acid, e.g., to a sequence shared by a plurality (e.g., all) amplicons immobilized to the substrate. For example, a sequencing primer may be complementary to a tag sequence, allowing the primer to be used for sequencing of any tag sequence containing daughter strand.

Broadly, sequencing methodologies can be divided into two approaches: shotgun sequencing and amplicon-specific sequencing. Shotgun sequencing includes sequencing methodologies capable of sequencing all the nucleic acids present in a fragmented library of nucleic acid (e.g., sheared fragments of genomic DNA). Shotgun sequencing can involve modifying the fragments by ligating one or more tag sequences, e.g., paired ends or bar codes, to the fragments, followed by sequencing the modified fragments. In some embodiments, shotgun sequencing methods sequence at least 1000, 2000, 3000, 5000, 10000, 20000, or 30000 distinct target nucleic acids. In some embodiments, sequencing primers for use in shotgun sequencing methodologies comprise a sequence complementary to and capable of annealing to a tag sequence (e.g., a tag sequence attached to one or more daughter strands, e.g., in a nucleic acid amplification step described herein). Amplicon-specific sequencing includes sequencing methodologies that sequence one or more specific nucleic acids of interest or detect one or more sequences of interest, e.g., in a larger population of sequences present in a sample. In some embodiments, sequencing primers for use in amplicon-specific sequencing methodologies comprise a sequence complementary to a target nucleic acid-specific sequence. The methods and devices of the disclosure are compatible with both shotgun sequencing approaches and amplicon-specific sequencing approaches.

In some embodiments, a sequencing primer is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases in length (and optionally no more than 60, 50, 40, or 30 bases in length). In some embodiments, a sequencing primer is 10-50, 15-50, 20-50, 25-50, 30-50, 35-50, 40-50, 45-50, 10-40, 15-40, 20-40, 25-40, 30-40, 35-40, 10-30, 15-30, 20-30, 25-30, 10-20, or 15-20 bases in length.

In some embodiments, a sequencing primer comprises a sequence that is also present in an amplification primer. For example, in an embodiment utilizing LAMP amplification, a sequencing primer may comprise, e.g., F3, F2, F1, B1c, B2c, B3c, B3, B2, B1, F1c, F2c, and/or F3c. In some embodiments, a sequencing primer comprises the entire sequence of an amplification primer. In some embodiments, a sequencing primer is identical to an amplification primer. In other embodiments, a sequencing primer has no sequences in common with any amplification primers utilized. In some embodiments, a sequencing primer anneals to a sequence in a substrate polynucleotide that is different than the sequences to which any amplification primer anneals. In some embodiments, a sequencing primer does not anneal or does not appreciably anneal to an amplification primer.

In some embodiments, a method or device described herein utilizes sequencing reagents comprising a plurality of sequencing primers. In some embodiments, the plurality of sequencing primers comprises at least a different sequencing primer for each target nucleic acid to be detected or sequenced. For example, in an embodiment where a method or device is configured to detect 10 different target nucleic acids, the sequencing reagents may comprise 10 or more sequencing primers. In some embodiments, the plurality of sequencing primers comprise multiple sequencing primers for each target nucleic acid molecule, e.g., with partially overlapping sequences complementary to different portions of a target nucleic acid molecule. In other embodiments, a method or device described herein utilizes sequencing reagents comprising a single sequencing primer, e.g., that anneals to a tag sequence present in the pool of substrate polynucleotides. Accordingly, in some embodiments, the sequencing reagents comprise 1, 2, 3, 4, 5, 6, 7, 8, 9, 10, 11, 12, 13, 14, 15, 16, 17, 18, 19, or 20 different sequencing primers. In some embodiments, the sequencing reagents comprise at least 1, 2, 5, 10, 15, 20, 25, 30, 40, or 50 different sequencing primers. In some embodiments, the sequencing reagents comprise 1-30, 1-25, 1-20, 1-15, 1-10, 1-9, 1-8, 1-7, 1-6, 1-5, 1-4, 1-3, 1-2, 2-30, 2-25, 2-20, 2-15, 2-10, 2-9, 2-8, 2-7, 2-6, 2-5, 2-4, 2-3, 5-30, 5-25, 5-20, 5-15, 5-10, 5-9, 5-8, 5-7, 5-6, 10-30, 10-25, 10-20, 10-15, 15-30, 15-25, 15-20, 20-30, 20-25, or 25-30 different sequencing primers. In some embodiments, a method or device described herein is configured to detect or sequence 1, 2, 3, 4, 5, 6, 7, 8, 9, 10, 11, 12, 13, 14, 15, 16, 17, 18, 19, or 20 different target nucleic acids. In some embodiments, a method or device described herein is configured to detect or sequence at least 1, 2, 5, 10, 15, 20, 25, 30, 40, or 50 different target nucleic acids. In some embodiments, a method or device described herein is configured to detect or sequence 1-30, 1-25, 1-20, 1-15, 1-10, 1-9, 1-8, 1-7, 1-6, 1-5, 1-4, 1-3, 1-2, 2-30, 2-25, 2-20, 2-15, 2-10, 2-9, 2-8, 2-7, 2-6, 2-5, 2-4, 2-3, 5-30, 5-25, 5-20, 5-15, 5-10, 5-9, 5-8, 5-7, 5-6, 10-30, 10-25, 10-20, 10-15, 15-30, 15-25, 15-20, 20-30, 20-25, or 25-30 different target nucleic acids.

In some embodiments, a method of nucleic acid sequencing comprises contacting a substrate polynucleotide with one or more sequencing reagents (e.g., a sequencing primer, a protected nucleotide, and/or a polymerase). In some embodiments, contacting a substrate polynucleotide with one or more sequencing reagents comprises adding the one or more sequencing reagents to a reservoir (e.g., an aqueous solution of the reservoir). In some embodiments, the substrate polynucleotide is a daughter strand (e.g., an amplicon) comprising the sequence of a target nucleic acid or a sequence complementary to a target nucleic acid.

In some embodiments, one or more (and, in some cases, all) sequencing reagents are in solid form (e.g., lyophilized, dried, crystallized, air jetted). In certain instances, at least one of the one or more sequencing agents is in the form of one or more beads and/or tablets. In some cases, one or more beads and/or tablets comprising one or more sequencing reagents may be stored in a cap and, in some cases, may be released from the cap (e.g., into the reservoir). In some embodiments, one or more (and, in some cases, all) sequencing reagents are in aqueous form. In certain instances, a method comprises transferring an aqueous solution comprising one or more sequencing reagents into the reservoir. In some embodiments, the reservoir comprising an aqueous solution and one or more sequencing reagents is provided to a user (e.g., as part of a consumable module).

In some embodiments, a method of nucleic acid sequencing comprises contacting a substrate polynucleotide with a protected nucleotide (e.g., a 3′-unblocked protected nucleotide). In some embodiments, a method of nucleic acid sequencing comprises contacting a substrate nucleotide with a polymerase. In some embodiments, a method of nucleic acid sequencing comprises contacting a substrate polynucleotide with a sequencing primer, e.g., such that the sequencing primer anneals to the substrate polynucleotide.

In some embodiments, a method of nucleic acid sequencing comprises adding one or more sequencing reagents (e.g., a protected nucleotide, a polymerase, one or more sequencing primers) to a reservoir at substantially the same time as one or more amplification reagents (e.g., RPA primers, a polymerase, a single-stranded binding protein, a reverse transcriptase, LAMP primers). In certain embodiments, a method of nucleic acid sequencing comprises adding one or more sequencing reagents to a reservoir prior to adding one or more amplification reagents or after adding one or more amplification reagents to the reservoir. In certain embodiments, a method of nucleic acid sequencing comprises adding one or more sequencing reagents after completion of an amplification reaction (e.g., an RPA reaction, a LAMP reaction, an RCA reaction, a WildFire reaction). In certain embodiments, a method of nucleic acid sequencing comprises adding one or more sequencing reagents prior to initiating an amplification reaction or during an amplification reaction. In some embodiments, once the reservoir comprises the sequencing reagents, no fluid transfer (e.g., no wash step) is required to complete the sequencing of a nucleic acid.

In some embodiments, a method for nucleic acid sequencing comprises using evanescent wave imaging to determine the identity of a nucleotide, e.g., a protected nucleotide (e.g., a 3′-unblocked protected nucleotide) incorporated into a sequencing primer. In some embodiments, the nucleotide is a protected nucleotide comprising a detectable moiety (e.g., a fluorophore) and a photocleavable terminating moiety.

In some embodiments, using evanescent wave imaging to determine the identity of the protected nucleotide incorporated into a sequencing primer comprises exposing the protected nucleotide to excitation light using an evanescent wave produced by total internal reflection in a substrate on which the substrate polynucleotide has been immobilized. In some embodiments, for example, excitation light may be emitted from one or more light sources of an evanescent wave imaging apparatus, and an evanescent wave may be produced at an interface or surface of the substrate by total internal reflection. The evanescent wave may have a field that extends a limited distance perpendicularly away from the substrate into a reservoir where the sequencing primer is annealed to an immobilized substrate polynucleotide. In some embodiments, the evanescent wave produced by total internal reflection of the excitation light is sufficient to produce a fluorescent emission from the detectable moiety (e.g., fluorophore) of the protected nucleotide. In some embodiments, the excitation light has a peak wavelength in the visible range of the electromagnetic spectrum or the UV range of the electromagnetic spectrum.

In some embodiments, using evanescent wave imaging to determine the identity of the protected nucleotide further comprises detecting the fluorescent emission from the detectable moiety of the protected nucleotide. In some embodiments, detecting the fluorescent emission is performed by an image sensor of an evanescent wave imaging apparatus described herein.

In some embodiments, using evanescent wave imaging to determine the identity of the protected nucleotide further comprises using one or more characteristics of the fluorescent emission (e.g., wavelength, intensity, lifetime) from the detectable moiety of the protected nucleotide to identify the protected nucleotide as “A,” “C,” “G,” “T,” or “U.”

Without wishing to be bound by a particular theory, the disclosure is directed, in part, to the discovery that localization of excitation to the surface of the substrate via use of the evanescent wave also enables a method of sequencing that does not require wash steps or other fluid transfer steps. Reagents in the aqueous solution, e.g., nucleotides comprising fluorescent detectable moieties, e.g., protected nucleotides described herein, are not substantially consumed by the evanescent wave, beyond the limited distance from the substrate into the reservoir, in contrast to methods of sequencing using direct excitation which may irradiate an entire sample or flow cell.

In some embodiments, a method comprises using evanescent wave imaging to cleave the photocleavable terminating moiety of the protected nucleotide. In some embodiments, using evanescent wave imaging to cleave the photocleavable terminating moiety of the protected nucleotide comprises exposing the protected nucleotide to an evanescent wave produced by total internal reflection of photocleavage light in the substrate.

In some embodiments, the photocleavage light may have a different wavelength than the excitation light, and therefore the evanescent wave produced by total internal reflection of the excitation light may not cause cleavage of the terminating moiety of the protected nucleotide, and vice versa. In some embodiments, the photocleavage light may have a peak wavelength in the UV range of the electromagnetic spectrum. In some embodiments, the photocleavage light may have a peak wavelength in the visible range of the electromagnetic spectrum.

In some embodiments, a method of sequencing described herein occurs under one or more reaction conditions. In some embodiments, devices described herein are configured to establish, maintain, and/or control one or more reaction conditions affecting sequencing using evanescent wave imaging. Reaction conditions may include, but are not limited to: temperature; the presence, identity of, and/or concentration of one or more buffers or salts; pH; and the concentrations (e.g., absolute or relative to one another) of sequencing primer(s).

In some embodiments, a first set of reaction conditions is maintained during a first phase of a method described herein (e.g., during nucleic acid amplification) and a second set of reaction conditions is maintained during a second phase of a method described herein (e.g., during sequencing using evanescent wave imaging). For example, in some embodiments, the temperature (e.g., during sequencing using evanescent wave imaging) is 32-60° C., 32-55° C., 32-50° C., 32-45° C., 32-40° C., 32-35° C., 35-60° C., 35-55° C., 35-50° C., 35-45° C., 35-40° C., 40-60° C., 40-55° C., 40-50° C., 40-45° C., 45-60° C., 45-55° C., 45-50° C., 50-60° C., 55-55° C., or 55-60° C.

### Reversible Termination and Incorporation Rates

The disclosure is directed, in part, to methods and devices that exercise control over the elongation of a sequencing primer using a substrate polynucleotide as a template.

In some embodiments, the relationships between the rate of incorporating a nucleotide (e.g., a protected nucleotide) into a sequencing primer (and optionally determining the identity of an incorporated nucleotide) and relieving reversible termination of elongation (e.g., cleaving a photocleavable terminating moiety of a protected nucleotide, e.g., thereby cleaving the detectable moiety free of the protected nucleotide) are important for the efficiency and accuracy of nucleic acid sequencing using evanescent wave imaging. In some embodiments, one or more features of the nucleic acid sequencing device are configured such that a polymerase extends a sequencing primer by a single protected nucleotide each time termination of elongation is reversed and/or to maximize the likelihood of extending by a single protected nucleotide each time termination of elongation is reversed. Without wishing to be bound by a particular theory, the disclosure is directed, in part, to the discovery that the rate at which reversible termination of elongation is relieved (e.g., the rate of cleavage of a photocleavable terminating moiety) must be substantially faster than the rate at which a nucleotide (e.g., a protected nucleotide) is incorporated into a sequencing primer for sequencing using evanescent wave imaging to be effective. When the rate of relieving termination is fast and the rate of incorporation is slow, a greater proportion of a pool of sequencing primers will remain synchronized, incorporating a single nucleotide each time termination is relieved. When the rate of relieving termination is comparable to the rate of incorporation, a lower proportion of a pool of sequencing primers will remain synchronized, and a significant minority of sequencing primers will have multiple nucleotides incorporated during a single termination relieving event. Accordingly in some embodiments, a device described herein is configured for or a method described herein comprises relieving reversible termination of elongation more quickly than incorporating a protected nucleotide into a sequencing primer (e.g., for a pool of sequencing primers).

For example, when the rates are comparable, a light source may expose a sequencing primer containing an incorporated protected nucleotide to light sufficient to induce cleavage of the photocleavable terminating moiety of the protected nucleotide, cleaving it and relieving termination of elongation. A polymerase may quickly add a further protected nucleotide while the sequencing primer is still exposed to light sufficient to induce cleavage of the photocleavable terminating moiety of the protected nucleotide, cleaving it and relieving termination of elongation again, e.g., without determining the identity of the further protected nucleotide and in contrast with the number of nucleotides added to other sequencing primers of the pool.

In a contrasting example, when the rate of incorporation is much slower than the rate of relieving termination, a light source may expose a sequencing primer containing an incorporated protected nucleotide to light sufficient to induce cleavage of the photocleavable terminating moiety of the protected nucleotide, cleaving it and relieving termination of elongation. By the time a polymerase incorporates a further protected nucleotide into the sequencing primer, the exposure to light sufficient to induce cleavage has ended and elongation is again terminated after a single nucleotide addition, allowing for determination of the identity of the further protected nucleotide.

In some embodiments, relieving termination for a pool of sequencing primers, e.g., in a spot, can be modeled by an exponential decay function, where r is the time constant (also referred to as the rate). In some embodiments, in the context of modeling relief of termination of elongation, τ (i.e., τ_{cleav}) is a representation of how rapidly cleavage of photocleavable terminating moieties is occurring. In some embodiments, τ (i.e., τ_{cleav}) corresponds to the time at which approximately ⅔ (e.g., approximately 63% or (1-1/e)) of photocleavable terminating moieties of incorporated protected nucleotides in a pool of sequencing primers have been cleaved. In some embodiments, the progress of nucleotide incorporation for a pool of sequencing primers, e.g., in a spot, can be modeled by an exponential decay function, where τ (i.e., τ_{inc}) is the time constant (also referred to as the rate). In some embodiments, in the context of modeling incorporation of a protected nucleotide, T is a representation of how rapidly incorporation of protected nucleotides is occurring. In some embodiments, τ (i.e., τ_{inc}) corresponds to the time at which approximately ⅔ (e.g., approximately 63% or (1-1/e)) of sequencing primers of a pool of sequencing primers have had a protected nucleotide added by polymerase. Times for relieving termination and/or incorporation can be measured from the time at which one or more light sources configured to reverse termination of elongation of a sequencing primer are activated (i.e., t=0 is when said one or more light sources begin emitting light). In some embodiments, it follows from these models that at time τ, approximately 63% of, e.g., photocleavable termination moieties have been cleaved, whereas at time 2τ approximately 86% of photocleavable termination moieties have been cleaved and at time 3τ approximately 95% of photocleavable termination moieties have been cleaved. Likewise, in some embodiments, it follows from these models that at time τ, approximately 63% of, e.g., sequencing primers of a pool have had a protected nucleotide incorporated by a polymerase, whereas at time 2τ approximately 86% of sequencing primers of a pool have had a protected nucleotide incorporated by a polymerase and at time 3τ approximately 95% of sequencing primers of a pool have had a protected nucleotide incorporated by a polymerase.

In some embodiments, a method described herein (e.g., a method of evanescent wave imaging) comprises relieving termination of elongation for a pool of sequencing primers such that relieving termination is achieved in time τ, 2τ, 3τ, 4τ, 5τ, or 6τ (e.g., resulting in a corresponding percentage of sequencing primers for which termination of elongation has been relieved). In some embodiments, a method described herein (e.g., a method of evanescent wave imaging) comprises relieving termination of elongation for a pool of sequencing primers such that relieving termination is achieved in time 3τ (e.g., resulting in a corresponding percentage of sequencing primers for which termination of elongation has been relieved). In some embodiments, a method or device is configured to result in a low τ of relieving termination of elongation (e.g., the lowest τ practicable τ), e.g., lower than the τ of incorporation of a nucleotide, e.g., sufficiently lower than the τ of incorporation of a nucleotide to efficiently sequence a target nucleic acid.

In some embodiments, a method described herein (e.g., a method of evanescent wave imaging) comprises incorporating individual nucleotides (e.g., protected nucleotides) into a pool of sequencing primers such that incorporation is achieved in time τ, 2τ, 3τ, 4τ, 5τ, or 6τ (e.g., resulting in a corresponding percentage of sequencing primers for which incorporation has occurred). In some embodiments, a method described herein (e.g., a method of evanescent wave imaging) comprises incorporating individual nucleotides (e.g., protected nucleotides) into a pool of sequencing primers such that incorporation is achieved in time 3τ (e.g., resulting in a corresponding percentage of sequencing primers for which incorporation has occurred). In some embodiments, a method or device is configured to result in a high τ of incorporation (e.g., the highest practicable τ), e.g., higher than the τ of relieving termination of elongation, e.g., sufficiently higher than the τ of relieving termination of elongation to efficiently sequence a target nucleic acid.

In some embodiments, a time (e.g., τ) of incorporation of a protected nucleotide into a sequencing primer is greater than a time (e.g., τ) of cleavage of a photocleavable terminating moiety of a protected nucleotide. The time (e.g., τ) of incorporation may be measured from a time when all sequencing reagents were added to the reservoir (for an initial step) or a time when a photocleavable terminating moiety was cleaved (for all subsequent steps) to a time that incorporation of the protected nucleotide (e.g., a threshold level of incorporation) was detected. The time (e.g., τ) of cleavage may be measured from a time photocleavage light was emitted to a time when no fluorescent emission from a detectable moiety was detected or fluorescent emission decreases below a threshold level. In some embodiments, time (e.g., τ) of incorporation of a protected nucleotide increases over time, e.g., as one or more sequencing reagents are consumed or the level of one or more sequencing reagents drops below a threshold level.

In some embodiments, a ratio of the rate (e.g., 1/τ) of cleavage of a photocleavable terminating moiety from a nucleotide to the rate (e.g., 1/τ) of incorporation of the nucleotide (e.g., protected nucleotide) is at least 30:1, at least 50:1, at least 100:1, at least 200:1, at least 250:1, at least 500:1, at least 800:1, at least 1000:1, at least 2000:1, at least 5000:1, or at least 10,000:1. In some embodiments, a ratio of the rate (e.g., 1/τ) of cleavage of a photocleavable terminating moiety from a nucleotide to the rate (e.g., 1/τ) of incorporation of the nucleotide (e.g., protected nucleotide) is in a range from 30:1 to 50:1, 30:1 to 100:1, 30:1 to 200:1, 30:1 to 500:1, 30:1 to 1000:1, 30:1 to 5000:1, 30:1 to 10,000:1, 50:1 to 100:1, 50:1 to 200:1, 50:1 to 500:1, 50:1 to 1000:1, 50:1 to 5000:1, 50:1 to 10,000:1, 100:1 to 200:1, 100:1 to 500:1, 100:1 to 1000:1, 100:1 to 5000:1, 100:1 to 10,000:1, 500:1 to 1000:1, 500:1 to 2000:1, 500:1 to 5000:1, 500:1 to 10,000:1, 1000:1 to 5000:1, 1000:1 to 10,000:1, or 5000:1 to 10,000:1.

In some embodiments, a ratio of the τ of incorporation of a nucleotide (e.g., a protected nucleotide) to the τ of cleavage of a photocleavable terminating moiety from the nucleotide is at least 30:1, at least 50:1, at least 100:1, at least 200:1, at least 250:1, at least 500:1, at least 800:1, at least 1000:1, at least 2000:1, at least 5000:1, or at least 10,000:1. In some embodiments, a ratio of the τ of incorporation of a nucleotide (e.g., a protected nucleotide) to the τ of cleavage of a photocleavable terminating moiety from the nucleotide is in a range from 30:1 to 50:1, 30:1 to 100:1, 30:1 to 200:1, 30:1 to 500:1, 30:1 to 1000:1, 30:1 to 5000:1, 30:1 to 10,000:1, 50:1 to 100:1, 50:1 to 200:1, 50:1 to 500:1, 50:1 to 1000:1, 50:1 to 5000:1, 50:1 to 10,000:1, 100:1 to 200:1, 100:1 to 500:1, 100:1 to 1000:1, 100:1 to 5000:1, 100:1 to 10,000:1, 500:1 to 1000:1, 500:1 to 2000:1, 500:1 to 5000:1, 500:1 to 10,000:1, 1000:1 to 5000:1, 1000:1 to 10,000:1, or 5000:1 to 10,000:1.

In some embodiments, the t of incorporation of nucleotides (e.g., protected nucleotides) into a pool of sequencing primers is less than about 15, 12, 10, 9, 8, 7, 6, 5, 4, 3, 2, or 1 minutes (and optionally at least about 5, 10, 20, 30, 40, 50, or 60 seconds). In some embodiments, the τ of incorporation of nucleotides (e.g., protected nucleotides) into a pool of sequencing primers is in a range of 0.1-15, 0.1-10, 0.1-7, 0.1-6, 0.1-5, 0.1-4, 0.1-3, 0.1-2, 0.1-1.5, 0.1-1, 0.1-0.75, 0.1-0.5, 0.1-0.25, 0.5-15, 0.5-10, 0.5-7, 0.5-6, 0.5-5, 0.5-4, 0.5-3, 0.5-2, 0.5-1.5, 0.5-1, 0.5-0.75, 1-15, 1-10, 1-7, 1-6, 1-5, 1-4, 1-3, 1-2, 1-1.5, 1.5-15, 1.5-10, 1.5-7, 1.5-6, 1.5-5, 1.5-4, 1.5-3, 1.5-2, 3-15, 3-10, 3-7, 3-6, 3-5, 3-4, 5-15, 5-10, 5-7, or 5-6 minutes.

In some embodiments, the τ of cleavage of the detectable moiety from the nucleotide (e.g., cleavage of a photocleavable terminating moiety) is less than about 5, 4, 3, 2.5, 2, 1.5, 1, 0.5, 0.45, 0.4, 0.35, 0.3, 0.25, 0.22, 0.2, 0.18, 0.16, 0.14, 0.12, 0.1, 0.08, 0.06, 0.04, 0.02, 0.01, 0.008, 0.006, 0.004, 0.002, or 0.001 seconds (s). In some embodiments, the τ of cleavage of the detectable moiety from the nucleotide (e.g., cleavage of a photocleavable terminating moiety) is in a range of 0.001-3, 0.001-2.5, 0.001-1.5, 0.001-1, 0.001-0.5, 0.001-0.3, 0.001-0.2, 0.001-0.15, 0.001-0.1, 0.001-0.05, 0.001-0.02, 0.001-0.01, 0.001-0.005, 0.01-3, 0.01-2.5, 0.01-1.5, 0.01-1, 0.01-0.5, 0.01-0.3, 0.01-0.2, 0.01-0.15, 0.01-0.1, 0.01-0.05, 0.01-0.02, 0.02-3, 0.02-2.5, 0.02-1.5, 0.02-1, 0.02-0.5, 0.02-0.3, 0.02-0.2, 0.02-0.15, 0.02-0.1, 0.02-0.05, 0.1-3, 0.1-2.5, 0.1-1.5, 0.1-1, 0.1-0.5, 0.1-0.3, 0.1-0.2, 0.1-0.15, 0.2-3, 0.2-2.5, 0.2-1.5, 0.2-1, 0.2-0.5, 0.2-0.3, 0.5-3, 0.5-2.5, 0.5-1.5, or 0.5-1 seconds.

A person of skill in the art will appreciate that the time (e.g., r or average time) of incorporation of nucleotides (e.g., protected nucleotides) into sequencing primers or the time (e.g., r or average time) of cleavage of detectable moieties from the nucleotides (e.g., protected nucleotides) can be adjusted by altering the configuration of a device described herein or the reaction conditions of the sequencing reaction (e.g., within the reservoir), e.g., using the guidance provided by the disclosure. For example, increasing the power of the one or more light sources that emit excitation light that produces an evanescent wave that effectively cleaves a photocleavable terminating moiety can decrease the time (e.g., r or average time) of cleavage of detectable moieties from the nucleotides (e.g., protected nucleotides), as can decreasing the distance between said one or more light sources and the substrate. As a further example, the concentrations of nucleotides (e.g., protected nucleotides), the concentration and/or characteristics of the polymerase (e.g., mutations or other modifications to the polymerase), and the temperature of the aqueous solution can be altered to modify the time (e.g., r or average time) of incorporation of nucleotides (e.g., protected nucleotides) into sequencing primers.

### Read Length, Cycle Time, & Iteration Time

In some embodiments, a device or a method of sequencing described herein is capable of sequencing a target nucleic acid having a length of at least 1, 2, 5, 10, 20, 25, 30, 40, 50, 60, 70, 80, 90, 100, 125, 150, 175, 200, 250, 300, 350, 400, 450, 500, 600, 700, 800, 900, 1000, 1500, 2000, 2500, 3000, 4000, 5000, or 10,000 bases. In certain embodiments, a device or a method of sequencing described herein is capable of sequencing a target nucleic acid having a length in a range from 1-2, 1-5, 1-10, 1-20, 1-25, 1-50, 1-100, 1-150, 1-200, 1-250, 1-500, 1-1000, 1-2000, 1-5000, 1-10,000, 2-5, 2-10, 2-20, 2-25, 2-50, 2-100, 2-150, 2-200, 2-250, 2-500, 2-1000, 2-2000, 2-5000, 2-10,000, 5-10, 5-20, 5-25, 5-50, 5-100, 5-150, 5-200, 5-250, 5-500, 5-1000, 5-2000, 5-5000, 5-10,000, 10-20, 10-25, 10-50, 10-100, 10-150, 10-200, 10-250, 10-500, 10-1000, 10-2000, 10-5000, 10-10,000, 25-50, 25-100, 25-150, 25-200, 25-250, 25-500, 25-1000, 25-2000, 25-5000, 25-10,000, 50-100, 50-150, 50-200, 50-250, 50-500, 50-1000, 50-2000, 50-5000, 50-10,000, 100-200, 100-250, 100-500, 100-1000, 100-2000, 100-5000, 100-10,000, 250-500, 250-1000, 250-2000, 250-5000, 250-10,000, 500-1000, 500-2000, 500-5000, 500-10,000, 1000-1000, 1000-10,000, or 5000-10,000 bases.

In some embodiments, a device or a method of sequencing described herein is capable of completing a read of nucleic acid sequencing, wherein a target nucleic acid molecule's sequence is determined, in no more than 10, 20, 30, 40, 50, 60, 70, 80, 90, 100, 110, or 120 minutes. In certain embodiments, a device or method of sequencing described herein is capable of completing a read of nucleic acid sequencing in 10-20, 10-30, 10-60, 10-90, 10-120, 20-30, 20-60, 20-90, 20-120, 30-60, 30-90, 30-120, 60-90, 60-120, or 90-120 minutes.

In some embodiments, a device or a method of sequencing described herein is capable of completing a cycle of nucleic acid sequencing, wherein a nucleotide (e.g., a protected nucleotide) is incorporated, elongation is terminated, the identity of the incorporated nucleotide is determined, and elongation termination is relieved in no more than 15, 12, 10, 9, 8, 7, 6, 5, 4, 3, 2, or 1 minutes. In some embodiments, a cycle of nucleic acid sequencing takes 0.1-15, 0.1-10, 0.1-7, 0.1-6, 0.1-5, 0.1-4, 0.1-3, 0.1-2, 0.1-1.5, 0.1-1, 0.1-0.75, 0.1-0.5, 0.1-0.25, 0.5-15, 0.5-10, 0.5-7, 0.5-6, 0.5-5, 0.5-4, 0.5-3, 0.5-2, 0.5-1.5, 0.5-1, 0.5-0.75, 1-15, 1-10, 1-7, 1-6, 1-5, 1-4, 1-3, 1-2, 1-1.5, 1.5-15, 1.5-10, 1.5-7, 1.5-6, 1.5-5, 1.5-4, 1.5-3, 1.5-2, 3-15, 3-10, 3-7, 3-6, 3-5, 3-4, 5-15, 5-10, 5-7, or 5-6 minutes.

### Wash Steps and Fluid Transfer

In some embodiments, a method of nucleic acid sequencing comprises adding one or more sequencing reagents (e.g., a protected nucleotide, a polymerase, one or more sequencing primers) to a reservoir after completion of amplification. In some such embodiments, a method comprises a wash step after completion of amplification and prior to addition of one or more sequencing reagents to the reservoir. In some embodiments, the wash step removes one or more (e.g., all or substantially all) amplification reagents from the reservoir. Without wishing to be bound by a particular theory, the presence of amplification reagents (e.g., non-protected nucleotides or amplification enzymes, e.g., nucleases or polymerases) may interfere with sequencing reagents, and thus in some embodiments efficiency and accuracy of sequencing can be improved by removal of the amplification reagents prior to sequencing.

In some embodiments, methods of nucleic acid sequencing described herein do not comprise a fluid transfer step, e.g., do not require a user to transfer a precise volume into or out of the reservoir. In some embodiments, methods of nucleic acid sequencing described herein do not comprise a fluid transfer step after sequencing reagents are added to the reservoir (e.g., the fluid transfer step(s) may be limited to washing amplification reagents out of the reservoir and/or addition of sequencing reagents to the reservoir). In some cases, such methods of nucleic acid sequencing advantageously reduce the amounts of costly reagents (e.g., modified nucleotides) needed. For example, such methods may use reduced amounts of costly reagents compared to flow cell-based methods of nucleic acid sequencing comprising one or more wash steps after incorporation of each nucleotide. In some cases, such methods of nucleic acid sequencing advantageously reduce the amount of time required to sequence a target nucleic acid (e.g., by avoiding the time needed to perform a wash step after incorporation of each nucleotide). In some cases, such methods of nucleic acid sequencing advantageously facilitate performance of the methods for layperson users without access to laboratory equipment. Such methods may also improve accuracy of the methods since fluid transfer steps may introduce error and result in failure of sequencing or false readouts.

### Software

In some embodiments, techniques described herein may be embodied in computer-executable instructions implemented as software, including as application software, system software, firmware, middleware, embedded code, or any other suitable type of computer code. For example, a processing system (e.g., 126) may be configured to control a nucleic acid sequencing device (e.g., a device comprising an evanescent wave imaging apparatus) according to executable code accessed from a memory device (e.g., 130). The executable code may be run or executed by one or more computer processors (e.g., 128) to control various electronics of the device, described herein, based on code modules that control one or more of the various electronics according to various sequencing procedures, described herein.

Such computer-executable instructions may be written using any of a number of suitable programming languages and/or programming or scripting tools, and also may be compiled as executable machine language code or intermediate code that is executed on a framework or virtual machine. Such computer-executable instructions may be stored on at least one non-transitory computer-readable storage medium, which may be executed by one or more computer processors to perform various aspects of the techniques described herein. The at least one computer-readable storage medium may include any one or any combination of: a magnetic medium (e.g., a hard disk drive, magnetic tape, etc.); an optical medium (e.g., a compact disk (CD), a Digital Versatile Disk (DVD), etc.); a persistent or non-persistent solid-state memory (e.g., a flash memory device, a magnetic RAM, etc.); and/or any other suitable storage medium that is a physical or tangible structure storing computer-executable code in a non-transitory state.

In some embodiments, a software application allows a user to control one or more parameters of a method or device described herein. In certain embodiments, a user may define a protocol comprising a number of cycles of visible light illumination, a wavelength for visible light illumination, an amount of time for visible light illumination, a number of cycles of image capture, an amount of time for image capture, a number of cycles of UV light illumination, a wavelength for UV light illumination, and/or an amount of time for UV light illumination. In some cases, once a protocol is defined, the user may specify whether and how many times the protocol should be repeated.

In some embodiments, a software application allows for dynamic modification of one or more parameters of a method based on real-time information (e.g., incorporation and/or cleavage information) received during performance of a method. In certain embodiments, the software application evaluates the extension of one or more sequencing primers annealed to substrate polynucleotides. In some cases, evaluating the extension of one or more sequencing primers comprises determining the percentage of sequencing primers that incorporated a protected nucleotide (e.g., by determining the percentage of detectable moieties that emitted light). In some embodiments, a user may use the software application to modify one or more parameters of sequencing primer extension based on the percentage of sequencing primers that incorporated a protected nucleotide. In certain embodiments, modifying one or more parameters of sequencing primer extension comprises increasing or decreasing an amount of time provided to incorporate a nucleotide and/or one or more amounts of time provided to determine the identity of a protected nucleotide (e.g., by providing a pulse of illumination to excite a detectable moiety of the protected nucleotide for an amount of time and capturing an image for another amount of time), increasing or decreasing wavelength (e.g., visible light wavelength) and/or power density of light emitted from a light source (e.g., to excite a detectable moiety of a protected nucleotide).

In certain embodiments, the software application evaluates the cleavage of photocleavable terminating moieties from protected nucleotides incorporated into one or more sequencing primers. In some cases, evaluating the cleavage of photocleavable terminating moieties comprises determining the percentage of photocleavable terminating moieties and detectable moieties that have been cleaved from incorporated protected nucleotides. In some embodiments, a user may use the software application to modify one or more parameters of protected nucleotide cleavage based on the percentage of photocleavable terminating moieties and detectable moieties that were cleaved. In certain embodiments, modifying one or more parameters of cleavage comprises increasing or decreasing an amount of time provided for cleavage of a protected nucleotide (e.g., by providing a pulse of illumination to cleave the photocleavable terminating moiety of the protected nucleotide), increasing or decreasing wavelength (e.g., UV wavelength) and/or power density of light emitted from a light source (e.g., to cleave a photocleavable terminating moiety of a protected nucleotide).

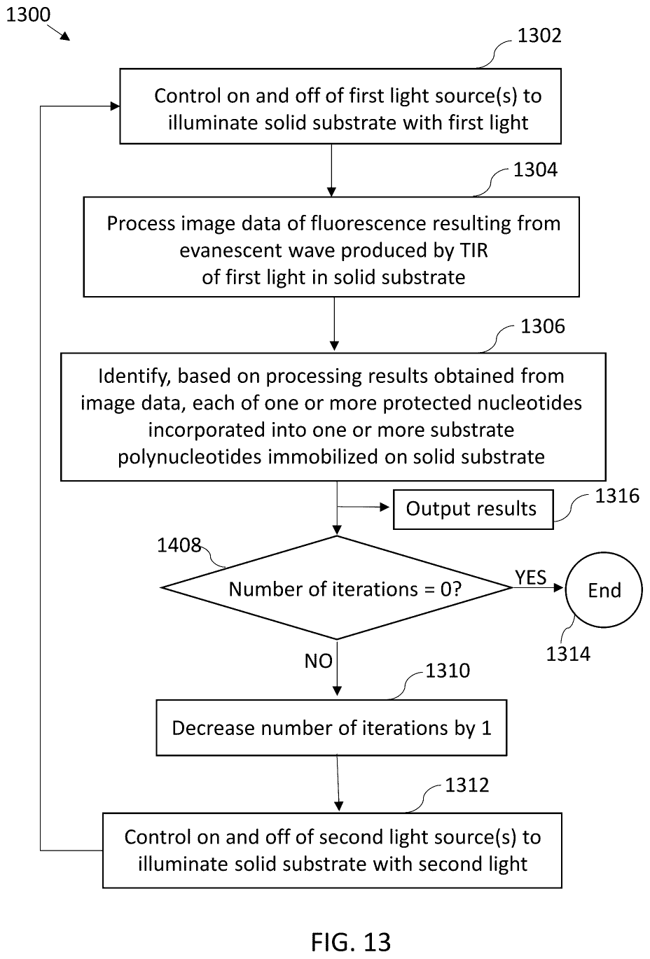

FIG. 13 shows a flow chart 1300 for a method of nucleic acid sequencing, according to some embodiments. In some embodiments, acts of the method may be performed via software code executed by one or more computer processors (e.g., 128). The software code may be stored on at least one non-transitory computer-readable storage medium accessible for execution by the one or more processors. In the method of flow chart 1300, one or more acts need not be performed in the order shown; one or more acts may be omitted; two or more acts may be performed concurrently; and/or one or more additional acts not explicitly shown may be performed before, during, or after one or more acts shown.

In some embodiments, the method of flow chart 1300 may be performed by a processing system (e.g., 126) coupled to a nucleic acid sequencing device. At act 1302, one or more first light sources may be turned on and off to enable a first light to be transmitted into a substrate to produce an evanescent wave at an interface of the substrate. As described above, the evanescent wave may cause detectable moieties of protected nucleotides, which have been incorporated into sequencing primers annealed to substrate polynucleotides immobilized on the substrate, to emit light that may be used to identify, for each of the protected nucleotides, whether the protected nucleotide is “A” or “C” or “G” of “T” or “U”. At act 1304, image data captured by an imaging system may be output from the imaging system to the processing system where it is determined, for example, whether each spot on the substrate emitted light and, if so, what one or more characteristics (e.g., wavelength, intensity, pulse width) of the emission light were. At act 1306, based on processing results obtained from the image data, each spot on the substrate may be assigned an identity (e.g., “A” or “C” or “G” of “T” or “U”) or may be given an error designation. For example, if the image data does not show any appreciable emission for a spot, that spot may be given an error code. In some embodiments, based on emission light intensity, the processing results may include a number of photons emitted for each spot, which may be correlated to a number of detectable moieties that emitted light at each spot. At act 1308, a decision is made as to whether an additional incorporation iteration is to be performed. For example, a sequencing operation may require a plurality of incorporation iterations to be performed so that additional protected nucleotides are incorporated, one by one, in each of the sequencing primers, to form a sequence of incorporated nucleotides for each sequencing primer. If, at act 1308, the number of iterations is not zero, i.e., at least one additional protected nucleotide is to be incorporated in each of the sequencing primers, the method proceeds to act 1310, where the number of iterations is decreased by one. At act 1312, one or more second light sources may be turned on and off to enable a second light to be transmitted into the substrate to produce a second evanescent wave at the interface of the substrate. As described above, the second evanescent wave may cause cleavage of photocleavable terminating moieties of the protected nucleotides, thus enabling incorporation of additional protected nucleotides into the sequencing primers. The method may then return to act 1302 to commence another iteration. At act 1308, if the number of iterations is determined to be zero, i.e., no further incorporation is needed, the method may end at act 1314. Optionally, at act 1316, identification results may be output. In some embodiments, a user may review the identification results to determine whether one or more errors occurred and, if so, whether further acts of the method should be aborted. In some embodiments, a user may determine from the identification results whether one or more variables of the method (e.g., illumination power of the first light source(s), duration of time of transmission of the first light into the substrate at act 1302, wait time between act 1312 and next act, etc.) should be adjusted and may manually adjust the variable(s). In some embodiments, at act 1316, the identification results may be processed automatically by the processing system to determine whether one or more variables of the method should be adjusted and to adjust the variable(s) automatically. In some embodiments, the processing system may abort further acts of the method if the identification results indicate a percentage of errors greater than a predetermined threshold. All references and publications cited herein are hereby incorporated by reference.

### EXAMPLES

The following examples are provided to further illustrate some embodiments of the present invention but are not intended to limit the scope of the invention; it will be understood by their exemplary nature that other procedures, methodologies, or techniques known to those skilled in the art may alternatively be used.

### Example 1: Nucleic Acid Amplification by Solid-Phase RPA Detected in an Exemplary Device

This Example demonstrates use of an exemplary nucleic acid amplification method, RPA, to elongate immobilized nucleic acid primers in an exemplary device of the disclosure.

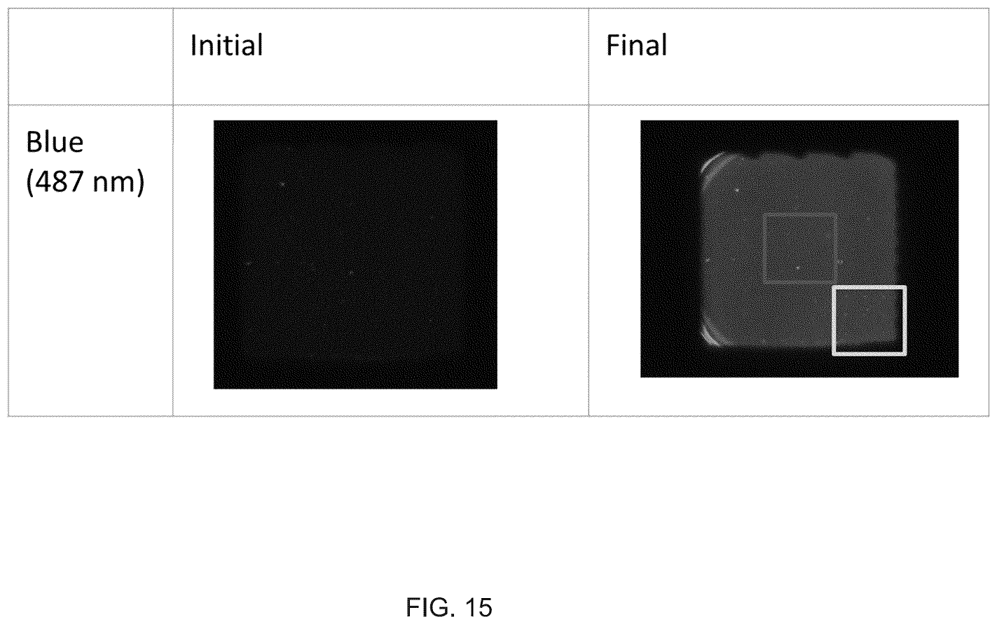

An exemplary substrate, a sapphire slide, was functionalized using APTES preparation. The slide was spotted (Sonoplot) using azide labeled reverse primers for SARS-COV-2 N gene or S gene target sequences to form spots containing the reverse primers immobilized to the slide. The slide was placed into exemplary device components (FIG. 14), forming the bottom of the reservoir and sandwiched between two silicone gaskets (exemplary isolation layers). After forming the reservoir, amplification reagents including forward RPA primer for the S gene, rehydration buffer, exemplary target nucleic acid (comprising SARS-COV-2 S gene sequence; procured by gBlock from IDT), water, TwistAmp® Basic (containing polymerase, dNTPs (normal, non-protected nucleotides), and associated reagents), and EvaGreen intercalating dye were prepared and mixed, and the amplification reagents were added to the reservoir. Magnesium acetate was added to the reservoir to begin RPA amplification. The EvaGreen intercalating dye emitted fluorescence in a range detectable after excitation using the evanescent wave produced by the 487 nm (Blue) LED channel of the exemplary apparatus. The slide was imaged at t=0 immediately after addition of amplification reagents and Mg (Initial) (FIG. 15). The reservoir and slide were maintained at 39° C. and every 30 minutes were imaged by the device up to a final image at two hours (Final) (FIG. 15).

The results showed spots appeared at Final on the slide as imaged by the blue LED channel in the location of the S gene reverse primer spots (yellow box) and no spots appeared in the location of the N gene reverse primer spots (purple box). This is consistent with the inclusion of forward RPA primer and target nucleic acid for the SARS-COV-2 S gene and the absence of forward RPA primer and target nucleic acid for the SARS-COV-2 N gene. The results show that solid-phase RPA amplified an exemplary target nucleic acid on a substrate in an exemplary device using evanescent wave imaging.

### Example 2: Sequencing a Short Known Nucleic Acid Using Evanescent Wave Imaging and an Exemplary Device

This Example demonstrates use of evanescent wave imaging to sequence three bases of a short known nucleic acid using an exemplary device of the disclosure.

An exemplary substrate, a sapphire slide, was functionalized using APTES preparation. The slide was spotted to form 9 spots as in Example 1 (Sonoplot) using a primer template duplex comprising a single-stranded portion of template such that the next three bases to be incorporated into the primer (according to Watson-Crick base-pairing) should be A, followed by G, followed by T. The slide was placed into exemplary device components (FIG. 14), forming the bottom of the reservoir and sandwiched between two silicone gaskets (exemplary isolation layers). The exemplary device components were operably linked with an exemplary apparatus of the disclosure, which coupled a 487 nm emitting LED (ROHM SMLD12E3N1WT86, 66 mW, 85 mcd) to one outer edge of the slide, 590 nm (LA YL20WP5, 12 mW) and 647 nm (Kingbright AP1608SECK, 75 mW, 160 mcd) emitting LEDs to another outer edge of the slide, and UV emitting LEDs (Light-Avenue UY20WP1, 365 nm, 100 mW) to the remaining two outer edges of the slide. A 500 nm longpass filter was attached to the camera tunnel of the apparatus. Buffer A, containing ThermoPol buffer, 4 mM ascorbic acid, and 0.3 U/μL Therminator polymerase, was added to the reservoir and the reservoir and slide incubated at room temperature for at least 15 minutes to load the primer template duplexes with polymerase. Buffer B, containing a pool of exemplary protected nucleotides containing exemplary photocleavable terminating groups and detectable moieties (dG-Z-AF488, dC-Z-AF532, dA-Z-CF594, and dU-Z-Atto647, with photocleavable terminating groups having Formula II wherein R₁ is a tert-butyl group, R₅ is an alkyne group leading to the fluorescent moiety, R₆ is a methoxy group, and non-designated R groups are H), ThermoPol buffer, and 4 mM ascorbic acid was added to the reservoir to initiate sequencing.

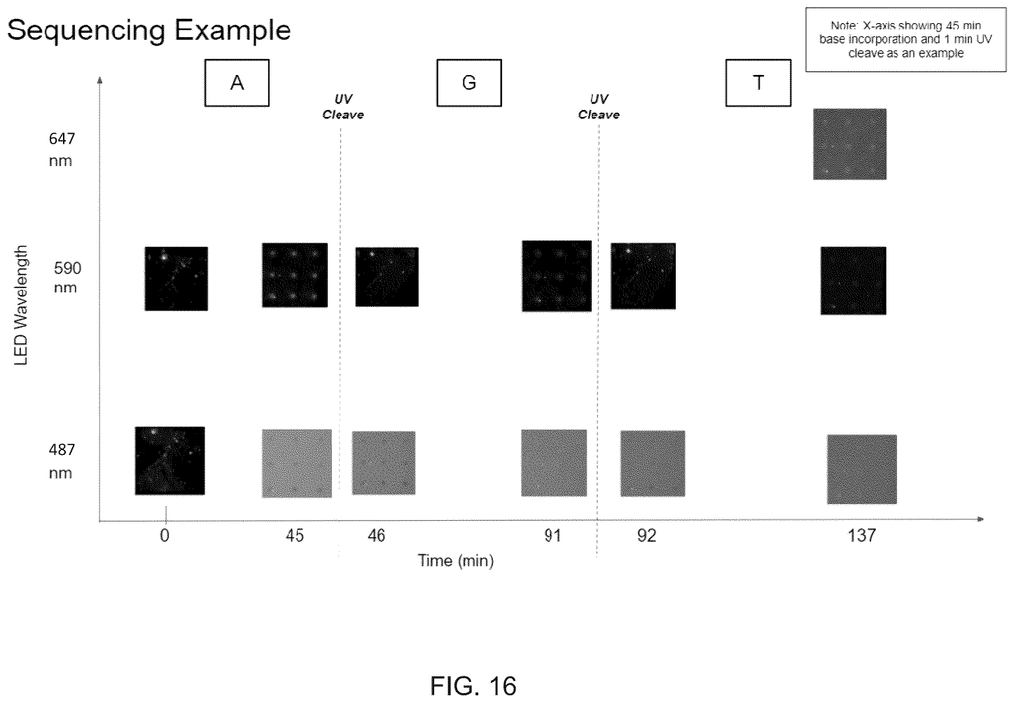

FIG. 16 shows slide images produced by evanescent wave imaging using either the 487 nm (Blue) LED channel or the 590/647 nm (Orange) LED channel of the exemplary device. The pool of exemplary protected nucleotides contained adenosine nucleotides and thymidine nucleotides containing detectable moieties capable of emitting light detected by the Orange LED channel, and guanosine nucleotides and cytosine nucleotides containing detectable moieties capable of emitting light detected by the Blue LED channel. Chronologically, slide images progress from left to right with the Blue LED channel (487 nm) images at the bottom and the Orange LED channel (590 nm) images at the top. Upon addition of Buffer B, evanescent wave imaging began immediately (t=0). At t=0, no signal was seen in slide images from either the Blue or Orange channels.

After approximately 45 minutes, positive signal was seen at the locations of the 9 spots in the image from the Orange channel, showing that adenosine nucleotides containing detectable moiety were incorporated into primers of the spots. The Blue channel slide image showed a blanket of background signal across the slide, with decreases in background signal at the locations of the 9 spots (negative signal). While not wishing to be bound by theory, it is thought that non-specific binding of guanosine and cytosine protected nucleotides to the slide may have produced the background signal, with the negative signal at the locations of the 9 spots resulting from electrostatic repulsion of said guanosine and cytosine protected nucleotides from the predominantly negatively charged primer template.

After the approximately 45 minute imaging, the UV emitting LEDs were activated for approximately 5 to 10 seconds of pulses to produce an evanescent wave that induced photocleavage of the incorporated adenosine nucleotide and reversed termination of elongation. After photocleavage (t=˜46 minutes), the Orange channel slide image showed no positive signal at the locations of the 9 spots, showing that photocleavage removed the detectable moieties from the incorporated adenosine nucleotides. The Blue channel slide image showed essentially no change after photocleavage, consistent with nucleotide incorporation having a slower rate than photocleavage. After another approximately 45 minutes (t=˜91 minutes), negative signal disappeared in the Blue channel slide image and positive signal appeared (yellow dots at the location of the 9 spots), showing that guanosine nucleotides containing detectable moiety were incorporated into primers of the spots. The Orange channel slide image showed faint positive signal at the locations of the 9 spots, which, without wishing to be bound by theory, may be a result of asynchronous N-1 incorporation (i.e., addition of adenosine nucleotides to primers that did not add adenosine in the initial round of elongation).

After the approximately 91 minute imaging, the UV emitting LEDs were again activated for approximately 5 to 10 seconds of pulses to produce an evanescent wave that induced photocleavage of the photocleavable moiety of the incorporated nucleotide and reversed termination of elongation. After photocleavage (t=˜92 minutes), the Blue channel slide image showed no positive signal at the locations of the 9 spots, showing that photocleavage removed the detectable moieties from the incorporated guanosine nucleotides. The Orange channel slide image showed essentially no positive signal after photocleavage; without wishing to be bound by theory, it is thought that photocleavage may have removed any detectable moieties associated with asynchronous N-1 incorporated adenosine nucleotides. After another approximately 45 minutes (t=˜137 minutes), positive signal appeared in the Orange channel slide image (specifically at the location of the 9 spots, particularly with 647 nm excitation corresponding to peak absorption of the detectable moiety of the thymidine nucleotide), showing that thymidine nucleotides were incorporated into primers of the spots. Positive signal appeared and negative signal disappeared at the locations of the spots in the Blue channel slide images as well, which, without wishing to be bound by theory, may be a result of asynchronous N-1 incorporation (i.e., addition of guanosine nucleotides to primers that did not add guanosine in the previous round of elongation).

These results demonstrate incorporation of exemplary protected nucleotides into a primer immobilized to a substrate, determination of the identity of the incorporated nucleotide using evanescent wave imaging, and control of termination of elongation using an evanescent wave using an exemplary device of the disclosure.

### Example 3: Measuring Incorporation of Exemplary Adenosine and Guanosine Protected Nucleotides

This example demonstrates incorporation of exemplary adenosine and guanosine protected nucleotides into an immobilized primer template on a substrate using an exemplary device of the disclosure.

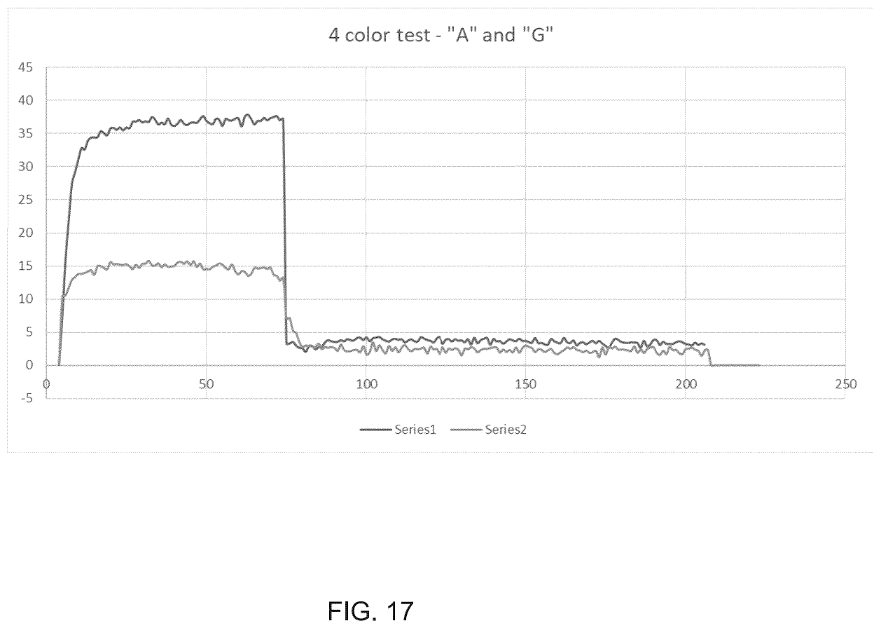

An exemplary substrate, a sapphire slide, was functionalized using APTES preparation. Slides were pre-treated with sulfo-NHS-acetate solution in order to passivate the slide surface, cap free amines, and prevent non-specific binding to the surface of the slide. The slide was spotted as in Example 1 (Sonoplot) using a primer template duplex comprising a single-stranded portion of template such that the next base to be incorporated into the primer (according to Watson-Crick base-pairing) should be A (Series1) or G (Series2). The slide was placed into exemplary device components (FIG. 14), forming the bottom of the reservoir and sandwiched between two silicone gaskets (exemplary isolation layers). The exemplary device components were operably linked with an exemplary apparatus of the disclosure, which coupled a 487 nm emitting LED to one outer edge of the slide, a 590 nm (LA YL20WP5, 12 mW) and 647 nm (Kingbright AP1608SECK, 75 mW, 160 mcd) emitting LEDs to another outer edge of the slide, and UV emitting LEDs (Light-Avenue UY20WP1, 365 nm, 100 mW) to the remaining two outer edges of the slide. Buffer A, containing ThermoPol buffer, 4 mM ascorbic acid, and 0.3 U/μL Therminator polymerase, was added to the reservoir and the reservoir and slide incubated at room temperature for at least 15 minutes to load the primer template duplexes with polymerase. Buffer B, containing a pool of exemplary protected nucleotides containing exemplary photocleavable terminating groups and detectable moieties (either dG-Z-AF488 or dA-Z-CF594, with photocleavable terminating groups having Formula II wherein R₁ is a tert-butyl group, R₅ is an alkyne group leading to the fluorescent moiety, R₆ is a methoxy group, and non-designated R groups are H), ThermoPol buffer, and 4 mM ascorbic acid was added to the reservoir to initiate incorporation and evanescent wave imaging was used to monitor mean fluorescence intensity corresponding to the peak fluorescence of the detectable moieties of the A and G nucleotides over time (FIG. 17). The x-axis is in units of cycles, which were set to an arbitrary 25 second length for the purposes of the experiment. The results show that mean fluorescence intensity reached a plateau after approximately 20 cycles for both A and G nucleotides.

At 75 cycles, UV light sources of the apparatus were activated to test photocleavage of the incorporated protected nucleotides. The results showed a precipitous decrease in fluorescence associated with the A or G nucleotides, showing that photocleavage removed the detectable moieties from the incorporated nucleotides.

### Example 4: Controlling and Detecting Incorporation of Three Adenosine Nucleotides Using an Exemplary Device

This example demonstrates sequential incorporation of exemplary adenosine protected nucleotides into an immobilized primer template on a substrate using an exemplary device of the disclosure.

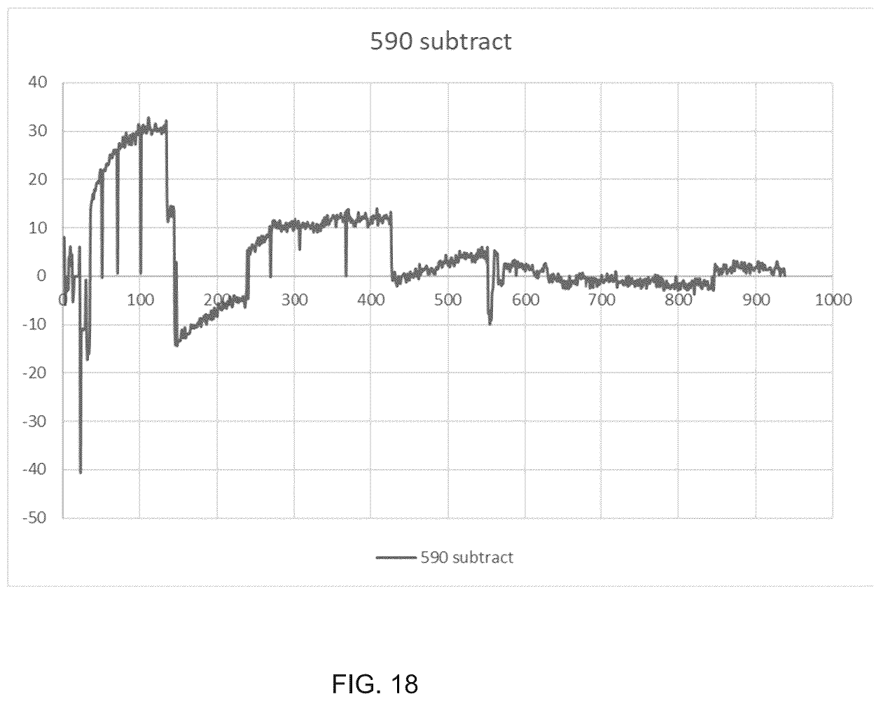

An exemplary substrate, a sapphire slide, was functionalized using APTES preparation. Slides were pre-treated with sulfo-NHS-acetate solution in order to passivate the slide surface, cap free amines, and prevent non-specific binding to the surface of the slide. The slide was spotted as in Example 1 (Sonoplot) using a primer template duplex comprising a single-stranded portion of template such that the next three bases to be incorporated into the primer (according to Watson-Crick base-pairing) should be A. The slide was placed into exemplary device components (FIG. 14), forming the bottom of the reservoir and sandwiched between two silicone gaskets (exemplary isolation layers). The exemplary device components were operably linked with an exemplary apparatus of the disclosure, which coupled a 487 nm emitting LED to one outer edge of the slide, a 590 nm (LA YL20WP5, 12 mW) and 647 nm (Kingbright AP1608SECK, 75 mW, 160 mcd) emitting LEDs to another outer edge of the slide, and UV emitting LEDs (Light-Avenue UY20WP1, 365 nm, 100 mW) to the remaining two outer edges of the slide. Buffer A, containing ThermoPol buffer, 4 mM ascorbic acid, and 0.3 U/μL Therminator polymerase, was added to the reservoir and the reservoir and slide incubated at room temperature for at least 15 minutes to load the primer template duplexes with polymerase. Buffer B, containing a pool of exemplary protected nucleotides containing exemplary photocleavable terminating groups and detectable moieties (dA-Z-CF594, with photocleavable terminating groups having Formula II wherein R₁ is a tert-butyl group, R₅ is an alkyne group leading to the fluorescent moiety, R₆ is a methoxy group, and non-designated R groups are H), ThermoPol buffer, and 4 mM ascorbic acid was added to the reservoir to initiate incorporation and evanescent wave imaging was used to monitor mean fluorescence intensity corresponding to the peak fluorescence of the detectable moieties of the A nucleotides over time (FIG. 18). The x-axis is in units of cycles, which were set to an arbitrary 25 second length for the purposes of the experiment.

The results showed that mean fluorescence intensity reached a plateau after approximately 100 cycles, corresponding to incorporation of the first adenosine protected nucleotide and termination of elongation. At cycle 140, UV light sources of the apparatus were activated, resulting in a sharp decrease in fluorescence as the detectable moiety of the incorporated nucleotide is removed by photocleavage. After photocleavage, fluorescence intensity increased until approximately cycle 270, a second plateau appeared, corresponding to incorporation of a second adenosine protected nucleotide and termination of elongation. At cycle 420, UV light sources of the apparatus were activated, resulting in a sharp decrease in fluorescence as the detectable moiety of the incorporated nucleotide is removed by photocleavage. After photocleavage, fluorescence intensity increased until at approximately cycle 520, a third plateau appeared, corresponding to incorporation of a third adenosine protected nucleotide and termination of elongation.

These results showed that an exemplary evanescent wave imaging apparatus can be used to monitor and control the incorporation of protected nucleotides, using an evanescent wave to induce and detect fluorescence from incorporated nucleotides and to induce photocleavage to relieve termination of elongation.

### Example 5: Measuring T of Incorporation and T of Cleavage of Exemplary Protected Nucleotide

This example demonstrates calculation of the τ of incorporation of an exemplary adenosine protected nucleotide into an immobilized primer template on a substrate and of the r of cleavage of the photocleavable moiety therefrom using an exemplary device of the disclosure.

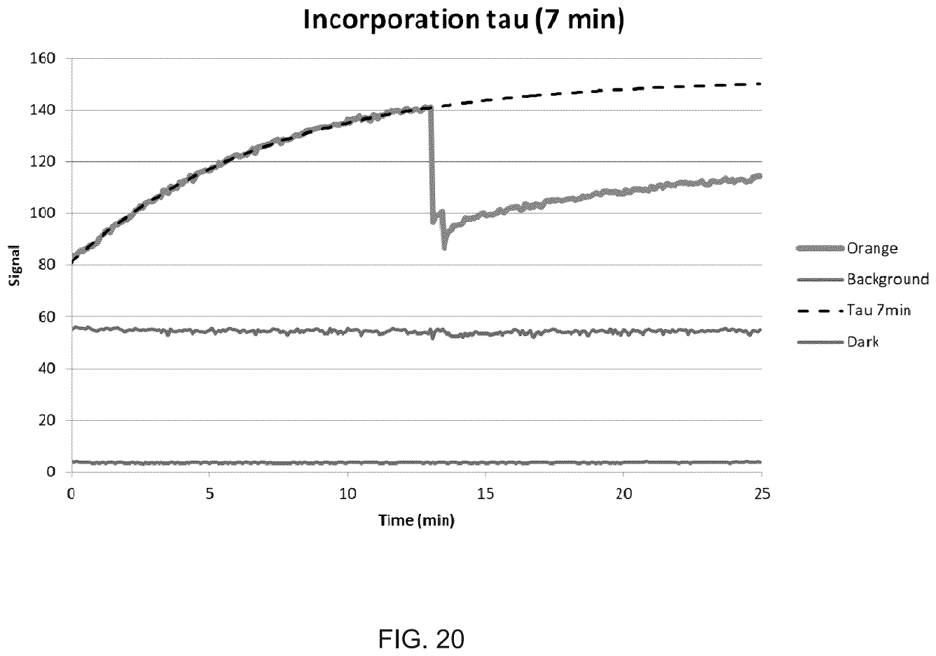

An exemplary substrate, a sapphire slide, was functionalized using APTES preparation. The slide was spotted as in Example 1 (Sonoplot) using a primer template duplex comprising a single-stranded portion of template such that the next base to be incorporated into the primer (according to Watson-Crick base-pairing) should be “A.” The slide was placed into exemplary device components, forming the bottom of the reservoir and sandwiched between two silicone gaskets (exemplary isolation layers). The exemplary device components were operably linked with an exemplary apparatus of the disclosure, which coupled a 487 nm emitting LED to one outer edge of the slide, a 590 nm (LA YL20WP5, 12 mW) and 647 nm (Kingbright AP1608SECK, 75 mW, 160 mcd) emitting LEDs to another outer edge of the slide, and UV emitting LEDs (Light-Avenue UY20WP1, 365 nm, 100 mW) to the remaining two outer edges of the slide. Buffer A, containing ThermoPol buffer, 4 mM ascorbic acid, and 0.3 U/μL Therminator polymerase, was added to the reservoir and the reservoir and slide incubated at room temperature for at least 15 minutes to load the primer template duplexes with polymerase. Buffer B, containing a pool of exemplary protected nucleotides containing exemplary photocleavable terminating groups and detectable moieties (dA-Z-CF594, with photocleavable terminating groups having Formula II wherein R₁ is a tert-butyl group, R₅ is an alkyne group leading to the fluorescent moiety, R₆ is a methoxy group, and non-designated R groups are H), ThermoPol buffer, and 4 mM ascorbic acid was added to the reservoir to initiate incorporation and evanescent wave imaging was used to monitor mean fluorescence intensity corresponding to the peak fluorescence of the detectable moieties of the A nucleotides over time (FIG. 20). Images were obtained approximately every 10 seconds.

The results showed an increase in Orange LED channel fluorescence over time, consistent with incorporation of an exemplary protected nucleotide into the primer. The τ of incorporation was calculated by fitting a single exponential asymptote to the fluorescence data, using the equation

$$A + {Be}^{- \frac{t}{τ}} ,$$

where λ=152, B=˜71, and τ=7 minutes. Accordingly, the τ of incorporation in this experiment monitoring incorporation of an exemplary protected nucleotide in an exemplary device of the disclosure was calculated to be 7 minutes.

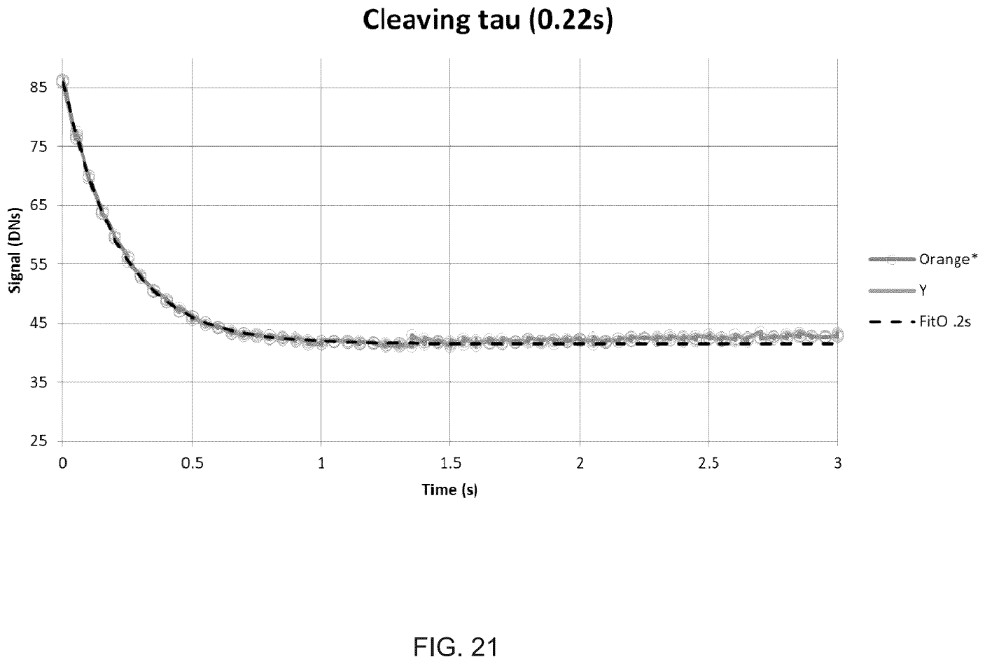

Approximately 14 minutes after addition of Buffer B, the UV emitting LEDs were activated for approximately 5 millisecond pulses to produce an evanescent wave that induced photocleavage of the photocleavable moiety of the incorporated nucleotide (seen in FIG. 21). The results showed a decrease in Orange LED channel fluorescence over time, consistent with cleavage of the photocleavable moiety and release of the detectable moiety of the protected nucleotide from the primer. The τ of cleavage was calculated by fitting a single exponential to the fluorescence data, using the equation

$$A + {Be}^{- \frac{t}{τ}} ,$$

where A=41.5, B=44.5, and τ=0.22 seconds. Accordingly, the T of cleavage in this experiment monitoring cleavage of a photocleavable moiety of an exemplary protected nucleotide in an exemplary device of the disclosure was calculated to be 0.22 seconds.

### Example 6: Immobilizing Primers on Substrates

This Example describes three methods for immobilizing primers on substrates to support surface amplification for sequencing. Method 1 is generally applicable for silicon oxide, and Methods 2 and 3 are generally applicable for silicon oxide-based substrates (e.g., quartz) and/or aluminum oxide-based substrates (e.g., sapphire).

### Method 1

In Method 1, substrate surfaces were mechanically polished and cleaned to obtain a largely defect-free, clean surface. A final gross cleaning by sonication in isopropyl alcohol was performed for 5 minutes, followed by washing with continuous flow of 18.2 MΩ·cm water over the substrate surfaces for 5 minutes. Excess water was blown off with nitrogen.

The substrate surfaces were then exposed to a 300 W reactive-ion etching (RIE) oxygen plasma for 10 minutes with 15 sccm (standard cubic centimeters per minute) oxygen flow to achieve a final cleaning and surface activation.

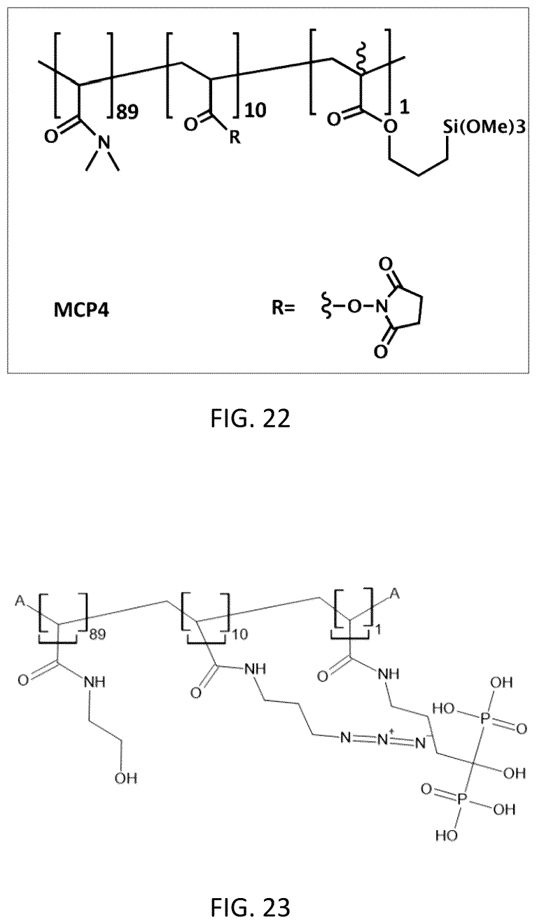

Immediately after plasma activation, the substrate surfaces were coated with the polymer MCP4 (Lucidant Polymers, https://www.lucidant.com/mcp-4-kit.html) according to the following protocol. The structure of the MCP4 polymer is shown in FIG. 22. To facilitate substrate handling, substrates were mounted in a custom holder that had an 8 mm diameter well on one side of the substrate. This substrate holder was used for all further treatments.

The MCP4 stock solution was diluted 1:50 with Coating Solution (Lucidant #COT1) (e.g., 1 mL of 50X MCP4 stock solution was added to 49 mL of 1×Coating Solution) and vortexed to mix. The 1×Coating Solution was prepared from concentrate (e.g., 20% concentrate (Lucidant 5XCOT1G and 80% filtered water). The slides were immersed in the diluted MCP4 solution for 30 minutes at room temperature. The slides were then washed individually in a large volume of water (e.g., for small numbers of slides, one slide at a time was grasped by forceps and swirled for a few seconds in 1 L deionized water). The slides were then immediately dried with a stream of nitrogen.

The slides were dried at 80° C. under high vacuum (less than 2 mm Hg) for 15 minutes. The slides were then stored inside a vacuum-sealed bag with a desiccant pack or in a desiccator. The slides were stored frozen (−20° C. or lower) until use. Under these conditions, the coated slides were stable for at least 1 year.

The MCP4 coated slides were then spotted with oligonucleotide primers. Reacting the coated substrates with oligonucleotides at various concentrations allowed for control of surface density of the resulting attached primers.

In general, a lower relative humidity (30-45%) was preferred. On days with high humidity, a tray of desiccant may help control relative humidity (rh). The substrates were baked at 80° C. for 15 minutes immediately before spotting. In some cases, 50 mM trehalose was added to the spotting buffer (e.g., Lucidant #SPT1) to increase uniformity within spots.

After spotting, remaining reactive groups were blocked by exposure to 1×Blocking Solution for 30 minutes at 50° C. The substrates were then rinsed with deionized water and dried.

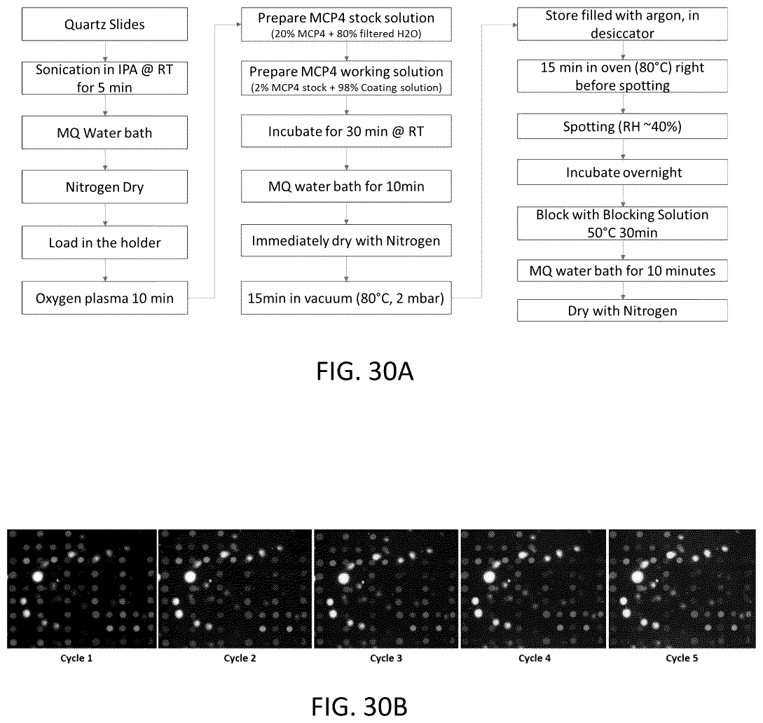

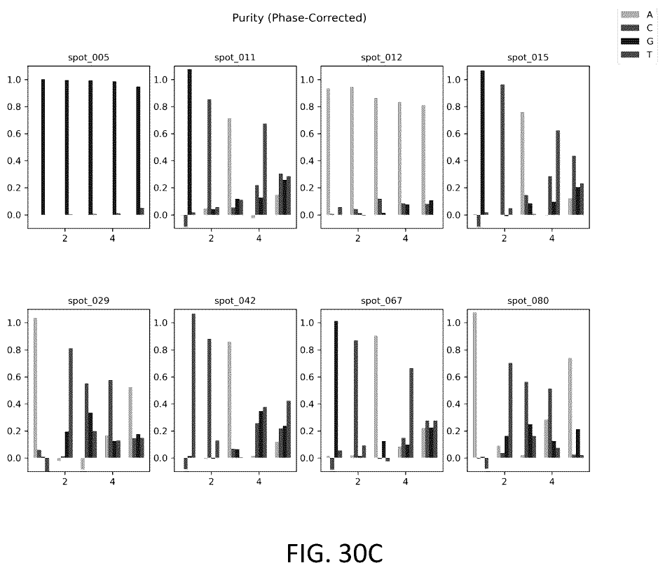

FIG. 30B shows images of slides prepared with Method 1 where the oligonucleotide concentration was 50 μM.

### Method 2

This method may be used for either silicon oxide-based substrates (e.g., quartz) or aluminum oxide-based substrates (e.g., sapphire).

### Cleaning

Similar to Method 1 above, all 8 surfaces of a substrate (10×10×0.5 mm) were chemically and/or mechanically polished to leave the substrate defect-free and clean of residue.

For c-face sapphire substrates, the following cleaning protocol was adopted. The sapphire slides were placed in a slide holder and sonicated in semiconductor grade acetone for 10 minutes. The slides were then sonicated in 18.2 MΩ·cm water for 10 minutes. The slides were then sonicated in semiconductor grade isopropanol for 10 minutes. Following sonication, the slides were dip rinsed in fresh 18.2 MΩ·cm water 3 times, refreshing the water every time. Fourth, the slides were stored in 10 mM HNO₃ until ready for use (minimum 30 minutes). The nitric acid treatment activates the aluminum oxide for reaction with zirconium oxide. Alternatively, the step of exposing the slides to HNO₃ may be replaced with a 10-minute 300 W RIE oxygen plasma treatment with 15 sccm of oxygen.

### Zirconium Oxide Deposition

A single layer of zirconium oxide was then deposited according to the following protocol. The substrate holders used in this protocol were all made from Teflon and were designed to securely hold the slides during the processing and cleaning steps (including curing in ovens and spray drying with clean dry nitrogen gas). A substrate holder used in this protocol could securely fit into a reaction vessel (e.g., a 20 mL scintillation vial) such that the lid could be attached without affecting the slide holder. The previously cleaned sapphire substrates were placed in the appropriate slide holder and, and the following steps were serially performed.

First, a reaction vessel (e.g., a virgin scintillation vial) and lid were dried in an oven for at least 10 minutes at 80° C. The reaction vessel was removed from the oven, and argon was dispensed into the reaction vessel to generate an inert atmosphere in the vessel (e.g., using a 10 second flow from the push button valve). The reaction vessel was charged with 9.9 mL of dry methoxyethanol. A needle with argon was inserted into a SureSeal bottle of methoxyethanol, and a 2-piece plastic syringe with a 22 gauge needle was used to remove 9.9 mL of methoxyethanol from the SureSeal bottle and add it to the reaction vessel. The needle with argon allowed the volume of solvent removed from the SureSeal bottle to be replaced with dry argon.

A bottle of 70% solution of zirconium (IV) propoxide in 1-propanol was opened, and dry argon was allowed to blanket the solution in the bottle. While flowing argon into the bottle, a 1000 μL pipette was used to remove 100 μL of zirconium (IV) propoxide solution from the bottle and add it to the methoxyethanol in the reaction vessel. The lid on the reaction vessel was closed, and the reaction vessel was shaken for 10 seconds.

While flowing argon into the reaction vessel, the slide holder with slides was inserted into the reaction vessel. Prior to insertion, the slide holder and slides were blown dry to avoid a significant volume of water from entering the reaction vessel. The slide holder and slides were not dried in an oven at an elevated temperature, as the surface-attached water is important for reactivity with the zirconium alkoxide. The reaction was allowed to proceed for a minimum of 16 hours (overnight).

The substrate holder was removed from the reaction vessel and dip rinsed in fresh methoxyethanol, followed by dip rinsing in 18.2 MΩ·cm water. The substrates were blown dry with clean dry nitrogen and baked in the oven for 4 hours at 80° C. This annealing step was important to ensure full reaction of surface groups with the zirconium alkoxide. The slides were then stored in a clean dry nitrogen atmosphere until ready for use.

### Phosphonate Deposition

The following coating procedure was used to coat the zirconia-activated substrates with polymers or small molecules with appropriate chemistry for the attachment of oligonucleotides

The zirconium oxide-coated slides were placed in appropriate slide holders (e.g., substrate holders made out of Teflon, as described above), and the following steps were performed serially.

First, a 0.2% w/v solution of a desired phosphonate ligand (e.g., a phosphonate-containing polymer or small molecule) in 18.2 MΩ·cm water was prepared. A phosphonate polymer having the structure shown in FIG. 23 was used in this Example. This phosphonate polymer had a similar structure to that of MCP4, except that the surface-active portion of the polymer was a bisphosphonate instead of a silane and the bioconjugation portion of the polymer was an azide rather than an NHS-activated ester. In addition, the inert backbone residue was hydroxyethyl acrylamide rather than the dimethyl acrylamide in MCP4. The relative proportions and overall molecular weight of the polymer may be adjusted to achieve optimal results and manufacturability. For a thicker, gel-like coating, the number of attachment points (e.g., bisphosphonate moieties) may be reduced. For higher oligonucleotide surface group density, the proportion of azide residues may be increased. A person of ordinary skill in the art would understand that there may be further variations in the composition of the polymer to optimize binding of oligonucleotides and surface amplification.

No buffer was added to the 0.2% w/v solution since a buffer may interfere with the binding of the phosphonate to the zirconium oxide. An amount of the solution sufficient to cover the slides in the slide holder (e.g., 10 mL of solution in the 20 mL scintillation vial) was added to the reaction vessel.

The slide holder was inserted into the vessel, and the vessel was capped with the appropriate lid. The phosphonate polymer was allowed to coat the zirconium oxide-coated slides for at least 12 hours at room temperature. The slide holder was removed and dip washed in fresh 18.2 MΩ·cm water 3 times. The slides were blown dry with dry clean nitrogen and stored under a dry nitrogen atmosphere until further use. The slides were not baked in an oven to dry.

In order to passivate the polymer-attached bisphosphonate moieties that did not bind to the Zr-activated surface, two types of passivation molecules were added (e.g., at around 0.2 w/v for 1 hour). One of the passivation molecules, Small Molecule 1, had the structure shown in FIG. 24. Small Molecule 1 is derived from alendronate and a short methoxy-polyethyleneglycol carboxylic acid. The second passivation molecule, Small Molecule 2, had the structure shown in FIG. 25. Small Molecule 2 is derived from alendronate and features a zwitterionic moiety.

In some cases, a passivation molecule may have a similar structure to Small Molecule 1, but the length of the PEG may be tuned to achieve improved amplification and/or sequencing results. In some cases, a passivation molecule may have a similar structure to Small Molecule 2, but the zwitterionic moiety may be replaced by any suitable zwitterionic moiety (e.g., a carboxy acid). In some cases, a mixture of various passivation molecules may be employed at varying proportions to improve amplification and/or sequencing results.

### Method 3

Method 3 used small molecules rather than the polymer of Method 2 to bind to Zr-activated substrates. The cleaning and zirconium oxide deposition steps were the same as described above regarding Method 2.

Method 3 used small molecules featuring:(1) alendronate for attachment to zirconium oxide; (2) varying lengths of spacing atoms (either carbon or a mixture of carbon and oxygen, such as in polyethylene glycol); and (3) an appropriate reactive moiety for bioconjugation, such as an azide for copper-catalyzed click chemistry or any number of reactive groups commonly used for conjugation to oligonucleotides (e.g., NHS-activated acids and primary amines on the oligonucleotides). The lateral spacing of azide moieties may be controlled by simultaneous deposition of passivation molecules, such as Small Molecules 1 and 2 in Method 2 above.

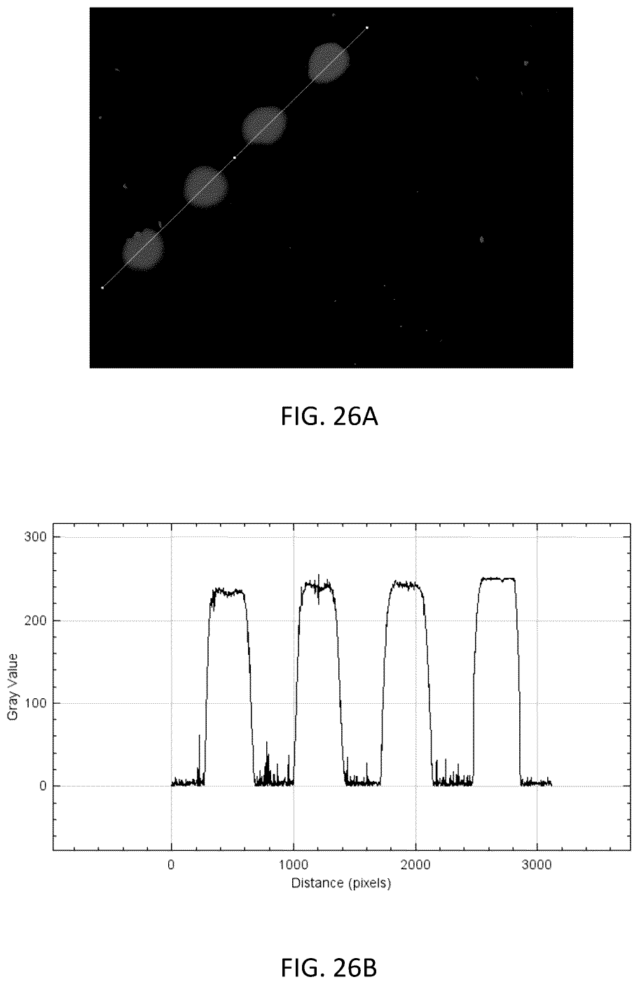

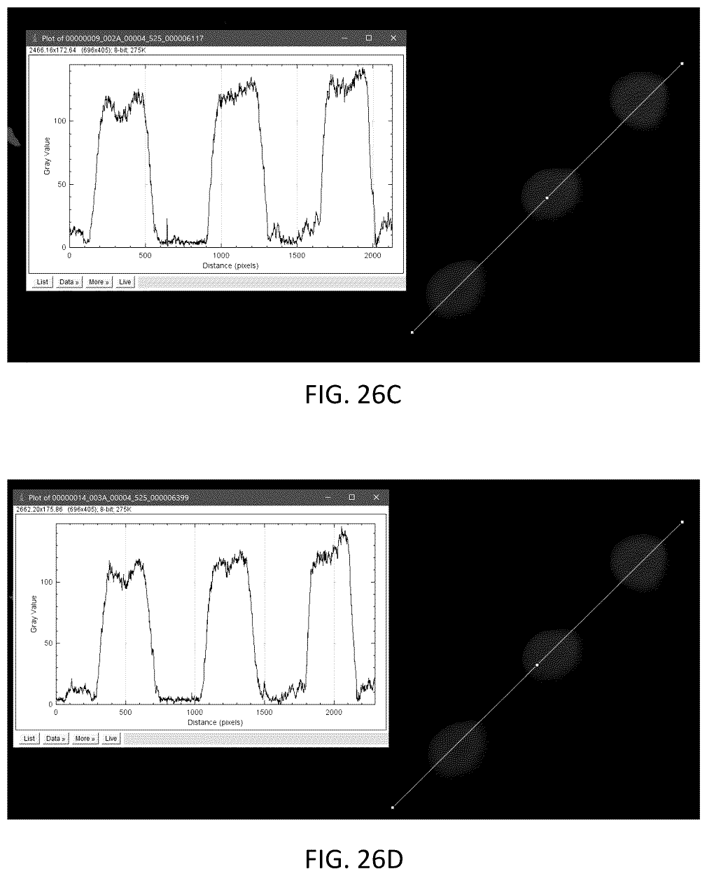

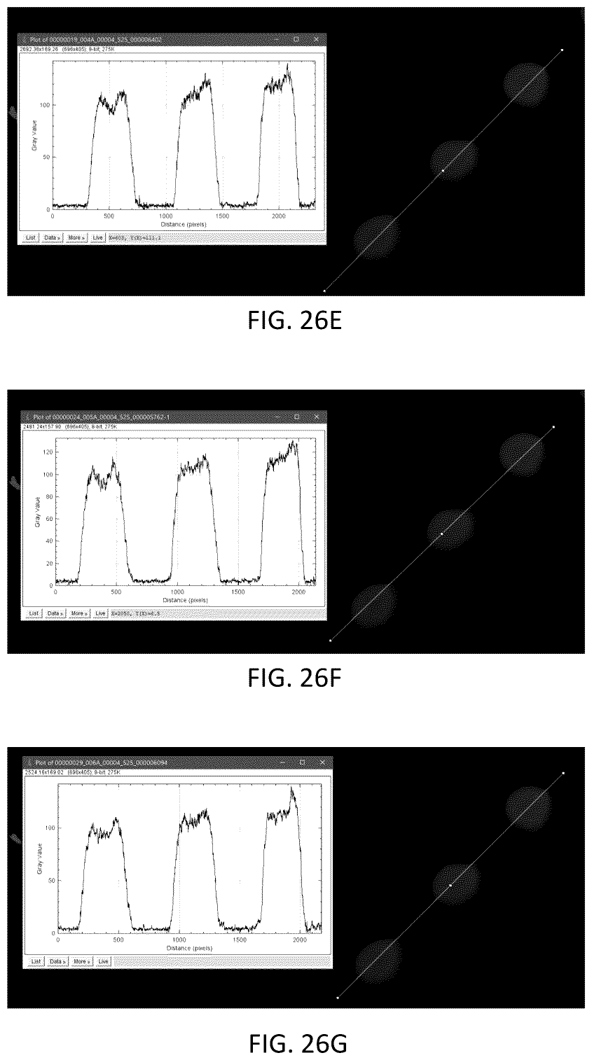

FIGS. 26A-26B shows fluorescently labeled oligonucleotides bound to a sapphire substrate via alendronate. The bound alendronate was reacted with succinic anhydride, and the resulting carboxylic acid was reacted with TSTU (N,N,N′,N′-Tetramethyl-O—(N-succinimidyl)uronium tetrafluoroborate). The resulting surface was then spotted with 5′-amino modified fluorescent oligonucleotides. FIG. 26A shows an image of the bound, fluorescently labeled oligonucleotides, and FIG. 26B shows a plot

After incubation at 75° C. (3 minutes at 75° C. with slow cooling to room temperature) in a hybridization buffer, the substrate was subjected to several harsh washing steps to test the stability of the attachment chemistry. In each case, there was heating to 75° C. for 5 minutes each.

First, the substrate was heated to 75° C. for 5 minutes in 1 mL of TE buffer (IDT). As shown in FIG. 26C, the fluorescently labeled oligonucleotides remained stable.

Second, the substrate was heated to 75° C. for 5 minutes in 1 mL of water. As shown in FIG. 26D, the fluorescently labeled oligonucleotides remained stable.

Third, the substrate was heated to 75° C. for 5 minutes in 1 mL of 100 mM NaOH. As shown in FIG. 26E, the fluorescently labeled oligonucleotides remained stable.

Fourth, the substrate was heated to 75° C. for 5 minutes in 1 mL of 1 M NaOH. As shown in FIG. 26F, the fluorescently labeled oligonucleotides remained stable.

Fifth, the substrate was heated to 75° C. for 5 minutes in 1 mL of 1 M HCl. As shown in FIG. 26G, the fluorescently labeled oligonucleotides remained stable.

In all cases, the signal remained strong even after several harsh chemical treatments. Some fluorescence loss was attributed to photobleaching.

### Example 7: Patterning Wells on Substrates Using CYTOP®

In this Example, CYTOP® was patterned to form wells on substrates and prevent total internal reflection evanescent light from hindering sequencing.

CYTOP® is an optically clear fluoropolymer that has a number of advantageous characteristics, including low adhesion to biological materials, low fluorescence, wettability control. nearly the same refractive index as water (n=1.33), electret properties. oil and water repellency, and chemical resistance. In addition. CYTOP® is highly transparent. with a higher than 95% transmission rate for visible light and a higher than 90% transmission rate for ultraviolet light. As a fluoropolymer that dissolves in fluorine-based solvents. it can be used as a thin film coating with a thickness of less than 1 prn. Thicker and/or multiple layers may also be applied to improve its moisture-proof and electret properties. Various coating methods can be used, such as spin coating, dip coating. spray coating, dispensing, and die coating, There are many types of CYTOP® that may be selected depending on the substrate and desired thickness.

In this Example, the following protocol was developed.

First, primers were applied to a substrate (e.g., a quartz or sapphire substrate) by dipping the substrate into a primer mix. The substrate was then moved back and forth in a beaker of deionized water for 3-5 seconds. The substrate was then blown dry with nitrogen.

Second, CYTOP® (e.g., CYTOP®809A) was spin-coated on the substrate. A spin recipe was selected based on desired thickness. For example, to achieve a 1 μm thick coating. CYTOP® was spin-coated at 500 RPM for 15 seconds and at 1000 RPM for 20 seconds. The substrate was rested in air for 10 minutes and then baked on a hotplate at 80° C. for 30 minutes. The substrate was subsequently baked on the hotplate at 180° C. for 30 minutes.

Third, the CYTOP®-coated substrate was pretreated with argon using an AutoGlow AG200 system. The coated substrates were exposed to argon plasma for 30 seconds at 100 W, 30 sccm, to modify the CYTOP® surface to allow for photoresist adhesion.

Fourth, a photoresist (e.g., a positive photoresist) was spin-coated onto the CYTOP®-coated substrate. AZ1505 and AZ9260 were used depending on the thickness of the CYTOP® coating. A patterned mask was then applied, with unmasked regions corresponding to wells. The photoresist was then exposed and developed.

Fifth, the AutoGlow AG200 was used to etch wells in the CYTOP® coating. O₂ plasma RIE, 300 W, 14 sccm O₂ was used at an etch rate of about 0.25 μm/min. The CYTOP® coating could be patterned with vertical sidewalls or sloped sidewalls by varying the resist development and etch combinations.

Sixth, the photoresist was stripped using an acetone spray. The substrates were rinsed with deionized water and dried under nitrogen.

In some cases, a surface treatment was further applied to the CYTOP® coating to improve CYTOP®'s wettability and adhesion. A 0.1 wt % solution of an aminosilane coupling agent, H₂NC₃H₆Si(OC₂H)₃ (Dow Corning, Z-6011), in 2-perfluorohexyl ethanol (C₆F₁₃C₂H₅OH, Apollo Scientific, PC6147) was prepared and stirred. The resulting solution was stored for a day. The solution was then spin-coated onto the CYTOP® coating and cured at 100° C. for 10 minutes.

### Mask V3

CYTOP® 809A was spin-coated on a 10 mm×10 mm quartz or sapphire substrate at 500 RPM for 5 seconds and 4000 RPM for 30 seconds to produce a CYTOP® coating with a thickness of 1.29 μm. The substrate was rested in air for 10 minutes, baked on a hotplate at 80° C. for 30 minutes, and then baked on the hotplate at 180° C. for 30 minutes.

The CYTOP® coating was exposed to argon plasma for 30 seconds at 100 W, 30 sccm, to modify the CYTOP® coating to allow for photoresist adhesion. An AZ 9260 positive photoresist was then spin-coated onto the CYTOP® coating at 5000 RPM to produce a 6 μm-thick photoresist layer. Alternatively. a 1.5 μm-thick layer of AZ 1505 photoresist could have been deposited by spin coating at 1000 RPM.

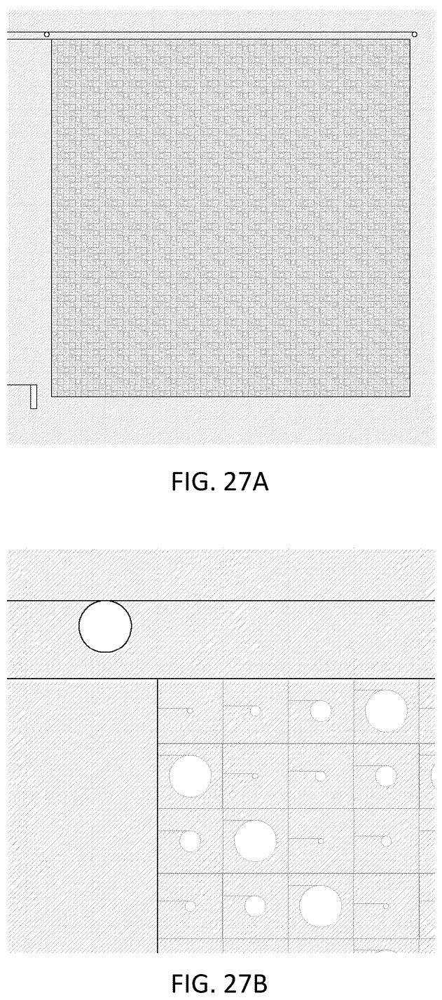

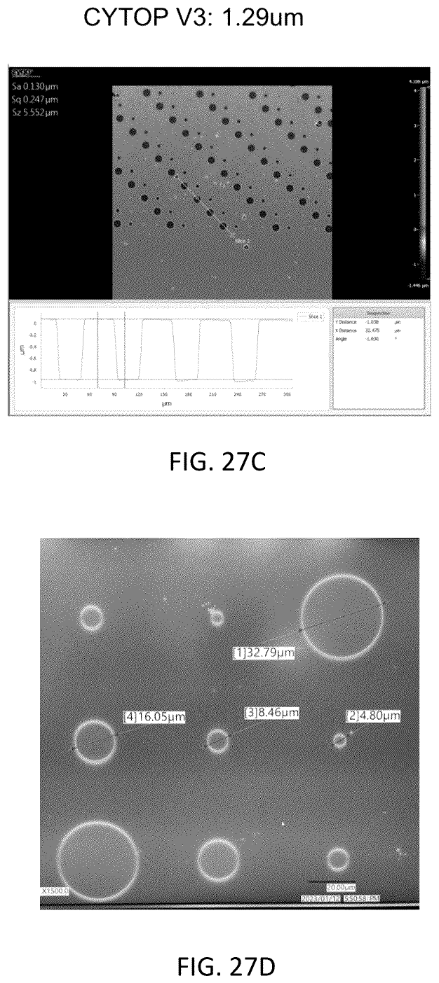

Mask V3 was applied to the photoresist layer. As shown in FIG. 27A, which shows the entire pattern for Mask V3, and FIG. 27B, which shows a zoomed in view of the upper left corner of the pattern for Mask V3, this mask is a mix of 4, 8, 16, and 32 μm size wells on a 50 μm pitch. With this mask, standard spotting methods could be used with much larger diameter spots (greater than 100 μm), and the combination of the two could fill multiple wells. The photoresist was then exposed and developed.

The AutoGlow AG200 was used to etch wells in the CYTOP® coating using O₂ plasma RIE, 300 W, 14 sccm O₂ at an etch rate of about 0.25 μm/min. The wells were etched to have sloped sidewalls. FIG. 27C shows optical profilometer measurements of the resulting wells.

FIG. 27) shows an optical image of the resulting wells.

### Mask V4

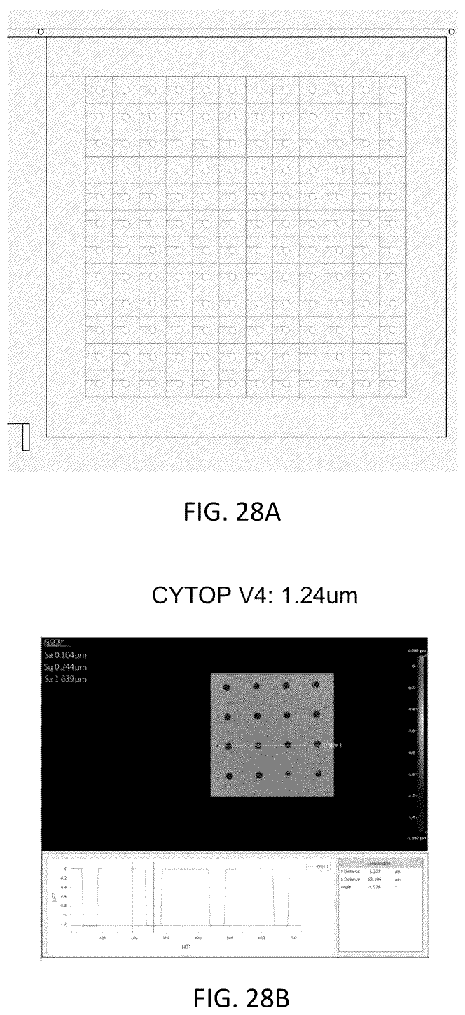

The same protocol used with respect to Mask V3 was used, except that the CYTOP® coating had a thickness of 1.24 μm and Mask V4 was applied. As shown in FIG. 28A, Mask V4 is a 12×12 array of 50 μm diameter wells on a 200 μm pitch. FIG. 28B shows optical profilometer measurements of the resulting wells. This layout could be aligned and spotted with the wells acting as a masking to spots larger than 50 μm.

### Mask VS

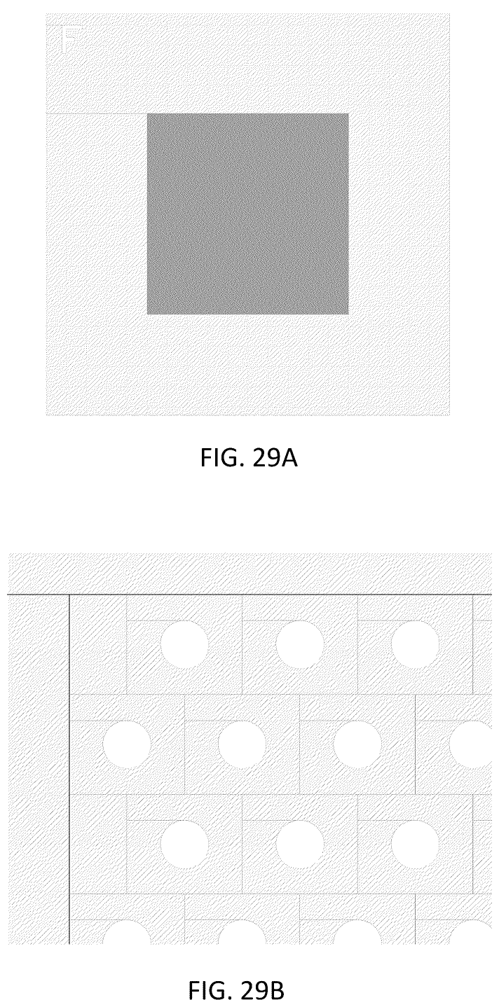

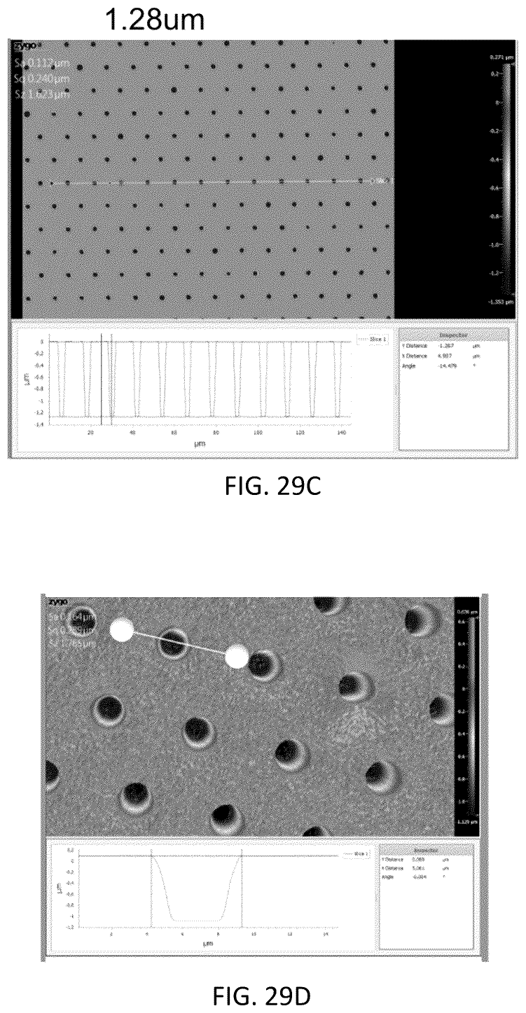

The same protocol used with respect to Mask V3 was used, except that the CYTOP® coating had a thickness of 1.28 μm and Mask V5 was applied. As shown in FIG. 29A, which shows the pattern of Mask V5, and FIG. 29B, which shows a zoomed in view of the pattern. Mask V5 comprises 5 μm wells on a hex pack grid array with 12 prn spacing between any two adjacent wells. FIG. 29C shows optical profilometer measurements of the resulting wells. FIG. 29D shows a zoomed in view of the wells and illustrates that the wells had sloped walls. This design could allow for more than 50,000 spots to be imaged with the device.

### Example 8: Sequencing by Synthesis with MCP4 Surface Chemistry

In this Example, it was demonstrated that a polymer comprising NHS groups could be coated onto quartz substrates, amine-modified oligonucleotides could be reacted with the NHS groups for immobilization to the surface of quartz substrates as sequencing templates, and the oligonucleotides could be sequenced via sequencing-by-synthesis methods.

In this Example, the polymer was a copolymer of N,N-dimethylacrylamide (DMA), acryloyloxysuccinimide (NAS), and 3-(trimethoxysilyl)propyl methacrylate (MAPS) referred to as MCP4 (Lucidant Polymers). FIG. 30A shows the workflow for this Example.

Quartz slides were sonicated in isopropanol at room temperature for 5 minutes. The slides were then immersed in Milli-Q water and dried under nitrogen. The slides were then loaded into a holder and exposed to oxygen plasma for 10 minutes.

An MCP4 stock solution comprising 20% MCP4 and 80% filtered H₂O was prepared, and an MCP4 working solution comprising 2% MCP4 stock solution and 98% Coating Solution (Lucidant) was prepared. The slides were incubated in the MCP4 working solution for 30 minutes at room temperature. The slides were then immersed in a Milli-Q water bath for 10 minutes and immediately dried with nitrogen. The slides were placed under vacuum for 15 minutes at 80° C. and 2 mbar and stored under argon in a desiccator until ready for spotting.

Immediately prior to spotting, the slides were heated at 80° C. for 15 minutes in an oven. Spotting of oligonucleotides was then performed at a relative humidity of about 40%. The spots were then incubated overnight. The spotted slides were then exposed to Blocking Solution (Lucidant) at 50° C. for 30 minutes. The spotted slides were then immersed in a Milli-Q water bath for 10 minutes and dried with nitrogen.

The spotted slides were then assembled into reservoirs for sequencing. FIG. 30B shows images of five cycles of sequencing (with the leftmost image corresponding to the first cycle and the rightmost image corresponding to the fifth cycle). FIG. 30C shows purity histograms for 8 spots for the five cycles of sequencing.

### Example 9: Sequencing by Synthesis with CYTOP® Laver

In this Example, the benefits of inclusion of a CYTOP® layer on sequencing by synthesis were demonstrated.

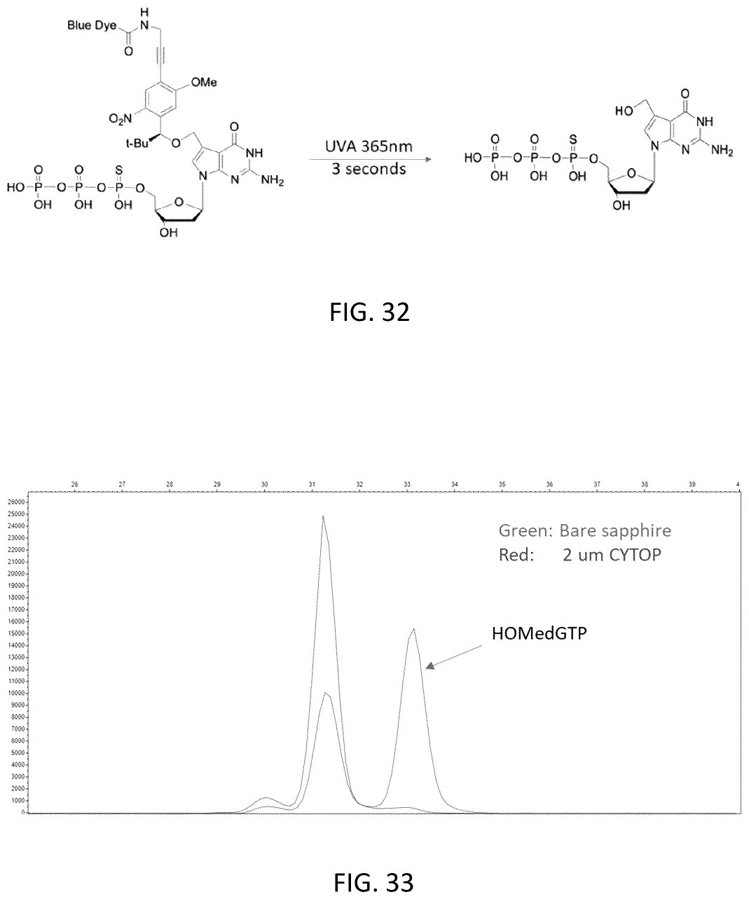

In sequencing methods described herein, protected nucleotides are incorporated by DNA polymerase into DNA immobilized to slides. After images are captured, 365 nm evanescent UV light is used to remove the photocleavable terminating moieties of the incorporated protected nucleotides located within 100 nm of the surface so that the next protected nucleotide could be incorporated. However, evanescent UV light and any scattered UV light may also cleave free protected nucleotides in the solution phase and generate hydroxymethyl dNTP (HOMedNTP). This may consume protected nucleotides and lower their effective concentration for sequencing. The generation of a HOMedNTP (e.g., a HOMedGTP) is shown in FIG. 32.

Previous research studies have demonstrated that HOMedNTP is more favored by Therminator DNA polymerase over protected nucleotides, with higher binding affinity and faster incorporation kinetics (Nucleic Acids Research, 2012, V40, N15, 7404-7415). The incorporation of HOMedNTP may result in leading phasing and decrease sequencing quality.

The effect of a CYTOP® layer in reducing generation of HOMedNTP was investigated. Quartz and sapphire slides were prepared with and without a ° μm-thick layer of CYTOP®. For the slides including the 2 μm-thick layer of CYTOP®, the CYTOP® was deposited as described in Example 7.

The slides were assembled into reservoirs for testing. A protected nucleotide dGTP having the structure shown in FIG. 31B was added at 2 μM in 120 μL Sequencing Buffer to the sapphire and quartz reservoirs, including those with and without the 2 μm-thick layer of CYTOP®. All reservoirs were loaded onto a sequencing instrument and exposed to 365 nm UV for 3 seconds. The solutions were then recovered for analysis.

A HOMedNTP measurement assay was applied to quantify HOMedGTP in the recovered solutions. In this assay, 50 fmol of oligo duplex (the DNA sequences of the template and primer are shown in Table 4) was conjugated to 5 μL Dynabeads™ M-270 Streptavidin magnetic beads (Thermofisher, #65305).

TABLE 4

| | SEQ ID | |
|---|---|---|
| | NO | Sequence |
| Template | 11 | /5BioK/TTTTTTTTTTCCATCTGTTCcagt |
| | | cATTGCGAGCTTGGCCTAATCACGGTCATAG |
| Primer | 12 | /5Alex532N/CTATGACCGTGATTAGGCCA |
| | | AGCTCGCAAT |

20 μL of reaction solutions were prepared containing 1× Isothermal Amplification buffer (New England Biolabs, #B0537), 0.2 U/μL Bst2.0 DNA polymerase (New England Biolabs, #M0537L), and 2 μL of UV-exposed solutions from the reservoirs. The reaction solutions were added to duplex bound beads and incubated at 65° C. for 10 minutes.

The beads were washed with 50 μL 1× Isothermal Amplification buffer three times. Twenty μL of Hi-Di Formamide (Thermofisher, #4401457) was added to the beads to denature the duplex and release the primer. wo μl of Formamide solution containing the primer was mixed with 8 μL Hi-Di Formamide and 0.1 μL 120 LIZ dye size standard (Thermofisher, #4324287). The mixture was loaded on SeqStudio Genetic Analyzer (Thermofisher, #A35646) for fragment size analysis.

FIG. 33 shows the analysis output from Genemapper software. The peak on the left was Alexa Fluor 532 labeled primer, and the peak on the right was HOMedGTP incorporation product. The trace in green is the sample from the sapphire reservoir without CYTOP and the trace in red is from the sapphire reservoir with 2 μm CYTOP. The results demonstrated that 2 μm CYTOP deposition on the surface significantly reduced the generation of HOMedGTP in sapphire slide reservoirs.

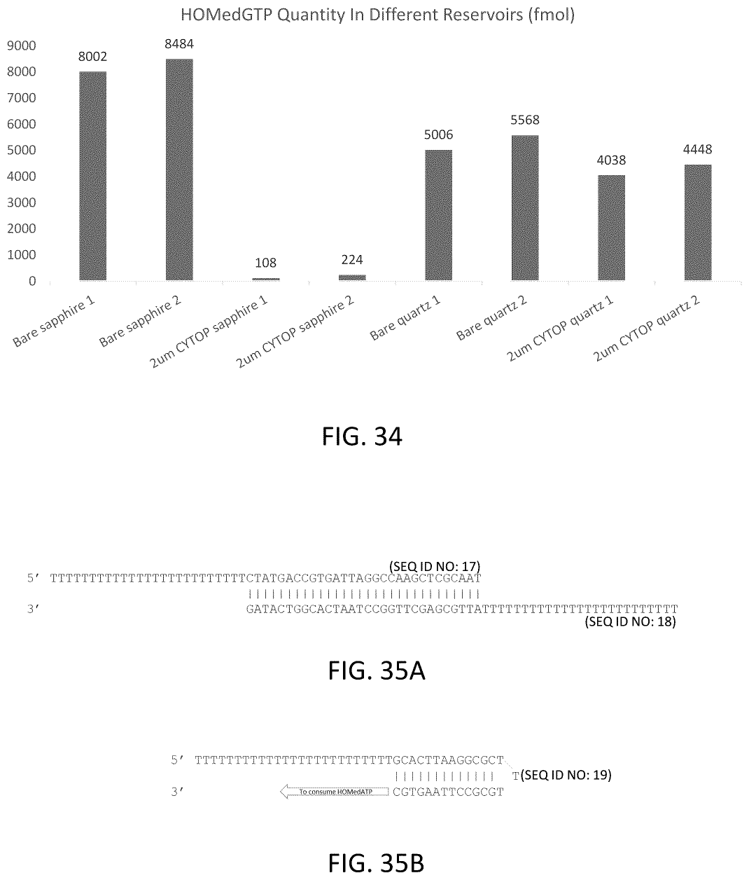

FIG. 34 shows integrated peak areas from Genemapper for different reservoirs. The first two bars are bare sapphire reservoirs, the next two bars are sapphire reservoirs with 2 μm CYTOP which reduced HOMedGTP 98% on average, the following two bars are quartz reservoirs, and the last two bars are quartz reservoirs with 2 μm CYTOP, which reduced HOMedGTP 20% on average.

### Example 10: One Pot Sequencing

The surface of a quartz slide (UQG Optics, UK) was activated with a silane, such as 3-glycidyloxypropyl) trimethoxysilane (Sigma-Aldrich, #440167). Other silanes, such as MCP4, can be used to functionalize the surface of the slide. The 5′ amine modified synthetic oligonucleotide templates were covalently bound to the silane. The unreacted silane was blocked with ethanolamine (Sigma-Aldrich, #398136).

The oligonucleotide templates can be hairpin or single-stranded DNA. Five examples of hairpin sequences are shown in Table 5.

TABLE 5

| SEQ ID | |
|---|---|
| NO | Sequence |
| 13 | /5AmMC6/TTTTTTTTTTTTGATGTTGTTGtgca |
| | CGACTTAAGGCGCTTGCGCCTTAAGTCG |
| 14 | /5AmMC6/TTTTTTTTTTTTGATGTTGTTGcatg |
| | CGACTTAAGGCGCTTGCGCCTTAAGTCG |
| 15 | /5AmMC6/TTTTTTTTTTTTGATGTTGTTGatgc |
| | CGACTTAAGGCGCTTGCGCCTTAAGTCG |
| 13 | /5AmMC6/TTTTTTTTTTTTGATGTTGTTGtgca |
| | CGACTTAAGGCGCTTGCGCCTTAAGTCG |
| 16 | /5AmMC6/TTTTTTTTTTTTGATGTTGTTGgcat |
| | CGACTTAAGGCGCTTGCGCCTTAAGTCG |

The slide was then assembled into a reservoir for sequencing reactions.

For single-stranded templates, 0.5 μM sequencing primer in 120 μL Sequencing Buffer (20 mM Tris·HCl, 0.1% Triton X-100, 10 mM ammonia sulfate, 10 mM potassium chloride, 8 mM magnesium sulfate, 1% PEG8000, 50 μM manganese chloride, 50 mM DTT, and 40 mM tetramethylammonium chloride) was added to the reservoir for hybridization. For hairpin templates, only 120 uL Sequencing Buffer was added to the reservoir. Hybridization occurred by heating the reservoir to 70° C. and incubating for 3 minutes and then cooling down to 25° C. at a rate of 5° C./min.

The reservoir was loaded on a sequencer described herein. After the hybridization solution was removed, 0.05 U/μL Therminator (New England Biolabs) in 120 μL Sequencing Buffer was added to the reservoir and incubated for 3 minutes. After the solution was removed, a volume of 120 μL sequencing reaction solution containing Sequencing Buffer, 500 nM protective nucleotide mix, and 50 nM DISCS (Dark nucleotide In-Situ Cleanup System) reagent was added to the reservoir.

DISCS was composed of oligonucleotide duplexes and Bst2.0 DNA polymerase (New England Biolabs). The oligonucleotide duplex can be cohesive, hairpin, or circular DNA (synthetic or plasmid DNA). Exemplary DNA duplex structures are shown in FIGS. 35A-35B.

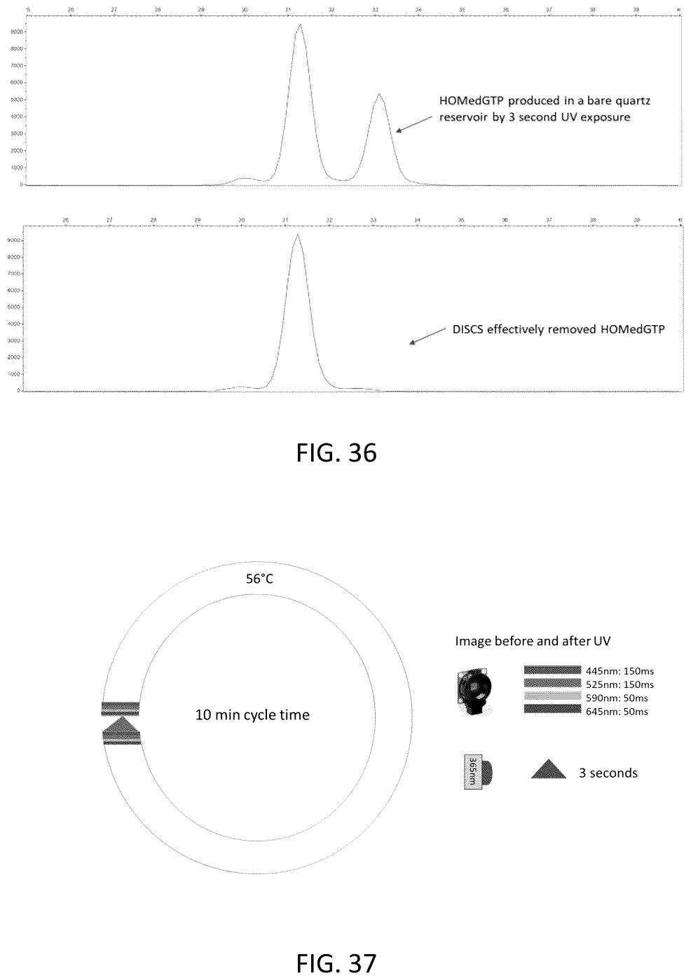

DISCS scavenges the cleavage products of protected nucleotides in the solution phase by UV's TIRF evanescent light or any scattered UV light. FIG. 36 demonstrates that DISCS completely removed the UV cleavage products of protected nucleotides.

Nucleic acid sequencing was initiated by increasing the reservoir's temperature to 56° C. After incubation for 10 minutes, fluorescence images of the incorporated protected nucleotide were taken by the sequencing system. A 365 nm UV LED then irradiated the substrate for 3 seconds to generate evanescent light to remove the terminating moiety on the protected nucleotide. As shown in FIG. 37, this 10-minute incubation, imaging, and 3-second UV exposure comprised one cycle. The sequencing continued until the desired number of cycles was achieved.

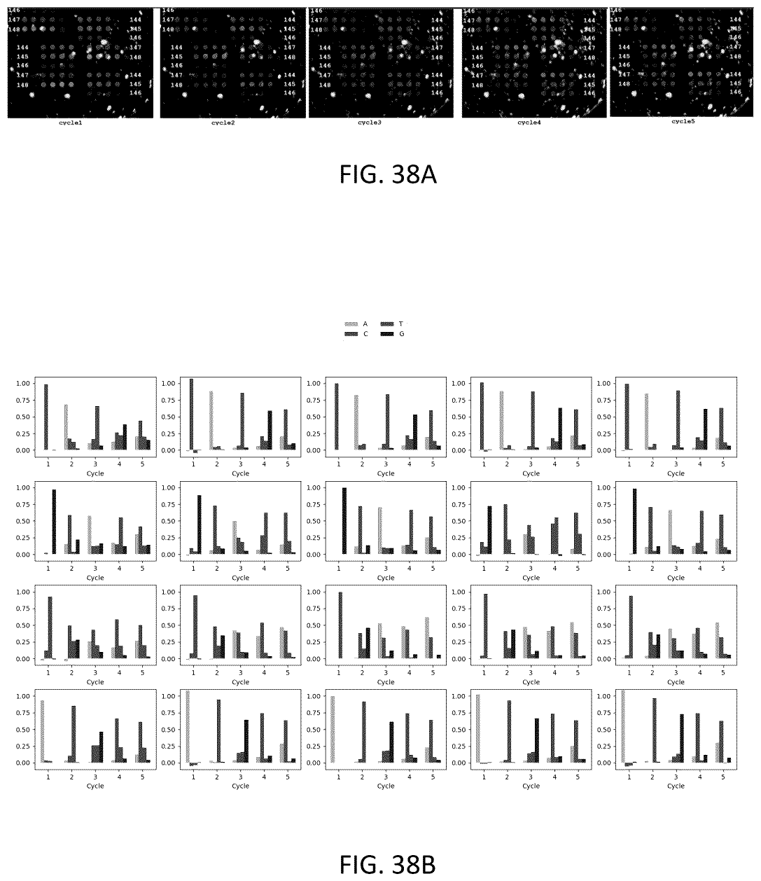

Composite sequencing images from five cycles of sequencing are shown in FIG. 38A. Purity histograms from the five cycles are shown in FIG. 38B.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. A method for preparing a sample for evanescent wave imaging, comprising:
isothermally amplifying a target nucleic acid present in the sample in a reservoir to produce one or more amplicons, wherein the one or more amplicons are immobilized to a bottom surface of the reservoir; and
contacting the one or more amplicons with an aqueous solution comprising sequencing reagents comprising a pool of 3′-unblocked protected nucleotides

2. The method of claim 1, comprising coupling the reservoir with a component of an evanescent wave imaging apparatus.

3. The method of claim 2, wherein coupling positions the reservoir above a detector.

4. The method of claim 3, wherein coupling aligns the one or more immobilized amplicons within view of the detector.

5. The method of claim 1, wherein isothermally amplifying a target nucleic acid sequence comprises using RPA, LAMP, RCA, or WildFire to amplify the target nucleic acid sequence.
